(19) United States
(12) Patent Application Publication
(10) Pub. No.: US 2024/0327845 A1
(43) Pub. Date: Oct. 3, 2024

(54) ANTISENSE OLIGONUCLEOTIDES TARGETING UNC13A

(71) Applicant: Hoffmann-La Roche Inc., Little Falls, NJ (US)

(72) Inventors: Katarzyna Chyzynska, Hørsholm (DK); Lars Joenson, Viby Sjaelland (DK); Bettina Nordbo, Hørsholm (DK); Jonas Vikesaa, Fredensborg (DK)

(21) Appl. No.: 18/746,978

(22) Filed: Jun. 18, 2024

Related U.S. Application Data
(63) Continuation of application No. PCT/EP2022/086915, filed on Dec. 20, 2022.

(30) Foreign Application Priority Data
Dec. 21, 2021 (EP) 21216561.7

(51) Int. Cl. C12N 15/113

(52) U.S. Cl. CPC C12N 15/113; C12N 2310/14; C12N 2310/321; C12N 2310/3231; C12N 2310/3341; C12N 2310/341; C12N 2320/33

(57) ABSTRACT

The present invention relates to antisense oligonucleotide splice modulators Unc-13 homolog A (UNC13A). These antisense oligonucleotide splice modulators are complementary, such as fully complementary, to the UNC13A precursor-mRNA, and are capable of increasing or restoring expression of UNC13A in TDP-43 depleted cells, such as for use in conditions and medical indications where TDP-43 is functionally depleted.

The present invention relates to antisense oligonucleotide splice modulators of Unc-13 homolog A (UNC13A). These antisense oligonucleotide splice modulators are complementary, such as fully complementary, to the UNC13A precursor-mRNA, and are capable of increasing or restoring expression of UNC13A in TDP-43 depleted cells, such as for use in conditions and medical indications where TDP-43 is functionally depleted.

### SEQUENCE LISTING

The instant application contains a Sequence Listing which has been submitted electronically in XML format and is hereby incorporated by reference in its entirety. Said XML copy, created on Jun. 14, 2024, is named 51551-020001_Sequence_Listing_6_14_24.xml and is 838,143 bytes in size.

### BACKGROUND

TAR DNA binding protein 43 (TDP-43) is a versatile RNA/DNA binding protein involved in RNA-related metabolism. Dysregulation of TDP-43 deposits act as inclusion bodies in the brain and spinal cord of patients with the motor neuron diseases: amyotrophic lateral sclerosis (ALS) and frontotemporal lobar degeneration (FTLD) (Prasad et al., Front. Mol. Neurosci., 2019).

TDP-43 depletion is indicated in a range of diseases, referred to as TDP-43 pathologies, and including for example diseases such as amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, autism, Hippocampal sclerosis dementia, Down syndrome, Huntington's disease, polyglutamine diseases, such as spinocerebellar ataxia 3, myopathies and Chronic Traumatic Encephalopathy.

UNC13A proteins bind to phorbol esters and diacylglycerol and are involved in release of neurotransmitters at synapses.

We show that splicing of UNC13A is controlled, at least in part, by TDP-43 due a to a TDP-43 binding site present within the UNC13A pre-mRNA sequence.

### SUMMARY OF INVENTION

The present inventors have surprisingly determined that UNC13A mRNA splicing changes if TDP-43 is depleted in a cell.

The inventors therefore hypothesised that modifying UNC13A splicing patterns, may be able to ameliorate the detrimental effects of TDP-43 depletion on neuronal cells.

Here the inventors have used antisense oligonucleotide UNC13A splice modulators to increase expression of UNC13A.

In one aspect the invention provides an antisense oligonucleotide Unc-13 homolog A (UNC13A) splice modulator, wherein said antisense oligonucleotide splice modulator is 8 to 40 nucleotides in length and comprises a contiguous nucleotide sequence of at least 8 nucleotides in length which is complementary to the UNC13A precursor-mRNA.

In some embodiments, the antisense oligonucleotide splice modulator may be capable of increasing the expression of Unc-13 homolog A ( ) in a TDP-43 depleted cell.

In some embodiments, the antisense oligonucleotide splice modulator may be capable of decreasing expression of an UNC13A mutant polypeptide, such as a splice variant of UNC13A, in a TDP-43 depleted cell.

The inventors have surprisingly determined that in TDP-43 depleted cells, increased expression of an UNC13A splice variant including an additional exon is observed. This leads to a decrease in production of the functionally active wild-type (WT) UNC13A polypeptide. In some embodiments the splice variant may therefore comprise a polypeptide sequence encoded by an additional exon, when compared to the wild-type UNC13A polypeptide sequence.

In some embodiments, the mutant UNC13A splice variant may comprise an insertion, such as an insertion of about 128 or/and 178 nucleotides, when compared to the conventionally spliced UNC13A mature mRNA.

In some embodiments the insertion may lead to a frameshift.

In some embodiments the insertion may lead to a premature stop codon, premature stop codons may target transcripts for nonsense mediated decay (NMD).

In some embodiments the contiguous nucleotide sequence of the antisense oligonucleotide splice modulator may be complementary to a splice enhancer site in the UNC13A precursor-mRNA.

In some embodiments the contiguous nucleotide sequence of the antisense oligonucleotide splice modulator may be complementary to a sequence selected from SEQ ID NOs: 554 to 558.

In some embodiments, the contiguous nucleotide sequence of the antisense oligonucleotide splice modulator may be complementary to a sequence selected from SEQ ID NOs: 8-279.

In some embodiments the contiguous nucleotide sequence of the antisense oligonucleotide splice modulator may be complementary to a sequence selected from SEQ ID NO: 62; SEQ ID NO: 66, SEQ ID NO: 68, SEQ ID NO: 70, SEQ ID NO: 72, SEQ ID NO: 73, SEQ ID NO: 74, SEQ ID NO: 76, SEQ ID NO: 112, SEQ ID NO: 114, SEQ ID NO: 115, SEQ ID NO: 117, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 162, SEQ ID NO: 163, SEQ ID NO: 178, SEQ ID NO: 179, SEQ ID NO: 183, SEQ ID NO: 187, SEQ ID NO: 189, SEQ ID NO: 192, SEQ ID NO: 193, SEQ ID NO: 214, SEQ ID NO: 247, SEQ ID NO: 252, SEQ ID NO: 254, SEQ ID NO: 258, SEQ ID NO: 273, SEQ ID NO: 276, SEQ ID NO: 277, SEQ ID NO: 278 and SEQ ID NO: 279.

In some embodiments the contiguous nucleotide sequence of the antisense oligonucleotide splice modulator may be complementary to a sequence selected from SEQ ID NO: 66, SEQ ID NO: 70, SEQ ID NO: 72, SEQ ID NO: 73, SEQ ID NO: 74, SEQ ID NO: 76, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 154, SEQ ID NO: 155, SEQ ID NO: 156, SEQ ID NO: 162, SEQ ID NO: 163, SEQ ID NO: 273, SEQ ID NO: 276, SEQ ID NO: 277 and SEQ ID NO: 279.

In some embodiments, the contiguous nucleotide sequence of the antisense oligonucleotide splice modulator may be a sequence selected from SEQ ID Nos 280-551, or at least 10 contiguous nucleotides thereof.

In some embodiments, the contiguous nucleotide sequence of the antisense oligonucleotide splice modulator may be a sequence selected from the group consisting of SEQ ID NO: 334; SEQ ID NO: 338; SEQ ID NO: 340; SEQ ID NO: 342; SEQ ID NO: 344; SEQ ID NO: 345; SEQ ID NO: 346; SEQ ID NO: 348; SEQ ID NO: 384; SEQ ID NO: 386; SEQ ID NO: 387; SEQ ID NO: 389; SEQ ID NO: 394; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 418; SEQ ID NO: 423; SEQ ID NO: 424; SEQ ID NO: 425; SEQ ID NO: 426, SEQ ID NO: 427, SEQ ID NO: 428, SEQ ID NO: 434, SEQ ID NO: 435, SEQ ID NO: 450, SEQ ID NO: 451, SEQ ID NO: 455, SEQ ID NO: 459, SEQ ID NO: 461, SEQ ID NO: 464, SEQ ID NO: 465, SEQ ID NO: 486, SEQ ID NO: 519, SEQ ID NO: 524, SEQ ID NO: 526, SEQ ID NO: 530, SEQ ID NO: 545, SEQ ID NO: 548, SEQ ID NO: 549, SEQ ID NO: 550 and SEQ ID NO: 551, or at least 10 contiguous nucleotides thereof.

In some embodiments, the contiguous nucleotide sequence of the antisense oligonucleotide splice modulator may be a sequence selected from the group consisting of SEQ ID NO: 338, SEQ ID NO: 342, SEQ ID NO: 344, SEQ ID NO: 345, SEQ ID NO: 346, SEQ ID NO: 348, SEQ ID NO: 394, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 418, SEQ ID NO: 423, SEQ ID NO: 424, SEQ ID NO: 426, SEQ ID NO: 427, SEQ ID NO: 428, SEQ ID NO: 434, SEQ ID NO: 435, SEQ ID NO: 545, SEQ ID NO: 548, SEQ ID NO: 549, and SEQ ID NO: 551, or at least 10 contiguous nucleotides thereof.

In some embodiments, the antisense oligonucleotide splice modulator may be at least 12 nucleotides in length, such as at least 13, 14, 15, 16, 17, 18, 19, 20, 21, 22, 23, 24, 25, 26, 27, 28, 29, 30, 31 or 32 nucleotides in length.

In some embodiments, the contiguous nucleotide sequence of the antisense oligonucleotide splice modulator may be the same length as the antisense oligonucleotide splice modulator.

In some embodiments, the antisense oligonucleotide splice modulator may comprise one or more modified nucleosides, such as a 2′ sugar modified nucleoside, which may be independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), or any combination thereof.

In some embodiments, the contiguous nucleotide sequence of the antisense oligonucleotide splice modulator may comprise 2′-O-methoxyethyl-RNA (2′-MOE) nucleosides, optionally linked by phosphorothioate internucleoside linkages.

In some embodiments, one or more of the modified nucleosides may be a locked nucleic acid nucleoside (LNA), such as an LNA nucleoside selected from the group consisting of constrained ethyl nucleoside (cEt), and B-D-oxy-LNA.

In some embodiments, the contiguous nucleotide sequence of the antisense oligonucleotide splice modulator may be at least 75%, such as at least 80%, at least 85%, at least 90% or at least 95%, complementary to the UNC13A precursor-mRNA sequence.

In other embodiments, the contiguous nucleotide sequence contiguous nucleotide sequence of the may be fully complementary to the UNC13A precursor-mRNA.

In some embodiments, the antisense oligonucleotide splice modulator may not comprise a region of more than 3, or more than 4, contiguous DNA nucleosides, and may not be capable of mediating RNAseH cleavage.

In some embodiments, one or more, or all, of the internucleoside linkages within the antisense oligonucleotide splice modulator may be modified. For examples, the modified internucleoside linkages may comprise a phosphorothioate linkage.

In some embodiments, the antisense oligonucleotide splice modulator may be covalently attached to at least one conjugate moiety.

In some embodiments, the antisense oligonucleotide splice modulator may be in the form of a pharmaceutically acceptable salt, such as a sodium salt or a potassium salt.

In another aspect there is provided a pharmaceutical composition comprising the antisense oligonucleotide splice modulator of the invention, and a pharmaceutically acceptable diluent, solvent, carrier, salt and/or adjuvant.

In another aspect there is provided a method, such as an in vivo or in vitro method, for increasing UNC13A expression in a cell, said method comprising administering an antisense oligonucleotide splice modulator or pharmaceutical composition of the invention, in an effective amount to said cell, which may express aberrant or exhibits depleted levels of TDP-43.

In another aspect the invention provides a method for treating or preventing a disease in a subject comprising administering a therapeutically or prophylactically effective amount of an antisense oligonucleotide splice modulator or pharmaceutical composition of the invention to a subject suffering from or susceptible to the disease.

In another aspect there is provided an antisense oligonucleotide splice modulator or a pharmaceutical composition of the invention for use as a medicament.

In another aspect there is provided an antisense oligonucleotide splice modulator or pharmaceutical composition of the invention for use in the treatment or prevention of disease in a subject.

In another aspect there is provided an antisense oligonucleotide splice modulator or pharmaceutical composition of the invention for the preparation of a medicament for treatment or prevention of a disease in a subject.

In all aspects of the invention, the disease may be a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

In particular embodiments, the disease may be a neurological disorder selected from the group consisting of amyotrophic lateral sclerosis (ALS) and frontotemporal lobar degeneration (FTLD).

### BRIEF DESCRIPTION OF FIGURES

FIG. 1 displays a screenshot from the CLC Genomics Workbench software where the NGS read mapping from the UNC13A gene can be seen. The inclusion of the new exons in the sample treated with compound A (SEQ ID 553) is shown. The arrows show the two possible uses of splice acceptor sites giving the inclusion of either a 128 or 178 bp exon both resulting in a frameshift and a transcript targeted for nonsense mediated decay.

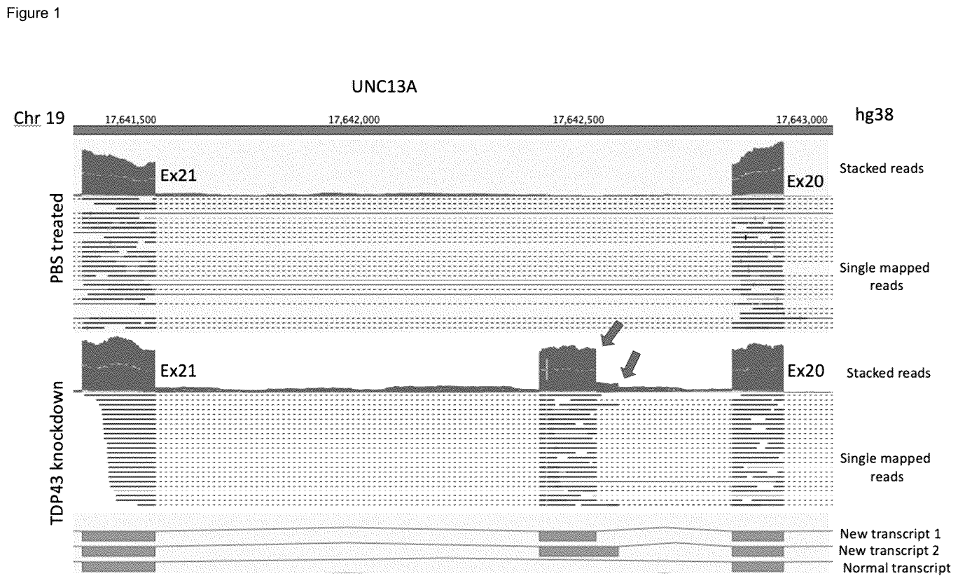

FIG. 2 shows a graphical presentation of the position of the screen ASOs binding site on the UNC13A pre mRNA and the ASO ability to restore the expression of UNC13A, by repressing the inclusion of the two novel exons. The position of the two novel exons which are being included in the UNC13A transcript is shown in the bottom with black bars. The position of the exon is shown according to the hg38 gene annotation and the UNC13A gene is expressed from the minus strand.

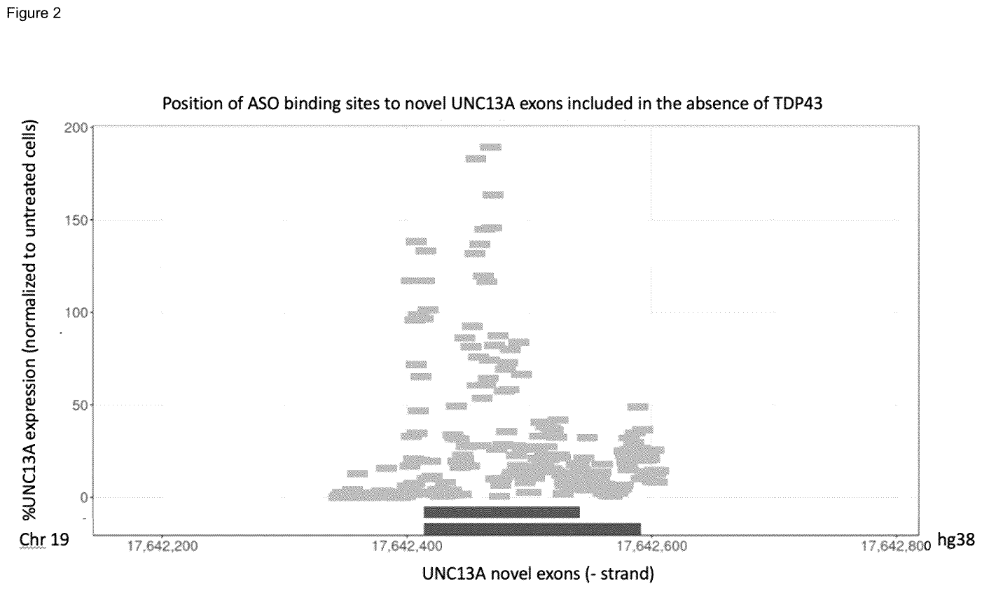

### DETAILED DESCRIPTION

The inventors have identified that the splicing of UNC13A is affected by TDP-43. This is thought to lead to the production of non-functional, or less functional, UNC13A in TDP-43 cells.

Without wishing to be bound by theory, it is considered that in TDP-43 depleted cells UNC13A may be spliced such that one or more additional exons, such as one or two additional exons, is included. This additional exon may be 128 or 178 nucleotides in length. The inclusion of one or more additional exons may lead to a frameshift. This may lead to a premature stop codon which may target the transcript for nonsense mediated decay. This would result in less wild-type UNC13A polypeptide. Such an alternatively spliced mRNA transcript is referred to herein as a “mutant UNC13A mRNA”, a “mutant UNC13A transcript”, a “splicing variant of UNC13A” or an “UNC13A splice variant”.

The inventors have also determined that production of an UNC13A splicing variant can be reduced using an antisense oligonucleotide splice modulator. Herein an antisense oligonucleotide splice modulator of the invention may also be referred to as an oligonucleotide of the invention or an antisense oligonucleotide of the invention.

The oligonucleotide splice modulators of the invention may target a splice enhancer site in the UNC13A precursor-mRNA. This may reduce alternative splicing, thereby increasing conventional splicing and the production of wild-type UNC13A protein.

Enhanced wild-type UNC13A expression is desirable to treat a range of disorders which are characterised by, or caused by, reduced expression of UNC13A. These include amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Splice Modulation

The antisense oligonucleotides of the invention are UNC13A splice modulators, that is they affect the splicing of UNC13A pre-mRNA. Herein the oligonucleotides of the invention may be referred to as “antisense oligonucleotide splice modulators”.

In some embodiments, the antisense oligonucleotide splice modulators of the invention may be complementary to the UNC13A precursor-mRNA.

In some embodiments, the UNC13A precursor-mRNA may have the sequence of SEQ ID NO 1. SEQ ID NO 1 is provided herein as a reference sequence and it will be understood that the target precursor-mRNA may be an allelic variant of SEQ ID NO 1, such as an allelic variant which comprises one or more polymorphisms.

In some embodiments, the antisense oligonucleotide splice modulator may be capable of increasing the expression of UNC13A in a TDP-43 depleted cell. Herein, it is anticipated that expression of wild-type, i.e. conventionally spliced, UNC13A which will be increased by exposure to the antisense oligonucleotide splice modulator of the invention.

Without wishing to be bound by theory, it is thought that the antisense oligonucleotide splice modulators of the invention may increase conventional splicing of UNC13A precursor-mRNA. This is thought to lead to an increase in the amount of conventionally spliced mature UNC13A mRNA, which in turn is thought to lead to an increase in the amount of wild-type UNC13A protein.

Herein the terms “wild-type” and “conventionally spliced” will be used interchangeably.

In some embodiments the wild-type (i.e. conventionally spliced) mature UNC13A mRNA sequence may have the sequence of SEQ ID NO: 2, or a fragment or variant thereof. SEQ ID NO 2 is provided herein as a reference sequence and it will be understood that the conventionally spliced UNC13A mRNA may be an allelic variant of SEQ ID NO 2, such as an allelic variant which comprises one or more polymorphisms.

In some embodiments, the wild-type UNC13A protein may have the sequence of SEQ ID NO: 3, or a fragment or variant thereof. SEQ ID NO 3 is provided herein as a reference sequence and it will be understood that the wild-type UNC13A protein may be an allelic variant of SEQ ID NO 3, such as an allelic variant which comprises one or more polymorphisms.

Herein, the term “increasing the expression of wild-type UNC13A” is understood to mean increasing conventionally spliced UNC13A mRNA levels, increasing wild-type UNC13A protein levels or increasing conventionally spliced UNC13A mRNA levels and wild-type UNC13A protein levels.

In certain embodiments, the antisense oligonucleotide splice modulators of the present invention may increase conventional splicing of UNC13A precursor-mRNA by at least about 10% compared to a control. More preferably the antisense oligonucleotide splice modulators of the present invention may increase conventional splicing of UNC13A precursor-mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, the antisense oligonucleotide splice modulators of the present invention may increase the amount of wild-type UNC13A protein by at least about 10% compared to a control. More preferably the antisense oligonucleotide splice modulators of the present invention may increase the amount of wild-type UNC13A protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, the antisense oligonucleotide splice modulators of the present invention may increase conventional splicing of UNC13A precursor-mRNA and increase the amount of wild-type UNC13A protein by at least about 10% compared to a control. More preferably the antisense oligonucleotide splice modulators of the present invention may increase conventional splicing of UNC13A precursor-mRNA and increase the amount of wild-type UNC13A protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

Preferably, the antisense oligonucleotide splice modulators of the present invention increase the amount of wild-type UNC13A by decreasing expression of a UNC13A mutant mRNA in a TDP-43 depleted cell.

The UNC13A mutant mRNA may be a splicing variant of UNC13A. Herein, the term “splicing variant” or “splice variant” includes, but is not limited to, a variant mature mRNA which includes one or more additional exons relative to the wild-type UNC13A mature mRNA sequence. The wild-type UNC13A mature mRNA sequence may be SEQ ID NO 2.

In some embodiments the inclusion of an additional exon within the UNC13A mature mRNA sequence may lead to a frameshift. This may lead to a premaute stop codon and nonsense mediated decay.

In some embodiments the inclusion of an additional exon may lead to the translation of an UNC13A mutant polypeptide.

In some embodiments, the UNC13A mutant polypeptide may be encoded by the nucleotide sequence of SEQ ID NO: 4, or a fragment or variant thereof. SEQ ID NO 4 is provided herein as a reference sequence and it will be understood that the nucleic acid sequence encoding the mutant UNC13A polypeptide may be an allelic variant of SEQ ID NO 4, such as an allelic variant which comprises one or more polymorphisms.

In other embodiments, the UNC13A mutant polypeptide may have the sequence of SEQ ID NO: 5, or a fragment or variant thereof. SEQ ID NO 5 is provided herein as a reference sequence and it will be understood that the mutant UNC13A polypeptide may be an allelic variant of SEQ ID NO 5, such as an allelic variant which comprises one or more polymorphisms.

In some embodiments, the UNC13A mutant polypeptide may be encoded by the nucleotide sequence of SEQ ID NO: 6, or a fragment or variant thereof. SEQ ID NO 6 is provided herein as a reference sequence and it will be understood that the nucleic acid sequence encoding the mutant UNC13A polypeptide may be an allelic variant of SEQ ID NO 6, such as an allelic variant which comprises one or more polymorphisms.

In other embodiments, the UNC13A mutant polypeptide may have the sequence of SEQ ID NO: 7, or a fragment or variant thereof. SEQ ID NO 7 is provided herein as a reference sequence and it will be understood that the mutant UNC13A polypeptide may be an allelic variant of SEQ ID NO 7, such as an allelic variant which comprises one or more polymorphisms.

Herein, the term “decreasing expression of an UNC13A mutant” is understood to mean decreasing alternatively spliced UNC13A mature mRNA levels, decreasing mutant UNC13A polypeptide levels, or decreasing alternatively spliced UNC13A mature mRNA levels and decreasing mutant UNC13A polypeptide levels. This term also encompasses decreasing the production of alternatively spliced UNC13A mRNA, even if the alternatively spliced mature mRNA is ultimately degraded through nonsense-mediated degradation.

In some embodiments the antisense oligonucleotide splice modulators of the invention are capable of decreasing the level of alternatively spliced UNC13A mature mRNA by at least 10% compared to a control. More preferably the antisense oligonucleotide splice modulators of the invention are capable of decreasing the level of alternatively spliced UNC13A mature mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In some embodiments the antisense oligonucleotide splice modulators of the invention are capable of decreasing mutant UNC13A polypeptide levels by at least 10% compared to a control. More preferably the antisense oligonucleotide splice modulators of the invention are capable of decreasing mutant UNC13A polypeptide levels by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In some embodiments the antisense oligonucleotide splice modulators of the invention are capable of decreasing alternatively spliced UNC13A mature mRNA levels and decreasing mutant UNC13A polypeptide levels by at least 10% compared to a control. More preferably the antisense oligonucleotide splice modulators of the invention are capable of decreasing alternatively spliced mature UNC13A mRNA levels and decreasing mutant UNC13A polypeptide levels by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

### Control

By the term “control”, when used in relation to measurements of the effect of an antisense oligonucleotide splice modulator, it is generally understood that the control is a cell that has not been exposed to the antisense oligonucleotide splice modulator of the invention.

Alternatively, an increase in the expression of wild-type UNC13A or a decrease in the expression of a UNC13A mutant may be determined by reference to the amount of wild-type and/or mutant UNC13A mRNA and/or polypeptide expressed before exposure to the antisense oligonucleotide splice modulator of the invention.

In other embodiments, the control may be a cell treated with a non-targeting oligonucleotide.

In some embodiments, the control may be a mock transfection, for example wherein cells are treated with PBS.

### Oligonucleotides

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The antisense oligonucleotide splice modulators of the invention are man-made, and are chemically synthesised, and are typically purified or isolated. The antisense oligonucleotide splice modulators of the invention may comprise one or more modified nucleosides such as 2′ sugar modified nucleosides. The antisense oligonucleotide splice modulators of the invention may comprise one or more modified internucleoside linkages, such as one or more phosphorothioate internucleoside linkages.

In some embodiments, the antisense oligonucleotide splice modulators of the invention are single stranded oligonucleotides.

In some embodiments, the antisense oligonucleotide splice modulators of the invention are 8 to 40 nucleotides in length.

In some embodiments, the antisense oligonucleotide splice modulators of the invention are 8 to 40 nucleotides in length and comprise a contiguous nucleotide sequence of 8 to 40 nucleotides.

In some embodiments, the antisense oligonucleotide splice modulators of the invention are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the antisense oligonucleotide splice modulators of the invention are at least 12 nucleotides in length.

In some embodiments the antisense oligonucleotide splice modulators of the invention are at least 14 nucleotides in length.

In some embodiments the antisense oligonucleotide splice modulators of the invention are at least 16 nucleotides in length.

In some embodiments the antisense oligonucleotide splice modulators of the invention are at least 18 nucleotides in length.

Preferably, the antisense oligonucleotide splice modulators of the invention are 16 to 20 nucleotides in length.

More preferably, the antisense oligonucleotide splice modulators of the invention are 18 to 20 nucleotides in length.

### Contiguous Nucleotide Sequence

The term “contiguous nucleotide sequence” as used herein refers to the region of the antisense oligonucleotide splice modulator of the invention which is complementary to a target nucleic acid, which may be or may comprise an oligonucleotide motif sequence. The term is used interchangeably herein with the term “contiguous nucleobase sequence”.

The antisense oligonucleotide splice modulator comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

It is understood that the contiguous nucleotide sequence of the antisense oligonucleotide splice modulator cannot be longer than the antisense oligonucleotide splice modulator as such and that the antisense oligonucleotide splice modulator cannot be shorter than the contiguous nucleotide sequence.

In some embodiments, the entire nucleotide sequence of the antisense oligonucleotide splice modulator of the invention is the contiguous nucleotide sequence.

The contiguous nucleotide sequence is the sequence of nucleotides in the antisense oligonucleotide splice modulator of the invention which are complementary to, and in some instances fully complementary to, the target nucleic acid, target sequence, or target site sequence.

In some embodiments, the contiguous nucleotide sequence is 8 to 40 nucleotides in length.

In some embodiments, the contiguous nucleotide sequence is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the contiguous nucleotide sequence is at least 12 nucleotides in length.

In some embodiments the contiguous nucleotide sequence is at least 14 nucleotides in length.

In some embodiments the contiguous nucleotide sequence is at least 16 nucleotides in length.

In some embodiments the contiguous nucleotide sequence is at least 18 nucleotides in length.

In a preferred embodiment the contiguous nucleotide sequence is 16 to 20 nucleotides in length.

More preferably, the contiguous nucleotide sequence is 18 to 20 nucleotides in length.

In some embodiments the antisense oligonucleotide splice modulator of the invention consists of the contiguous nucleotide sequence.

In some embodiments the antisense oligonucleotide splice modulator of the invention is the contiguous nucleotide sequence.

### Antisense Oligonucleotide Splice Modulator Targeting UNC13A Precursor-mRNA

The antisense oligonucleotide splice modulators of the invention comprise a contiguous nucleotide sequence which is complementary to the UNC13A precursor-mRNA.

The UNC13A precursor-mRNA may be described as the target for the contiguous nucleotide sequence or for the antisense oligonucleotide splice modulator. Put another way, the antisense oligonucleotide splice modulator targets the UNC13A precursor-mRNA.

In some embodiments the target sequence may have the sequence of SEQ ID NO 1, or a fragment thereof. SEQ ID NO 1 is provided herein as a reference sequence and it will be understood that the UNC13A precursor-mRNA sequence may be an allelic variant of SEQ ID NO 1, such as an allelic variant which comprises one or more polymorphisms. This applies equally to all sequences identified as target sequences herein.

In one aspect, the invention relates to an antisense oligonucleotide splice modulator wherein said antisense oligonucleotide splice modulator is 8 to 40 nucleotides in length and comprises a contiguous nucleotide sequence of at least 8 nucleotides in length which is complementary to SEQ ID NO 1.

In some embodiments, the antisense oligonucleotide splice modulator of the invention comprises a contiguous sequence which is at least about 75% complementary, at least about 80% complementary, at least about 85% complementary, at least about 90% complementary, at least about 95% complementary, or fully complementary (i.e. 100% complementary) to SEQ ID NO 1. Here, complementarity is determined across the length of the contiguous nucleotide sequence.

In some embodiments the antisense oligonucleotide splice modulator of the invention comprises a contiguous sequence which is at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary or fully complementary (i.e. 100% complementary) to SEQ ID NO 1.

In some embodiments, the antisense oligonucleotide splice modulator of the invention comprises a contiguous sequence which may comprise one, two or three mismatches between the contiguous nucleotide sequence and the target nucleic acid.

In a preferred embodiment the oligonucleotide of the invention, or contiguous nucleotide sequence thereof is fully complementary (100% complementary) to SEQ ID NO 1, across the length of the contiguous nucleotide sequence.

In one embodiment the contiguous nucleotide sequence is complementary to a splice enhancer site in the UNC13A precursor-mRNA.

An aspect of the present invention relates to an antisense oligonucleotide splice modulator, which comprises a contiguous nucleotide sequence of 8 to 40 nucleotides in length which is complementary to SEQ ID NO 554.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 554.

In a preferred embodiment the antisense oligonucleotide splice modulator of the invention comprises a contiguous sequence which is fully complementary (i.e. 100% complementary) to SEQ ID NO 554.

An aspect of the present invention relates to an antisense oligonucleotide splice modulator, which comprises a contiguous nucleotide sequence of 8 to 40 nucleotides in length which is complementary to SEQ ID NO 555.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 555.

In a preferred embodiment the antisense oligonucleotide splice modulator of the invention comprises a contiguous sequence which is fully complementary (i.e. 100% complementary) to SEQ ID NO 555.

An aspect of the present invention relates to an antisense oligonucleotide splice modulator, which comprises a contiguous nucleotide sequence of 8 to 40 nucleotides in length which is complementary to SEQ ID NO 556.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 556.

In a preferred embodiment the antisense oligonucleotide splice modulator of the invention comprises a contiguous sequence which is fully complementary (i.e. 100% complementary) to SEQ ID NO 556.

An aspect of the present invention relates to an antisense oligonucleotide splice modulator, which comprises a contiguous nucleotide sequence of 8 to 40 nucleotides in length which is complementary to SEQ ID NO 557.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 557.

In a preferred embodiment the antisense oligonucleotide splice modulator of the invention comprises a contiguous sequence which is fully complementary (i.e. 100% complementary) to SEQ ID NO 557.

An aspect of the present invention relates to an antisense oligonucleotide splice modulator, which comprises a contiguous nucleotide sequence of 8 to 40 nucleotides in length which is complementary to SEQ ID NO 558.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 558.

In a preferred embodiment the antisense oligonucleotide splice modulator of the invention comprises a contiguous sequence which is fully complementary (i.e. 100% complementary) to SEQ ID NO 558.

In one embodiment the target sequence is SEQ ID NO 554. Put another way, in some embodiments the contiguous nucleic acid is complementary to SEQ ID NO 554.

In one embodiment the target sequence is SEQ ID NO 555. Put another way, in some embodiments the contiguous nucleic acid is complementary to SEQ ID NO 555.

In one embodiment the target sequence is SEQ ID NO 556. Put another way, in some embodiments the contiguous nucleic acid is complementary to SEQ ID NO 556.

In one embodiment the target sequence is SEQ ID NO 557. Put another way, in some embodiments the contiguous nucleic acid is complementary to SEQ ID NO 557.

In one embodiment the target sequence is SEQ ID NO 558. Put another way, in some embodiments the contiguous nucleic acid is complementary to SEQ ID NO 558.

In some embodiments, the antisense oligonucleotide splice modulator of the invention comprises a contiguous nucleotide sequence of 8 to 40 nucleotides in length with at least 75% complementary, such as at least 80%, at least 85%, at least 90% or at least 95% or 100% complementarity, to a target nucleic acid region selected from the group consisting of SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, SEQ ID NO 90, SEQ ID NO 91, SEQ ID NO 92, SEQ ID NO 93, SEQ ID NO 94, SEQ ID NO 95, SEQ ID NO 96, SEQ ID NO 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278 and SEQ ID NO: 279.

In some embodiments the target sequence is selected from the group consisting of SEQ ID NO: 62; SEQ ID NO: 66, SEQ ID NO: 68, SEQ ID NO: 70, SEQ ID NO: 72, SEQ ID NO: 73, SEQ ID NO: 74, SEQ ID NO: 76, SEQ ID NO: 112, SEQ ID NO: 114, SEQ ID NO: 115, SEQ ID NO: 117, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 162, SEQ ID NO: 163, SEQ ID NO: 178, SEQ ID NO: 179, SEQ ID NO: 183, SEQ ID NO: 187, SEQ ID NO: 189, SEQ ID NO: 192, SEQ ID NO: 193, SEQ ID NO: 214, SEQ ID NO: 247, SEQ ID NO: 252, SEQ ID NO: 254, SEQ ID NO: 258, SEQ ID NO: 273, SEQ ID NO: 276, SEQ ID NO: 277, SEQ ID NO: 278, and SEQ ID NO: 279. Put another way, in some embodiments the contiguous nucleic acid is complementary to a sequence selected from the group consisting of SEQ ID NO: 62; SEQ ID NO: 66, SEQ ID NO: 68, SEQ ID NO: 70, SEQ ID NO: 72, SEQ ID NO: 73, SEQ ID NO: 74, SEQ ID NO: 76, SEQ ID NO: 112, SEQ ID NO: 114, SEQ ID NO: 115, SEQ ID NO: 117, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 162, SEQ ID NO: 163, SEQ ID NO: 178, SEQ ID NO: 179, SEQ ID NO: 183, SEQ ID NO: 187, SEQ ID NO: 189, SEQ ID NO: 192, SEQ ID NO: 193, SEQ ID NO: 214, SEQ ID NO: 247, SEQ ID NO: 252, SEQ ID NO: 254, SEQ ID NO: 258, SEQ ID NO: 273, SEQ ID NO: 276, SEQ ID NO: 277, SEQ ID NO: 278, and SEQ ID NO: 279.

In some embodiments the target sequence is selected from the group consisting of SEQ ID NO: 66, SEQ ID NO: 70, SEQ ID NO: 72, SEQ ID NO: 73, SEQ ID NO: 74, SEQ ID NO: 76, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 154, SEQ ID NO: 155, SEQ ID NO: 156, SEQ ID NO: 162, SEQ ID NO: 163, SEQ ID NO: 273, SEQ ID NO: 276, SEQ ID NO: 277, and SEQ ID NO: 279. Put another way, in some embodiments the contiguous nucleic acid is complementary to sequence selected from the group consisting of SEQ ID NO: 66, SEQ ID NO: 70, SEQ ID NO: 72, SEQ ID NO: 73, SEQ ID NO: 74, SEQ ID NO: 76, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 154, SEQ ID NO: 155, SEQ ID NO: 156, SEQ ID NO: 162, SEQ ID NO: 163, SEQ ID NO: 273, SEQ ID NO: 276, SEQ ID NO: 277, and SEQ ID NO: 279.

In one embodiment the target sequence is SEQ ID NO 66, or a fragment thereof. Put another way, in some embodiments the contiguous nucleic acid is complementary to SEQ ID NO 66, or a fragment thereof.

In another embodiment the contiguous nucleotide sequence may be fully complementary to SEQ ID NO 66.

In one embodiment the target sequence is SEQ ID NO 70, or a fragment thereof. Put another way, in some embodiments the contiguous nucleic acid is complementary to SEQ ID N 70, or a fragment thereof.

In another embodiment the contiguous nucleotide sequence may be fully complementary to SEQ ID NO 70.

In one embodiment the target sequence is SEQ ID NO 72, or a fragment thereof. Put another way, in some embodiments the contiguous nucleic acid is complementary to SEQ ID NO 72, or a fragment thereof.

In another embodiment the contiguous nucleotide sequence may be fully complementary to SEQ ID NO 72.

In one embodiment the target sequence is SEQ ID NO 73, or a fragment thereof. Put another way, in some embodiments the contiguous nucleic acid is complementary to SEQ ID NO 73, or a fragment thereof.

In another embodiment the contiguous nucleotide sequence may be fully complementary to SEQ ID NO 73.

In one embodiment the target sequence is SEQ ID NO 74, or a fragment thereof. Put another way, in some embodiments the contiguous nucleic acid is complementary to SEQ ID NO 74, or a fragment thereof.

In another embodiment the contiguous nucleotide sequence may be fully complementary to SEQ ID NO 74.

In one embodiment the target sequence is SEQ ID NO 76, or a fragment thereof. Put another way, in some embodiments the contiguous nucleic acid is complementary to SEQ ID NO 76, or a fragment thereof.

In another embodiment the contiguous nucleotide sequence may be fully complementary to SEQ ID NO 76.

In one embodiment the target sequence is SEQ ID N 122, or a fragment thereof. Put another way, in some embodiments the contiguous nucleic acid is complementary to SEQ ID NO 122, or a fragment thereof.

In another embodiment the contiguous nucleotide sequence may be fully complementary to SEQ ID NO 122.

In one embodiment the target sequence is SEQ ID NO 125, or a fragment thereof. Put another way, in some embodiments the contiguous nucleic acid is complementary to SEQ ID NO 125, or a fragment thereof.

In another embodiment the contiguous nucleotide sequence may be fully complementary to SEQ ID NO 125.

In one embodiment the target sequence is SEQ ID NO 126, or a fragment thereof. Put another way, in some embodiments the contiguous nucleic acid is complementary to SEQ ID N 126, or a fragment thereof.

In another embodiment the contiguous nucleotide sequence may be fully complementary to SEQ ID NO 126.

In one embodiment the target sequence is SEQ ID NO 127, or a fragment thereof. Put another way, in some embodiments the contiguous nucleic acid is complementary to SEQ ID N 127, or a fragment thereof.

In another embodiment the contiguous nucleotide sequence may be fully complementary to SEQ ID NO 127.

In one embodiment the target sequence is SEQ ID NO 128, or a fragment thereof. Put another way, in some embodiments the contiguous nucleic acid is complementary to SEQ ID NO 128, or a fragment thereof.

In another embodiment the contiguous nucleotide sequence may be fully complementary to SEQ ID NO 128.

In one embodiment the target sequence is SEQ ID NO 129, or a fragment thereof. Put another way, in some embodiments the contiguous nucleic acid is complementary to SEQ ID NO 129, or a fragment thereof.

In another embodiment the contiguous nucleotide sequence may be fully complementary to SEQ ID NO 129.

In one embodiment the target sequence is SEQ ID NO 130, or a fragment thereof. Put another way, in some embodiments the contiguous nucleic acid is complementary to SEQ ID NO 130, or a fragment thereof.

In another embodiment the contiguous nucleotide sequence may be fully complementary to SEQ ID NO 130.

In one embodiment the target sequence is SEQ ID NO 131, or a fragment thereof. Put another way, in some embodiments the contiguous nucleic acid is complementary to SEQ ID NO 131, or a fragment thereof.

In another embodiment the contiguous nucleotide sequence may be fully complementary to SEQ ID NO 131.

In one embodiment the target sequence is SEQ ID NO 133, or a fragment thereof. Put another way, in some embodiments the contiguous nucleic acid is complementary to SEQ ID NO 133, or a fragment thereof.

In another embodiment the contiguous nucleotide sequence may be fully complementary to SEQ ID NO 133.

In one embodiment the target sequence is SEQ ID NO 134, or a fragment thereof. Put another way, in some embodiments the contiguous nucleic acid is complementary to SEQ ID NO 134, or a fragment thereof.

In another embodiment the contiguous nucleotide sequence may be fully complementary to SEQ ID NO 134.

In one embodiment the target sequence is SEQ ID NO 135, or a fragment thereof. Put another way, in some embodiments the contiguous nucleic acid is complementary to SEQ ID NO 135, or a fragment thereof.

In another embodiment the contiguous nucleotide sequence may be fully complementary to SEQ ID NO 135.

In one embodiment the target sequence is SEQ ID NO 136, or a fragment thereof. Put another way, in some embodiments the contiguous nucleic acid is complementary to SEQ ID NO 136, or a fragment thereof.

In another embodiment the contiguous nucleotide sequence may be fully complementary to SEQ ID NO 136.

In one embodiment the target sequence is SEQ ID NO 138, or a fragment thereof. Put another way, in some embodiments the contiguous nucleic acid is complementary to SEQ ID NO 138, or a fragment thereof.

In another embodiment the contiguous nucleotide sequence may be fully complementary to SEQ ID NO 138.

In one embodiment the target sequence is SEQ ID NO 139, or a fragment thereof. Put another way, in some embodiments the contiguous nucleic acid is complementary to SEQ ID NO 139, or a fragment thereof.

In another embodiment the contiguous nucleotide sequence may be fully complementary to SEQ ID NO 138.

In one embodiment the target sequence is SEQ ID NO 140, or a fragment thereof. Put another way, in some embodiments the contiguous nucleic acid is complementary to SEQ ID NO 140, or a fragment thereof.

In another embodiment the contiguous nucleotide sequence may be fully complementary to SEQ ID NO 140.

In one embodiment the target sequence is SEQ ID NO 141, or a fragment thereof. Put another way, in some embodiments the contiguous nucleic acid is complementary to SEQ ID NO 141, or a fragment thereof.

In another embodiment the contiguous nucleotide sequence may be fully complementary to SEQ ID NO 141.

In one embodiment the target sequence is SEQ ID NO 142, or a fragment thereof. Put another way, in some embodiments the contiguous nucleic acid is complementary to SEQ ID NO 142, or a fragment thereof.

In another embodiment the contiguous nucleotide sequence may be fully complementary to SEQ ID NO 142.

In one embodiment the target sequence is SEQ ID NO 143, or a fragment thereof. Put another way, in some embodiments the contiguous nucleic acid is complementary to SEQ ID NO 143, or a fragment thereof.

In another embodiment the contiguous nucleotide sequence may be fully complementary to SEQ ID NO 143.

In one embodiment the target sequence is SEQ ID NO 146, or a fragment thereof. Put another way, in some embodiments the contiguous nucleic acid is complementary to SEQ ID NO 146, or a fragment thereof.

In another embodiment the contiguous nucleotide sequence may be fully complementary to SEQ ID NO 146.

In one embodiment the target sequence is SEQ ID NO 151, or a fragment thereof. Put another way, in some embodiments the contiguous nucleic acid is complementary to SEQ ID NO 151, or a fragment thereof.

In another embodiment the contiguous nucleotide sequence may be fully complementary to SEQ ID NO 151.

In one embodiment the target sequence is SEQ ID NO 152, or a fragment thereof. Put another way, in some embodiments the contiguous nucleic acid is complementary to SEQ ID NO 152, or a fragment thereof.

In another embodiment the contiguous nucleotide sequence may be fully complementary to SEQ ID NO 152.

In one embodiment the target sequence is SEQ ID NO 154, or a fragment thereof. Put another way, in some embodiments the contiguous nucleic acid is complementary to SEQ ID NO 154, or a fragment thereof.

In another embodiment the contiguous nucleotide sequence may be fully complementary to SEQ ID NO 154.

In one embodiment the target sequence is SEQ ID NO 155, or a fragment thereof. Put another way, in some embodiments the contiguous nucleic acid is complementary to SEQ ID NO 155, or a fragment thereof.

In another embodiment the contiguous nucleotide sequence may be fully complementary to SEQ ID NO 155.

In one embodiment the target sequence is SEQ ID NO 156, or a fragment thereof. Put another way, in some embodiments the contiguous nucleic acid is complementary to SEQ ID NO 156, or a fragment thereof.

In another embodiment the contiguous nucleotide sequence may be fully complementary to SEQ ID NO 156.

In one embodiment the target sequence is SEQ ID NO 162, or a fragment thereof. Put another way, in some embodiments the contiguous nucleic acid is complementary to SEQ ID NO 162, or a fragment thereof.

In another embodiment the contiguous nucleotide sequence may be fully complementary to SEQ ID NO 162.

In one embodiment the target sequence is SEQ ID NO 163, or a fragment thereof. Put another way, in some embodiments the contiguous nucleic acid is complementary to SEQ ID NO 163, or a fragment thereof.

In another embodiment the contiguous nucleotide sequence may be fully complementary to SEQ ID NO 163.

In one embodiment the target sequence is SEQ ID NO 273, or a fragment thereof. Put another way, in some embodiments the contiguous nucleic acid is complementary to SEQ ID NO 273, or a fragment thereof.

In another embodiment the contiguous nucleotide sequence may be fully complementary to SEQ ID NO 273.

In one embodiment the target sequence is SEQ ID NO 276, or a fragment thereof. Put another way, in some embodiments the contiguous nucleic acid is complementary to SEQ ID NO 276, or a fragment thereof.

In another embodiment the contiguous nucleotide sequence may be fully complementary to SEQ ID NO 276.

In one embodiment the target sequence is SEQ ID NO 277, or a fragment thereof. Put another way, in some embodiments the contiguous nucleic acid is complementary to SEQ ID NO 277, or a fragment thereof.

In another embodiment the contiguous nucleotide sequence may be fully complementary to SEQ ID NO 277.

In one embodiment the target sequence is SEQ ID NO 279, or a fragment thereof. Put another way, in some embodiments the contiguous nucleic acid is complementary to SEQ ID NO 279, or a fragment thereof.

In another embodiment the contiguous nucleotide sequence may be fully complementary to SEQ ID NO 279.

In some embodiments the fragment of any of the target sequences may be at least 8, at least 9, at least 10, at least 11, at least 12, at least 13, at least 14, at least 15, at least 16, or at least 17 contiguous nucleotides, preferably at least 10 contiguous nucleotides thereof.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term “complementarity” encompasses Watson Crick base-paring between non-modified and modified nucleobases (see for example Hirao et al., 2012, Accounts of Chemical Research, 45, 2055 and Bergstrom, 2009, Curr. Protoc. Nucleic Acid Chem., 37, 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention, the term “complementary” requires the antisense oligonucleotide splice modulator, or contiguous nucleotide sequence thereof, to be at least about 75% complementary, at least about 80% complementary, at least about 85% complementary, at least about 90% complementary, or at least about 95% complementary to the target sequence, e.g. the UNC13A precursor-mRNA. In some embodiments the antisense oligonucleotide splice modulator, or contiguous sequence thereof, may be at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary, or 100% complementary to the target sequence, e.g. the UNC13A precursor-mRNA.

In some embodiments, the antisense oligonucleotide splice modulator, or contiguous nucleotide sequence thereof, of the invention may include one, two, three or more mismatches, wherein a mis-match is a nucleotide within the antisense oligonucleotide splice modulator, or contiguous nucleotide sequence thereof, which does not base pair with its target.

The term “fully complementary”, refers to 100% complementarity.

In some embodiments the antisense oligonucleotide splice modulator is fully complementary to the target sequence.

In some embodiments the contiguous nucleotide sequence is fully complementary to the target sequence.

### Identity

The term “identity” as used herein, refers to the proportion of nucleotides (expressed in percent) of a contiguous nucleotide sequence in a nucleic acid molecule (e.g. antisense oligonucleotide splice modulator) which across the contiguous nucleotide sequence, are identical to a reference sequence (e.g. a sequence motif).

The percentage of identity is thus calculated by counting the number of aligned nucleobases that are identical (a match) between two sequences (in the contiguous nucleotide sequence of the antisense oligonucleotide splice modulator of the invention and in the reference sequence), dividing that number by the total number of nucleotides in the contiguous nucleotide sequence and multiplying by 100. Therefore, percentage of identity=(matches×100)/length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

It is therefore to be understood that there is a relationship between identity and complementarity such that contiguous nucleotide sequences within the antisense oligonucleotide splice modulators of the invention that are complementary to a target sequence also share a percentage of identity with said target sequence.

### Hybridization

The terms “hybridizing” or “hybridizes” as used herein are to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RT ln(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbour model as described by SantaLucia, 1998, Proc Natl Acad Sci USA. 95:1460-1465 using appropriately derived thermodynamic parameters described by Sugimoto et al., 1995, Biochemistry 34:11211-11216 and McTigue et al., 2004, Biochemistry 43:5388-5405.

In some embodiments, antisense oligonucleotide splice modulators of the present invention hybridize to a target nucleic acid with estimated ΔG° values below-10 kcal for oligonucleotides that are 10-30 nucleotides in length.

In some embodiments the degree or strength of hybridization is measured by the standard state Gibbs free energy ΔG°. The antisense oligonucleotide splice modulators may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the antisense oligonucleotide splice modulators hybridize to a target nucleic acid with an estimated ΔG° value of −10 to −60 kcal, such as −12 to −40, such as from −15 to −30 kcal, or −16 to −27 kcal such as −18 to −25 kcal.

### Antisense Oligonucleotide Splice Modulators

The antisense oligonucleotide of the invention is an antisense oligonucleotide splice modulator comprising a contiguous nucleotide sequence which is complementary to the UNC13A precursor-mRNA.

In some embodiments the contiguous nucleotide sequence is a sequence selected from the group consisting of SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550 and SEQ ID NO: 551, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In some embodiments the contiguous nucleotide sequence is a sequence selected from the group consisting of SEQ ID NO: 334; SEQ ID NO: 338; SEQ ID NO: 340; SEQ ID NO: 342; SEQ ID NO: 344; SEQ ID NO: 345; SEQ ID NO: 346; SEQ ID NO: 348; SEQ ID NO: 384; SEQ ID NO: 386; SEQ ID NO: 387; SEQ ID NO: 389; SEQ ID NO: 394; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 418; SEQ ID NO: 423; SEQ ID NO: 424; SEQ ID NO: 425; SEQ ID NO: 426, SEQ ID NO: 427, SEQ ID NO: 428, SEQ ID NO: 434, SEQ ID NO: 435, SEQ ID NO: 450, SEQ ID NO: 451, SEQ ID NO: 455, SEQ ID NO: 459, SEQ ID NO: 461, SEQ ID NO: 464, SEQ ID NO: 465, SEQ ID NO: 486, SEQ ID NO: 519, SEQ ID NO: 524, SEQ ID NO: 526, SEQ ID NO: 530, SEQ ID NO: 545, SEQ ID NO: 548, SEQ ID NO: 549, SEQ ID NO: 550, and SEQ ID NO: 551, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In some embodiments the contiguous nucleotide sequence is a sequence selected from the group consisting of SEQ ID NO: 338, SEQ ID NO: 342, SEQ ID NO: 344, SEQ ID NO: 345, SEQ ID NO: 346, SEQ ID NO: 348, SEQ ID NO: 394, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 418, SEQ ID NO: 423, SEQ ID NO: 424, SEQ ID NO: 426, SEQ ID NO: 427, SEQ ID NO: 428, SEQ ID NO: 434, SEQ ID NO: 435, SEQ ID NO: 545, SEQ ID NO: 548, SEQ ID NO: 549 and SEQ ID NO: 551, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In one embodiment the contiguous nucleotide sequence comprises SEQ ID NO 338, or a fragment thereof.

In one embodiment the contiguous nucleotide sequence comprises SEQ ID NO 342, or a fragment thereof.

In one embodiment the contiguous nucleotide sequence comprises SEQ ID NO 344, or a fragment thereof.

In one embodiment the contiguous nucleotide sequence comprises SEQ ID NO 345, or a fragment thereof.

In one embodiment the contiguous nucleotide sequence comprises SEQ ID NO 346, or a fragment thereof.

In one embodiment the contiguous nucleotide sequence comprises SEQ ID NO 348, or a fragment thereof.

In one embodiment the contiguous nucleotide sequence comprises SEQ ID NO 394, or a fragment thereof.

In one embodiment the contiguous nucleotide sequence comprises SEQ ID NO 397, or a fragment thereof.

In one embodiment the contiguous nucleotide sequence comprises SEQ ID NO 398, or a fragment thereof.

In one embodiment the contiguous nucleotide sequence comprises SEQ ID NO 399, or a fragment thereof.

In one embodiment the contiguous nucleotide sequence comprises SEQ ID NO 400, or a fragment thereof.

In one embodiment the contiguous nucleotide sequence comprises SEQ ID NO 401, or a fragment thereof.

In one embodiment the contiguous nucleotide sequence comprises SEQ ID NO 402, or a fragment thereof.

In one embodiment the contiguous nucleotide sequence comprises SEQ ID NO 403, or a fragment thereof.

In one embodiment the contiguous nucleotide sequence comprises SEQ ID NO 405, or a fragment thereof.

In one embodiment the contiguous nucleotide sequence comprises SEQ ID NO 406, or a fragment thereof.

In one embodiment the contiguous nucleotide sequence comprises SEQ ID NO 407, or a fragment thereof.

In one embodiment the contiguous nucleotide sequence comprises SEQ ID NO 408, or a fragment thereof.

In one embodiment the contiguous nucleotide sequence comprises SEQ ID NO 409, or a fragment thereof.

In one embodiment the contiguous nucleotide sequence comprises SEQ ID NO 410, or a fragment thereof.

In one embodiment the contiguous nucleotide sequence comprises SEQ ID NO 411, or a fragment thereof.

In one embodiment the contiguous nucleotide sequence comprises SEQ ID NO 412, or a fragment thereof.

In one embodiment the contiguous nucleotide sequence comprises SEQ ID NO 413, or a fragment thereof.

In one embodiment the contiguous nucleotide sequence comprises SEQ ID NO 414, or a fragment thereof.

In one embodiment the contiguous nucleotide sequence comprises SEQ ID NO 415, or a fragment thereof.

In one embodiment the contiguous nucleotide sequence comprises SEQ ID NO 418, or a fragment thereof.

In one embodiment the contiguous nucleotide sequence comprises SEQ ID NO 423, or a fragment thereof.

In one embodiment the contiguous nucleotide sequence comprises SEQ ID NO 424, or a fragment thereof.

In one embodiment the contiguous nucleotide sequence comprises SEQ ID NO 426, or a fragment thereof.

In one embodiment the contiguous nucleotide sequence comprises SEQ ID NO 427, or a fragment thereof.

In one embodiment the contiguous nucleotide sequence comprises SEQ ID NO 428, or a fragment thereof.

In one embodiment the contiguous nucleotide sequence comprises SEQ ID NO 434, or a fragment thereof.

In one embodiment the contiguous nucleotide sequence comprises SEQ ID NO 435, or a fragment thereof.

In one embodiment the contiguous nucleotide sequence comprises SEQ ID NO 545, or a fragment thereof.

In one embodiment the contiguous nucleotide sequence comprises SEQ ID NO 548, or a fragment thereof.

In one embodiment the contiguous nucleotide sequence comprises SEQ ID NO 549, or a fragment thereof.

In one embodiment the contiguous nucleotide sequence comprises SEQ ID NO 551, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

### Nucleotides and Nucleosides

Nucleotides and nucleosides are the building blocks of oligonucleotides and polynucleotides and, for the purposes of the present invention, include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides).

Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety.

Advantageously, the antisense oligonucleotide splice modulators according to the invention may comprise one or more modified nucleosides.

In some embodiments the antisense oligonucleotide splice modulator or the contiguous nucleotide sequence thereof (motif sequence) can be modified to, for example, increase nuclease resistance and/or binding affinity to the target nucleic acid. Advantageously, high affinity modified nucleosides are used.

Advantageously, one or more of the modified nucleosides of the antisense oligonucleotide splice modulator according to the invention may comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the antisense oligonucleotide splice modulators according to the invention include LNA, 2′-O-MOE, 2′oMe and morpholino nucleoside analogues.

### Modified Internucleoside Linkage

Advantageously, the antisense oligonucleotide splice modulators according to the invention comprise one or more modified internucleoside linkages.

The term “modified internucleoside linkage” is defined as generally understood by the skilled person as linkages, other than phosphodiester (PO) linkages, which covalently couple two nucleosides together. The antisense oligonucleotide splice modulator of the invention may therefore comprise one or more modified internucleoside linkages such as one or more phosphorothioate internucleoside linkages.

In some embodiments at least 50% of the internucleoside linkages in the antisense oligonucleotide splice modulators according to the invention, or the contiguous nucleotide sequence thereof, are phosphorothioate, such as at least 60%, such as at least 70%, such as at least 75%, such as at least 80%, such as at least 90% or more. In some embodiments all of the internucleoside linkages of the antisense oligonucleotide splice modulators of the invention, or contiguous nucleotide sequence thereof, are phosphorothioate.

In a further embodiment, the antisense oligonucleotide splice modulators according to the invention, or the contiguous nucleotide sequence thereof, comprise at least one modified internucleoside linkage. It is advantageous if at least 75%, such as all, of the internucleoside linkages within the contiguous nucleotide sequence are phosphorothioate or boranophosphate internucleoside linkages.

Advantageously, all the internucleoside linkages of the contiguous nucleotide sequence of the antisense oligonucleotide splice modulators according to the invention may be phosphorothioate, or all the internucleoside linkages of the antisense oligonucleotide splice modulators according to the invention may be phosphorothioate linkages.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention, the term nucleobase also encompasses modified nucleobases which may differ from naturally occurring nucleobases, but which are functional during nucleic acid hybridisation. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al. (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine.

### Modified Oligonucleotide

The antisense oligonucleotide splice modulators of the invention may be modified oligonucleotides.

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide splice modulators according to the invention to be or to comprise chimeric oligonucleotides.

In some embodiments, antisense oligonucleotide splice modulators according to the invention, or contiguous nucleotide sequence thereof, may include modified nucleobases, which function as the shown nucleobase in base pairing, for example 5-methyl cytosine may be used in place of methyl cytosine. Inosine may be used as a universal base.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into an oligonucleotide sequence is generally termed oligonucleotide design.

In an embodiment, the antisense oligonucleotide splice modulators according to the invention comprise at least 1 modified nucleoside, such as at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 modified nucleosides.

Suitable modifications are described herein under the headings “modified nucleoside”, “high affinity modified nucleosides”, “sugar modifications”, “2′ sugar modifications” and “Locked nucleic acids (LNA)”.

### High Affinity Modified Nucleosides

A high affinity modified nucleoside is a modified nucleoside which, when incorporated into the oligonucleotide enhances the affinity of the oligonucleotide for its complementary target, for example as measured by the melting temperature (Tm). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3 (2), 203-213).

### Sugar Modifications

The antisense oligonucleotide splice modulators according to the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′oMe), 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3 (2), 203-213, and Deleavey and Damha, Chemistry and Biology 2012, 19, 937. Below are illustrations of some 2′ substituted modified nucleosides.

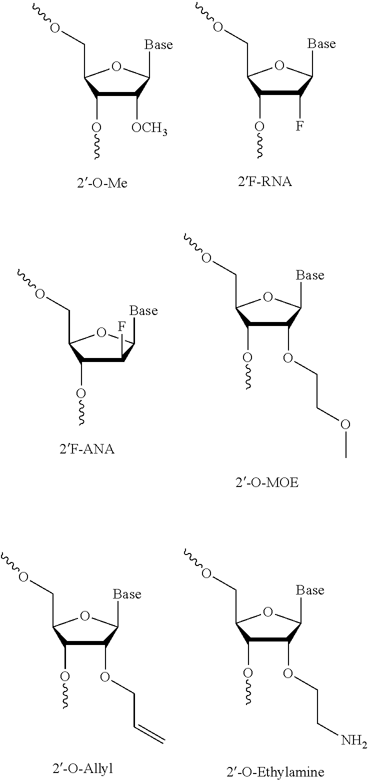

In relation to the present invention, 2′ substituted sugar modified nucleosides does not include 2′ bridged nucleosides like LNA.

In one embodiment, the antisense oligonucleotide splice modulators according to the invention may comprise one or more sugar modified nucleosides, such as 2′ sugar modified nucleosides. Preferably the antisense oligonucleotide splice modulators according to the invention comprises one or more 2′ sugar modified nucleoside independently selected from the group consisting of 2′-O-alkyl-RNA, 2′-O-methyl-RNA (2′oMe), 2′-alkoxy-RNA, 2′-O-methoxyethyl-RNA (2′MOE), 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, and Mitsuoka et al., Nucleic Acids Research 2009, 37 (4), 1225-1238, and Wan and Seth, J. Medical Chemistry 2016, 59, 9645-9667.

Further non limiting, exemplary LNA nucleosides are disclosed in Scheme 1.

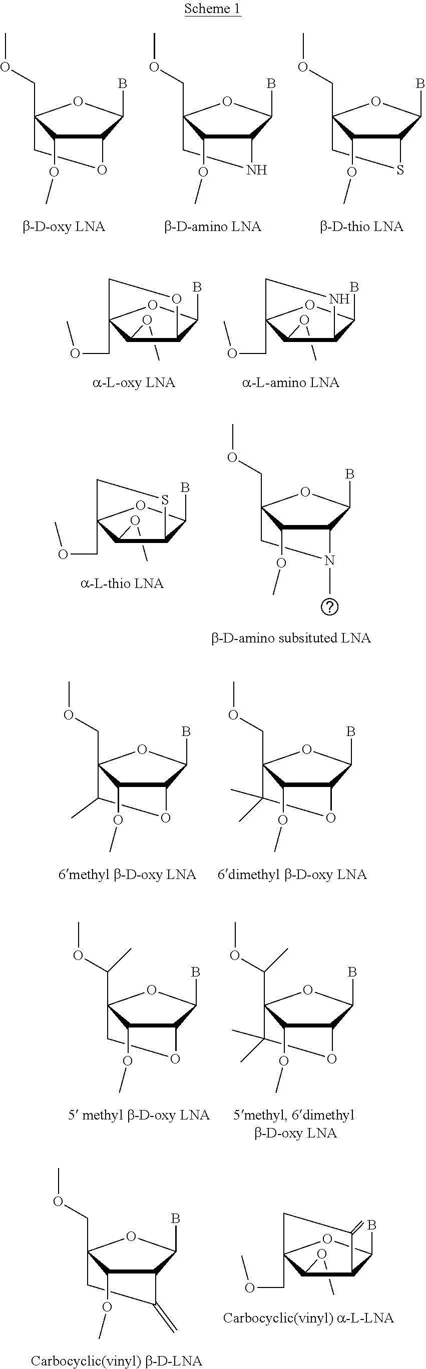

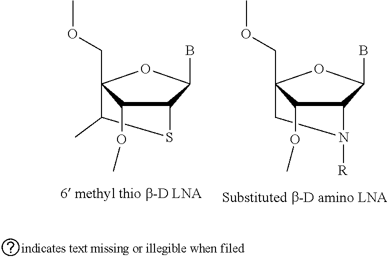

Particular LNA nucleosides are beta-D-oxy-LNA, 6′-methyl-beta-D-oxy LNA such as(S)-6′-methyl-beta-D-oxy-LNA (ScET) and ENA.

A particularly advantageous LNA is beta-D-oxy-LNA.

### Morpholino Oligonucleotides

In some embodiments, the antisense oligonucleotide splice modulators of the invention comprise or consist of morpholino nucleosides (i.e. are Morpholino oligomers and phosphorodiamidate Morpholino oligomer (PMO)). Splice modulating morpholino oligonucleotides have been approved for clinical use—see for example eteplirsen, a 30 nucleotide morpholino oligonucleotide targeting a frame shift mutation in DMD, used to treat Duchenne muscular dystrophy. Morpholino oligonucleotides have nucleases attached to six membered morpholino rings rather ribose, such as methylenemorpholine rings linked through phosphorodiamidate groups, for example as illustrated by the following illustration of 4 consecutive morpholino nucleotides:

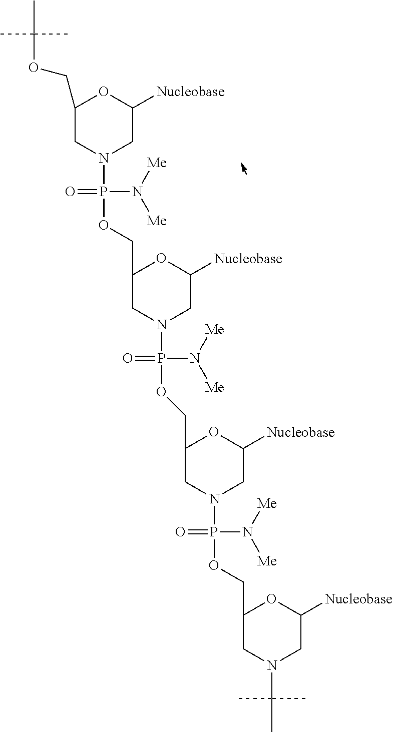

In some embodiments, antisense oligonucleotide splice modulators according to the invention may be, for example 8 to 40 morpholino nucleotides in length, such as morpholino 16 to 20 nucleotides in length, such as 18 to 20 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNase H, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective function as a splice modulator, degradation of the precursor-mRNA is not desirable, and as such it is preferable to avoid the RNaseH degradation of the target. Therefore, the antisense oligonucleotide splice modulators of the invention are preferably not RNase H recruiting gapmer oligonucleotides.

RNase H recruitment may be avoided by limiting the number of contiguous DNA nucleotides in the antisense oligonucleotide splice modulator-therefore mixmer and totalmer designs may be used. Advantageously, in some embodiments, the antisense oligonucleotide splice modulators of the invention, or the contiguous nucleotide sequences thereof, does not comprise more than 3 contiguous DNA nucleosides.

Further, advantageously, in some embodiments, the antisense oligonucleotide splice modulators of the invention, or the contiguous nucleotide sequences thereof, do not comprise more than 4 contiguous DNA nucleosides. Further advantageously, in some embodiments, the antisense oligonucleotide splice modulators of the invention, or contiguous nucleotide sequences thereof, do not comprise more than 2 contiguous DNA nucleosides.

### Mixmers and Totalmers

For use as a splice modulator it is often advantageous to use antisense oligonucleotides which do not recruit RNAase H and do not cause destruction of target pre-cursor-RNA. As RNase H activity requires a contiguous sequence of DNA nucleotides, RNase H recruitment may be prevented by designing oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternate between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

In one embodiment, the mixmer may comprise or consist of nucleosides that alternate between 1, 2 or 3 sequential DNA nucleosides, followed by 1 or 2 sequential LNA nucleosides.

A totalmer is an oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, or may for example comprise only 2′oMe nucleosides, which are reported to be effective for therapeutic use.

Alternatively, a mixmer may comprise a mixture of modified nucleosides, such as MLMLMLMLMLMLMLMLMLML, wherein L=LNA and M=2′-O-MOE nucleosides. Advantageously, the internucleoside nucleosides in mixmers and totalmers may be phosphorothioate, or a majority of nucleoside linkages in mixmers may be phosphorothioate.

Mixmers and totalmers may comprise other internucleoside linkages, such as phosphodiester or phosphorodithioate, by way of example.

In some embodiments, the antisense oligonucleotide splice modulator is or comprises an oligonucleotide mixmer or totalmer. In some embodiments, the contiguous nucleotide sequence is a mixmer or a totalmer.

### Region D′ or D″ in an Oligonucleotide

The antisense oligonucleotide splice modulators of the invention may in some embodiments comprise the contiguous nucleotide sequences of the oligonucleotides which are complementary to the target nucleic acid, such as a mixmer or totalmer region, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the antisense oligonucleotide splice modulators of the invention may comprise a region D′ and/or D″ in addition to the contiguous nucleotide sequence, which may constitute a mixmer or a totalmer.

In some embodiments the internucleoside linkage positioned between region D′ or D″ and the mixmer or totalmer region may be a phosphodiester linkage.

### Conjugates

The invention encompasses an antisense oligonucleotide splice modulator covalently attached to at least one conjugate moiety. In some embodiments this may be referred to as a conjugate of the invention.

In some embodiments, the invention provides an antisense oligonucleotide splice modulator covalently attached to at least one conjugate moiety.

The term “conjugate” as used herein refers to an antisense oligonucleotide splice modulator which is covalently linked to a non-nucleotide moiety (conjugate moiety or region C or third region). The conjugate moiety may be covalently linked to the antisense oligonucleotide splice modulator, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the antisense oligonucleotide splice modulator directly or through a linking moiety (e.g. linker or tether). Linkers serve to covalently connect a third region, e.g. a conjugate moiety (Region C), to a first region, e.g. an antisense oligonucleotide splice modulator or contiguous nucleotide sequence complementary to the target nucleic acid (region A).

In some embodiments of the invention, the conjugate or antisense oligonucleotide splice modulator of the invention may optionally comprise a linker region (second region or region B and/or region Y) which is positioned between the antisense oligonucleotide splice modulator or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The antisense oligonucleotide splice modulator conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Salts

The term “salts” as used herein conforms to its generally known meaning, i.e. an ionic assembly of anions and cations.

In some embodiments, the antisense oligonucleotide splice modulator of the invention may be in the form of a pharmaceutically acceptable salt. Put another way, the invention provides for pharmaceutically acceptable salts of the antisense oligonucleotide splice modulator of the invention.

In some embodiments the pharmaceutically acceptable salt may be a sodium salt or a potassium salt.

The invention provides for a pharmaceutically acceptable sodium salt of the antisense oligonucleotide splice modulator of the invention.

The invention provides for a pharmaceutically acceptable potassium salt of the antisense oligonucleotide splice modulator of the invention.

### Delivery of Antisense Oligonucleotide Splice Modulators

The invention provides for antisense oligonucleotide splice modulators of the invention wherein the antisense oligonucleotide splice modulators are encapsulated in a lipid-based delivery vehicle, covalently linked to or encapsulated in a dendrimer, or conjugated to an aptamer.

This may be for the purpose of delivering the antisense oligonucleotide splice modulators of the invention to the targeted cells and/or to improve the pharmacokinetics of the antisense oligonucleotide splice modulator.

Examples of lipid-based delivery vehicles include oil-in-water emulsions, micelles, liposomes, and lipid nanoparticles.

### Pharmaceutical Compositions

The invention provides for a pharmaceutical composition comprising the antisense oligonucleotide splice modulator of the invention, and a pharmaceutically acceptable diluent, solvent, carrier, salt and/or adjuvant.

The invention provides for a pharmaceutical composition comprising the antisense oligonucleotide splice modulator of the invention, and a pharmaceutically acceptable salt.

For example, the salt may comprise a metal cation, such as a sodium salt or a potassium salt.

The invention provides for a pharmaceutical composition of the invention, wherein the pharmaceutical composition comprises an antisense oligonucleotide splice modulator of the invention, and an aqueous diluent or solvent.

The invention provides for a solution, such as a phosphate buffered saline solution of the antisense oligonucleotide splice modulator of the invention. In some embodiments, the solution, such as phosphate buffered saline solution, of the invention is a sterile solution.

### Method for Increasing UNC13A Expression

The invention provides for a method for enhancing, upregulating or restoring the expression of wild-type UNC13A in a cell, such as a cell which is expressing UNC13A, said method comprising administering an antisense oligonucleotide splice modulator of the invention, or A pharmaceutical composition of the invention in an effective amount to said cell.

In some embodiments the method is an in vitro method.

In some embodiments the method is an in vivo method.

In some embodiments, the cell is an animal cell, preferably a mammalian cell such as a mouse cell, rat cell, hamster cell, or monkey cell, or preferably a human cell.

In some embodiments, the cell is a mammalian cell.

In some embodiments, the cell is a human cell.

In some embodiments the cell is part of, or derived from, a subject suffering from or susceptible to a disease associated with reduced expression of wild-type UNC13A. Such diseases include but are not limited amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Treatment

The term “treatment” as used herein refers to both treatment of an existing disease (e.g. a disease or disorder as herein referred to), or prevention of a disease, i.e. prophylaxis. It will therefore be recognized that treatment, as referred to herein may in some embodiments be prophylactic.

The invention provides for a method for treating or preventing a disease, comprising administering a therapeutically or prophylactically effective amount of an antisense oligonucleotide splice modulator of the invention or a pharmaceutical composition of the invention to a subject suffering from or susceptible to a disease.

The disease may be associated with reduced expression of wild-type UNC13A.

In some embodiments, the invention provides for a method for treating or preventing a disease associated with reduced expression of wild-type UNC13A, comprising administering a therapeutically or prophylactically effective amount of an antisense oligonucleotide splice modulator of the invention or a pharmaceutical composition of the invention to a subject suffering from or susceptible to a disease associated with reduced expression of wild-type UNC13A.

In one embodiment, the disease is a neurological disorder.

In one embodiment the disease is selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

In some embodiments, the subject is an animal, preferably a mammal such as a mouse, rat, hamster, or monkey, or human.

In some embodiments, the subject is a human.

The invention provides for an antisense oligonucleotide splice modulator of the invention for use as a medicament.

The invention provides for an antisense oligonucleotide splice modulator of the invention for the preparation of a medicament.

The invention provides for an antisense oligonucleotide splice modulator of the invention for use in therapy.

The invention provides for a pharmaceutical composition of the invention for use as a medicament.

The invention provides for a pharmaceutical composition of the invention for the preparation of a medicament.

The invention provides for a pharmaceutical composition of the invention for use in therapy.

The invention provides for an antisense oligonucleotide splice modulator of the invention for use as a medicament in the treatment of a neurological disorder.

The invention provides for an antisense oligonucleotide splice modulator of the invention for use as a medicament in the treatment of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for the use of an antisense oligonucleotide splice modulator of the invention for the preparation of a medicament for the treatment or prevention of a neurological disorder.

The invention provides for the use of an antisense oligonucleotide splice modulator of the invention for the preparation of a medicament for the treatment or prevention of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for a pharmaceutical composition of the invention for use as a medicament in the treatment of a neurological disorder.

The invention provides for a pharmaceutical composition of the invention for use as a medicament in the treatment of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for the use of a pharmaceutical composition of the invention for the preparation of a medicament for the treatment or prevention of a neurological disorder.

The invention provides for the use of a pharmaceutical composition of the invention for the preparation of a medicament for the treatment or prevention of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Administration

The antisense oligonucleotide splice modulator of the invention or the pharmaceutical composition of the invention may be administered topically (such as, to the skin, inhalation, ophthalmic or otic) or enteral (such as, orally or through the gastrointestinal tract) or parenterally (such as, intravenous, subcutaneous, intra-muscular, intracerebral, intracerebroventricular or intrathecal).

In a preferred embodiment the antisense oligonucleotide splice modulator of the invention or pharmaceutical composition of the invention is administered by a parenteral route including intravenous, intraarterial, subcutaneous, intraperitoneal or intramuscular injection or infusion, intrathecal or intracranial, e.g., intracerebral or intraventricular, administration. In one embodiment the antisense oligonucleotide splice modulator of the invention is administered intracerebrally or intracerebroventricularly. In another embodiment the antisense oligonucleotide splice modulator of the invention is administered intrathecally.

The invention also provides for the use of the antisense oligonucleotide splice modulator of the invention or pharmaceutical composition of the invention as described for the preparation of a medicament wherein the medicament is in a dosage form for intrathecal administration.

The invention also provides for the use of the antisense oligonucleotide splice modulator of the invention or pharmaceutical composition of the invention as described for the preparation of a medicament wherein the medicament is in a dosage form for intracerebral or intraventricular administration.

The invention also provides for the use of the antisense oligonucleotide splice modulator of the invention or pharmaceutical composition of the invention as described for the preparation of a medicament wherein the medicament is in a dosage form for intracerebroventricular administration.

### Combination Therapies

In some embodiments, the antisense oligonucleotide splice modulator of the invention or pharmaceutical composition of the invention is for use in a combination treatment with one or more other therapeutic agents.

### EXAMPLES

### Example 1: Identification of UNC13A as a Novel Target for TDP43 mRNA Splice Regulation

One hallmark feature of ALS disease is the presence of cytoplasmic aggregated TDP43 protein in a small fraction of the patient's neuronal cells. The consequence of cytoplasmic aggregation of TDP43 is that it becomes depleted in the cell nucleus, and hence can't perform its normal function here.

TDP43 has been shown to affect mRNA splicing. In order to identify new genes whose mRNAs are regulated by the presence of TDP43, we did a knockdown of TDP43 in a neuronal cell model. RNA sequencing was performed on the cells, and de novo transcript analysis was performed to identify affected genes with new splice patterns.

Human glutamatergic neurons (Fujifilms) were plated at 60,000 viable cells together with 10,000 viable Astrocytes (Fujifilms) in 96-well plates coated with Laminin and Poly(ethyleneimine) solution (Sigma Aldrich) in 200 μl culture medium (day-1).

To knockdown TDP-43, compound A (SEQ ID 553) was added to the culture medium at 5 UM on day 0, in other wells PBS was added instead as control. Half the cell culture medium was changed 3 times a week during the whole experiment (day 2, 5, 7, 10, 12, 14 & 17). The cells were harvested on day 20 using Magnapure lysis buffer (Roche) and RNA was isolation on MagNA pure 96 system (Roche) according to the manufacturer's instructions including DNase treatment step. NGS libraries was prepared from 100 ng of total RNA using the KAPA mRNA HyperPrep Kit Illumina® Platforms (Roche). Libraries were subjected to paired-end sequencing on a NovaSeq6000 sequencer (Illumina) with 150-bp read length. Data analysis was carried out using CLC Genomics Workbench 21. Data was first analyzed by running large gap mapping analysis using hg38 genome assembly, followed by transcript discovery. Predicted novel splice events were examined by manual visual inspection to identify real splice events.

The inclusion of a novel 128 (mutant transcript 1) or 178 (mutant transcript 2) base pair exon in UNC13A upon loss of TDP43 was discovered. The first and last base in the new exons is; mutant transcript 1: (17,642,541 and 17,642,414), mutant transcript 2 (17,642,591 and 17,642,414) according to the hg38 human gene annotation with the UNC13A being placed in the minus orientation. This results in the inclusion of a 128 bp or 178 bp exon, which in both cases results in a frameshift and a pre-mature stop codon that then would target the transcripts for nonsense mediated decay (NMD).

### Example 2: Rescue of Erroneous UNC13A mRNA Splicing Caused by the Lack of TDP43 Using ASO

Here we show ASOs ability to induce proper splicing on the TDP43 target UNC13A.

Human glutamatergic neurons (Fujifilms) were plated at 60000 viable cells together with 10.000 viable Astrocytes (Fujifilms) per 96-well plates coated with Laminin and Poly(ethyleneimine) solution (Sigma Aldrich) in 200 ul Culture medium (day-1). To knockdown TDP-43, compound A (SEQ ID 553) was added to the culture medium at 5 uM on day 0 (Except for four control wells per plate). Half the cell culture medium was changed 3 times a week during the whole experiment (day 2, 5, 7, 9, 12, 14, 16, 19) . . . . The ASOs targeting the cryptic UNC13A exons were added to the culture medium on day 5 at 10 uM. 272 different ASOs were added in total (SEQ ID 280-551). 10 wells per plate received only the compound A (SEQ ID 553) to serve as a baseline reference. The experiments were run in duplicate.

The cells were harvested on day 20 using Magnapure lysis buffer (Roche) and RNA was isolated on MagNA pure 96 system (Roche) according to the manufacturer's instructions including DNase treatment step. The purified RNA was denatured 30 sek at 90 before cDNA synthesis. cDNA was created using the iScript Advanced cDNA Synthesis Kit for RT-qPCR (Biorad) according to the manufacturer's instructions.

Measurements of the expression levels of the target genes was done by droplet digital PCR using the QX1 system (Bio-Rad) together with the QX1 software stand edition. The pcr-probe assays used to measure the expressed of normally spliced target mRNA was designed to span the two exons, where in-between the new “mutant” exons would occur.

Data shown in Table 1 was normalized to the expression of the house keeping gene HPRT1, and finally normalized to the average expression value of the four control wells (PBS) that didn't receive any TDP43 knock-down or CA-repeat ASO.

The following PCR probe assay synthesized at (Integrated DNA technologies (IDT)) were used:

TARDBP:
Primer 1: CAGCTCATCCTCAGTCATGTC,
Primer 2: GATGGTGTGACTGCAAACTTC,
Probe:
/5Cy5/CAGCGCCCCACAAACACTTTTCT/3IAbRQSp/,
UNC13A wt (ex20-ex21):
Primer 1:
GATCAAAGGCGAGGAGAAGG ,
Primer 2:
TGGCATCTGGGATCTTCAC,
Probe:
/56-FAM/ACCTGTCTG/ZEN/CATGAGAACCTGTTCCACTTC/
3IABKFQ/

The following CY5.5 labelled HPRT1 probe was purchased from BioRad: dHsaCPE13136107.

TABLE 1
Production of TDP43 and UNC13A WT following exposure
to oligonucleotides. Data shown in Table 1 shows the
percentile of gene expression compared to control cells.
Data was normalized to the expression of the house keeping
gene HPRT1, and finally normalized to the average expression
value of the control wells (PBS) that didn't receive
any TDP43 knock-down. KD (“knockdown”, describes
wells that only received treatment with the gapmer ASO
that degrades the TDP43 mRNA)

| Oligo SEQ ID NO | Norm UNC13A | Norm TDP43 |
|---|---|---|
| PBS | 90.5 | 105.0 |
| PBS | 88.6 | 104.8 |
| PBS | 87.8 | 104.6 |
| PBS | 102.8 | 98.8 |
| PBS | 111.1 | 101.2 |
| PBS | 92.1 | 98.9 |
| PBS | 86.7 | 97.0 |
| PBS | 88.3 | 96.7 |
| PBS | 131.4 | 105.9 |
| | 96.8 | 95.6 |
| | 100.8 | 89.5 |
| | 123.0 | 109.3 |
| | 122.4 | 100.7 |
| | 91.5 | 92.2 |
| | 3.5 | 4.7 |
| | 4.6 | 4.3 |
| | 4.2 | 3.2 |
| | 3.0 | 6.5 |
| | 7.6 | 7.3 |
| | 4.9 | 4.3 |
| | 8.0 | 3.7 |
| | 8.5 | 4.7 |
| | 7.4 | 7.9 |
| | 3.0 | 6.1 |
| | 3.3 | 3.6 |
| | 3.3 | 2.8 |
| | 3.8 | 4.8 |
| | 6.6 | 7.2 |
| | 6.2 | 4.2 |
| | 5.0 | 6.9 |
| | 7.0 | 6.5 |
| | 4.9 | 8.0 |
| | 7.3 | 5.5 |
| | 7.1 | 3.8 |
| | 6.8 | 7.4 |
| | 4.5 | 7.5 |
| | 3.5 | 9.2 |
| | 5.9 | 7.6 |
| | 2.3 | 7.1 |
| | 3.8 | 5.8 |
| | 4.6 | 3.5 |
| | 8.0 | 8.0 |
| | 6.1 | 7.3 |
| | 4.2 | 4.1 |
| | 5.2 | 4.0 |
| | 5.1 | 2.8 |
| | 4.6 | 7.1 |
| | 7.5 | 5.2 |
| | 5.2 | 9.2 |
| | 3.6 | 6.2 |
| | 7.2 | 5.8 |
| | 4.1 | 5.2 |
| | 6.3 | 5.7 |
| | 6.0 | 10.7 |
| | 7.7 | 6.3 |
| | 7.5 | 9.8 |
| | 3.0 | 9.3 |
| | 6.3 | 8.9 |
| | 3.4 | 4.3 |
| | 4.7 | 6.6 |
| | 4.6 | 6.4 |
| | 9.4 | 8.1 |
| KD | 4.4 | 4.8 |
| KD | 6.4 | 9.0 |
| KD | 4.9 | 8.9 |
| KD | 6.6 | 8.8 |
| KD | 4.0 | 2.6 |
| KD | 6.2 | 8.2 |
| KD | 6.3 | 8.2 |
| KD | 7.1 | 8.0 |
| KD | 7.9 | 6.7 |
| KD | 19.0 | 6.5 |
| KD | 2.4 | 3.9 |
| KD | 5.8 | 7.9 |
| KD | 3.5 | 5.0 |
| KD | 4.6 | 5.1 |
| KD | 5.3 | 7.0 |
| KD | 6.8 | 3.5 |
| KD | 16.6 | 5.7 |
| KD | 4.0 | 5.6 |
| KD | 4.9 | 4.4 |
| KD | 2.6 | 2.4 |
| KD | 9.2 | 5.6 |
| KD | 6.6 | 5.8 |
| KD | 7.7 | 5.1 |
| KD | 5.9 | 5.5 |
| SEQ ID NO: 280 | 0.0 | 6.6 |
| SEQ ID NO: 281 | 1.0 | 8.4 |
| SEQ ID NO: 282 | 1.0 | 7.3 |
| SEQ ID NO: 283 | 2.8 | 16.2 |
| SEQ ID NO: 284 | 1.0 | 11.4 |
| SEQ ID NO: 285 | 1.5 | 9.1 |
| SEQ ID NO: 286 | 1.3 | 10.2 |
| SEQ ID NO: 287 | 0.7 | 4.6 |
| SEQ ID NO: 288 | 0.4 | 7.5 |
| SEQ ID NO: 289 | 4.5 | 14.8 |
| SEQ ID NO: 290 | 0.0 | 9.7 |
| SEQ ID NO: 291 | 1.0 | 6.8 |
| SEQ ID NO: 292 | 1.8 | 8.3 |
| SEQ ID NO: 293 | 13.1 | 18.9 |
| SEQ ID NO: 294 | 1.0 | 3.4 |
| SEQ ID NO: 295 | 0.8 | 6.5 |
| SEQ ID NO: 296 | 0.5 | 7.6 |
| SEQ ID NO: 297 | 1.0 | 2.5 |
| SEQ ID NO: 298 | 1.2 | 4.3 |
| SEQ ID NO: 299 | 2.4 | 4.4 |
| SEQ ID NO: 300 | 0.9 | 7.7 |
| SEQ ID NO: 301 | 1.0 | 4.1 |
| SEQ ID NO: 302 | 0.9 | 4.3 |
| SEQ ID NO: 303 | 0.0 | 2.5 |
| SEQ ID NO: 304 | 1.5 | 4.9 |
| SEQ ID NO: 305 | 0.7 | 3.3 |
| SEQ ID NO: 306 | 0.9 | 4.2 |
| SEQ ID NO: 307 | 0.2 | 3.3 |
| SEQ ID NO: 308 | 1.8 | 4.6 |
| SEQ ID NO: 309 | 1.4 | 4.7 |
| SEQ ID NO: 310 | 3.8 | 5.4 |
| SEQ ID NO: 311 | 2.6 | 6.0 |
| SEQ ID NO: 312 | 2.3 | 3.1 |
| SEQ ID NO: 313 | 3.3 | 11.1 |
| SEQ ID NO: 314 | 15.9 | 5.7 |
| SEQ ID NO: 315 | 0.9 | 4.0 |
| SEQ ID NO: 316 | 1.6 | 2.7 |
| SEQ ID NO: 317 | 1.1 | 5.2 |
| SEQ ID NO: 318 | 1.5 | 4.8 |
| SEQ ID NO: 319 | 2.3 | 6.1 |
| SEQ ID NO: 320 | 2.5 | 6.2 |
| SEQ ID NO: 321 | 1.3 | 7.2 |
| SEQ ID NO: 322 | 0.0 | 10.7 |
| SEQ ID NO: 323 | 0.8 | 4.4 |
| SEQ ID NO: 324 | 2.2 | 6.0 |
| SEQ ID NO: 325 | 0.0 | 6.2 |
| SEQ ID NO: 326 | 0.4 | 4.5 |
| SEQ ID NO: 327 | 0.8 | 7.8 |
| SEQ ID NO: 328 | 0.4 | 4.4 |
| SEQ ID NO: 329 | 0.3 | 6.7 |
| SEQ ID NO: 330 | 1.9 | 6.0 |
| SEQ ID NO: 331 | 6.3 | 5.6 |
| SEQ ID NO: 332 | 3.7 | 4.6 |
| SEQ ID NO: 333 | 3.7 | 5.2 |
| SEQ ID NO: 334 | 33.1 | 5.0 |
| SEQ ID NO: 335 | 2.4 | 4.8 |
| SEQ ID NO: 336 | 21.0 | 9.9 |
| SEQ ID NO: 337 | 7.8 | 8.3 |
| SEQ ID NO: 338 | 72.0 | 5.2 |
| SEQ ID NO: 339 | 3.0 | 4.5 |
| SEQ ID NO: 340 | 46.8 | 4.4 |
| SEQ ID NO: 341 | 20.3 | 5.1 |
| SEQ ID NO: 342 | 65.6 | 4.6 |
| SEQ ID NO: 343 | 2.2 | 5.8 |
| SEQ ID NO: 344 | 96.5 | 4.0 |
| SEQ ID NO: 345 | 117.2 | 4.2 |
| SEQ ID NO: 346 | 133.3 | 4.4 |
| SEQ ID NO: 347 | 10.4 | 9.3 |
| SEQ ID NO: 348 | 101.3 | 3.2 |
| SEQ ID NO: 349 | 19.8 | 1.7 |
| SEQ ID NO: 350 | 11.6 | 9.4 |
| SEQ ID NO: 351 | 1.9 | 4.8 |
| SEQ ID NO: 352 | 5.4 | 7.6 |
| SEQ ID NO: 353 | 1.2 | 3.2 |
| SEQ ID NO: 354 | 2.0 | 5.0 |
| SEQ ID NO: 355 | 1.2 | 5.1 |
| SEQ ID NO: 356 | 2.5 | 5.5 |
| SEQ ID NO: 357 | 2.4 | 5.4 |
| SEQ ID NO: 358 | 2.5 | 3.5 |
| SEQ ID NO: 359 | 2.3 | 3.1 |
| SEQ ID NO: 360 | 1.1 | 6.6 |
| SEQ ID NO: 361 | 5.9 | 3.3 |
| SEQ ID NO: 362 | 3.2 | 7.4 |
| SEQ ID NO: 363 | 1.1 | 3.7 |
| SEQ ID NO: 364 | 2.1 | 4.7 |
| SEQ ID NO: 365 | 2.1 | 7.1 |
| SEQ ID NO: 366 | 4.3 | 4.0 |
| SEQ ID NO: 367 | 20.8 | 5.4 |
| SEQ ID NO: 368 | 1.2 | 5.9 |
| SEQ ID NO: 369 | 2.4 | 13.1 |
| SEQ ID NO: 370 | 1.7 | 5.9 |
| SEQ ID NO: 371 | 1.7 | 4.7 |
| SEQ ID NO: 372 | 4.4 | 14.5 |
| SEQ ID NO: 373 | 1.6 | 10.2 |
| SEQ ID NO: 374 | 0.6 | 8.1 |
| SEQ ID NO: 375 | 3.0 | 6.7 |
| SEQ ID NO: 376 | 1.3 | 10.9 |
| SEQ ID NO: 377 | 0.8 | 10.7 |
| SEQ ID NO: 378 | 8.4 | 8.7 |
| SEQ ID NO: 379 | 2.2 | 5.0 |
| SEQ ID NO: 380 | 3.5 | 6.3 |
| SEQ ID NO: 381 | 1.5 | 7.7 |
| SEQ ID NO: 382 | 1.1 | 4.4 |
| SEQ ID NO: 383 | 4.2 | 6.4 |
| SEQ ID NO: 384 | 33.8 | 5.3 |
| SEQ ID NO: 385 | 1.4 | 6.8 |
| SEQ ID NO: 386 | 32.2 | 8.9 |
| SEQ ID NO: 387 | 49.4 | 5.1 |
| SEQ ID NO: 388 | 19.7 | 4.9 |
| SEQ ID NO: 389 | 31.6 | 4.2 |
| SEQ ID NO: 390 | 16.4 | 5.5 |
| SEQ ID NO: 391 | 1.7 | 5.4 |
| SEQ ID NO: 392 | 22.8 | 10.1 |
| SEQ ID NO: 393 | 21.0 | 4.4 |
| SEQ ID NO: 394 | 86.2 | 6.4 |
| SEQ ID NO: 395 | 27.6 | 5.9 |
| SEQ ID NO: 396 | 17.1 | 6.9 |
| SEQ ID NO: 397 | 81.6 | 6.3 |
| SEQ ID NO: 398 | 92.6 | 4.3 |
| SEQ ID NO: 399 | 131.9 | 4.6 |
| SEQ ID NO: 400 | 183.2 | 6.0 |
| SEQ ID NO: 401 | 60.7 | 6.5 |
| SEQ ID NO: 402 | 76.0 | 5.2 |
| SEQ ID NO: 403 | 136.9 | 4.4 |
| SEQ ID NO: 404 | 28.1 | 6.8 |
| SEQ ID NO: 405 | 53.7 | 4.7 |
| SEQ ID NO: 406 | 119.8 | 2.4 |
| SEQ ID NO: 407 | 144.9 | 6.4 |
| SEQ ID NO: 408 | 60.8 | 2.9 |
| SEQ ID NO: 409 | 116.7 | 5.7 |
| SEQ ID NO: 410 | 64.7 | 5.0 |
| SEQ ID NO: 411 | 74.4 | 3.3 |
| SEQ ID NO: 412 | 189.4 | 5.2 |
| SEQ ID NO: 413 | 145.7 | 5.3 |
| SEQ ID NO: 414 | 163.4 | 4.5 |
| SEQ ID NO: 415 | 82.2 | 6.1 |
| SEQ ID NO: 416 | 10.1 | 5.1 |
| SEQ ID NO: 417 | 26.1 | 5.1 |
| SEQ ID NO: 418 | 87.5 | 6.0 |
| SEQ ID NO: 419 | 0.5 | 5.0 |
| SEQ ID NO: 420 | 6.6 | 5.1 |
| SEQ ID NO: 421 | 10.4 | 6.0 |
| SEQ ID NO: 422 | 28.6 | 7.6 |
| SEQ ID NO: 423 | 57.3 | 7.3 |
| SEQ ID NO: 424 | 69.5 | 4.6 |
| SEQ ID NO: 425 | 35.8 | 4.6 |
| SEQ ID NO: 426 | 73.0 | 3.7 |
| SEQ ID NO: 427 | 58.3 | 3.5 |
| SEQ ID NO: 428 | 80.0 | 5.7 |
| SEQ ID NO: 429 | 12.2 | 9.0 |
| SEQ ID NO: 430 | 13.7 | 3.0 |
| SEQ ID NO: 431 | 28.0 | 2.0 |
| SEQ ID NO: 432 | 22.9 | 3.0 |
| SEQ ID NO: 433 | 17.3 | 3.8 |
| SEQ ID NO: 434 | 83.9 | 4.8 |
| SEQ ID NO: 435 | 66.7 | 3.8 |
| SEQ ID NO: 436 | 26.2 | 4.8 |
| SEQ ID NO: 437 | 16.5 | 2.7 |
| SEQ ID NO: 438 | 8.3 | 4.8 |
| SEQ ID NO: 439 | 2.8 | 3.3 |
| SEQ ID NO: 440 | 13.9 | 3.1 |
| SEQ ID NO: 441 | 16.1 | 8.9 |
| SEQ ID NO: 442 | 11.8 | 5.9 |
| SEQ ID NO: 443 | 2.9 | 2.6 |
| SEQ ID NO: 444 | 11.9 | 7.0 |
| SEQ ID NO: 445 | 27.4 | 9.8 |
| SEQ ID NO: 446 | 10.6 | 8.1 |
| SEQ ID NO: 447 | 16.7 | 3.4 |
| SEQ ID NO: 448 | 26.7 | 7.3 |
| SEQ ID NO: 449 | 21.2 | 8.0 |
| SEQ ID NO: 450 | 33.2 | 14.2 |
| SEQ ID NO: 451 | 40.7 | 16.9 |
| SEQ ID NO: 452 | 20.2 | 10.6 |
| SEQ ID NO: 453 | 19.0 | 13.5 |
| SEQ ID NO: 454 | 22.8 | 8.1 |
| SEQ ID NO: 455 | 38.5 | 7.8 |
| SEQ ID NO: 456 | 27.5 | 8.5 |
| SEQ ID NO: 457 | 14.1 | 4.8 |
| SEQ ID NO: 458 | 20.7 | 5.6 |
| SEQ ID NO: 459 | 37.4 | 5.2 |
| SEQ ID NO: 460 | 15.7 | 4.9 |
| SEQ ID NO: 461 | 36.6 | 2.7 |
| SEQ ID NO: 462 | 14.2 | 8.2 |
| SEQ ID NO: 463 | 22.2 | 3.0 |
| SEQ ID NO: 464 | 32.6 | 3.8 |
| SEQ ID NO: 465 | 41.9 | 3.3 |
| SEQ ID NO: 466 | 23.2 | 6.7 |
| SEQ ID NO: 467 | 21.6 | 4.8 |
| SEQ ID NO: 468 | 12.3 | 8.1 |
| SEQ ID NO: 469 | 1.8 | 4.3 |
| SEQ ID NO: 470 | 8.3 | 5.3 |
| SEQ ID NO: 471 | 6.3 | 2.8 |
| SEQ ID NO: 472 | 12.4 | 3.5 |
| SEQ ID NO: 473 | 4.4 | 3.3 |
| SEQ ID NO: 474 | 9.0 | 5.9 |
| SEQ ID NO: 475 | 5.5 | 4.5 |
| SEQ ID NO: 476 | 5.4 | 4.0 |
| SEQ ID NO: 477 | 13.4 | 4.1 |
| SEQ ID NO: 478 | 17.8 | 3.6 |
| SEQ ID NO: 479 | 6.6 | 4.0 |
| SEQ ID NO: 480 | 18.3 | 2.0 |
| SEQ ID NO: 481 | 15.1 | 2.8 |
| SEQ ID NO: 482 | 9.0 | 4.3 |
| SEQ ID NO: 483 | 8.6 | 4.2 |
| SEQ ID NO: 484 | 21.5 | 5.4 |
| SEQ ID NO: 485 | 3.9 | 4.2 |
| SEQ ID NO: 486 | 32.3 | 3.6 |
| SEQ ID NO: 487 | 13.8 | 6.3 |
| SEQ ID NO: 488 | 8.5 | 3.4 |
| SEQ ID NO: 489 | 11.4 | 3.2 |
| SEQ ID NO: 490 | 12.8 | 6.6 |
| SEQ ID NO: 491 | 12.2 | 6.4 |
| SEQ ID NO: 492 | 5.8 | 4.5 |
| SEQ ID NO: 493 | 11.6 | 6.1 |
| SEQ ID NO: 494 | 8.2 | 3.9 |
| SEQ ID NO: 495 | 13.5 | 5.3 |
| SEQ ID NO: 496 | 9.7 | 5.5 |
| SEQ ID NO: 497 | 13.8 | 3.8 |
| SEQ ID NO: 498 | 18.2 | 5.9 |
| SEQ ID NO: 499 | 2.3 | 6.4 |
| SEQ ID NO: 500 | 7.5 | 4.5 |
| SEQ ID NO: 501 | 2.3 | 3.7 |
| SEQ ID NO: 502 | 1.3 | 2.0 |
| SEQ ID NO: 503 | 2.5 | 4.9 |
| SEQ ID NO: 504 | 0.8 | 5.5 |
| SEQ ID NO: 505 | 5.5 | 2.4 |
| SEQ ID NO: 506 | 0.7 | 2.3 |
| SEQ ID NO: 507 | 5.6 | 3.1 |
| SEQ ID NO: 508 | 2.3 | 2.1 |
| SEQ ID NO: 509 | 2.3 | 4.2 |
| SEQ ID NO: 510 | 2.5 | 3.5 |
| SEQ ID NO: 511 | 4.0 | 4.7 |
| SEQ ID NO: 512 | 6.9 | 6.8 |
| SEQ ID NO: 513 | 4.5 | 2.4 |
| SEQ ID NO: 514 | 6.8 | 4.2 |
| SEQ ID NO: 515 | 25.3 | 10.1 |
| SEQ ID NO: 516 | 22.8 | 6.2 |
| SEQ ID NO: 517 | 19.2 | 6.0 |
| SEQ ID NO: 518 | 15.2 | 6.3 |
| SEQ ID NO: 519 | 32.3 | 7.6 |
| SEQ ID NO: 520 | 25.9 | 8.9 |
| SEQ ID NO: 521 | 22.2 | 4.8 |
| SEQ ID NO: 522 | 23.7 | 6.8 |
| SEQ ID NO: 523 | 29.7 | 9.6 |
| SEQ ID NO: 524 | 34.9 | 16.7 |
| SEQ ID NO: 525 | 24.6 | 5.6 |
| SEQ ID NO: 526 | 48.8 | 10.4 |
| SEQ ID NO: 527 | 24.0 | 9.6 |
| SEQ ID NO: 528 | 26.7 | 9.0 |
| SEQ ID NO: 529 | 10.4 | 7.9 |
| SEQ ID NO: 530 | 36.6 | 10.1 |
| SEQ ID NO: 531 | 25.1 | 13.0 |
| SEQ ID NO: 532 | 11.0 | 6.3 |
| SEQ ID NO: 533 | 20.2 | 6.2 |
| SEQ ID NO: 534 | 14.0 | 5.7 |
| SEQ ID NO: 535 | 23.0 | 9.2 |
| SEQ ID NO: 536 | 21.0 | 7.1 |
| SEQ ID NO: 537 | 13.0 | 8.9 |
| SEQ ID NO: 538 | 8.7 | 4.8 |
| SEQ ID NO: 539 | 25.8 | 8.6 |
| SEQ ID NO: 540 | 14.8 | 6.1 |
| SEQ ID NO: 541 | 13.2 | 5.7 |
| SEQ ID NO: 542 | 8.5 | 4.3 |
| SEQ ID NO: 543 | 14.5 | 5.3 |
| SEQ ID NO: 544 | 17.1 | 6.0 |
| SEQ ID NO: 545 | 117.2 | 3.0 |
| SEQ ID NO: 546 | 1.5 | 4.2 |
| SEQ ID NO: 547 | 7.3 | 6.7 |
| SEQ ID NO: 548 | 95.8 | 2.2 |
| SEQ ID NO: 549 | 138.4 | 6.7 |
| SEQ ID NO: 550 | 34.9 | 6.0 |
| SEQ ID NO: 551 | 98.8 | 5.9 |

TABLE 2
COMPOUND TABLE

| Natural | Oligo | | | Target |
|---|---|---|---|---|
| analog | SEQ ID | | Target | SEQ |
| sequence | NO | HELM | sequence | ID NO |
| 5′- | SEQ ID | RNA1{[LR]([5meC])[sP].[dR](A)[P].[LR](T)[s | TGGATGG | SEQ ID NO: |
| CATCTGT | NO: 280 | P].[dR](C)[sP].[dR](T)[sP].[LR](G)[sP].[dR](T) | ATGGACA | 8 |
| CCATCCA | | [sP].[dR](C)[sP].[dR](C)[sP].[LR](A)[sP].[dR]( | GATG | |
| TCCA-3′ | | T)[sP].[dR](C)[sP].[dR](C)[sP].[LR](A)[sP].[d | | |
| | | R](T)[sP].[dR](C)[sP].[LR]([5meC])[sP].[LR](A | | |
| | | )}$$$$V2.0 | | |
| 5′- | SEQ ID | RNA1{[LR](A)[sP].[dR](T)[sP].[dR](C)[sP].[dR | ATGGATG | SEQ ID NO: |
| ATCTGTC | NO: 281 | ](T)[sP].[LR](G)[sP].[dR](T)[sP].[dR](C)[sP].[d | GATGGAC | 9 |
| CATCCAT | | R](C)[sP].[LR](A)[sP].[dR](T)[sP].[dR](C)[sP]. | AGAT | |
| CCAT-3′ | | [dR](C)[sP].[LR](A)[sP].[dR](T)[sP].[dR](C)[s | | |
| | | P].[dR](C)[sP].[LR](A)[sP].[LR](T)}$$$$V2.0 | | |
| 5′- | SEQ ID | RNA1{[LR](T)[sP].[dR](C)[sP].[dR](T)[sP].[LR | GATGGAT | SEQ ID NO: |
| TCTGTCC | NO: 282 | ](G)[sP].[dR](T)[sP].[dR](C)[sP].[dR](C)[sP].[ | GGATGGA | 10 |
| ATCCATC | | LR](A)[sP].[dR](T)[sP].[dR](C)[sP].[dR](C)[sP | CAGA | |
| CATC-3′ | | ].[LR](A)[sP].[dR](T)[sP].[dR](C)[sP].[LR]([5m | | |
| | | eC])[sP].[dR](A)[sP].[LR](T)[sP].[LR]([5meC]) | | |
| | | }$$$$V2.0 | | |
| 5′- | SEQ ID | RNA1{[LR]([5meC])[sP].[dR](T)[sP].[LR](G)[s | TGATGGA | SEQ ID NO: |
| CTGTCCA | NO: 283 | P].[dR](T)[sP].[dR](C)[sP].[dR](C)[sP].[LR](A) | TGGATGG | 11 |
| TCCATCC | | [sP].[dR](T)[sP].[dR](C)[sP].[dR](C)[sP].[LR]( | ACAG | |
| ATCA-3′ | | A)[P].[dR](T)[sP].[dR](C)[sP].[dR](C)[sP].[L | | |
| | | R](A)[sP].[dR](T)[sP].[LR]([5meC])[sP].[LR](A | | |
| | | )}$$$$V2.0 | | |
| 5′- | SEQ ID | RNA1{[LR](T)[sP].[dR](G)[sP].[LR](T)[sP].[dR | ATGATGG | SEQ ID NO: |
| TGTCCAT | NO: 284 | ](C)[sP].[dR](C)[sP].[LR](A)[sP].[dR](T)[sP].[d | ATGGATG | 12 |
| CCATCCA | | R](C)[sP].[dR](C)[sP].[LR](A)[P].[dR](T)[sP]. | GACA | |
| TCAT-3′ | | [dR](C)[sP].[dR](C)[sP].[LR](A)[sP].[dR](T)[s | | |
| | | P].[dR](C)[sP].[LR](A)[sP].[LR](T)}$$$$V2.0 | | |
| 5′- | SEQ ID | RNA1{[LR](T)[sP].[dR](C)[sP].[dR](C)[sP].[L | GGATGAT | SEQ ID NO: |
| TCCATCC | NO: 285 | R](A)[sP].[dR](T)[sP].[dR](C)[sP].[dR](C)[sP]. | GGATGGA | 13 |
| ATCCATC | | [LR](A)[sP].[dR](T)[sP].[dR](C)[sP].[dR](C)[s | TGGA | |
| ATCC-3′ | | P].[LR](A)[sP].[dR](T)[sP].[dR](C)[sP].[LR](A) | | |
| | | [sP].[dR](T)[sP].[LR]([5meC])[sP].[LR]([5meC | | |
| | | ])}$$$$V2.0 | | |
| 5′- | SEQ ID | RNA1{[LR]([5meC])[sP].[dR](C)[sP].[LR](A)[s | TGGATGA | SEQ ID NO: |
| CCATCCA | NO: 286 | P].[dR](T)[sP].[dR](C)[sP].[dR](C)[sP].[LR](A) | TGGATGG | 14 |
| TCCATCA | | [sP].[dR](T)[sP].[dR](C)[sP].[dR](C)[sP].[LR]( | ATGG | |
| TCCA-3′ | | A)[sP].[dR](T)[sP].[dR](C)[sP].[LR](A)[sP].[dR | | |
| | | ](T)[sP].[dR](C)[sP].[LR]([5meC])[sP].[LR](A)} | | |
| | | $$$$V2.0 | | |
| 5′- | SEQ ID | RNA1{[LR]([5meC])[sP].[dR](A)[sP].[LR](T)[s | ATGGATG | SEQ ID NO: |
| CATCCAT | NO: 287 | P].[dR](C)[sP].[dR](C)[sP].[LR](A)[sP].[dR](T) | ATGGATG | 15 |
| CCATCAT | | [sP].[dR](C)[sP].[dR](C)[P].[LR](A)[sP].[dR]( | GATG | |
| CCAT-3′ | | T)[sP].[dR](C)[sP].[LR](A)[sP].[dR](T)[sP].[dR | | |
| | | ](C)[sP].[dR](C)[sP].[LR](A)[sP].[LR](T)}$$$$ | | |
| | | V2.0 | | |
| 5′- | SEQ ID | RNA1{[LR](T)[sP].[dR](C)[sP].[dR](C)[sP].[L | AGATGGA | SEQ ID NO: |
| TCCATCC | NO: 288 | R](A)[sP].[dR](T)[sP].[dR](C)[sP].[dR](C)[sP]. | TGATGGA | 16 |
| ATCATCC | | [LR](A)[sP].[dR](T)[sP].[dR](C)[sP].[LR](A)[s | TGGA | |
| ATCT-3′ | | P].[dR](T)[sP].[dR](C)[sP].[dR](C)[sP].[LR](A) | | |
| | | [sP].[dR](T)[sP].[LR]([5meC])[P].[LR](T)}$$$ | | |
| | | $V2.0 | | |
| 5′- | SEQ ID | RNA1{[LR]([5meC])[sP].[dR](C)[sP].[LR](A)[s | TAGATGG | SEQ ID NO: |
| CCATCCA | NO: 289 | P].[dR](T)[sP].[dR](C)[sP].[dR](C)[sP].[LR](A) | ATGATGG | 17 |
| TCATCCA | | [sP].[dR](T)[sP].[dR](C)[sP].[LR](A)[sP].[dR]( | ATGG | |
| TCTA-3′ | | T)[sP].[dR](C)[sP].[dR](C)[sP].[LR](A)[sP].[d | | |
| | | R](T)[sP].[dR](C)[sP].[LR](T)[sP].[LR](A)}$$$ | | |
| | | $V2.0 | | |
| 5′- | SEQ ID | RNA1{[LR]([5meC])[sP].[dR](A)[sP].[LR](T)[s | CTAGATG | SEQ ID NO: |
| CATCCAT | NO: 290 | P].[dR](C)[sP].[dR](C)[sP].[LR](A)[sP].[dR](T) | GATGATG | 18 |
| CATCCAT | | [sP].[dR](C)[sP].[LR](A)[sP].[dR](T)[sP].[dR]( | GATG | |
| CTAG-3′ | | C)[sP].[dR](C)[sP].[LR](A)[sP].[dR](T)[sP].[d | | |
| | | R](C)[sP].[dR](T)[P].[LR](A)[sP].[LR](G)}$$$ | | |
| | | $V2.0 | | |
| 5′- | SEQ ID | RNA1{[LR](A)[sP].[dR](T)[sP].[dR](C)[sP].[dR | GCTAGAT | SEQ ID NO: |
| ATCCATC | NO: 291 | ](C)[sP].[LR](A)[sP].[dR](T)[sP].[dR](C)[sP].[d | GGATGAT | 19 |
| ATCCATC | | R](A)[sP].[LR](T)[sP].[dR](C)[sP].[dR](C)[sP]. | GGAT | |
| TAGC-3′ | | [dR](A)[sP].[LR](T)[sP].[dR](C)[sP].[dR](T)[sP | | |
| | | ].[dR](A)[sP].[LR](G)[sP].[LR]([5meC])}$$$$V | | |
| | | 2.0 | | |
| 5′- | SEQ ID | RNA1{[LR](T)[sP].[dR](C)[sP].[dR](C)[sP].[L | GGCTAGA | SEQ ID NO: |
| TCCATCA | NO: 292 | R](A)[sP].[dR](T)[sP].[dR](C)[sP].[LR](A)[sP]. | TGGATGA | 20 |
| TCCATCT | | [dR](T)[sP].[dR](C)[sP].[dR](C)[sP].[LR](A)[s | TGGA | |
| AGCC-3′ | | P].[dR](T)[sP].[dR](C)[sP].[dR](T)[sP].[LR](A) | | |
| | | [sP].[dR](G)[sP].[LR]([5meC])[sP].[LR]([5me | | |
| | | C])}$$$$V2.0 | | |
| 5′- | SEQ ID | RNA1{[LR]([5meC])[sP].[dR](C)[sP].[LR](A)[s | TGGCTAG | SEQ ID NO: |
| CCATCAT | NO: 293 | P].[dR](T)[sP].[dR](C)[sP].[LR](A)[sP].[dR](T) | ATGGATG | 21 |
| CCATCTA | | [sP].[dR](C)[sP].[dR](C)[sP].[LR](A)[sP].[dR]( | ATGG | |
| GCCA-3′ | | T)[sP].[dR](C)[sP].[dR](T)[sP].[LR](A)[sP].[dR | | |
| | | ](G)[sP].[dR](C)[sP].[LR]([5meC])[sP].[LR](A) | | |
| | | }$$$$V2.0 | | |
| 5′- | SEQ ID | RNA1{[LR]([5meC])[sP].[dR](A)[sP].[dR](T)[s | GTGGCTA | SEQ ID NO: |
| CATCATC | NO: 294 | P].[dR](C)[sP].[LR](A)[sP].[dR](T)[sP].[dR](C) | GATGGAT | 22 |
| CATCTAG | | [sP].[dR](C)[sP].[LR](A)[sP].[dR](T)[sP].[dR]( | GATG | |
| CCAC-3′ | | C)[sP].[dR](T)[sP].[LR](A)[sP].[dR](G)[sP].[d | | |
| | | R](C)[sP].[dR](C)[sP].[LR](A)[sP].[LR]([5meC | | |
| | | ])}$$$$V2.0 | | |
| 5′- | SEQ ID | RNA1{[LR](A)[sP].[dR](T)[sP].[dR](C)[sP].[LR | CGTGGCT | SEQ ID NO: |
| ATCATCC | NO: 295 | ](A)[sP].[dR](T)[sP].[dR](C)[sP].[dR](C)[sP].[L | AGATGGA | 23 |
| ATCTAGC | | R](A)[sP].[dR](T)[sP].[dR](C)[sP].[dR](T)[sP].[ | TGAT | |
| CACG-3′ | | LR](A)[sP].[dR](G)[sP].[dR](C)[sP].[dR](C)[sP | | |
| | | ].[LR](A)[sP].[LR]([5meC])[sP].[LR](G)}$$$$V | | |
| | | 2.0 | | |
| 5′- | SEQ ID | RNA1{[LR](T)[sP].[dR](C)[sP].[LR](A)[sP].[dR | TCGTGGC | SEQ ID NO: |
| TCATCCA | NO: 296 | ](T)[sP].[dR](C)[sP].[dR](C)[sP].[LR](A)[sP].[d | TAGATGG | 24 |
| TCTAGCC | | R](T)[sP].[dR](C)[sP].[dR](T)[sP].[LR](A)[sP].[ | ATGA | |
| ACGA-3′ | | dR](G)[sP].[dR](C)[sP].[dR](C)[P].[LR](A)[sP | | |
| | | ].[dR](C)[sP].[LR](G)[sP].[LR](A)}$$$$V2.0 | | |
| 5′- | SEQ ID | RNA1{[LR]([5meC])[sP].[dR](A)[sP].[LR](T)[s | TTCGTGG | SEQ ID NO: |
| CATCCAT | NO: 297 | P].[dR](C)[sP].[dR](C)[sP].[LR](A)[sP].[dR](T) | CTAGATG | 25 |
| CTAGCCA | | [sP].[dR](C)[sP].[dR](T)[sP].[LR](A)[sP].[dR]( | GATG | |
| CGAA-3′ | | G)[sP].[dR](C)[sP].[dR](C)[sP].[LR](A)[sP].[d | | |
| | | R]([5meC])[sP].[dR](G)[sP].[LR](A)[sP].[LR]( | | |
| | | A)}$$$$V2.0 | | |
| 5′- | SEQ ID | RNA1{[LR](A)[sP].[dR](T)[sP].[dR](C)[sP].[dR | ATTCGTG | SEQ ID NO: |
| ATCCATC | NO: 298 | ](C)[sP].[LR](A)[sP].[dR](T)[sP].[dR](C)[sP].[d | GCTAGAT | 26 |
| TAGCCAC | | R](T)[sP].[LR](A)[sP].[dR](G)[sP].[dR](C)[sP]. | GGAT | |
| GAAT-3′ | | [dR](C)[sP].[LR](A)[sP].[dR]([5meC])[sP].[dR] | | |
| | | (G)[sP].[dR](A)[sP].[LR](A)[sP].[LR](T)}$$$$ | | |
| | | V2.0 | | |
| 5′- | SEQ ID | RNA1{[LR](T)[sP].[dR](C)[P].[dR](C)[sP].[L | GATTCGT | SEQ ID NO: |
| TCCATCT | NO: 299 | R](A)[sP].[dR](T)[sP].[dR](C)[sP].[dR](T)[sP].[ | GGCTAGA | 27 |
| AGCCACG | | LR](A)[sP].[dR](G)[sP].[dR](C)[sP].[dR](C)[sP | TGGA | |
| AATC-3′ | | ].[LR](A)[sP].[dR](C)[sP].[LR](G)[sP].[dR](A)[ | | |
| | | sP].[dR](A)[sP].[LR](T)[sP].[LR]([5meC])}$$$ | | |
| | | $V2.0 | | |
| 5′- | SEQ ID | RNA1{[LR]([5meC])[sP].[dR](C)[sP].[LR](A)[s | AGATTCG | SEQ ID NO: |
| CCATCTA | NO: 300 | P].[dR](T)[sP].[dR](C)[P].[dR](T)[sP].[LR](A) | TGGCTAG | 28 |
| GCCACGA | | [sP].[dR](G)[sP].[dR](C)[sP].[dR](C)[sP].[LR]( | ATGG | |
| ATCT-3′ | | A)[sP].[dR](C)[sP].[LR](G)[sP].[dR](A)[sP].[d | | |
| | | R](A)[sP].[dR](T)[sP].[LR]([5meC])[sP].[LR](T | | |
| | | )}$$$$V2.0 | | |
| 5′- | SEQ ID | RNA1{[LR]([5meC])[P].[dR](A)[sP].[LR](T)[s | TAGATTC | SEQ ID NO: |
| CATCTAG | NO: 301 | P].[dR](C)[sP].[dR](T)[sP].[LR](A)[sP].[dR](G) | GTGGCTA | 29 |
| CCACGAA | | [sP].[dR](C)[sP].[dR](C)[sP].[LR](A)[sP].[dR]([ | GATG | |
| TCTA-3′ | | 5meC])[sP].[dR](G)[sP].[dR](A)[P].[LR](A)[s | | |
| | | P].[dR](T)[sP].[dR](C)[sP].[LR](T)[sP].[LR](A) | | |
| | | }$$$$V2.0 | | |
| 5′- | SEQ ID | RNA1{[LR](A)[sP].[dR](T)[sP].[dR](C)[sP].[dR | GTAGATT | SEQ ID NO: |
| ATCTAGC | NO: 302 | ](T)[sP].[LR](A)[sP].[dR](G)[sP].[dR](C)[sP].[L | CGTGGCT | 30 |
| CACGAAT | | R]([5meC])[sP].[dR](A)[sP].[LR]([5meC])[sP].[ | AGAT | |
| CTAC-3′ | | dR](G)[sP].[dR](A)[sP].[LR](A)[sP].[dR](T)[sP | | |
| | | ].[dR](C)[sP].[dR](T)[sP].[LR](A)[sP].[LR]([5m | | |
| | | eC])}$$$$V2.0 | | |
| 5′- | SEQ ID | RNA1{[LR]([5meC])[sP].[dR](T)[sP].[LR](A)[s | GGGTAGA | SEQ ID NO: |
| CTAGCCA | NO: 303 | P].[dR](G)[sP].[dR](C)[sP].[dR](C)[sP].[LR](A | TTCGTGG | 31 |
| CGAATCT | | )[sP].[dR]([5meC])[sP].[dR](G)[sP].[dR](A)[sP | CTAG | |
| ACCC-3′ | | ].[LR](A)[sP].[dR](T)[sP].[dR](C)[sP].[dR](T)[s | | |
| | | P].[LR](A)[sP].[dR](C)[sP].[LR]([5meC])[sP].[ | | |
| | | LR]([5meC])}$$$$V2.0 | | |
| 5′- | SEQ ID | RNA1{[LR](T)[sP].[dR](A)[sP].[dR](G)[sP].[d | TGGGTAG | SEQ ID NO: |
| TAGCCAC | NO: 304 | R](C)[sP].[LR]([5meC])[sP].[dR](A)[sP].[dR]( | ATTCGTG | 32 |
| GAATCTA | | C)[sP].[LR](G)[sP].[dR](A)[P].[LR](A)[sP].[d | GCTA | |
| CCCA-3′ | | R](T)[sP].[dR](C)[sP].[dR](T)[sP].[LR](A)[sP].[ | | |
| | | dR](C)[sP].[dR](C)[sP].[LR]([5meC])[sP].[LR]( | | |
| | | A)}$$$$V2.0 | | |
| 5′- | SEQ ID | RNA1{[LR](A)[sP].[dR](G)[sP].[dR](C)[sP].[d | GTGGGTA | SEQ ID NO: |
| AGCCACG | NO: 305 | R](C)[sP].[LR](A)[sP].[dR]([5meC])[sP].[dR]( | GATTCGT | 33 |
| AATCTAC | | G)[sP].[dR](A)[sP].[LR](A)[sP].[dR](T)[sP].[d | GGCT | |
| CCAC-3′ | | R](C)[sP].[dR](T)[sP].[LR](A)[sP].[dR](C)[sP]. | | |
| | | [dR](C)[sP].[dR](C)[sP].[LR](A)[sP].[LR]([5me | | |
| | | C])}$$$$V2.0 | | |
| 5′- | SEQ ID | RNA1{[LR](G)[sP].[dR](C)[sP].[dR](C)[sP].[L | GGTGGGT | SEQ ID NO: |
| GCCACGA | NO: 306 | R](A)[sP].[dR]([5meC])[sP].[dR](G)[sP].[dR]( | AGATTCG | 34 |
| ATCTACC | | A)[sP].[LR](A)[sP].[dR](T)[sP].[dR](C)[sP].[dR | TGGC | |
| CACC-3′ | | ](T)[sP].[LR](A)[sP].[dR](C)[sP].[dR](C)[sP].[d | | |
| | | R](C)[sP].[LR](A)[sP].[LR]([5meC])[sP].[LR]([ | | |
| | | 5meC])}$$$$V2.0 | | |
| 5′- | SEQ ID | RNA1{[LR]([5meC])[sP].[dR](C)[sP].[LR](A)[s | TGGTGGG | SEQ ID NO: |
| CCACGAA | NO: 307 | P].[dR]([5meC])[sP].[dR](G)[sP].[dR](A)[sP].[ | TAGATTC | 35 |
| TCTACCC | | LR](A)[P].[dR](T)[sP].[dR](C)[sP].[dR](T)[sP] | GTGG | |
| ACCA-3′ | | [LR](A)[sP].[dR](C)[sP].[dR](C)[sP].[dR](C)[s | | |
| | | P].[LR](A)[sP].[dR](C)[P].[LR]([5meC])[sP].[ | | |
| | | LR](A)}$$$$V2.0 | | |
| 5′- | SEQ ID | RNA1{[LR]([5meC])[sP].[dR](A)[sP].[dR](C)[s | TTGGTGG | SEQ ID NO: |
| CACGAAT | NO: 308 | P].[LR](G)[P].[dR](A)[sP].[LR](A)[sP].[dR](T) | GTAGATT | 36 |
| CTACCCA | | [sP].[dR](C)[sP].[dR](T)[sP].[LR](A)[sP].[dR]( | CGTG | |
| CCAA-3′ | | C)[sP].[dR](C)[sP].[dR](C)[sP].[LR](A)[sP].[d | | |
| | | R](C)[sP].[dR](C)[sP].[LR](A)[sP].[LR](A)}$$$ | | |
| | | $V2.0 | | |
| 5′- | SEQ ID | RNA1{[LR](A)[sP].[dR]([5meC])[sP].[dR](G)[s | GTTGGTG | SEQ ID NO: |
| ACGAATC | NO: 309 | P].[dR](A)[sP].[LR](A)[sP].[dR](T)[sP].[dR](C) | GGTAGAT | 37 |
| TACCCAC | | [sP].[dR](T)[sP].[LR](A)[sP].[dR](C)[sP].[dR]( | TCGT | |
| CAAC-3′ | | C)[sP].[dR](C)[sP].[LR](A)[sP].[dR](C)[sP].[d | | |
| | | R](C)[sP].[dR](A)[sP].[LR](A)[sP].[LR]([5meC | | |
| | | ])}$$$$V2.0 | | |
| 5′- | SEQ ID | RNA1{[LR](A)[sP].[dR](A)[sP].[LR](T)[sP].[dR | TGAGTTG | SEQ ID NO: |
| AATCTAC | NO: 310 | ](C)[sP].[dR](T)[sP].[LR](A)[sP].[dR](C)[sP].[d | GTGGGTA | 38 |
| CCACCAA | | R](C)[sP].[dR](C)[P].[LR](A)[sP].[dR](C)[sP]. | GATT | |
| CTCA-3′ | | [dR](C)[sP].[LR](A)[P].[dR](A)[sP].[dR](C)[s | | |
| | | P].[dR](T)[sP].[LR]([5meC])[sP].[LR](A)}$$$$ | | |
| | | V2.0 | | |
| 5′- | SEQ ID | RNA1{[LR](A)[sP].[dR](T)[sP].[dR](C)[sP].[dR | ATGAGTT | SEQ ID NO: |
| ATCTACC | NO: 311 | ](T)[sP].[LR](A)[sP].[dR](C)[sP].[dR](C)[sP].[d | GGTGGGT | 39 |
| CACCAAC | | R](C)[P].[LR](A)[sP].[dR](C)[sP].[dR](C)[sP]. | AGAT | |
| TCAT-3′ | | [dR](A)[sP].[LR](A)[sP].[dR](C)[sP].[dR](T)[s | | |
| | | P].[dR](C)[sP].[LR](A)[sP].[LR](T)}$$$$V2.0 | | |
| 5′- | SEQ ID | RNA1{[LR](T)[sP].[dR](C)[sP].[dR](T)[sP].[LR | GATGAGT | SEQ ID NO: |
| TCTACCC | NO: 312 | ](A)[sP].[dR](C)[sP].[dR](C)[sP].[dR](C)[P].[ | TGGTGGG | 40 |
| ACCAACT | | LR](A)[sP].[dR](C)[P].[dR](C)[sP].[LR](A)[sP | TAGA | |
| CATC-3′ | | ].[dR](A)[sP].[dR](C)[sP].[LR](T)[sP].[dR](C)[s | | |
| | | P].[dR](A)[sP].[LR](T)[sP].[LR]([5meC])}$$$$ | | |
| | | V2.0 | | |
| 5′- | SEQ ID | RNA1{[LR]([5meC])[sP].[dR](T)[sP].[LR](A)[s | GGATGAG | SEQ ID NO: |
| CTACCCA | NO: 313 | P].[dR](C)[sP].[dR](C)[sP].[dR](C)[sP].[LR](A) | TTGGTGG | 41 |
| CCAACTC | | [sP].[dR](C)[sP].[dR](C)[sP].[dR](A)[sP].[LR]( | GTAG | |
| ATCC-3′ | | A)[sP].[dR](C)[sP].[dR](T)[sP].[dR](C)[sP].[L | | |
| | | R](A)[sP].[dR](T)[sP].[LR]([5meC])[sP].[LR]([ | | |
| | | 5meC])}$$$$V2.0 | | |
| 5′- | SEQ ID | RNA1{[LR](T)[sP].[dR](A)[sP].[dR](C)[sP].[dR | TGGATGA | SEQ ID NO: |
| TACCCAC | NO: 314 | ](C)[sP].[LR]([5meC])[sP].[dR](A)[sP].[dR](C)[ | GTTGGTG | 42 |
| CAACTCA | | sP].[dR](C)[sP].[LR](A)[sP].[dR](A)[sP].[dR]( | GGTA | |
| TCCA-3′ | | C)[sP].[LR](T)[sP].[dR](C)[P].[LR](A)[sP].[d | | |
| | | R](T)[sP].[dR](C)[sP].[LR]([5meC])[sP].[LR](A | | |
| | | )}$$$$V2.0 | | |
| 5′- | SEQ ID | RNA1{[LR](A)[sP].[dR](C)[sP].[dR](C)[sP].[d | ATGGATG | SEQ ID NO: |
| ACCCACC | NO: 315 | R](C)[sP].[LR](A)[sP].[dR](C)[sP].[dR](C)[sP]. | AGTTGGT | 43 |
| AACTCAT | | [dR](A)[sP].[LR](A)[sP].[dR](C)[sP].[dR](T)[s | GGGT | |
| CCAT-3′ | | P].[dR](C)[sP].[LR](A)[sP].[dR](T)[sP].[dR](C) | | |
| | | [sP].[dR](C)[sP].[LR](A)[sP].[LR](T)}$$$$V2. | | |
| | | 0 | | |
| 5′- | SEQ ID | RNA1{[LR]([5meC])[sP].[dR](C)[sP].[dR](C)[s | GATGGAT | SEQ ID NO: |
| CCCACCA | NO: 316 | P].[LR](A)[sP].[dR](C)[sP].[dR](C)[sP].[dR](A) | GAGTTGG | 44 |
| ACTCATC | | [sP].[LR](A)[sP].[dR](C)[sP].[dR](T)[sP].[dR]( | TGGG | |
| CATC-3′ | | C)[sP].[LR](A)[sP].[dR](T)[sP].[dR](C)[sP].[L | | |
| | | R]([5meC])[sP].[dR](A)[sP].[LR](T)[sP].[LR]([ | | |
| | | 5meC])}$$$$V2.0 | | |
| 5′- | SEQ ID | RNA1{[LR]([5meC])[sP].[dR](C)[sP].[LR](A)[s | AGATGGA | SEQ ID NO: |
| CCACCAA | NO: 317 | P].[dR](C)[sP].[dR](C)[sP].[dR](A)[sP].[LR](A) | TGAGTTG | 45 |
| CTCATCC | | [sP].[dR](C)[sP].[dR](T)[sP].[dR](C)[sP].[LR]( | GTGG | |
| ATCT-3′ | | A)[sP].[dR](T)[sP].[dR](C)[sP].[dR](C)[sP].[L | | |
| | | R](A)[sP].[dR](T)[sP].[LR]([5meC])[P].[LR](T | | |
| | | )}$$$$V2.0 | | |
| 5′- | SEQ ID | RNA1{[LR]([5meC])[sP].[dR](A)[sP].[dR](C)[s | TAGATGG | SEQ ID NO: |
| CACCAAC | NO: 318 | P].[dR](C)[sP].[LR](A)[sP].[dR](A)[sP].[dR](C) | ATGAGTT | 46 |
| TCATCCA | | [sP].[LR](T)[sP].[dR](C)[sP].[LR](A)[sP].[dR]( | GGTG | |
| TCTA-3′ | | T)[sP].[dR](C)[sP].[dR](C)[sP].[LR](A)[sP].[d | | |
| | | R](T)[sP].[dR](C)[sP].[LR](T)[sP].[LR](A)}$$$ | | |
| | | $V2.0 | | |
| 5′- | SEQ ID | RNA1{[LR](A)[sP].[dR](C)[sP].[LR]([5meC])[s | ATAGATG | SEQ ID NO: |
| ACCAACT | NO: 319 | P].[dR](A)[sP].[LR](A)[sP].[dR](C)[sP].[dR](T) | GATGAGT | 47 |
| CATCCAT | | [sP].[dR](C)[sP].[LR](A)[sP].[dR](T)[sP].[dR]( | TGGT | |
| CTAT-3′ | | C)[sP].[dR](C)[sP].[LR](A)[sP].[dR](T)[sP].[d | | |
| | | R](C)[sP].[dR](T)[sP].[LR](A)[sP].[LR](T)}$$$ | | |
| | | $V2.0 | | |
| 5′- | SEQ ID | RNA1{[LR]([5meC])[P].[dR](C)[sP].[dR](A)[s | GATAGAT | SEQ ID NO: |
| CCAACTC | NO: 320 | P].[LR](A)[sP].[dR](C)[sP].[dR](T)[sP].[dR](C) | GGATGAG | 48 |
| ATCCATC | | [sP].[LR](A)[sP].[dR](T)[sP].[dR](C)[sP].[dR]( | TTGG | |
| TATC-3′ | | C)[sP].[LR](A)[sP].[dR](T)[sP].[dR](C)[sP].[d | | |
| | | R](T)[sP].[LR](A)[sP].[LR](T)[sP].[LR]([5meC] | | |
| | | )}$$$$V2.0 | | |
| 5′- | SEQ ID | RNA1{[LR]([5meC])[sP].[dR](A)[sP].[LR](A)[s | GGATAGA | SEQ ID NO: |
| CAACTCA | NO: 321 | P].[dR](C)[sP].[dR](T)[sP].[dR](C)[sP].[LR](A) | TGGATGA | 49 |
| TCCATCT | | [sP].[dR](T)[sP].[dR](C)[sP].[dR](C)[sP].[LR]( | GTTG | |
| ATCC-3′ | | A)[sP].[dR](T)[sP].[dR](C)[sP].[dR](T)[sP].[LR | | |
| | | ](A)[sP].[dR](T)[sP].[LR]([5meC])[sP].[LR]([5 | | |
| | | meC])}$$$$V2.0 | | |
| 5′- | SEQ ID | RNA1{[LR](A)[sP].[dR](A)[sP].[dR](C)[sP].[L | TGGATAG | SEQ ID NO: |
| AACTCAT | NO: 322 | R](T)[sP].[dR](C)[sP].[LR](A)[sP].[dR](T)[sP].[ | ATGGATG | 50 |
| CCATCTA | | dR](C)[sP].[dR](C)[sP].[LR](A)[sP].[dR](T)[sP | AGTT | |
| TCCA-3′ | | ].[dR](C)[sP].[dR](T)[sP].[LR](A)[sP].[dR](T)[s | | |
| | | P].[dR](C)[sP].[LR]([5meC])[sP].[LR](A)}$$$$ | | |
| | | V2.0 | | |
| 5′- | SEQ ID | RNA1{[LR](A)[sP].[dR](C)[sP].[dR](T)[sP].[dR | ATGGATA | SEQ ID NO: |
| ACTCATC | NO: 323 | ](C)[sP].[LR](A)[sP].[dR](T)[sP].[dR](C)[sP].[d | GATGGAT | 51 |
| CATCTAT | | R](C)[sP].[LR](A)[sP].[dR](T)[sP].[dR](C)[sP]. | GAGT | |
| CCAT-3′ | | [dR](T)[P].[LR](A)[sP].[dR](T)[sP].[dR](C)[sP | | |
| | | ].[dR](C)[sP].[LR](A)[sP].[LR](T)}$$$$V2.0 | | |
| 5′- | SEQ ID | RNA1{[LR]([5meC])[sP].[dR](T)[sP].[dR](C)[s | GATGGAT | SEQ ID NO: |
| CTCATCC | NO: 324 | P].[LR](A)[sP].[dR](T)[sP].[dR](C)[sP].[dR](C) | AGATGGA | 52 |
| ATCTATC | | [sP].[LR](A)[sP].[dR](T)[sP].[dR](C)[sP].[dR]( | TGAG | |
| CATC-3′ | | T)[sP].[LR](A)[sP].[dR](T)[sP].[dR](C)[sP].[LR | | |
| | | ]([5meC])[sP].[dR](A)[sP].[LR](T)[sP].[LR]([5 | | |
| | | meC])}$$$$V2.0 | | |
| 5′- | SEQ ID | RNA1{[LR](T)[sP].[dR](C)[sP].[LR](A)[sP].[dR | GGATGGA | SEQ ID NO: |
| TCATCCA | NO: 325 | ](T)[sP].[dR](C)[sP].[dR](C)[sP].[LR](A)[sP].[d | TAGATGG | 53 |
| TCTATCC | | R](T)[sP].[dR](C)[sP].[dR](T)[sP].[LR](A)[sP].[ | ATGA | |
| ATCC-3′ | | dR](T)[sP].[dR](C)[sP].[dR](C)[sP].[LR](A)[sP | | |
| | | ].[dR](T)[sP].[LR]([5meC])[sP].[LR]([5meC])}$ | | |
| | | $$$V2.0 | | |
| 5′- | SEQ ID | RNA1{[LR]([5meC])[sP].[dR](A)[sP].[LR](T)[s | TGGATGG | SEQ ID NO: |
| CATCCAT | NO: 326 | P].[dR](C)[sP].[dR](C)[sP].[LR](A)[sP].[dR](T) | ATAGATG | 54 |
| CTATCCA | | [sP].[dR](C)[sP].[dR](T)[sP].[LR](A)[sP].[dR]( | GATG | |
| TCCA-3′ | | T)[sP].[dR](C)[sP].[dR](C)[sP].[LR](A)[sP].[d | | |
| | | R](T)[sP].[dR](C)[sP].[LR]([5meC])[sP].[LR](A | | |
| | | )}$$$$V2.0 | | |
| 5′- | SEQ ID | RNA1{[LR](A)[sP].[dR](T)[sP].[dR](C)[sP].[dR | ATGGATG | SEQ ID NO: |
| ATCCATC | NO: 327 | ](C)[sP].[LR](A)[sP].[dR](T)[sP].[dR](C)[sP].[d | GATAGAT | 55 |
| TATCCAT | | R](T)[sP].[LR](A)[sP].[dR](T)[sP].[dR](C)[sP].[ | GGAT | |
| CCAT-3′ | | dR](C)[sP].[LR](A)[sP].[dR](T)[sP].[dR](C)[sP | | |
| | | ].[dR](C)[aP].[LR](A)[sP].[LR](T)$$$$V2.0 | | |
| 5′- | SEQ ID | RNA1{[LR](T)[sP].[dR](C)[sP].[dR](C)[sP].[L | CATGGAT | SEQ ID NO: |
| TCCATCT | NO: 328 | R](A)[sP].[dR](T)[sP].[dR](C)[sP].[dR](T)[sP].[ | GGATAGA | 56 |
| ATCCATC | | LR](A)[sP].[dR](T)[sP].[dR](C)[sP].[dR](C)[sP | TGGA | |
| CATG-3′ | | ].[LR](A)[sP].[dR](T)[sP].[dR](C)[sP].[LR]([5m | | |
| | | eC])[sP].[dR](A)[sP].[LR](T)[sP].[LR](G)}$$$$ | | |
| | | V2.0 | | |
| 5′- | SEQ ID | RNA1{[LR]([5meC])[sP].[dR](C)[sP].[LR](A)[s | ACATGGA | SEQ ID NO: |
| CCATCTA | NO: 329 | P].[dR](T)[sP].[dR](C)[sP].[dR](T)[sP].[LR](A) | TGGATAG | 57 |
| TCCATCC | | [sP].[dR](T)[sP].[dR](C)[sP].[dR](C)[sP].[LR]( | ATGG | |
| ATGT-3′ | | A)[sP].[dR](T)[sP].[dR](C)[sP].[dR](C)[sP].[L | | |
| | | R](A)[sP].[dR](T)[sP].[LR](G)[sP].[LR](T)}$$$ | | |
| | | $V2.0 | | |
| 5′- | SEQ ID | RNA1{[LR]([5meC])[sP].[dR](A)[sP].[LR](T)[s | TACATGG | SEQ ID NO: |
| CATCTAT | NO: 330 | P].[dR](C)[sP].[dR](T)[sP].[LR](A)[sP].[dR](T) | ATGGATA | 58 |
| CCATCCA | | [sP].[dR](C)[sP].[dR](C)[sP].[LR](A)[sP].[dR]( | GATG | |
| TGTA-3′ | | T)[sP].[dR](C)[sP].[dR](C)[sP].[LR](A)[sP].[d | | |
| | | R](T)[sP].[dR](G)[sP].[LR](T)[sP].[LR](A)}$$$ | | |
| | | $V2.0 | | |
| 5′- | SEQ ID | RNA1{[LR](A)[sP].[dR](T)[sP].[LR]([5meC])[s | GTACATG | SEQ ID NO: |
| ATCTATC | NO: 331 | P].[dR](T)[sP].[dR](A)[sP].[LR](T)[sP].[dR](C) | GATGGAT | 59 |
| CATCCAT | | [sP].[dR](C)[sP].[LR](A)[sP].[dR](T)[sP].[dR]( | AGAT | |
| GTAC-3′ | | C)[sP].[dR](C)[sP].[LR](A)[sP].[dR](T)[sP].[d | | |
| | | R](G)[sP].[dR](T)[sP].[LR](A)[sP].[LR]([5meC | | |
| | | ])}$$$$V2.0 | | |
| 5′- | SEQ ID | RNA1{[LR](T)[sP].[dR](C)[sP].[dR](T)[sP].[LR | AGTACAT | SEQ ID NO: |
| TCTATCC | NO: 332 | ](A)[sP].[dR](T)[sP].[dR](C)[sP].[dR](C)[sP].[L | GGATGGA | 60 |
| ATCCATG | | R](A)[sP].[dR](T)[sP].[dR](C)[sP].[dR](C)[sP]. | TAGA | |
| TACT-3′ | | [LR](A)[sP].[dR](T)[sP].[LR](G)[sP].[dR](T)[s | | |
| | | P].[dR](A)[sP].[LR]([5meC])[P].[LR](T)}$$$$ | | |
| | | V2.0 | | |
| 5′- | SEQ ID | RNA1{[LR]([5meC])[sP].[dR](T)[sP].[LR](A)[s | GAGTACA | SEQ ID NO: |
| CTATCCA | NO: 333 | P].[dR](T)[sP].[dR](C)[sP].[dR](C)[sP].[LR](A) | TGGATGG | 61 |
| TCCATGT | | [sP].[dR](T)[sP].[dR](C)[sP].[dR](C)[sP].[LR]( | ATAG | |
| ACTC-3′ | | A)[sP].[dR](T)[sP].[dR](G)[sP].[dR](T)[sP].[LR | | |
| | | ](A)[sP].[dR](C)[sP].[LR](T)[sP].[LR]([5meC])} | | |
| | | $$$$V2.0 | | |
| 5′- | SEQ ID | RNA1{[LR](T)[P].[dR](A)[sP].[LR](T)[sP].[dR | TGAGTAC | SEQ ID NO: |
| TATCCAT | NO: 334 | ](C)[sP].[dR](C)[sP].[LR](A)[sP].[dR](T)[sP].[d | ATGGATG | 62 |
| CCATGTA | | R](C)[sP].[dR](C)[sP].[LR](A)[sP].[dR](T)[sP]. | GATA | |
| CTCA-3′ | | [dR](G)[sP].[dR](T)[sP].[LR](A)[sP].[dR](C)[s | | |
| | | P].[dR](T)[sP].[LR]([5meC])[sP].[LR](A)}$$$$ | | |
| | | V2.0 | | |
| 5′- | SEQ ID | RNA1{[LR](A)[sP].[dR](T)[sP].[dR](C)[sP].[dR | GTGAGTA | SEQ ID NO: |
| ATCCATC | NO: 335 | ](C)[sP].[LR](A)[sP].[dR](T)[sP].[dR](C)[sP].[d | CATGGAT | 63 |
| CATGTAC | | R](C)[sP].[LR](A)[sP].[dR](T)[sP].[dR](G)[sP]. | GGAT | |
| TCAC-3′ | | [dR](T)[sP].[LR](A)[sP].[dR](C)[sP].[dR](T)[sP | | |
| | | ].[dR](C)[sP].[LR](A)[sP].[LR]([5meC])}$$$$V | | |
| | | 2.0 | | |
| 5′- | SEQ ID | RNA1{[LR](T)[sP].[dR](C)[sP].[dR](C)[sP].[L | GGTGAGT | SEQ ID NO: |
| TCCATCC | NO: 336 | R](A)[sP].[dR](T)[sP].[dR](C)[sP].[dR](C)[sP]. | ACATGGA | 64 |
| ATGTACT | | [LR](A)[sP].[dR](T)[sP].[dR](G)[sP].[dR](T)[s | TGGA | |
| CACC-3′ | | P].[LR](A)[sP].[dR](C)[sP].[dR](T)[sP].[dR](C) | | |
| | | [sP].[LR](A)[sP].[LR]([5meC])[sP].[LR]([5meC | | |
| | | ])}$$$$V2.0 | | |
| 5′- | SEQ ID | RNA1{[LR]([5meC])[sP].[dR](C)[sP].[LR](A)[s | GGGTGAG | SEQ ID NO: |
| CCATCCA | NO: 337 | P].[dR](T)[sP].[dR](C)[sP].[dR](C)[sP].[LR](A) | TACATGG | 65 |
| TGTACTC | | [sP].[dR](T)[sP].[dR](G)[sP].[dR](T)[sP].[LR]( | ATGG | |
| ACCC-3′ | | A)[sP].[dR](C)[sP].[dR](T)[sP].[dR](C)[sP].[L | | |
| | | R](A)[sP].[dR](C)[sP].[LR]([5meC])[sP].[LR]([ | | |
| | | 5meC])}$$$$V2.0 | | |
| 5′- | SEQ ID | RNA1{[LR]([5meC])[sP].[dR](A)[sP].[LR](T)[s | TGGGTGA | SEQ ID NO: |
| CATCCAT | NO: 338 | P].[dR](C)[sP].[dR](C)[sP].[LR](A)[sP].[dR](T) | GTACATG | 66 |
| GTACTCA | | [sP].[dR](G)[sP].[dR](T)[sP].[LR](A)[sP].[dR]( | GATG | |
| CCCA-3′ | | C)[sP].[dR](T)[sP].[dR](C)[sP].[LR](A)[sP].[d | | |
| | | R](C)[sP].[dR](C)[sP].[LR]([5meC])[sP].[LR]( | | |
| | | A)}$$$$V2.0 | | |
| 5′- | SEQ ID | RNA1{[LR](A)[sP].[dR](T)[sP].[dR](C)[sP].[dR | ATGGGTG | SEQ ID NO: |
| ATCCATG | NO: 339 | ](C)[sP].[LR](A)[sP].[dR](T)[sP].[dR](G)[sP].[d | AGTACAT | 67 |
| TACTCAC | | R](T)[sP].[LR](A)[sP].[dR](C)[sP].[dR](T)[sP].[ | GGAT | |
| CCAT-3′ | | dR](C)[sP].[LR](A)[sP].[dR](C)[sP].[dR](C)[sP | | |
| | | ].[dR](C)[sP].[LR](A)[sP].[LR](T)}$$$$V2.0 | | |
| 5′- | SEQ ID | RNA1{[LR](T)[sP].[dR](C)[sP].[dR](C)[sP].[L | GATGGGT | SEQ ID NO: |
| TCCATGT | NO: 340 | R](A)[sP].[dR](T)[sP].[dR](G)[sP].[dR](T)[sP]. | GAGTACA | 68 |
| ACTCACC | | [LR](A)[sP].[dR](C)[sP].[dR](T)[sP].[dR](C)[s | TGGA | |
| CATC-3′ | | P].[LR](A)[sP].[dR](C)[sP].[dR](C)[sP].[LR]([5 | | |
| | | meC])[sP].[dR](A)[sP].[LR](T)[sP].[LR]([5me | | |
| | | C])}$$$$V2.0 | | |
| 5′- | SEQ ID | RNA1{[LR]([5meC])[sP].[dR](C)[sP].[LR](A)[s | AGATGGG | SEQ ID NO: |
| CCATGTA | NO: 341 | P].[dR](T)[sP].[dR](G)[sP].[dR](T)[sP].[LR](A) | TGAGTAC | 69 |
| CTCACCC | | [sP].[dR](C)[sP].[dR](T)[sP].[dR](C)[sP].[LR]( | ATGG | |
| ATCT-3′ | | A)[sP].[dR](C)[sP].[dR](C)[sP].[dR](C)[sP].[L | | |
| | | R](A)[sP].[dR](T)[sP].[LR]([5meC])[sP].[LR](T | | |
| | | )}$$$$V2.0 | | |
| 5′- | SEQ ID | RNA1{[LR]([5meC])[sP].[dR](A)[sP].[dR](T)[s | GAGATGG | SEQ ID NO: |
| CATGTAC | NO: 342 | P].[LR](G)[sP].[dR](T)[sP].[LR](A)[P].[dR](C) | GTGAGTA | 70 |
| TCACCCA | | [sP].[dR](T)[sP].[dR](C)[sP].[LR](A)[sP].[dR]( | CATG | |
| TCTC-3′ | | C)[sP].[dR](C)[sP].[dR](C)[sP].[LR](A)[sP].[d | | |
| | | R](T)[sP].[dR](C)[sP].[LR](T)[sP].[LR]([5meC] | | |
| | | )}$$$$V2.0 | | |
| 5′- | SEQ ID | RNA1{[LR](A)[sP].[dR](T)[sP].[dR](G)[sP].[d | AGAGATG | SEQ ID NO: |
| ATGTACT | NO: 343 | R](T)[sP].[LR](A)[sP].[dR](C)[sP].[dR](T)[sP].[ | GGTGAGT | 71 |
| CACCCAT | | dR](C)[sP].[LR](A)[sP].[dR](C)[sP].[dR](C)[sP | ACAT | |
| CTCT-3′ | | ].[dR](C)[sP].[LR](A)[sP].[dR](T)[sP].[dR](C)[s | | |
| | | P].[dR](T)[sP].[LR]([5meC])[sP].[LR](T)}$$$$ | | |
| | | V2.0 | | |
| 5′- | SEQ ID | RNA1{[LR](T)[sP].[dR](G)[sP].[dR](T)[sP].[LR | GAGAGAT | SEQ ID NO: |
| TGTACTC | NO: 344 | ](A)[sP].[dR](C)[sP].[dR](T)[sP].[dR](C)[sP].[L | GGGTGAG | 72 |
| ACCCATC | | R](A)[sP].[dR](C)[sP].[dR](C)[sP].[dR](C)[sP]. | TACA | |
| TCTC-3′ | | [LR](A)[sP].[dR](T)[sP].[dR](C)[sP].[LR](T)[sP | | |
| | | ].[dR](C)[sP].[LR](T)[sP].[LR]([5meC])}$$$$V | | |
| | | 2.0 | | |
| 5′- | SEQ ID | RNA1{[LR](G)[sP].[dR](T)[sP].[LR](A)[sP].[d | GGAGAGA | SEQ ID NO: |
| GTACTCA | NO: 345 | R](C)[sP].[dR](T)[sP].[dR](C)[sP].[LR](A)[sP]. | TGGGTGA | 73 |
| CCCATCT | | [dR](C)[sP].[dR](C)[sP].[dR](C)[sP].[LR](A)[s | GTAC | |
| CTCC-3′ | | P].[dR](T)[sP].[dR](C)[sP].[LR](T)[sP].[dR](C) | | |
| | | [sP].[dR](T)[sP].[LR]([5meC])[sP].[LR]([5meC | | |
| | | ])}$$$$V2.0 | | |
| 5′- | SEQ ID | RNA1{[LR](T)[sP].[dR](A)[sP].[dR](C)[sP].[LR | TGGAGAG | SEQ ID NO: |
| TACTCAC | NO: 346 | ](T)[sP].[dR](C)[sP].[LR](A)[sP].[dR](C)[sP].[d | ATGGGTG | 74 |
| CCATCTC | | R](C)[sP].[dR](C)[sP].[LR](A)[sP].[dR](T)[sP]. | AGTA | |
| TCCA-3′ | | [dR](C)[sP].[LR](T)[sP].[dR](C)[sP].[dR](T)[s | | |
| | | P].[dR](C)[sP].[LR]([5meC])[sP].[LR](A)}$$$$ | | |
| | | V2.0 | | |
| 5′- | SEQ ID | RNA1{[LR](A)[sP].[dR](C)[sP].[dR](T)[sP].[dR | ATGGAGA | SEQ ID NO: |
| ACTCACC | NO: 347 | ](C)[sP].[LR](A)[sP].[dR](C)[sP].[dR](C)[sP].[ | GATGGGT | 75 |
| CATCTCT | | dR](C)[sP].[LR](A)[sP].[dR](T)[sP].[dR](C)[sP | GAGT | |
| CCAT-3′ | | ].[dR](T)[sP].[LR]([5meC])[sP].[dR](T)[sP].[dR | | |
| | | ](C)[sP].[dR](C)[sP].[LR](A)[sP].[LR](T)}$$$$ | | |
| | | V2.0 | | |
| 5′- | SEQ ID | RNA1{[LR]([5meC])[sP].[dR](T)[sP].[dR](C)[s | GATGGAG | SEQ ID NO: |
| CTCACCC | NO: 348 | P].[LR](A)[sP].[dR](C)[sP].[dR](C)[sP].[dR](C) | AGATGGG | 76 |
| ATCTCTC | | [sP].[LR](A)[sP].[dR](T)[sP].[dR](C)[sP].[LR]( | TGAG | |
| CATC-3′ | | T)[sP].[dR](C)[sP].[dR](T)[sP].[dR](C)[sP].[LR | | |
| | | ]([5meC])[sP].[dR](A)[sP].[LR](T)[sP].[LR]([5 | | |
| | | meC])}$$$$V2.0 | | |
| 5′- | SEQ ID | RNA1{[LR]([5meC])[sP].[dR](A)[sP].[dR](C)[s | TGGATGG | SEQ ID NO: |
| CACCCAT | NO: 349 | P].[dR](C)[sP].[LR]([5meC])[sP].[dR](A)[sP].[ | AGAGATG | 77 |
| CTCTCCA | | LR](T)[sP].[dR](C)[sP].[dR](T)[sP].[dR](C)[sP | GGTG | |
| TCCA-3′ | | ].[LR](T)[sP].[dR](C)[sP].[dR](C)[sP].[LR](A)[s | | |
| | | P].[dR](T)[sP].[dR](C)[sP].[LR]([5meC])[sP].[ | | |
| | | LR](A)}$$$$V2.0 | | |
| 5′- | SEQ ID | RNA1{[LR](A)[sP].[dR](C)[sP].[dR](C)[sP].[d | ATGGATG | SEQ ID NO: |
| ACCCATC | NO: 350 | R](C)[sP].[LR](A)[sP].[dR](T)[sP].[dR](C)[sP]. | GAGAGAT | 78 |
| TCTCCAT | | [dR](T)[sP].[LR]([5meC])[sP].[dR](T)[sP].[dR] | GGGT | |
| CCAT-3′ | | (C)[sP].[dR](C)[sP].[LR](A)[sP].[dR](T)[sP].[d | | |
| | | R](C)[sP].[dR](C)[sP].[LR](A)[sP].[LR](T)}$$$ | | |
| | | $V2.0 | | |
| 5′- | SEQ ID | RNA1{[LR]([5meC])[sP].[dR](C)[sP].[dR](C)[s | CATGGAT | SEQ ID NO: |
| CCCATCT | NO: 351 | P].[dR](A)[sP].[LR](T)[sP].[dR](C)[sP].[dR](T) | GGAGAGA | 79 |
| CTCCATC | | [sP].[dR](C)[sP].[LR](T)[sP].[dR](C)[sP].[dR]( | TGGG | |
| CATG-3′ | | C)[sP].[dR](A)[sP].[LR](T)[sP].[dR](C)[sP].[d | | |
| | | R](C)[sP].[dR](A)[sP].[LR](T)[sP].[LR](G)}$$$ | | |
| | | $V2.0 | | |
| 5′- | SEQ ID | RNA1{[LR]([5meC])[sP].[dR](C)[sP].[LR](A)[s | GCATGGA | SEQ ID NO: |
| CCATCTC | NO: 352 | P].[dR](T)[sP].[dR](C)[sP].[dR](T)[sP].[LR]([5 | TGGAGAG | 80 |
| TCCATCC | | meC])[sP].[dR](T)[sP].[dR](C)[sP].[dR](C)[sP] | ATGG | |
| ATGC-3′ | | .[LR](A)[sP].[dR](T)[sP].[dR](C)[sP].[dR](C)[s | | |
| | | P].[LR](A)[sP].[LR](T)[sP].[dR]([In])[sP].[LR]([ | | |
| | | 5meC])}$$$$V2.0 | | |
| 5′- | SEQ ID | RNA1{[LR]([5meC])[sP].[dR](A)[sP].[LR](T)[s | AGCATGG | SEQ ID NO: |
| CATCTCT | NO: 353 | P].[dR](C)[sP].[dR](T)[sP].[dR](C)[sP].[LR](T) | ATGGAGA | 81 |
| CCATCCA | | [sP].[dR](C)[sP].[dR](C)[sP].[LR](A)[sP].[dR]( | GATG | |
| TGCT-3′ | | T)[sP].[dR](C)[sP].[dR](C)[sP].[LR](A)[sP].[d | | |
| | | R](T)[sP].[dR]([In])[sP].[LR]([5meC])[sP].[LR]( | | |
| | | T)}$$$$V2.0 | | |
| 5′- | SEQ ID | RNA1{[LR](A)[sP].[dR](T)[sP].[dR](C)[sP].[dR | AAGCATG | SEQ ID NO: |
| ATCTCTC | NO: 354 | ](T)[sP].[LR]([5meC])[sP].[dR](T)[sP].[dR](C)[ | GATGGAG | 82 |
| CATCCAT | | sP].[dR](C)[sP].[LR](A)[sP].[dR](T)[sP].[dR]( | AGAT | |
| GCTT-3′ | | C)[sP].[dR](C)[sP].[LR](A)[sP].[dR](T)[sP].[d | | |
| | | R]([In])[sP].[dR](C)[sP].[LR](T)[sP].[LR](T)}$$ | | |
| | | $$V2.0 | | |
| 5′- | SEQ ID | RNA1{[LR](T)[sP].[dR](C)[sP].[dR](T)[sP].[dR | AAAGCAT | SEQ ID NO: |
| TCTCTCC | NO: 355 | ](C)[sP].[LR](T)[sP].[dR](C)[sP].[dR](C)[sP].[L | GGATGGA | 83 |
| ATCCATG | | R](A)[sP].[dR](T)[sP].[dR](C)[sP].[dR](C)[sP]. | GAGA | |
| CTTT-3′ | | [LR](A)[sP].[dR](T)[sP].[dR]([In])[sP].[dR](C)[ | | |
| | | sP].[dR](T)[sP].[LR](T)[sP].[LR](T)$$$$V2.0 | | |
| 5′- | SEQ ID | RNA1{[LR]([5meC])[sP].[dR](T)[sP].[dR](C)[s | AAAAGCA | SEQ ID NO: |
| CTCTCCA | NO: 356 | P].[LR](T)[sP].[dR](C)[sP].[dR](C)[sP].[LR](A) | TGGATGG | 84 |
| TCCATGC | | [sP].[dR](T)[sP].[dR](C)[sP].[dR](C)[sP].[LR]( | AGAG | |
| TTTT-3′ | | A)[sP].[dR](T)[sP].[dR]([In])[sP].[dR](C)[sP].[L | | |
| | | R](T)[sP].[dR](T)[sP].[LR](T)[sP].[LR](T)}$$$ | | |
| | | $V2.0 | | |
| 5′- | SEQ ID | RNA1{[LR](T)[sP].[dR](C)[sP].[LR](T)[sP].[dR | TAAAAGC | SEQ ID NO: |
| TCTCCAT | NO: 357 | ](C)[sP].[dR](C)[sP].[LR](A)[sP].[dR](T)[sP].[d | ATGGATG | 85 |
| CCATGCT | | R](C)[sP].[dR](C)[sP].[LR](A)[sP].[dR](T)[sP]. | GAGA | |
| TTTA-3′ | | [dR]([In])[sP].[dR](C)[sP].[LR](T)[sP].[dR](T)[ | | |
| | | sP].[dR](T)[sP].[LR](T)[sP].[LR](A)}$$$$V2.0 | | |
| 5′- | SEQ ID | RNA1{[LR]([5meC])[P].[dR](T)[sP].[dR](C)[s | ATAAAAG | SEQ ID NO: |
| CTCCATC | NO: 358 | P].[dR](C)[sP].[LR](A)[sP].[dR](T)[sP].[dR](C) | CATGGAT | 86 |
| CATGCTT | | [sP].[dR](C)[sP].[LR](A)[sP].[dR](T)[sP].[dR]([ | GGAG | |
| TTAT-3′ | | In])[sP].[dR](C)[sP].[LR](T)[sP].[dR](T)[sP].[d | | |
| | | R](T)[sP].[dR](T)[sP].[LR](A)[sP].[LR](T)}$$$ | | |
| | | $V2.0 | | |
| 5′- | SEQ ID | RNA1{[LR](T)[sP].[dR](C)[sP].[dR](C)[sP].[L | GATAAAA | SEQ ID NO: |
| TCCATCC | NO: 359 | R](A)[sP].[dR](T)[sP].[dR](C)[sP].[dR](C)[sP]. | GCATGGA | 87 |
| ATGCTTT | | [LR](A)[sP].[dR](T)[sP].[dR]([In])[sP].[dR](C)[ | TGGA | |
| TATC-3′ | | sP].[LR](T)[sP].[dR](T)[sP].[LR](T)[sP].[dR](T | | |
| | | )[sP].[dR](A)[sP].[LR](T)[sP].[LR]([5meC])}$$ | | |
| | | $$V2.0 | | |
| 5′- | SEQ ID | RNA1{[LR]([5meC])[sP].[dR](C)[sP].[LR](A)[s | AGATAAA | SEQ ID NO: |
| CCATCCA | NO: 360 | P].[dR](T)[sP].[dR](C)[sP].[dR](C)[sP].[LR](A) | AGCATGG | 88 |
| TGCTTTT | | [sP].[dR](T)[sP].[dR]([In])[sP].[dR](C)[sP].[LR] | ATGG | |
| ATCT-3′ | | (T)[sP].[dR](T)[sP].[dR](T)[sP].[dR](T)[sP].[L | | |
| | | R](A)[sP].[dR](T)[sP].[LR]([5meC])[sP].[LR](T | | |
| | | )}$$$$V2.0 | | |
| 5′- | SEQ ID | RNA1{[LR]([5meC])[sP].[dR](A)[sP].[LR](T)[s | TAGATAA | SEQ ID NO: |
| CATCCAT | NO: 361 | P].[dR](C)[sP].[dR](C)[sP].[LR](A)[sP].[dR](T) | AAGCATG | 89 |
| GCTTTTA | | [sP].[dR]([In])[sP].[dR](C)[sP].[LR](T)[sP].[dR] | GATG | |
| TCTA-3′ | | (T)[sP].[dR](T)[sP].[dR](T)[sP].[LR](A)[sP].[d | | |
| | | R](T)[sP].[dR](C)[sP].[LR](T)[sP].[LR](A)}$$$ | | |
| | | $V2.0 | | |
| 5′- | SEQ ID | RNA1{[LR](A)[sP].[dR](T)[sP].[dR](C)[sP].[dR | GTAGATA | SEQ ID NO: |
| ATCCATG | NO: 362 | ](C)[sP].[LR](A)[sP].[dR](T)[sP].[dR]([In])[sP]. | AAAGCAT | 90 |
| CTTTTATC | | [LR]([5meC])[sP].[dR](T)[sP].[dR](T)[sP].[LR] | GGAT | |
| TAC-3′ | | (T)[sP].[dR](T)[sP].[dR](A)[sP].[dR](T)[sP].[L | | |
| | | R]([5meC])[sP].[dR](T)[sP].[LR](A)[sP].[LR]([ | | |
| | | 5meC])}$$$$V2.0 | | |
| 5′- | SEQ ID | RNA1{[LR](T)[sP].[dR](C)[sP].[dR](C)[sP].[L | AGTAGAT | SEQ ID NO: |
| TCCATGC | NO: 363 | R](A)[sP].[dR](T)[sP].[dR]([In])[sP].[dR](C)[sP | AAAAGCA | 91 |
| TTTTATCT | | ].[LR](T)[sP].[dR](T)[sP].[dR](T)[sP].[dR](T)[s | TGGA | |
| ACT-3′ | | P].[LR](A)[sP].[dR](T)[sP].[dR](C)[sP].[dR](T) | | |
| | | [sP].[LR](A)[sP].[LR]([5meC])[sP].[LR](T)}$$$ | | |
| | | $V2.0 | | |
| 5′- | SEQ ID | RNA1{[LR]([5meC])[sP].[dR](C)[sP].[LR](A)[s | GAGTAGA | SEQ ID NO: |
| CCATGCT | NO: 364 | P].[dR](T)[sP].[dR]([In])[sP].[dR](C)[sP].[LR]( | TAAAAGC | 92 |
| TTTATCTA | | T)[sP].[dR](T)[sP].[dR](T)[sP].[dR](T)[sP].[LR | ATGG | |
| CTC-3′ | | ](A)[sP].[dR](T)[sP].[dR](C)[sP].[dR](T)[sP].[L | | |
| | | R](A)[sP].[dR](C)[sP].[LR](T)[sP].[LR]([5meC] | | |
| | | )}$$$$V2.0 | | |
| 5′- | SEQ ID | RNA1{[LR]([5meC])[sP].[dR](A)[sP].[LR](T)[s | TGAGTAG | SEQ ID NO: |
| CATGCTT | NO: 365 | P].[dR]([In])[sP].[dR](C)[sP].[LR](T)[sP].[dR]( | ATAAAAG | 93 |
| TTATCTA | | T)[sP].[dR](T)[sP].[dR](T)[sP].[LR](A)[sP].[dR | CATG | |
| CTCA-3′ | | ](T)[sP].[dR](C)[sP].[dR](T)[sP].[LR](A)[sP].[d | | |
| | | R](C)[sP].[dR](T)[sP].[LR]([5meC])[sP].[LR](A | | |
| | | )}$$$$V2.0 | | |
| 5′- | SEQ ID | RNA1{[LR](A)[sP].[LR](T)[sP].[dR]([In])[sP].[d | ATGAGTA | SEQ ID NO: |
| ATGCTTT | NO: 366 | R](C)[sP].[dR](T)[sP].[LR](T)[sP].[dR](T)[sP].[ | GATAAAA | 94 |
| TATCTAC | | LR](T)[sP].[LR](A)[sP].[dR](T)[sP].[dR](C)[sP] | GCAT | |
| TCAT-3′ | | .[dR](T)[sP].[LR](A)[sP].[dR](C)[sP].[dR](T)[s | | |
| | | P].[dR](C)[sP].[LR](A)[P].[LR](T)}$$$$V2.0 | | |
| 5′- | SEQ ID | RNA1{[LR](T)[sP].[dR]([In])[sP].[dR](C)[sP].[d | GATGAGT | SEQ ID NO: |
| TGCTTTT | NO: 367 | R](T)[sP].[LR](T)[sP].[dR](T)[sP].[dR](T)[sP].[ | AGATAAA | 95 |
| ATCTACT | | LR](A)[sP].[dR](T)[sP].[LR]([5meC])[sP].[dR]( | AGCA | |
| CATC-3′ | | T)[sP].[LR](A)[sP].[dR](C)[sP].[dR](T)[sP].[LR | | |
| | | ]([5meC])[sP].[dR](A)[sP].[LR](T)[sP].[LR]([5 | | |
| | | meC])}$$$$V2.0 | | |
| 5′- | SEQ ID | RNA1{[LR](G)[sP].[dR](C)[sP].[dR](T)[sP].[L | TGATGAG | SEQ ID NO: |
| GCTTTTA | NO: 368 | R](T)[sP].[dR](T)[sP].[dR](T)[sP].[LR](A)[sP].[ | TAGATAA | 96 |
| TCTACTC | | dR](T)[sP].[dR](C)[sP].[dR](T)[sP].[LR](A)[sP] | AAGC | |
| ATCA-3′ | | .[dR](C)[sP].[LR](T)[sP].[dR](C)[sP].[dR](A)[s | | |
| | | P].[dR](T)[sP].[LR]([5meC])[sP].[LR](A)}$$$$ | | |
| | | V2.0 | | |
| 5′- | SEQ ID | RNA1{[LR]([5meC])[sP].[dR](C)[sP].[dR](C)[s | GATGGAT | SEQ ID NO: |
| CCCATCT | NO: 369 | P].[dR](A)[P].[LR](T)[sP].[dR](C)[sP].[dR](T) | GGAGAGA | 97 |
| CTCCATC | | [sP].[dR](C)[sP].[LR](T)[sP].[dR](C)[sP].[dR]( | TGGG | |
| CATC-3′ | | C)[sP].[dR](A)[sP].[LR](T)[sP].[dR](C)[sP].[d | | |
| | | R](C)[sP].[dR](A)[sP].[LR](T)[sP].[LR]([5meC] | | |
| | | )}$$$$V2.0 | | |
| 5′- | SEQ ID | RNA1{[LR]([5meC])[sP].[dR](A)[P].[LR](T)[s | AGGATGG | SEQ ID NO: |
| CATCTCT | NO: 370 | P].[dR](C)[sP].[dR](T)[sP].[dR](C)[sP].[LR](T) | ATGGAGA | 98 |
| CCATCCA | | [sP].[dR](C)[sP].[dR](C)[sP].[LR](A)[sP].[dR]( | GATG | |
| TCCT-3′ | | T)[sP].[dR](C)[sP].[dR](C)[sP].[LR](A)[sP].[d | | |
| | | R](T)[sP].[dR](C)[P].[LR]([5meC])[sP].[LR](T | | |
| | | )}$$$$V2.0 | | |
| 5′- | SEQ ID | RNA1{[LR](A)[sP].[dR](T)[sP].[dR](C)[sP].[dR | AAGGATG | SEQ ID NO: |
| ATCTCTC | NO: 371 | ](T)[sP].[LR]([5meC])[sP].[dR](T)[sP].[dR](C)[ | GATGGAG | 99 |
| CATCCAT | | sP].[dR](C)[sP].[LR](A)[sP].[dR](T)[P].[dR]( | AGAT | |
| CCTT-3′ | | C)[sP].[dR](C)[sP].[LR](A)[sP].[dR](T)[sP].[d | | |
| | | R](C)[sP].[dR](C)[sP].[LR](T)[sP].[LR](T)}$$$ | | |
| | | $V2.0 | | |
| 5′- | SEQ ID | RNA1{[LR](T)[sP].[dR](C)[sP].[dR](T)[sP].[dR | AAAGGAT | SEQ ID NO: |
| TCTCTCC | NO: 372 | ](C)[sP].[LR](T)[sP].[dR](C)[P].[dR](C)[sP].[L | GGATGGA | 100 |
| ATCCATC | | R](A)[sP].[dR](T)[sP].[dR](C)[sP].[dR](C)[sP]. | GAGA | |
| CTTT-3′ | | [LR](A)[sP].[dR](T)[sP].[LR]([5meC])[sP].[dR] | | |
| | | (C)[sP].[dR](T)[sP].[LR](T)[sP].[LR](T)}$$$$V | | |
| | | 2.0 | | |
| 5′- | SEQ ID | RNA1{[LR]([5meC])[sP].[dR](T)[sP].[dR](C)[s | AAAAGGA | SEQ ID NO: |
| CTCTCCA | NO: 373 | P].[LR](T)[sP].[dR](C)[sP].[dR](C)[sP].[LR](A) | TGGATGG | 101 |
| TCCATCC | | [sP].[dR](T)[sP].[dR](C)[sP].[dR](C)[sP].[LR]( | AGAG | |
| TTTT-3′ | | A)[sP].[dR](T)[sP].[dR](C)[sP].[dR](C)[sP].[L | | |
| | | R](T)[sP].[dR](T)[sP].[LR](T)[sP].[LR](T)}$$$ | | |
| | | $V2.0 | | |
| 5′- | SEQ ID | RNA1{[LR](T)[sP].[dR](C)[sP].[LR](T)[sP].[dR | TAAAAGG | SEQ ID NO: |
| TCTCCAT | NO: 374 | ](C)[sP].[dR](C)[sP].[LR](A)[sP].[dR](T)[sP].[d | ATGGATG | 102 |
| CCATCCT | | R](C)[sP].[dR](C)[sP].[LR](A)[sP].[dR](T)[sP]. | GAGA | |
| TTTA-3′ | | [dR](C)[sP].[dR](C)[sP].[LR](T)[sP].[dR](T)[s | | |
| | | P].[dR](T)[sP].[LR](T)[sP].[LR](A)}$$$$V2.0 | | |
| 5′- | SEQ ID | RNA1{[LR]([5meC])[sP].[dR](T)[sP].[dR](C)[s | ATAAAAG | SEQ ID NO: |
| CTCCATC | NO: 375 | P].[dR](C)[sP].[LR](A)[sP].[dR](T)[sP].[dR](C) | GATGGAT | 103 |
| CATCCTT | | [sP].[dR](C)[sP].[LR](A)[sP].[dR](T)[sP].[dR]( | GGAG | |
| TTAT-3′ | | C)[sP].[dR](C)[sP].[LR](T)[sP].[dR](T)[sP].[dR | | |
| | | ](T)[sP].[dR](T)[sP].[LR](A)[sP].[LR](T)}$$$$ | | |
| | | V2.0 | | |
| 5′- | SEQ ID | RNA1{[LR](T)[sP].[dR](C)[sP].[dR](C)[sP].[L | GATAAAA | SEQ ID NO: |
| TCCATCC | NO: 376 | R](A)[sP].[dR](T)[sP].[dR](C)[sP].[dR](C)[sP]. | GGATGGA | 104 |
| ATCCTTTT | | [LR](A)[sP].[dR](T)[sP].[dR](C)[sP].[dR](C)[s | TGGA | |
| ATC-3′ | | P].[LR](T)[sP].[dR](T)[sP].[LR](T)[sP].[dR](T)[ | | |
| | | sP].[dR](A)[sP].[LR](T)[sP].[LR]([5meC])}$$$ | | |
| | | $V2.0 | | |
| 5′- | SEQ ID | RNA1{[LR]([5meC])[sP].[dR](C)[sP].[LR](A)[s | AGATAAA | SEQ ID NO: |
| CCATCCA | NO: 377 | P].[dR](T)[sP].[dR](C)[sP].[dR](C)[sP].[LR](A) | AGGATGG | 105 |
| TCCTTTTA | | [sP].[dR](T)[sP].[dR](C)[sP].[dR](C)[sP].[LR]( | ATGG | |
| TCT-3′ | | T)[sP].[dR](T)[sP].[dR](T)[sP].[dR](T)[sP].[LR | | |
| | | ](A)[sP].[dR](T)[sP].[LR]([5meC])[sP].[LR](T)} | | |
| | | $$$$V2.0 | | |
| 5′- | SEQ ID | RNA1{[LR]([5meC])[sP].[dR](A)[sP].[LR](T)[s | TAGATAA | SEQ ID NO: |
| CATCCAT | NO: 378 | P].[dR](C)[sP].[dR](C)[sP].[LR](A)[sP].[dR](T) | AAGGATG | 106 |
| CCTTTTAT | | [sP].[dR](C)[sP].[dR](C)[sP].[LR](T)[sP].[dR]( | GATG | |
| CTA-3′ | | T)[sP].[dR](T)[sP].[dR](T)[sP].[LR](A)[sP].[dR | | |
| | | ](T)[sP].[dR](C)[sP].[LR](T)[sP].[LR](A)}$$$$ | | |
| | | V2.0 | | |
| 5′- | SEQ ID | RNA1{[LR](A)[P].[dR](T)[sP].[dR](C)[sP].[dR | GTAGATA | SEQ ID NO: |
| ATCCATC | NO: 379 | ](C)[sP].[LR](A)[sP].[dR](T)[sP].[dR](C)[sP].[L | AAAGGAT | 107 |
| CTTTTATC | | R]([5meC])[sP].[dR](T)[sP].[dR](T)[sP].[LR](T | GGAT | |
| TAC-3′ | | )[sP].[dR](T)[sP].[dR](A)[sP].[dR](T)[sP].[LR]( | | |
| | | [5meC])[sP].[dR](T)[sP].[LR](A)[sP].[LR]([5m | | |
| | | eC])}$$$$V2.0 | | |
| 5′- | SEQ ID | RNA1{[LR](T)[P].[dR](C)[sP].[dR](C)[sP].[L | AGTAGAT | SEQ ID NO: |
| TCCATCC | NO: 380 | R](A)[sP].[dR](T)[sP].[dR](C)[sP].[dR](C)[sP]. | AAAAGGA | 108 |
| TTTTATCT | | [LR](T)[sP].[dR](T)[sP].[dR](T)[sP].[dR](T)[sP | TGGA | |
| ACT-3′ | | ].[LR](A)[sP].[dR](T)[sP].[dR](C)[sP].[dR](T)[s | | |
| | | P].[LR](A)[sP].[LR]([5meC])[sP].[LR](T)}$$$$ | | |
| | | V2.0 | | |
| 5′- | SEQ ID | RNA1{[LR]([5meC])[sP].[dR](C)[sP].[LR](A)[s | GAGTAGA | SEQ ID NO: |
| CCATCCT | NO: 381 | P].[dR](T)[sP].[dR](C)[sP].[dR](C)[sP].[LR](T) | TAAAAGG | 109 |
| TTTATCTA | | [sP].[dR](T)[sP].[dR](T)[sP].[dR](T)[sP].[LR]( | ATGG | |
| CTC-3′ | | A)[sP].[dR](T)[sP].[dR](C)[sP].[dR](T)[sP].[LR | | |
| | | ](A)[sP].[dR](C)[sP].[LR](T)[sP].[LR]([5meC])} | | |
| | | $$$$V2.0 | | |
| 5′- | SEQ ID | RNA1{[LR]([5meC])[P].[dR](A)[sP].[LR](T)[s | TGAGTAG | SEQ ID NO: |
| CATCCTT | NO: 382 | P].[dR](C)[sP].[dR](C)[sP].[LR](T)[sP].[dR](T) | ATAAAAG | 110 |
| TTATCTA | | [sP].[dR](T)[sP].[dR](T)[sP].[LR](A)[sP].[dR]( | GATG | |
| CTCA-3′ | | T)[sP].[dR](C)[sP].[dR](T)[sP].[LR](A)[sP].[dR | | |
| | | ](C)[sP].[dR](T)[sP].[LR]([5meC])[sP].[LR](A)} | | |
| | | $$$$V2.0 | | |
| 5′- | SEQ ID | RNA1{[LR](A)[sP].[dR](T)[sP].[LR]([5meC])[s | ATGAGTA | SEQ ID NO: |
| ATCCTTTT | NO: 383 | P].[dR](C)[sP].[dR](T)[sP].[LR](T)[sP].[dR](T)[ | GATAAAA | 111 |
| ATCTACT | | SP].[LR](T)[P].[LR](A)[sP].[dR](T)[sP].[dR](C | GGAT | |
| CAT-3′ | | )[sP].[dR](T)[sP].[LR](A)[sP].[dR](C)[sP].[dR]( | | |
| | | T)[sP].[dR](C)[sP].[LR](A)[sP].[LR](T)}$$$$V | | |
| | | 2.0 | | |
| 5′- | SEQ ID | RNA1{[LR](T)[sP].[dR](C)[sP].[dR](C)[sP].[d | GATGAGT | SEQ ID NO: |
| TCCTTTTA | NO: 384 | R](T)[sP].[LR](T)[sP].[dR](T)[sP].[dR](T)[sP].[ | AGATAAA | 112 |
| TCTACTC | | LR](A)[P].[dR](T)[sP].[LR]([5meC])[sP].[dR]( | AGGA | |
| ATC-3′ | | T)[sP].[LR](A)[sP].[dR](C)[sP].[dR](T)[sP].[LR | | |
| | | ]([5meC])[sP].[dR](A)[sP].[LR](T)[P].[LR]([5 | | |
| | | meC])}$$$$V2.0 | | |
| 5′- | SEQ ID | RNA1{[LR]([5meC])[sP].[dR](C)[sP].[dR](T)[s | TGATGAG | SEQ ID NO: |
| CCTTTTAT | NO: 385 | P].[LR](T)[sP].[dR](T)[sP].[dR](T)[sP].[LR](A)[ | TAGATAA | 113 |
| CTACTCA | | sP].[dR](T)[sP].[dR](C)[sP].[dR](T)[sP].[LR](A | AAGG | |
| TCA-3′ | | )[sP].[dR](C)[sP].[LR](T)[sP].[dR](C)[sP].[dR]( | | |
| | | A)[sP].[dR](T)[sP].[LR]([5meC])[sP].[LR](A)}$ | | |
| | | $$$V2.0 | | |
| 5′- | SEQ ID | RNA1{[LR]([5meC])[sP].[LR](T)[sP].[dR](T)[s | GTGATGA | SEQ ID NO: |
| CTTTTATC | NO: 386 | P].[dR](T)[sP].[dR](T)[sP].[LR](A)[sP].[dR](T)[ | GTAGATA | 114 |
| TACTCAT | | sP].[LR]([5meC])[sP].[dR](T)[sP].[LR](A)[sP].[ | AAAG | |
| CAC-3′ | | LR]([5meC])[sP].[dR](T)[sP].[dR](C)[sP].[LR]( | | |
| | | A)[sP].[dR](T)[sP].[dR](C)[sP].[LR](A)[sP].[LR | | |
| | | ]([5meC])}$$$$V2.0 | | |
| 5′- | SEQ ID | RNA1{[LR](T)[sP].[LR](T)[sP].[dR](T)[sP].[dR | AGTGATG | SEQ ID NO: |
| TTTTATCT | NO: 387 | ](T)[sP].[LR](A)[sP].[dR](T)[sP].[LR]([5meC])[ | AGTAGAT | 115 |
| ACTCATC | | sP].[dR](T)[sP].[LR](A)[sP].[dR](C)[sP].[LR](T | AAAA | |
| ACT-3′ | | )[sP].[dR](C)[sP].[LR](A)[sP].[dR](T)[sP].[dR]( | | |
| | | C)[sP].[LR](A)[sP].[LR]([5meC])[sP].[LR](T)}$ | | |
| | | $$$V2.0 | | |
| 5′- | SEQ ID | RNA1{[LR](T)[sP].[dR](T)[sP].[dR](T)[sP].[LR | GAGTGAT | SEQ ID NO: |
| TTTATCTA | NO: 388 | ](A)[sP].[dR](T)[sP].[LR]([5meC])[sP].[dR](T)[ | GAGTAGA | 116 |
| CTCATCA | | sP].[dR](A)[sP].[dR](C)[sP].[LR](T)[sP].[LR]([ | TAAA | |
| CTC-3′ | | 5meC])[sP].[dR](A)[sP].[LR](T)[sP].[dR](C)[s | | |
| | | P].[dR](A)[sP].[dR](C)[sP].[LR](T)[sP].[LR]([5 | | |
| | | meC])}$$$$V2.0 | | |
| 5′- | SEQ ID | RNA1{[LR](T)[P].[dR](T)[sP].[LR](A)[sP].[dR | TGAGTGA | SEQ ID NO: |
| TTATCTA | NO: 389 | ](T)[sP].[dR](C)[P].[LR](T)[sP].[dR](A)[sP].[d | TGAGTAG | 117 |
| CTCATCA | | R](C)[sP].[dR](T)[sP].[LR]([5meC])[sP].[dR](A | ATAA | |
| CTCA-3′ | | )[sP].[LR](T)[sP].[dR](C)[sP].[LR](A)[sP].[dR]( | | |
| | | C)[sP].[dR](T)[sP].[LR]([5meC])[sP].[LR](A)}$ | | |
| | | $$$V2.0 | | |
| 5′- | SEQ ID | RNA1{[LR](T)[sP].[dR](A)[sP].[LR](T)[sP].[dR | ATGAGTG | SEQ ID NO: |
| TATCTAC | NO: 390 | ](C)[sP].[dR](T)[sP].[dR](A)[P].[LR]([5meC])[ | ATGAGTA | 118 |
| TCATCAC | | SP].[LR](T)[sP].[dR](C)[sP].[LR](A)[sP].[dR](T | GATA | |
| TCAT-3′ | | )[sP].[dR](C)[P].[LR](A)[sP].[dR](C)[P].[dR] | | |
| | | (T)[sP].[dR](C)[sP].[LR](A)[sP].[LR](T)}$$$$V | | |
| | | 2.0 | | |
| 5′- | SEQ ID | RNA1{[LR](A)[sP].[dR](T)[sP].[dR](C)[sP].[dR | AATGAGT | SEQ ID NO: |
| ATCTACT | NO: 391 | ](T)[sP].[LR](A)[sP].[dR](C)[sP].[dR](T)[sP].[d | GATGAGT | 119 |
| CATCACT | | R](C)[sP].[LR](A)[sP].[LR](T)[sP].[dR](C)[sP]. | AGAT | |
| CATT-3′ | | [LR](A)[sP].[LR]([5meC])[sP].[dR](T)[sP].[dR] | | |
| | | (C)[sP].[dR](A)[sP].[LR](T)[sP].[LR](T)}$$$$V | | |
| | | 2.0 | | |
| 5′- | SEQ ID | RNA1{[LR](T)[sP].[dR](C)[sP].[dR](T)[sP].[LR | GAATGAG | SEQ ID NO: |
| TCTACTC | NO: 392 | ](A)[sP].[LR]([5meC])[sP].[dR](T)[sP].[dR](C)[ | TGATGAG | 120 |
| ATCACTC | | sP].[dR](A)[sP].[LR](T)[sP].[dR](C)[sP].[LR](A | TAGA | |
| ATTC-3′ | | )[sP].[dR](C)[sP].[dR](T)[sP].[dR](C)[P].[LR]( | | |
| | | A)[sP].[dR](T)[sP].[LR](T)[sP].[LR]([5meC])}$ | | |
| | | $$$V2.0 | | |
| 5′- | SEQ ID | RNA1{[LR]([5meC])[sP].[dR](T)[sP].[dR](A)[s | TGAATGA | SEQ ID NO: |
| CTACTCA | NO: 393 | P].[dR](C)[sP].[LR](T)[sP].[dR](C)[sP].[LR](A) | GTGATGA | 121 |
| TCACTCA | | [sP].[dR](T)[sP].[dR](C)[sP].[LR](A)[sP].[dR]( | GTAG | |
| TTCA-3′ | | C)[sP].[dR](T)[sP].[dR](C)[sP].[LR](A)[sP].[d | | |
| | | R](T)[sP].[dR](T)[sP].[LR]([5meC])[sP].[LR](A | | |
| | | )}$$$$V2.0 | | |
| 5′- | SEQ ID | RNA1{[LR](T)[sP].[dR](A)[sP].[dR](C)[sP].[LR | ATGAATG | SEQ ID NO: |
| TACTCAT | NO: 394 | ](T)[sP].[dR](C)[sP].[LR](A)[sP].[dR](T)[sP].[d | AGTGATG | 122 |
| CACTCAT | | R](C)[sP].[LR](A)[P].[dR](C)[sP].[dR](T)[sP]. | AGTA | |
| TCAT-3′ | | [LR]([5meC])[sP].[dR](A)[sP].[LR](T)[sP].[LR] | | |
| | | (T)[sP].[dR](C)[sP].[LR](A)[sP].[LR](T)}$$$$V | | |
| | | 2.0 | | |
| 5′- | SEQ ID | RNA1{[LR](A)[sP].[dR](C)[sP].[LR](T)[sP].[dR | GATGAAT | SEQ ID NO: |
| ACTCATC | NO: 395 | ](C)[sP].[dR](A)[sP].[LR](T)[sP].[dR](C)[sP].[L | GAGTGAT | 123 |
| ACTCATT | | R](A)[sP].[dR](C)[sP].[dR](T)[sP].[dR](C)[sP]. | GAGT | |
| CATC-3′ | | [LR](A)[sP].[dR](T)[sP].[dR](T)[sP].[dR](C)[sP | | |
| | | ].[LR](A)[sP].[LR](T)[sP].[LR]([5meC])}$$$$V | | |
| | | 2.0 | | |
| 5′- | SEQ ID | RNA1{[LR]([5meC])[sP].[dR](A)[sP].[LR](T)[s | ACAGATG | SEQ ID NO: |
| CATCACT | NO: 396 | P].[dR](C)[sP].[dR](A)[sP].[dR](C)[sP].[LR](T) | AATGAGT | 124 |
| CATTCAT | | [sP].[dR](C)[P].[LR](A)[sP].[dR](T)[sP].[dR]( | GATG | |
| CTGT-3′ | | T)[sP].[dR](C)[sP].[LR](A)[sP].[dR](T)[sP].[dR | | |
| | | ](C)[sP].[dR](T)[sP].[LR](G)[sP].[LR](T)}$$$$ | | |
| | | V2.0 | | |
| 5′- | SEQ ID | RNA1{[LR](A)[sP].[dR](T)[sP].[dR](C)[sP].[LR | AACAGAT | SEQ ID NO: |
| ATCACTC | NO: 397 | ](A)[sP].[dR](C)[sP].[LR](T)[sP].[dR](C)[sP].[L | GAATGAG | 125 |
| ATTCATC | | R](A)[sP].[dR](T)[sP].[LR](T)[sP].[dR](C)[sP].[ | TGAT | |
| TGTT-3′ | | LR](A)[sP].[dR](T)[sP].[dR](C)[sP].[dR](T)[sP] | | |
| | | .[LR](G)[sP].[LR](T)[sP].[LR](T)}$$$$V2.0 | | |
| 5′- | SEQ ID | RNA1{[LR](T)[sP].[dR](C)[sP].[dR](A)[sP].[dR | GAACAGA | SEQ ID NO: |
| TCACTCA | NO: 398 | ](C)[sP].[LR](T)[sP].[dR](C)[sP].[LR](A)[sP].[d | TGAATGA | 126 |
| TTCATCT | | R](T)[sP].[LR](T)[sP].[dR](C)[sP].[dR](A)[sP].[ | GTGA | |
| GTTC-3′ | | LR](T)[sP].[dR](C)[sP].[dR](T)[sP].[LR](G)[sP | | |
| | | ].[dR](T)[sP].[LR](T)[sP].[LR]([5meC])}$$$$V | | |
| | | 2.0 | | |
| 5′- | SEQ ID | RNA1{[LR](A)[sP].[dR](C)[sP].[LR](T)[sP].[dR | TTGAACA | SEQ ID NO: |
| ACTCATT | NO: 399 | ](C)[sP].[LR](A)[sP].[LR](T)[sP].[dR](T)[sP].[d | GATGAAT | 127 |
| CATCTGT | | R](C)[sP].[LR](A)[sP].[dR](T)[sP].[dR](C)[sP]. | GAGT | |
| TCAA-3′ | | [dR](T)[sP].[LR](G)[sP].[LR](T)[sP].[dR](T)[sP | | |
| | | ].[dR](C)[sP].[LR](A)[sP].[LR](A)}$$$$V2.0 | | |
| 5′- | SEQ ID | RNA1{[LR]([5meC])[sP].[dR](T)[sP].[dR](C)[s | ATTGAAC | SEQ ID NO: |
| CTCATTC | NO: 400 | P].[LR](A)[sP].[dR](T)[sP].[dR](T)[sP].[LR]([5 | AGATGAA | 128 |
| ATCTGTT | | meC])[sP].[dR](A)[sP].[dR](T)[sP].[LR]([5me | TGAG | |
| CAAT-3′ | | C])[sP].[dR](T)[sP].[LR](G)[sP].[dR](T)[sP].[L | | |
| | | R](T)[sP].[dR](C)[sP].[LR](A)[sP].[LR](A)[sP]. | | |
| | | [LR](T)}$$$$V2.0 | | |
| 5′- | SEQ ID | RNA1{[LR](T)[sP].[dR](C)[sP].[dR](A)[sP].[LR | GATTGAA | SEQ ID NO: |
| TCATTCA | NO: 401 | ](T)[sP].[dR](T)[sP].[dR](C)[sP].[dR](A)[sP].[L | CAGATGA | 129 |
| TCTGTTC | | R](T)[sP].[dR](C)[sP].[LR](T)[sP].[dR](G)[sP]. | ATGA | |
| AATC-3′ | | [LR](T)[sP].[LR](T)[sP].[dR](C)[sP].[dR](A)[sP | | |
| | | ].[dR](A)[sP].[LR](T)[sP].[LR]([5meC])}$$$$V | | |
| | | 2.0 | | |
| 5′- | SEQ ID | RNA1{[LR]([5meC])[sP].[dR](A)[sP].[LR](T)[s | TGATTGA | SEQ ID NO: |
| CATTCAT | NO: 402 | P].[dR](T)[sP].[LR]([5meC])[sP].[dR](A)[sP].[d | ACAGATG | 130 |
| CTGTTCA | | R](T)[sP].[LR]([5meC])[sP].[dR](T)[P].[dR](G | AATG | |
| ATCA-3′ | | )[sP].[dR](T)[sP].[LR](T)[sP].[dR](C)[sP].[LR]( | | |
| | | A)[sP].[dR](A)[sP].[dR](T)[sP].[LR]([5meC])[s | | |
| | | P].[LR](A)}$$$$V2.0 | | |
| 5′- | SEQ ID | RNA1{[LR](A)[sP].[dR](T)[sP].[dR](T)[sP].[dR | ATGATTG | SEQ ID NO: |
| ATTCATC | NO: 403 | ](C)[sP].[LR](A)[sP].[LR](T)[sP].[dR](C)[sP].[L | AACAGAT | 131 |
| TGTTCAA | | R](T)[sP].[dR](G)[sP].[dR](T)[sP].[LR](T)[sP].[ | GAAT | |
| TCAT-3′ | | dR](C)[sP].[dR](A)[sP].[LR](A)[sP].[LR](T)[sP | | |
| | | ].[dR](C)[sP].[LR](A)[sP].[LR](T)$$$$V2.0 | | |
| 5′- | SEQ ID | RNA1{[LR](T)[sP].[LR](T)[sP].[dR](C)[sP].[dR | AATGATT | SEQ ID NO: |
| TTCATCT | NO: 404 | ](A)[sP].[dR](T)[sP].[LR]([5meC])[sP].[LR](T)[ | GAACAGA | 132 |
| GTTCAAT | | sP].[dR](G)[sP].[dR](T)[sP].[LR](T)[sP].[dR]( | TGAA | |
| CATT-3′ | | C)[sP].[dR](A)[sP].[LR](A)[sP].[dR](T)[sP].[dR | | |
| | | ](C)[sP].[dR](A)[sP].[LR](T)[sP].[LR](T)}$$$$ | | |
| | | V2.0 | | |
| 5′- | SEQ ID | RNA1{[LR](T)[sP].[dR](C)[sP].[LR](A)[sP].[dR | GAATGAT | SEQ ID NO: |
| TCATCTG | NO: 405 | ](T)[sP].[dR](C)[sP].[dR](T)[sP].[LR](G)[sP].[d | TGAACAG | 133 |
| TTCAATC | | R](T)[sP].[LR](T)[sP].[LR]([5meC])[sP].[dR](A | ATGA | |
| ATTC-3′ | | )[sP].[dR](A)[sP].[dR](T)[sP].[LR]([5meC])[sP] | | |
| | | [dR](A)[sP].[LR](T)[sP].[LR](T)[sP].[LR]([5me | | |
| | | C])}$$$$V2.0 | | |
| 5′- | SEQ ID | RNA1{[LR]([5meC])[sP].[dR](A)[sP].[dR](T)[s | TGAATGA | SEQ ID NO: |
| CATCTGT | NO: 406 | P].[LR]([5meC])[sP].[dR](T)[sP].[dR](G)[sP].[ | TTGAACA | 134 |
| TCAATCA | | dR](T)[sP].[LR](T)[P].[dR](C)[sP].[LR](A)[sP] | GATG | |
| TTCA-3′ | | .[dR](A)[sP].[dR](T)[sP].[LR]([5meC])[sP].[dR | | |
| | | ](A)[sP].[LR](T)[sP].[dR](T)[sP].[LR]([5meC])[ | | |
| | | SP].[LR](A)}$$$$V2.0 | | |
| 5′- | SEQ ID | RNA1{[LR](A)[sP].[LR](T)[sP].[dR](C)[sP].[LR | ATGAATG | SEQ ID NO: |
| ATCTGTT | NO: 407 | ](T)[sP].[dR](G)[sP].[dR](T)[sP].[LR](T)[sP].[d | ATTGAAC | 135 |
| CAATCAT | | R](C)[sP].[dR](A)[sP].[LR](A)[sP].[LR](T)[sP]. | AGAT | |
| TCAT-3′ | | [dR](C)[sP].[LR](A)[sP].[dR](T)[sP].[dR](T)[sP | | |
| | | ].[dR](C)[sP].[LR](A)[sP].[LR](T)}$$$$V2.0 | | |
| 5′- | SEQ ID | RNA1{[LR](T)[sP].[dR](C)[sP].[LR](T)[sP].[dR | AATGAAT | SEQ ID NO: |
| TCTGTTC | NO: 408 | ](G)[sP].[LR](T)[sP].[LR](T)[sP].[dR](C)[sP].[d | GATTGAA | 136 |
| AATCATT | | R](A)[sP].[LR](A)[sP].[dR](T)[sP].[dR](C)[sP]. | CAGA | |
| CATT-3′ | | [LR](A)[sP].[LR](T)[sP].[dR](T)[sP].[dR](C)[sP | | |
| | | ].[dR](A)[sP].[LR](T)[sP].[LR](T)}$$$$V2.0 | | |
| 5′- | SEQ ID | RNA1{[LR]([5meC])[sP].[dR](T)[sP].[dR](G)[s | GAATGAA | SEQ ID NO: |
| CTGTTCA | NO: 409 | P].[dR](T)[sP].[LR](T)[sP].[dR](C)[sP].[LR](A) | TGATTGA | 137 |
| ATCATTC | | [sP].[dR](A)[sP].[LR](T)[sP].[dR](C)[sP].[dR]( | ACAG | |
| ATTC-3′ | | A)[P].[LR](T)[sP].[dR](T)[sP].[LR]([5meC])[s | | |
| | | P].[LR](A)[sP].[dR](T)[sP].[LR](T)[sP].[LR]([5 | | |
| | | meC])}$$$$V2.0 | | |
| 5′- | SEQ ID | RNA1{[LR](T)[sP].[LR](G)[sP].[dR](T)[sP].[LR | TGAATGA | SEQ ID NO: |
| TGTTCAA | NO: 410 | ](T)[sP].[dR](C)[sP].[dR](A)[sP].[LR](A)[sP].[d | ATGATTG | 138 |
| TCATTCA | | R](T)[sP].[dR](C)[sP].[dR](A)[sP].[LR](T)[sP].[ | AACA | |
| TTCA-3′ | | dR](T)[P].[LR]([5meC])[sP].[dR](A)[sP].[dR]( | | |
| | | T)[sP].[dR](T)[sP].[LR]([5meC])[sP].[LR](A)}$ | | |
| | | $$$V2.0 | | |
| 5′- | SEQ ID | RNA1{[LR](G)[sP].[dR](T)[sP].[dR](T)[sP].[LR | ATGAATG | SEQ ID NO: |
| GTTCAAT | NO: 411 | ]([5meC])[sP].[LR](A)[sP].[dR](A)[sP].[dR](T)[ | AATGATT | 139 |
| CATTCAT | | SP].[LR]([5meC])[sP].[dR](A)[sP].[LR](T)[sP].[ | GAAC | |
| TCAT-3′ | | dR](T)[sP].[LR]([5meC])[sP].[dR](A)[sP].[dR]( | | |
| | | T)[sP].[LR](T)[sP].[dR](C)[sP].[LR](A)[sP].[LR | | |
| | | ((T)}$$$$V2.0 | | |
| 5′- | SEQ ID | RNA1{[LR](T)[sP].[LR](T)[sP].[dR](C)[sP].[dR | AATGAAT | SEQ ID NO: |
| TTCAATC | NO: 412 | ](A)[sP].[dR](A)[sP].[LR](T)[sP].[LR]([5meC])[ | GAATGAT | 140 |
| ATTCATT | | sP].[dR](A)[sP].[dR](T)[sP].[LR](T)[sP].[LR]([ | TGAA | |
| CATT-3′ | | 5meC])[sP].[dR](A)[sP].[dR](T)[sP].[LR](T)[s | | |
| | | P].[dR](C)[P].[LR](A)[sP].[LR](T)[sP].[LR](T) | | |
| | | }$$$$V2.0 | | |
| 5′- | SEQ ID | RNA1{[LR](T)[sP].[dR](C)[sP].[dR](A)[sP].[LR | GAATGAA | SEQ ID NO: |
| TCAATCA | NO: 413 | ](A)[sP].[LR](T)[sP].[dR](C)[sP].[dR](A)[sP].[d | TGAATGA | 141 |
| TTCATTC | | R](T)[sP].[LR](T)[P].[LR]([5meC])[sP].[dR](A | TTGA | |
| ATTC-3′ | | )[sP].[dR](T)[sP].[LR](T)[sP].[LR]([5meC])[sP] | | |
| | | .[dR](A)[sP].[dR](T)[sP].[LR](T)[sP].[LR]([5me | | |
| | | C])}$$$$V2.0 | | |
| 5′- | SEQ ID | RNA1{[LR]([5meC])[sP].[dR](A)[sP].[dR](A)[s | TGAATGA | SEQ ID NO: |
| CAATCAT | NO: 414 | P].[LR](T)[sP].[dR](C)[sP].[LR](A)[sP].[dR](T) | ATGAATG | 142 |
| TCATTCA | | [sP].[dR](T)[sP].[LR]([5meC])[sP].[dR](A)[sP]. | ATTG | |
| TTCA-3′ | | [LR](T)[sP].[LR](T)[sP].[dR](C)[sP].[LR](A)[sP | | |
| | | ].[LR](T)[sP].[dR](T)[sP].[LR]([5meC])[sP].[LR | | |
| | | [(A)}$$$$V2.0 | | |
| 5′- | SEQ ID | RNA1{[LR](A)[sP].[LR](A)[sP].[dR](T)[sP].[LR | GTGAATG | SEQ ID NO: |
| AATCATT | NO: 415 | ]([5meC])[sP].[dR](A)[sP].[dR](T)[sP].[dR](T)[ | AATGAAT | 143 |
| CATTCAT | | SP].[LR]([5meC])[sP].[dR](A)[sP].[LR](T)[sP].[ | GATT | |
| TCAC-3′ | | LR](T)[sP].[dR](C)[sP].[dR](A)[sP].[LR](T)[sP] | | |
| | | .[dR](T)[sP].[LR]([5meC])[sP].[LR](A)[sP].[LR | | |
| | | [[5meC])}$$$$V2.0 | | |
| 5′- | SEQ ID | RNA1{[LR](A)[sP].[dR](T)[sP].[dR](C)[sP].[dR | GGTGAAT | SEQ ID NO: |
| ATCATTC | NO: 416 | ](A)[sP].[LR](T)[sP].[dR](T)[sP].[dR](C)[sP].[L | GAATGAA | 144 |
| ATTCATT | | R](A)[sP].[dR](T)[sP].[LR](T)[sP].[dR](C)[sP].[ | TGAT | |
| CACC-3′ | | dR](A)[sP].[LR](T)[sP].[dR](T)[sP].[dR](C)[sP] | | |
| | | .[dR](A)[sP].[LR]([5meC])[sP].[LR]([5meC])}$ | | |
| | | $$$V2.0 | | |
| 5′- | SEQ ID | RNA1{[LR](T)[sP].[dR](C)[sP].[LR](A)[sP].[dR | TGGTGAA | SEQ ID NO: |
| TCATTCA | NO: 417 | ](T)[sP].[dR](T)[sP].[dR](C)[sP].[LR](A)[sP].[d | TGAATGA | 145 |
| TTCATTC | | R](T)[sP].[dR](T)[sP].[LR]([5meC])[sP].[dR](A | ATGA | |
| ACCA-3′ | | )[sP].[dR](T)[sP].[LR](T)[sP].[dR](C)[sP].[dR]( | | |
| | | A)[sP].[dR](C)[sP].[LR]([5meC])[sP].[LR](A)}$ | | |
| | | $$$V2.0 | | |
| 5′- | SEQ ID | RNA1{[LR]([5meC])[sP].[dR](A)[sP].[LR](T)[s | CTGGTGA | SEQ ID NO: |
| CATTCAT | NO: 418 | P].[dR](T)[sP].[LR]([5meC])[sP].[dR](A)[sP].[L | ATGAATG | 146 |
| TCATTCA | | R](T)[sP].[dR](T)[sP].[dR](C)[sP].[LR](A)[sP].[ | AATG | |
| CCAG-3′ | | dR](T)[sP].[dR](T)[sP].[dR](C)[sP].[LR](A)[sP] | | |
| | | .[dR](C)[sP].[dR](C)[sP].[LR](A)[sP].[LR](G)}$ | | |
| | | $$$V2.0 | | |
| 5′- | SEQ ID | RNA1{[LR](A)[sP].[dR](T)[sP].[dR](T)[sP].[dR | GCTGGTG | SEQ ID NO: |
| ATTCATT | NO: 419 | ](C)[sP].[LR](A)[sP].[dR](T)[sP].[dR](T)[sP].[d | AATGAAT | 147 |
| CATTCAC | | R](C)[sP].[LR](A)[sP].[dR](T)[sP].[dR](T)[sP].[ | GAAT | |
| CAGC-3′ | | dR](C)[sP].[LR](A)[sP].[dR](C)[sP].[dR](C)[sP | | |
| | | ].[dR](A)[sP].[LR](G)[P].[LR]([5meC])}$$$$V | | |
| | | 2.0 | | |
| 5′- | SEQ ID | RNA1{[LR](T)[sP].[dR](T)[sP].[dR](C)[sP].[LR | TGCTGGT | SEQ ID NO: |
| TTCATTC | NO: 420 | ](A)[sP].[dR](T)[sP].[dR](T)[sP].[dR](C)[sP].[L | GAATGAA | 148 |
| ATTCACC | | R](A)[sP].[dR](T)[sP].[dR](T)[sP].[dR](C)[sP].[ | TGAA | |
| AGCA-3′ | | LR](A)[sP].[dR](C)[sP].[dR](C)[sP].[LR](A)[sP | | |
| | | ].[dR](G)[sP].[LR]([5meC])[sP].[LR](A)}$$$$V | | |
| | | 2.0 | | |
| 5′- | SEQ ID | RNA1{[LR](T)[sP].[dR](C)[sP].[LR](A)[sP].[dR | ATGCTGG | SEQ ID NO: |
| TCATTCA | NO: 421 | ](T)[sP].[dR](T)[sP].[dR](C)[sP].[LR](A)[sP].[d | TGAATGA | 149 |
| TTCACCA | | R](T)[sP].[dR](T)[sP].[dR](C)[sP].[LR](A)[sP].[ | ATGA | |
| GCAT-3′ | | dR](C)[sP].[dR](C)[sP].[LR](A)[sP].[dR](G)[sP | | |
| | | ].[dR](C)[sP].[LR](A)[sP].[LR](T)$$$$V2.0 | | |
| 5′- | SEQ ID | RNA1{[LR]([5meC])[sP].[dR](A)[sP].[dR](T)[s | AATGCTG | SEQ ID NO: |
| CATTCAT | NO: 422 | P].[LR](T)[sP].[dR](C)[sP].[LR](A)[sP].[dR](T) | GTGAATG | 150 |
| TCACCAG | | [sP].[dR](T)[sP].[dR](C)[sP].[LR](A)[sP].[dR]( | AATG | |
| CATT-3′ | | C)[sP].[dR](C)[sP].[LR](A)[sP].[dR](G)[sP].[d | | |
| | | R](C)[sP].[dR](A)[sP].[LR](T)[sP].[LR](T)}$$$ | | |
| | | $V2.0 | | |
| 5′- | SEQ ID | RNA1{[LR](A)[sP].[dR](T)[sP].[LR](T)[sP].[dR | AAATGCT | SEQ ID NO: |
| ATTCATT | NO: 423 | ](C)[sP].[LR](A)[P].[dR](T)[sP].[LR](T)[sP].[d | GGTGAAT | 151 |
| CACCAGC | | R](C)[sP].[LR](A)[sP].[dR](C)[sP].[dR](C)[sP]. | GAAT | |
| ATTT-3′ | | [dR](A)[sP].[LR](G)[sP].[dR](C)[sP].[LR](A)[s | | |
| | | P].[dR](T)[sP].[LR](T)[sP].[LR](T)}$$$$V2.0 | | |
| 5′- | SEQ ID | RNA1{[LR](T)[sP].[dR](T)[sP].[dR](C)[sP].[LR | TAAATGC | SEQ ID NO: |
| TTCATTC | NO: 424 | ](A)[sP].[dR](T)[sP].[dR](T)[sP].[LR]([5meC])[ | TGGTGAA | 152 |
| ACCAGCA | | sP].[dR](A)[sP].[dR](C)[sP].[dR](C)[sP].[LR]( | TGAA | |
| TTTA-3′ | | A)[sP].[dR](G)[sP].[dR](C)[P].[LR](A)[sP].[d | | |
| | | R](T)[sP].[dR](T)[sP].[LR](T)[P].[LR](A)}$$$ | | |
| | | $V2.0 | | |
| 5′- | SEQ ID | RNA1{[LR](T)[sP].[dR](C)[sP].[dR](A)[sP].[LR | ATAAATG | SEQ ID NO: |
| TCATTCA | NO: 425 | ](T)[sP].[dR](T)[sP].[dR](C)[sP].[LR](A)[sP].[d | CTGGTGA | 153 |
| CCAGCAT | | R](C)[sP].[dR](C)[sP].[dR](A)[sP].[LR](G)[sP]. | ATGA | |
| TTAT-3′ | | [dR](C)[sP].[dR](A)[sP].[LR](T)[sP].[dR](T)[sP | | |
| | | ].[dR](T)[sP].[LR](A)[sP].[LR](T)}$$$$V2.0 | | |
| 5′- | SEQ ID | RNA1{[LR]([5meC])[sP].[dR](A)[sP].[dR](T)[s | AATAAAT | SEQ ID NO: |
| CATTCAC | NO: 426 | P].[LR](T)[sP].[dR](C)[sP].[LR](A)[sP].[dR](C) | GCTGGTG | 154 |
| CAGCATT | | [sP].[dR](C)[P].[LR](A)[sP].[dR](G)[sP].[dR]( | AATG | |
| TATT-3′ | | C)[sP].[dR](A)[sP].[LR](T)[sP].[dR](T)[sP].[LR | | |
| | | ](T)[sP].[dR](A)[sP].[LR](T)[sP].[LR](T)}$$$$ | | |
| | | V2.0 | | |
| 5′- | SEQ ID | RNA1{[LR](A)[sP].[dR](T)[sP].[dR](T)[sP].[LR | GAATAAA | SEQ ID NO: |
| ATTCACC | NO: 427 | ]([5meC])[sP].[dR](A)[sP].[dR](C)[sP].[LR]([5 | TGCTGGT | 155 |
| AGCATTT | | meC])[sP].[dR](A)[sP].[dR](G)[sP].[LR]([5me | GAAT | |
| ATTC-3′ | | C])[sP].[dR](A)[sP].[dR](T)[sP].[LR](T)[sP].[d | | |
| | | R](T)[sP].[LR](A)[sP].[dR](T)[sP].[LR](T)[sP].[ | | |
| | | LR]([5meC])}$$$$V2.0 | | |
| 5′- | SEQ ID | RNA1{[LR](T)[sP].[dR](T)[sP].[dR](C)[sP].[LR | TGAATAA | SEQ ID NO: |
| TTCACCA | NO: 428 | ](A)[sP].[dR](C)[sP].[dR](C)[sP].[LR](A)[sP].[d | ATGCTGG | 156 |
| GCATTTA | | R](G)[sP].[dR](C)[sP].[LR](A)[sP].[dR](T)[sP]. | TGAA | |
| TTCA-3′ | | [LR](T)[sP].[dR](T)[sP].[LR](A)[sP].[dR](T)[sP | | |
| | | ].[dR](T)[sP].[LR]([5meC])[sP].[LR](A)}$$$$V | | |
| | | 2.0 | | |
| 5′- | SEQ ID | RNA1{[LR](T)[sP].[dR](C)[sP].[LR](A)[sP].[dR | TTGAATA | SEQ ID NO: |
| TCACCAG | NO: 429 | ](C)[sP].[dR](C)[sP].[dR](A)[sP].[LR](G)[sP].[ | AATGCTG | 157 |
| CATTTATT | | dR](C)[sP].[LR](A)[sP].[dR](T)[sP].[dR](T)[sP] | GTGA | |
| CAA-3′ | | .[dR](T)[sP].[LR](A)[sP].[dR](T)[sP].[dR](T)[s | | |
| | | P].[LR]([5meC])[sP].[LR](A)[sP].[LR](A)}$$$$ | | |
| | | V2.0 | | |
| 5′- | SEQ ID | RNA1{[LR]([5meC])[sP].[dR](A)[sP].[dR](C)[s | GTTGAAT | SEQ ID NO: |
| CACCAGC | NO: 430 | P].[dR](C)[sP].[LR](A)[sP].[dR](G)[sP].[dR](C | AAATGCT | 158 |
| ATTTATTC | | )[sP].[LR](A)[sP].[dR](T)[sP].[dR](T)[sP].[LR]( | GGTG | |
| AAC-3′ | | T)[sP].[LR](A)[sP].[dR](T)[sP].[dR](T)[sP].[dR | | |
| | | ](C)[sP].[LR](A)[sP].[LR](A)[sP].[LR]([5meC])} | | |
| | | $$$$V2.0 | | |
| 5′- | SEQ ID | RNA1{[LR]([5meC])[sP].[dR](C)[sP].[dR](A)[s | TTGTTGA | SEQ ID NO: |
| CCAGCAT | NO: 431 | P].[dR](G)[sP].[LR]([5meC])[sP].[LR](A)[sP].[ | ATAAATG | 159 |
| TTATTCAA | | dR](T)[sP].[dR](T)[sP].[LR](T)[sP].[dR](A)[sP] | CTGG | |
| CAA-3′ | | .[LR](T)[sP].[dR](T)[sP].[dR](C)[sP].[LR](A)[s | | |
| | | P].[LR](A)[sP].[dR](C)[sP].[LR](A)[sP].[LR](A) | | |
| | | }$$$$V2.0 | | |
| 5′- | SEQ ID | RNA1{[LR]([5meC])[sP].[LR](A)[sP].[dR](G)[s | TTTGTTG | SEQ ID NO: |
| CAGCATT | NO: 432 | P].[LR]([5meC])[sP].[dR](A)[sP].[dR](T)[sP].[L | AATAAAT | 160 |
| TATTCAA | | R](T)[sP].[LR](T)[sP].[dR](A)[sP].[dR](T)[sP].[ | GCTG | |
| CAAA-3′ | | LR](T)[sP].[LR]([5meC])[sP].[dR](A)[sP].[dR]( | | |
| | | A)[P].[LR]([5meC])[sP].[dR](A)[sP].[LR](A)[s | | |
| | | P].[LR](A)}$$$$V2.0 | | |
| 5′- | SEQ ID | RNA1{[LR](A)[sP].[LR](G)[sP].[dR](C)[sP].[d | GTTTGTT | SEQ ID NO: |
| AGCATTT | NO: 433 | R](A)[sP].[dR](T)[sP].[LR](T)[sP].[LR](T)[sP].[ | GAATAAA | 161 |
| ATTCAAC | | dR](A)[sP].[LR](T)[sP].[LR](T)[sP].[dR](C)[sP] | TGCT | |
| AAAC-3′ | | .[LR](A)[sP].[dR](A)[sP].[LR]([5meC])[sP].[LR | | |
| | | ](A)[sP].[dR](A)[sP].[LR](A)[sP].[LR]([5meC])} | | |
| | | $$$$V2.0 | | |
| 5′- | SEQ ID | RNA1{[LR](G)[sP].[LR]([5meC])[sP].[dR](A)[s | AGTTTGT | SEQ ID NO: |
| GCATTTA | NO: 434 | P].[LR](T)[sP].[LR](T)[sP].[dR](T)[sP].[dR](A)[ | TGAATAA | 162 |
| TTCAACA | | sP].[LR](T)[sP].[LR](T)[sP].[dR](C)[sP].[LR](A | ATGC | |
| AACT-3′ | | )[sP].[dR](A)[sP].[dR](C)[P].[LR](A)[sP].[dR]( | | |
| | | A)[sP].[dR](A)[sP].[LR]([5meC])[sP].[LR](T)}$ | | |
| | | $$$V2.0 | | |
| 5′- | SEQ ID | RNA1{[LR](A)[sP].[LR](T)[sP].[dR](T)[sP].[LR | CTAGTTT | SEQ ID NO: |
| ATTTATTC | NO: 435 | ](T)[sP].[LR](A)[sP].[dR](T)[sP].[LR](T)[sP].[L | GTTGAAT | 163 |
| AACAAAC | | R]([5meC])[sP].[dR](A)[P].[dR](A)[sP].[LR]([ | AAAT | |
| TAG-3′ | | 5meC])[sP].[dR](A)[sP].[LR](A)[sP].[LR](A)[s | | |
| | | P].[dR](C)[sP].[LR](T)[sP].[LR](A)[sP].[LR](G) | | |
| | | }$$$$V2.0 | | |
| 5′- | SEQ ID | RNA1{[LR](T)[sP].[LR](T)[sP].[dR](T)[sP].[LR | ACTAGTT | SEQ ID NO: |
| TTTATTCA | NO: 436 | ](A)[sP].[dR](T)[sP].[LR](T)[sP].[LR]([5meC])[ | TGTTGAA | 164 |
| ACAAACT | | sP].[dR](A)[sP].[dR](A)[sP].[LR]([5meC])[sP].[ | TAAA | |
| AGT-3′ | | LR](A)[sP].[dR](A)[sP].[LR](A)[sP].[LR]([5me | | |
| | | C])[sP].[dR](T)[sP].[dR](A)[sP].[LR](G)[sP].[L | | |
| | | R](T)}$$$$V2.0 | | |
| 5′- | SEQ ID | RNA1{[LR](T)[sP].[LR](T)[sP].[dR](A)[sP].[dR | AACTAGT | SEQ ID NO: |
| TTATTCAA | NO: 437 | ](T)[sP].[LR](T)[sP].[LR]([5meC])[sP].[dR](A)[ | TTGTTGA | 165 |
| CAAACTA | | SP].[LR](A)[sP].[LR]([5meC])[sP].[dR](A)[sP].[ | ATAA | |
| GTT-3′ | | LR](A)[sP].[dR](A)[sP].[LR]([5meC])\|sP].[dR]( | | |
| | | T)[sP].[dR](A)[sP].[LR](G)[P].[LR](T)[sP].[LR | | |
| | | ](T)}$$$$V2.0 | | |
| 5′- | SEQ ID | RNA1{[LR](T)[sP].[LR](A)[sP].[dR](T)[sP].[dR | GAACTAG | SEQ ID NO: |
| TATTCAA | NO: 438 | ](T)[sP].[LR]([5meC])[sP].[LR](A)[sP].[dR](A)[ | TTTGTTG | 166 |
| CAAACTA | | sP].[LR]([5meC])[sP].[LR](A)[sP].[dR](A)[sP].[ | AATA | |
| GTTC-3′ | | LR](A)[sP].[dR](C)[sP].[dR](T)[sP].[dR](A)[sP | | |
| | | ].[LR](G)[sP].[dR](T)[sP].[LR](T)[sP].[LR]([5m | | |
| | | eC])}$$$$V2.0 | | |
| 5′- | SEQ ID | RNA1{[LR](A)[sP].[LR](T)[sP].[dR](T)[sP].[dR | GGAACTA | SEQ ID NO: |
| ATTCAAC | NO: 439 | ](C)[sP].[dR](A)[sP].[LR](A)[sP].[dR](C)[sP].[d | GTTTGTT | 167 |
| AAACTAG | | R](A)[sP].[LR](A)[sP].[dR](A)[sP].[LR]([5meC] | GAAT | |
| TTCC-3′ | | )[sP].[dR](T)[sP].[dR](A)[sP].[dR](G)[sP].[LR]( | | |
| | | T)[sP].[dR](T)[sP].[LR]([5meC])[sP].[LR]([5m | | |
| | | eC])}$$$$V2.0 | | |
| 5′- | SEQ ID | RNA1{[LR](T)[sP].[dR](T)[sP].[LR]([5meC])[s | AGGAACT | SEQ ID NO: |
| TTCAACA | NO: 440 | P].[dR](A)[sP].[dR](A)[sP].[LR]([5meC])[sP].[ | AGTTTGT | 168 |
| AACTAGT | | dR](A)[sP].[dR](A)[sP].[LR](A)[sP].[dR](C)[sP | TGAA | |
| TCCT-3′ | | ].[LR](T)[sP].[dR](A)[sP].[dR](G)[sP].[LR](T)[s | | |
| | | P].[dR](T)[sP].[dR](C)[sP].[LR]([5meC])[sP].[ | | |
| | | LR](T)}$$$$V2.0 | | |
| 5′- | SEQ ID | RNA1{[LR](T)[sP].[dR](C)[sP].[LR](A)[sP].[dR | CAGGAAC | SEQ ID NO: |
| TCAACAA | NO: 441 | ](A)[sP].[dR](C)[sP].[LR](A)[sP].[dR](A)[sP].[L | TAGTTTG | 169 |
| ACTAGTT | | R](A)[sP].[dR](C)[sP].[dR](T)[sP].[dR](A)[sP]. | TTGA | |
| CCTG-3′ | | [LR](G)[sP].[dR](T)[sP].[LR](T)[sP].[dR](C)[s | | |
| | | P].[LR]([5meC])[sP].[LR](T)[sP].[LR](G)}$$$$ | | |
| | | V2.0 | | |
| 5′- | SEQ ID | RNA1{[LR]([5meC])[sP].[dR](A)[sP].[dR](A)[s | CCAGGAA | SEQ ID NO: |
| CAACAAA | NO: 442 | P].[dR](C)[sP].[LR](A)[sP].[dR](A)[sP].[dR](A) | CTAGTTT | 170 |
| CTAGTTC | | [sP].[LR]([5meC])[sP].[dR](T)[sP].[LR](A)[sP]. | GTTG | |
| CTGG-3′ | | [dR](G)[sP].[dR](T)[sP].[dR](T)[sP].[LR]([5me | | |
| | | C])[sP].[dR](C)[sP].[dR](T)[sP].[LR](G)[sP].[L | | |
| | | R](G)}$$$$V2.0 | | |
| 5′- | SEQ ID | RNA1{[LR](A)[sP].[dR](A)[sP].[dR](C)[sP].[d | CCCAGGA | SEQ ID NO: |
| AACAAAC | NO: 443 | R](A)[sP].[LR](A)[sP].[dR](A)[sP].[dR](C)[sP]. | ACTAGTT | 171 |
| TAGTTCC | | [dR](T)[sP].[LR](A)[sP].[dR](G)[sP].[dR](T)[s | TGTT | |
| TGGG-3′ | | P].[dR](T)[sP].[LR]([5meC])[sP].[dR](C)[sP].[ | | |
| | | dR](T)[sP].[dR](G)[P].[LR](G)[sP].[LR](G)}$ | | |
| | | $$$V2.0 | | |
| 5′- | SEQ ID | RNA1{[LR](A)[sP].[dR](C)[sP].[dR](A)[sP].[L | CCCCAGG | SEQ ID NO: |
| ACAAACT | NO: 444 | R](A)[sP].[dR](A)[sP].[dR](C)[sP].[dR](T)[sP]. | AACTAGT | 172 |
| AGTTCCT | | [LR](A)[sP].[dR](G)[sP].[dR](T)[sP].[LR](T)[s | TTGT | |
| GGGG-3′ | | P].[dR](C)[sP].[dR](C)[sP].[dR](T)[sP].[LR](G) | | |
| | | [sP].[dR](G)[sP].[LR](G)[sP].[LR](G)}$$$$V2. | | |
| | | 0 | | |
| 5′- | SEQ ID | RNA1{[LR]([5meC])[sP].[dR](A)[sP].[LR](A)[s | TCCCCAG | SEQ ID NO: |
| CAAACTA | NO: 445 | P].[dR](A)[sP].[dR](C)[P].[dR](T)[sP].[LR](A) | GAACTAG | 173 |
| GTTCCTG | | [sP].[dR](G)[sP].[dR](T)[sP].[LR](T)[sP].[dR]( | TTTG | |
| GGGA-3′ | | C)[sP].[dR](C)[sP].[dR](T)[sP].[LR](G)[sP].[d | | |
| | | R](G)[sP].[dR](G)[sP].[LR](G)[sP].[LR](A)}$$ | | |
| | | $$V2.0 | | |
| 5′- | SEQ ID | RNA1{[LR](A)[sP].[dR](A)[sP].[dR](A)[sP].[dR | ATCCCCA | SEQ ID NO: |
| AAACTAG | NO: 446 | ](C)[sP].[LR](T)[sP].[dR](A)[sP].[dR](G)[sP].[d | GGAACTA | 174 |
| TTCCTGG | | R](T)[sP].[LR](T)[sP].[dR](C)[sP].[dR](C)[sP]. | GTTT | |
| GGAT-3′ | | [dR](T)[sP].[LR](G)[sP].[dR](G)[sP].[dR](G)[s | | |
| | | P].[dR](G)[sP].[LR](A)[sP].[LR](T)}$$$$V2.0 | | |
| 5′- | SEQ ID | RNA1{[LR](A)[sP].[dR](A)[sP].[dR](C)[sP].[d | TATCCCC | SEQ ID NO: |
| AACTAGT | NO: 447 | R](T)[sP].[LR](A)[sP].[dR](G)[sP].[dR](T)[sP]. | AGGAACT | 175 |
| TCCTGGG | | [LR](T)[sP].[dR](C)[sP].[dR](C)[sP].[dR](T)[s | AGTT | |
| GATA-3′ | | P].[LR](G)[sP].[dR](G)[sP].[dR](G)[sP].[LR](G | | |
| | | )[sP].[dR](A)[sP].[LR](T)[sP].[LR](A)}$$$$V2. | | |
| | | 0 | | |
| 5′- | SEQ ID | RNA1{[LR](A)[sP].[dR](C)[sP].[dR](T)[sP].[LR | TTATCCC | SEQ ID NO: |
| ACTAGTT | NO: 448 | ](A)[sP].[dR](G)[sP].[dR](T)[sP].[LR](T)[sP].[d | CAGGAAC | 176 |
| CCTGGGG | | R](C)[sP].[dR](C)[sP].[dR](T)[sP].[LR](G)[sP]. | TAGT | |
| ATAA-3′ | | [dR](G)[sP].[dR](G)[sP].[dR](G)[sP].[LR](A)[s | | |
| | | P].[dR](T)[sP].[LR](A)[sP].[LR](A)}$$$$V2.0 | | |
| 5′- | SEQ ID | RNA1{[LR]([5meC])[sP].[dR](T)[sP].[LR](A)[s | CTTATCC | SEQ ID NO: |
| CTAGTTC | NO: 449 | P].[dR](G)[sP].[dR](T)[sP].[LR](T)[sP].[dR](C) | CCAGGAA | 177 |
| CTGGGGA | | [sP].[dR](C)[sP].[dR](T)[sP].[LR](G)[sP].[dR]( | CTAG | |
| TAAG-3′ | | G)[sP].[dR](G)[sP].[dR](G)[sP].[LR](A)[sP].[d | | |
| | | R](T)[sP].[dR](A)[sP].[LR](A)[sP].[LR](G)}$$$ | | |
| | | $V2.0 | | |
| 5′- | SEQ ID | RNA1{[LR](T)[sP].[dR](A)[sP].[dR](G)[sP].[d | TCTTATC | SEQ ID NO: |
| TAGTTCC | NO: 450 | R](T)[sP].[LR](T)[sP].[dR](C)[sP].[dR](C)[sP]. | CCCAGGA | 178 |
| TGGGGAT | | [dR](T)[sP].[LR](G)[sP].[dR](G)[sP].[dR](G)[s | ACTA | |
| AAGA-3′ | | P].[dR](G)[sP].[LR](A)[sP].[dR](T)[sP].[dR](A) | | |
| | | [sP].[dR](A)[sP].[LR](G)[sP].[LR](A)}$$$$V2. | | |
| | | 0 | | |
| 5′- | SEQ ID | RNA1{[LR](A)[sP].[dR](G)[sP].[dR](T)[sP].[L | CTCTTAT | SEQ ID NO: |
| AGTTCCT | NO: 451 | R](T)[sP].[dR](C)[sP].[dR](C)[sP].[dR](T)[sP]. | CCCCAGG | 179 |
| GGGGATA | | [LR](G)[sP].[dR](G)[sP].[dR](G)[sP].[dR](G)[s | AACT | |
| AGAG-3′ | | P].[LR](A)[sP].[dR](T)[sP].[LR](A)[sP].[dR](A) | | |
| | | [sP].[dR](G)[sP].[LR](A)[sP].[LR](G)}$$$$V2. | | |
| | | 0 | | |
| 5′- | SEQ ID | RNA1{[LR](G)[sP].[dR](T)[sP].[LR](T)[sP].[dR | ACTCTTA | SEQ ID NO: |
| GTTCCTG | NO: 452 | ](C)[sP].[dR](C)[sP].[dR](T)[sP].[LR](G)[sP].[ | TCCCCAG | 180 |
| GGGATAA | | dR](G)[sP].[dR](G)[sP].[dR](G)[sP].[LR](A)[s | GAAC | |
| GAGT-3′ | | P].[dR](T)[sP].[LR](A)[sP].[dR](A)[sP].[dR](G) | | |
| | | [sP].[dR](A)[sP].[LR](G)[sP].[LR](T)}$$$$V2. | | |
| | | 0 | | |
| 5′- | SEQ ID | RNA1{[LR](T)[sP].[dR](T)[sP].[dR](C)[sP].[dR | AACTCTT | SEQ ID NO: |
| TTCCTGG | NO: 453 | ](C)[P].[LR](T)[sP].[dR](G)[sP].[dR](G)[sP].[ | ATCCCCA | 181 |
| GGATAAG | | dR](G)[sP].[LR](G)[sP].[dR](A)[sP].[dR](T)[sP | GGAA | |
| AGTT-3′ | | ].[dR](A)[sP].[LR](A)[sP].[dR](G)[sP].[dR](A)[ | | |
| | | sP].[dR](G)[sP].[LR](T)[sP].[LR](T)$$$$V2.0 | | |
| 5′- | SEQ ID | RNA1{[LR](T)[sP].[dR](C)[sP].[dR](C)[sP].[d | GAACTCT | SEQ ID NO: |
| TCCTGGG | NO: 454 | R](T)[sP].[LR](G)[sP].[dR](G)[sP].[dR](G)[sP] | TATCCCC | 182 |
| GATAAGA | | .[dR](G)[sP].[LR](A)[P].[dR](T)[sP].[dR](A)[s | AGGA | |
| GTTC-3′ | | P].[dR](A)[sP].[LR](G)[sP].[dR](A)[sP].[dR](G | | |
| | | )[sP].[dR](T)[sP].[LR](T)[sP].[LR]([5meC])}$$ | | |
| | | $$V2.0 | | |
| 5′- | SEQ ID | RNA1{[LR]([5meC])[sP].[dR](C)[sP].[dR](T)[s | AGAACTC | SEQ ID NO: |
| CCTGGGG | NO: 455 | P].[LR](G)[sP].[dR](G)[sP].[dR](G)[sP].[dR](G | TTATCCC | 183 |
| ATAAGAG | | )[sP].[LR](A)[sP].[dR](T)[sP].[LR](A)[sP].[dR]( | CAGG | |
| TTCT-3′ | | A)[sP].[dR](G)[sP].[dR](A)[sP].[LR](G)[sP].[d | | |
| | | R](T)[sP].[dR](T)[sP].[LR]([5meC])[sP].[LR](T | | |
| | | )}$$$$V2.0 | | |
| 5′- | SEQ ID | RNA1{[LR]([5meC])[sP].[dR](T)[sP].[LR](G)[s | AAGAACT | SEQ ID NO: |
| CTGGGGA | NO: 456 | P].[dR](G)[sP].[dR](G)[sP].[dR](G)[sP].[LR](A | CTTATCC | 184 |
| TAAGAGT | | )[sP].[dR](T)[sP].[LR](A)[sP].[dR](A)[sP].[dR]( | CCAG | |
| TCTT-3′ | | G)[sP].[dR](A)[sP].[LR](G)[sP].[dR](T)[sP].[d | | |
| | | R](T)[sP].[dR](C)[sP].[LR](T)[sP].[LR](T)}$$$ | | |
| | | $V2.0 | | |
| 5′- | SEQ ID | RNA1{[LR](T)[sP].[dR](G)[sP].[dR](G)[sP].[L | AAAGAAC | SEQ ID NO: |
| TGGGGAT | NO: 457 | R](G)[sP].[dR](G)[sP].[dR](A)[sP].[dR](T)[sP]. | TCTTATC | 185 |
| AAGAGTT | | [LR](A)[sP].[dR](A)[sP].[LR](G)[sP].[dR](A)[s | CCCA | |
| CTTT-3′ | | P].[dR](G)[sP].[dR](T)[sP].[LR](T)[sP].[dR](C) | | |
| | | [sP].[dR](T)[sP].[LR](T)[sP].[LR](T)}$$$$V2.0 | | |
| 5′- | SEQ ID | RNA1{[LR](G)[sP].[dR](G)[sP].[dR](G)[sP].[d | GAAAGAA | SEQ ID NO: |
| GGGGATA | NO: 458 | R](G)[sP].[LR](A)[sP].[dR](T)[sP].[LR](A)[sP]. | CTCTTAT | 186 |
| AGAGTTC | | [dR](A)[sP].[LR](G)[sP].[dR](A)[sP].[dR](G)[s | CCCC | |
| TTTC-3′ | | P].[dR](T)[sP].[LR](T)[sP].[dR](C)[sP].[dR](T)[ | | |
| | | sP].[dR](T)[sP].[LR](T)[sP].[LR]([5meC])}$$$ | | |
| | | $V2.0 | | |
| 5′- | SEQ ID | RNA1{[LR](G)[sP].[dR](G)[sP].[dR](G)[sP].[L | GGAAAGA | SEQ ID NO: |
| GGGATAA | NO: 459 | R](A)[sP].[dR](T)[sP].[dR](A)[sP].[dR](A)[sP].[ | ACTCTTA | 187 |
| GAGTTCT | | LR](G)[sP].[dR](A)[sP].[dR](G)[P].[dR](T)[sP | TCCC | |
| TTCC-3′ | | ].[LR](T)[sP].[dR](C)[sP].[dR](T)[sP].[LR](T)[s | | |
| | | P].[dR](T)[sP].[LR]([5meC])[sP].[LR]([5meC]) | | |
| | | }$$$$V2.0 | | |
| 5′- | SEQ ID | RNA1{[LR](G)[sP].[dR](G)[sP].[dR](A)[sP].[d | TGGAAAG | SEQ ID NO: |
| GGATAAG | NO: 460 | R](T)[sP].[LR](A)[sP].[dR](A)[sP].[dR](G)[sP]. | AACTCTT | 188 |
| AGTTCTT | | [dR](A)[sP].[LR](G)[sP].[dR](T)[sP].[dR](T)[s | ATCC | |
| TCCA-3′ | | P].[dR](C)[sP].[LR](T)[sP].[dR](T)[sP].[dR](T)[ | | |
| | | sP].[dR](C)[sP].[LR]([5meC])[sP].[LR](A)}$$$ | | |
| | | $V2.0 | | |
| 5′- | SEQ ID | RNA1{[LR](G)[sP].[dR](A)[sP].[dR](T)[sP].[L | CTGGAAA | SEQ ID NO: |
| GATAAGA | NO: 461 | R](A)[sP].[dR](A)[sP].[LR](G)[P].[dR](A)[sP]. | GAACTCT | 189 |
| GTTCTTT | | [LR](G)[sP].[dR](T)[sP].[LR](T)[sP].[dR](C)[s | TATC | |
| CCAG-3′ | | P].[LR](T)[sP].[dR](T)[sP].[LR](T)[sP].[dR](C)[ | | |
| | | sP].[dR](C)[sP].[LR](A)[sP].[LR](G)}$$$$V2.0 | | |
| 5′- | SEQ ID | RNA1{[LR](A)[sP].[dR](T)[sP].[dR](A)[sP].[dR | CCTGGAA | SEQ ID NO: |
| ATAAGAG | NO: 462 | ](A)[sP].[LR](G)[sP].[dR](A)[sP].[dR](G)[sP].[ | AGAACTC | 190 |
| TTCTTTC | | dR](T)[sP].[LR](T)[sP].[dR](C)[sP].[dR](T)[sP] | TTAT | |
| CAGG-3′ | | .[dR](T)[sP].[LR](T)[sP].[dR](C)[sP].[dR](C)[s | | |
| | | P].[dR](A)[sP].[LR](G)[sP].[LR](G)}$$$$V2.0 | | |
| 5′- | SEQ ID | RNA1{[LR](T)[P].[dR](A)[sP].[dR](A)[sP].[LR | TCCTGGA | SEQ ID NO: |
| TAAGAGT | NO: 463 | ](G)[sP].[dR](A)[sP].[dR](G)[sP].[dR](T)[sP].[ | AAGAACT | 191 |
| TCTTTCC | | LR](T)[sP].[dR](C)[sP].[dR](T)[sP].[LR](T)[sP] | CTTA | |
| AGGA-3′ | | .[dR](T)[sP].[dR](C)[sP].[dR](C)[sP].[LR](A)[s | | |
| | | P].[dR](G)[sP].[LR](G)[sP].[LR](A)}$$$$V2.0 | | |
| 5′- | SEQ ID | RNA1{[LR](A)[sP].[dR](A)[sP].[dR](G)[sP].[L | TTCCTGG | SEQ ID NO: |
| AAGAGTT | NO: 464 | R](A)[sP].[dR](G)[sP].[dR](T)[sP].[dR](T)[sP]. | AAAGAAC | 192 |
| CTTTCCA | | [LR]([5meC])[sP].[dR](T)[sP].[dR](T)[sP].[LR] | TCTT | |
| GGAA-3′ | | (T)[sP].[dR](C)[sP].[LR]([5meC])[sP].[dR](A)[ | | |
| | | sP].[dR](G)[sP].[dR](G)[sP].[LR](A)[sP].[LR]( | | |
| | | A)}$$$$V2.0 | | |
| 5′- | SEQ ID | RNA1{[LR](A)[sP].[dR](G)[sP].[dR](A)[sP].[d | TTTCCTG | SEQ ID NO: |
| AGAGTTC | NO: 465 | R](G)[sP].[LR](T)[sP].[dR](T)[sP].[LR]([5meC] | GAAAGAA | 193 |
| TTTCCAG | | )[sP].[dR](T)[sP].[dR](T)[sP].[LR](T)[sP].[dR]( | CTCT | |
| GAAA-3′ | | C)[sP].[dR](C)[sP].[LR](A)[sP].[dR](G)[sP].[d | | |
| | | R](G)[sP].[dR](A)[sP].[LR](A)[P].[LR](A)}$$$ | | |
| | | $V2.0 | | |
| 5′- | SEQ ID | RNA1{[LR](G)[sP].[dR](A)[sP].[dR](G)[sP].[L | GTTTCCT | SEQ ID NO: |
| GAGTTCT | NO: 466 | R](T)[sP].[dR](T)[sP].[dR](C)[sP].[dR](T)[sP].[ | GGAAAGA | 194 |
| TTCCAGG | | LR](T)[sP].[dR](T)[sP].[LR]([5meC])[sP].[dR]( | ACTC | |
| AAAC-3′ | | C)[sP].[dR](A)[sP].[dR](G)[sP].[LR](G)[sP].[d | | |
| | | R](A)[sP].[dR](A)[sP].[LR](A)[sP].[LR]([5meC] | | |
| | | )}$$$$V2.0 | | |
| 5′- | SEQ ID | RNA1{[LR](A)[sP].[dR](G)[sP].[dR](T)[sP].[L | GGTTTCC | SEQ ID NO: |
| AGTTCTT | NO: 467 | R](T)[sP].[dR](C)[sP].[dR](T)[sP].[LR](T)[sP].[ | TGGAAAG | 195 |
| TCCAGGA | | dR](T)[sP].[dR](C)[sP].[dR](C)[sP].[LR](A)[sP | AACT | |
| AACC-3′ | | ].[dR](G)[sP].[dR](G)[sP].[dR](A)[sP].[LR](A)[ | | |
| | | sP].[dR](A)[P].[LR]([5meC])[sP].[LR]([5meC] | | |
| | | )}$$$$V2.0 | | |
| 5′- | SEQ ID | RNA1{[LR](G)[sP].[dR](T)[sP].[LR](T)[sP].[dR | GGGTTTC | SEQ ID NO: |
| GTTCTTT | NO: 468 | ](C)[sP].[dR](T)[sP].[LR](T)[sP].[dR](T)[sP].[d | CTGGAAA | 196 |
| CCAGGAA | | R](C)[sP].[dR](C)[P].[LR](A)[sP].[dR](G)[sP]. | GAAC | |
| ACCC-3′ | | [dR](G)[sP].[dR](A)[sP].[LR](A)[sP].[dR](A)[s | | |
| | | P].[dR](C)[sP].[LR]([5meC])[sP].[LR]([5meC]) | | |
| | | }$$$$V2.0 | | |
| 5′- | SEQ ID | RNA1{[LR](T)[sP].[dR](T)[sP].[dR](C)[sP].[dR | TGGGTTT | SEQ ID NO: |
| TTCTTTC | NO: 469 | ](T)[sP].[LR](T)[sP].[dR](T)[sP].[dR](C)[sP].[d | CCTGGAA | 197 |
| CAGGAAA | | R](C)[sP].[LR](A)[sP].[dR](G)[sP].[dR](G)[sP] | AGAA | |
| CCCA-3′ | | .[dR](A)[sP].[LR](A)[sP].[dR](A)[sP].[dR](C)[s | | |
| | | P].[dR](C)[sP].[LR]([5meC])[sP].[LR](A)}$$$$ | | |
| | | V2.0 | | |
| 5′- | SEQ ID | RNA1{[LR](T)[sP].[dR](C)[sP].[dR](T)[sP].[LR | CTGGGTT | SEQ ID NO: |
| TCTTTCC | NO: 470 | ](T)[sP].[dR](T)[sP].[dR](C)[sP].[dR](C)[sP].[L | TCCTGGA | 198 |
| AGGAAAC | | R](A)[sP].[dR](G)[sP].[dR](G)[sP].[LR](A)[sP]. | AAGA | |
| CCAG-3′ | | [dR](A)[sP].[LR](A)[sP].[dR](C)[sP].[dR](C)[s | | |
| | | P].[dR](C)[sP].[LR](A)[sP].[LR](G)$$$$V2.0 | | |
| 5′- | SEQ ID | RNA1{[LR]([5meC])[sP].[dR](T)[sP].[LR](T)[s | CCTGGGT | SEQ ID NO: |
| CTTTCCA | NO: 471 | P].[dR](T)[sP].[dR](C)[sP].[dR](C)[sP].[LR](A) | TTCCTGG | 199 |
| GGAAACC | | [sP].[dR](G)[sP].[dR](G)[sP].[dR](A)[sP].[LR]( | AAAG | |
| CAGG-3′ | | A)[sP].[dR](A)[sP].[dR](C)[sP].[dR](C)[sP].[L | | |
| | | R]([5meC])[sP].[dR](A)[sP].[LR](G)[sP].[LR]( | | |
| | | G)}$$$$V2.0 | | |
| 5′- | SEQ ID | RNA1{[LR](T)[sP].[dR](C)[sP].[dR](C)[sP].[L | CTGCCTG | SEQ ID NO: |
| TCCAGGA | NO: 472 | R](A)[sP].[dR](G)[sP].[dR](G)[sP].[LR](A)[sP]. | GGTTTCC | 200 |
| AACCCAG | | [dR](A)[sP].[LR](A)[sP].[dR](C)[sP].[dR](C)[s | TGGA | |
| GCAG-3′ | | P].[dR](C)[sP].[LR](A)[sP].[dR](G)[sP].[dR](G | | |
| | | )[sP].[dR](C)[sP].[LR](A)[sP].[LR](G)}$$$$V2. | | |
| | | 0 | | |
| 5′- | SEQ ID | RNA1{[LR]([5meC])[sP].[dR](C)[P].[LR](A)[s | GCTGCCT | SEQ ID NO: |
| CCAGGAA | NO: 473 | P].[dR](G)[sP].[dR](G)[sP].[dR](A)[P].[LR](A | GGGTTTC | 201 |
| ACCCAGG | | )[sP].[dR](A)[sP].[dR](C)[sP].[dR](C)[sP].[LR] | CTGG | |
| CAGC-3′ | | ([5meC])[sP].[dR](A)[sP].[dR](G)[sP].[dR](G)[ | | |
| | | sP].[LR]([5meC])[sP].[dR](A)[sP].[LR](G)[sP]. | | |
| | | LR]([5meC])}$$$$V2.0 | | |
| 5′- | SEQ ID | RNA1{[LR]([5meC])[sP].[dR](A)[sP].[dR](G)[s | AGCTGCC | SEQ ID NO: |
| CAGGAAA | NO: 474 | P].[dR](G)[sP].[LR](A)[sP].[dR](A)[sP].[LR](A) | TGGGTTT | 202 |
| CCCAGGC | | [sP].[dR](C)[sP].[dR](C)[sP].[dR](C)[sP].[LR]( | CCTG | |
| AGCT-3′ | | A)[sP].[dR](G)[sP].[dR](G)[sP].[dR](C)[sP].[L | | |
| | | R](A)[sP].[dR](G)[sP].[LR]([5meC])[sP].[LR]( | | |
| | | T)}$$$$V2.0 | | |
| 5′- | SEQ ID | RNA1{[LR](A)[sP].[dR](G)[sP].[dR](G)[sP].[d | CAGCTGC | SEQ ID NO: |
| AGGAAAC | NO: 475 | R](A)[P].[LR](A)[sP].[dR](A)[sP].[dR](C)[sP]. | CTGGGTT | 203 |
| CCAGGCA | | [dR](C)[sP].[LR]([5meC])[sP].[dR](A)[sP].[dR] | TCCT | |
| GCTG-3′ | | (G)[sP].[dR](G)[sP].[LR]([5meC])[sP].[dR](A)[ | | |
| | | sP].[dR](G)[sP].[dR](C)[sP].[LR](T)[sP].[LR]( | | |
| | | G)}$$$$V2.0 | | |
| 5′- | SEQ ID | RNA1{[LR](G)[sP].[dR](G)[sP].[dR](A)[sP].[d | CCAGCTG | SEQ ID NO: |
| GGAAACC | NO: 476 | R](A)[sP].[LR](A)[sP].[dR](C)[sP].[dR](C)[sP]. | CCTGGGT | 204 |
| CAGGCAG | | [dR](C)[sP].[LR](A)[sP].[dR](G)[sP].[dR](G)[s | TTCC | |
| CTGG-3′ | | P].[dR](C)[sP].[LR](A)[sP].[dR](G)[sP].[dR](C | | |
| | | )[sP].[dR](T)[sP].[LR](G)[sP].[LR](G)}$$$$V2. | | |
| | | 0 | | |
| 5′- | SEQ ID | RNA1{[LR](G)[sP].[dR](A)[sP].[dR](A)[sP].[L | TCCAGCT | SEQ ID NO: |
| GAAACCC | NO: 477 | R](A)[sP].[dR](C)[sP].[dR](C)[sP].[dR](C)[sP]. | GCCTGGG | 205 |
| AGGCAGC | | [LR](A)[sP].[dR](G)[sP].[dR](G)[sP].[dR](C)[s | TTTC | |
| TGGA-3′ | | P].[LR](A)[sP].[dR](G)[sP].[dR](C)[sP].[LR](T) | | |
| | | [sP].[dR](G)[sP].[LR](G)[sP].[LR](A)}$$$$V2. | | |
| | | 0 | | |
| 5′- | SEQ ID | RNA1{[LR](A)[sP].[dR](A)[sP].[LR](A)[sP].[dR | TTCCAGC | SEQ ID NO: |
| AAACCCA | NO: 478 | ](C)[sP].[dR](C)[sP].[dR](C)[sP].[LR](A)[sP].[ | TGCCTGG | 206 |
| GGCAGCT | | dR](G)[sP].[dR](G)[sP].[dR](C)[P].[LR](A)[s | GTTT | |
| GGAA-3′ | | P].[dR](G)[sP].[dR](C)[sP].[dR](T)[sP].[LR](G | | |
| | | )[sP].[dR](G)[sP].[LR](A)[sP].[LR](A)}$$$$V2. | | |
| | | 0 | | |
| 5′- | SEQ ID | RNA1{[LR](A)[sP].[dR](A)[sP].[dR](C)[sP].[d | CTTCCAG | SEQ ID NO: |
| AACCCAG | NO: 479 | R](C)[sP].[LR]([5meC])[sP].[dR](A)[sP].[dR]( | CTGCCTG | 207 |
| GCAGCTG | | G)[sP].[dR](G)[sP].[LR]([5meC])[sP].[dR](A)[s | GGTT | |
| GAAG-3′ | | P].[dR](G)[sP].[dR](C)[sP].[LR](T)[sP].[dR](G | | |
| | | )[sP].[dR](G)[sP].[dR](A)[sP].[LR](A)[sP].[LR] | | |
| | | (G)}$$$$V2.0 | | |
| 5′- | SEQ ID | RNA1{[LR](A)[sP].[dR](C)[sP].[dR](C)[sP].[d | TCTTCCA | SEQ ID NO: |
| ACCCAGG | NO: 480 | R](C)[sP].[LR](A)[sP].[dR](G)[sP].[dR](G)[sP] | GCTGCCT | 208 |
| CAGCTGG | | .[dR](C)[sP].[LR](A)[sP].[dR](G)[sP].[dR](C)[s | GGGT | |
| AAGA-3′ | | P].[dR](T)[sP].[LR](G)[sP].[dR](G)[sP].[dR](A) | | |
| | | [sP].[dR](A)[sP].[LR](G)[sP].[LR](A)}$$$$V2. | | |
| | | 0 | | |
| 5′- | SEQ ID | RNA1{[LR]([5meC])[sP].[dR](C)[sP].[dR](C)[s | CTCTTCC | SEQ ID NO: |
| CCCAGGC | NO: 481 | P].[LR](A)[P].[dR](G)[sP].[dR](G)[sP].[dR](C | AGCTGCC | 209 |
| AGCTGGA | | )[sP].[LR](A)[sP].[dR](G)[sP].[dR](C)[sP].[dR] | TGGG | |
| AGAG-3′ | | (T)[sP].[LR](G)[sP].[dR](G)[sP].[LR](A)[sP].[d | | |
| | | R](A)[sP].[dR](G)[sP].[LR](A)[sP].[LR](G)}$$$ | | |
| | | $V2.0 | | |
| 5′- | SEQ ID | RNA1{[LR]([5meC])[sP].[dR](C)[sP].[LR](A)[s | TCTCTTC | SEQ ID NO: |
| CCAGGCA | NO: 482 | P].[dR](G)[sP].[dR](G)[sP].[dR](C)[sP].[LR](A | CAGCTGC | 210 |
| GCTGGAA | | )[sP].[dR](G)[sP].[dR](C)[sP].[dR](T)[sP].[LR] | CTGG | |
| GAGA-3′ | | (G)[sP].[dR](G)[sP].[LR](A)[sP].[dR](A)[sP].[d | | |
| | | R](G)[sP].[dR](A)[sP].[LR](G)[sP].[LR](A)}$$$ | | |
| | | $V2.0 | | |
| 5′- | SEQ ID | RNA1{[LR]([5meC])[sP].[dR](A)[sP].[dR](G)[s | GTCTCTT | SEQ ID NO: |
| CAGGCAG | NO: 483 | P].[dR](G)[sP].[LR]([5meC])[sP].[dR](A)[sP].[ | CCAGCTG | 211 |
| CTGGAAG | | dR](G)[sP].[dR](C)[sP].[LR](T)[sP].[dR](G)[sP | CCTG | |
| AGAC-3′ | | ].[dR](G)[sP].[dR](A)[sP].[LR](A)[sP].[dR](G)[ | | |
| | | sP].[dR](A)[sP].[dR](G)[sP].[LR](A)[sP].[LR]([ | | |
| | | 5meC])}$$$$V2.0 | | |
| 5′- | SEQ ID | RNA1{[LR](A)[sP].[dR](G)[sP].[dR](G)[sP].[d | TGTCTCT | SEQ ID NO: |
| AGGCAGC | NO: 484 | R](C)[sP].[LR](A)[sP].[dR](G)[sP].[dR](C)[sP]. | TCCAGCT | 212 |
| TGGAAGA | | [dR](T)[sP].[LR](G)[sP].[dR](G)[sP].[dR](A)[s | GCCT | |
| GACA-3′ | | P].[dR](A)[sP].[LR](G)[sP].[dR](A)[sP].[dR](G | | |
| | | )[sP].[dR](A)[sP].[LR]([5meC])[sP].[LR](A)}$$ | | |
| | | $$V2.0 | | |
| 5′- | SEQ ID | RNA1{[LR](G)[sP].[dR](C)[sP].[dR](A)[sP].[d | TATGTCT | SEQ ID NO: |
| GCAGCTG | NO: 485 | R](G)[sP].[LR]([5meC])[sP].[dR](T)[sP].[dR]( | CTTCCAG | 213 |
| GAAGAGA | | G)[sP].[dR](G)[sP].[LR](A)[sP].[dR](A)[sP].[d | CTGC | |
| CATA-3′ | | R](G)[sP].[dR](A)[sP].[LR](G)[sP].[dR](A)[sP]. | | |
| | | [dR](C)[sP].[dR](A)[sP].[LR](T)[sP].[LR](A)}$ | | |
| | | $$$V2.0 | | |
| 5′- | SEQ ID | RNA1{[LR]([5meC])[sP].[dR](A)[sP].[dR](G)[s | GTATGTC | SEQ ID NO: |
| CAGCTGG | NO: 486 | P].[dR](C)[sP].[LR](T)[sP].[dR](G)[sP].[dR](G | TCTTCCA | 214 |
| AAGAGAC | | )[sP].[LR](A)[sP].[dR](A)[sP].[dR](G)[sP].[LR] | GCTG | |
| ATAC-3′ | | (A)[sP].[dR](G)[sP].[LR](A)[sP].[dR](C)[sP].[L | | |
| | | R](A)[sP].[dR](T)[sP].[LR](A)[sP].[LR]([5meC] | | |
| | | )}$$$$V2.0 | | |
| 5′- | SEQ ID | RNA1{[LR](A)[sP].[dR](G)[sP].[dR](C)[sP].[d | GGTATGT | SEQ ID NO: |
| AGCTGGA | NO: 487 | R](T)[sP].[LR](G)[P].[dR](G)[sP].[LR](A)[sP]. | CTCTTCC | 215 |
| AGAGACA | | [dR](A)[sP].[dR](G)[sP].[dR](A)[sP].[LR](G)[s | AGCT | |
| TACC-3′ | | P].[dR](A)[sP].[dR](C)[sP].[LR](A)[sP].[dR](T) | | |
| | | [sP].[dR](A)[sP].[LR]([5meC])[sP].[LR]([5meC | | |
| | | ])}$$$$V2.0 | | |
| 5′- | SEQ ID | RNA1{[LR](G)[sP].[dR](C)[sP].[dR](T)[sP].[d | GGGTATG | SEQ ID NO: |
| GCTGGAA | NO: 488 | R](G)[sP].[LR](G)[sP].[dR](A)[sP].[dR](A)[sP]. | TCTCTTC | 216 |
| GAGACAT | | [dR](G)[sP].[LR](A)[sP].[dR](G)[sP].[dR](A)[s | CAGC | |
| ACCC-3′ | | P].[dR](C)[sP].[LR](A)[sP].[dR](T)[sP].[dR](A) | | |
| | | [sP].[dR](C)[sP].[LR]([5meC])[sP].[LR]([5meC | | |
| | | ])}$$$$V2.0 | | |
| 5′- | SEQ ID | RNA1{[LR]([5meC])[sP].[dR](T)[sP].[dR](G)[s | TGGGTAT | SEQ ID NO: |
| CTGGAAG | NO: 489 | P].[dR](G)[sP].[LR](A)[sP].[dR](A)[sP].[dR](G | GTCTCTT | 217 |
| AGACATA | | )[sP].[dR](A)[sP].[LR](G)[sP].[dR](A)[sP].[dR] | CCAG | |
| CCCA-3′ | | (C)[sP].[LR](A)[sP].[dR](T)[sP].[LR](A)[sP].[d | | |
| | | R](C)[sP].[dR](C)[sP].[LR]([5meC])[sP].[LR]( | | |
| | | A)}$$$$V2.0 | | |
| 5′- | SEQ ID | RNA1{[LR](T)[P].[dR](G)[sP].[dR](G)[sP].[L | CTGGGTA | SEQ ID NO: |
| TGGAAGA | NO: 490 | R](A)[sP].[dR](A)[sP].[dR](G)[sP].[dR](A)[sP]. | TGTCTCT | 218 |
| GACATAC | | [LR](G)[sP].[dR](A)[sP].[dR](C)[sP].[LR](A)[s | TCCA | |
| CCAG-3′ | | P].[dR](T)[sP].[LR](A)[sP].[dR](C)[sP].[dR](C) | | |
| | | [sP].[dR](C)[sP].[LR](A)[sP].[LR](G)}$$$$V2. | | |
| | | 0 | | |
| 5′- | SEQ ID | RNA1{[LR](G)[sP].[dR](G)[sP].[LR](A)[sP].[d | TCTGGGT | SEQ ID NO: |
| GGAAGAG | NO: 491 | R](A)[sP].[dR](G)[sP].[dR](A)[sP].[LR](G)[sP]. | ATGTCTC | 219 |
| ACATACC | | [dR](A)[sP].[dR](C)[sP].[LR](A)[sP].[dR](T)[s | TTCC | |
| CAGA-3′ | | P].[dR](A)[sP].[dR](C)[sP].[LR]([5meC])[sP].[ | | |
| | | dR](C)[sP].[dR](A)[sP].[LR](G)[sP].[LR](A)}$$ | | |
| | | $$V2.0 | | |
| 5′- | SEQ ID | RNA1{[LR](G)[sP].[dR](A)[sP].[dR](A)[sP].[L | GTCTGGG | SEQ ID NO: |
| GAAGAGA | NO: 492 | R](G)[sP].[dR](A)[sP].[dR](G)[sP].[LR](A)[sP]. | TATGTCT | 220 |
| CATACCC | | [dR](C)[sP].[LR](A)[sP].[dR](T)[sP].[dR](A)[s | CTTC | |
| AGAC-3′ | | P].[dR](C)[sP].[LR]([5meC])[sP].[dR](C)[sP].[ | | |
| | | dR](A)[sP].[dR](G)[sP].[LR](A)[sP].[LR]([5me | | |
| | | C])}$$$$V2.0 | | |
| 5′- | SEQ ID | RNA1{[LR](A)[sP].[dR](A)[sP].[dR](G)[sP].[d | TGTCTGG | SEQ ID NO: |
| AAGAGAC | NO: 493 | R](A)[sP].[LR](G)[sP].[dR](A)[P].[dR](C)[sP]. | GTATGTC | 22 |
| ATACCCA | | [LR](A)[sP].[dR](T)[sP].[LR](A)[sP].[dR](C)[s | TCTT | |
| GACA-3′ | | P].[dR](C)[sP].[dR](C)[sP].[LR](A)[sP].[dR](G | | |
| | | )[sP].[dR](A)[sP].[LR]([5meC])[sP].[LR](A)}$$ | | |
| | | $$V2.0 | | |
| 5′- | SEQ ID | RNA1{[LR](A)[sP].[dR](G)[sP].[dR](A)[sP].[d | GTGTCTG | SEQ ID NO: |
| AGAGACA | NO: 494 | R](G)[sP].[LR](A)[sP].[dR](C)[sP].[dR](A)[sP]. | GGTATGT | 222 |
| TACCCAG | | [dR](T)[sP].[LR](A)[sP].[dR](C)[sP].[dR](C)[s | CTCT | |
| ACAC-3′ | | P].[dR](C)[sP].[LR](A)[sP].[dR](G)[sP].[dR](A) | | |
| | | [sP].[dR](C)[sP].[LR](A)[sP].[LR]([5meC])}$$ | | |
| | | $$V2.0 | | |
| 5′- | SEQ ID | RNA1{[LR](G)[sP].[dR](A)[sP].[dR](G)[sP].[L | TGTGTCT | SEQ ID NO: |
| GAGACAT | NO: 495 | R](A)[sP].[dR](C)[sP].[dR](A)[sP].[dR](T)[sP]. | GGGTATG | 223 |
| ACCCAGA | | [LR](A)[sP].[dR](C)[sP].[dR](C)[sP].[dR](C)[s | TCTC | |
| CACA-3′ | | P].[LR](A)[sP].[dR](G)[sP].[dR](A)[sP].[dR](C) | | |
| | | [sP].[LR](A)[sP].[LR]([5meC])[sP].[LR](A)}$$$ | | |
| | | $V2.0 | | |
| 5′- | SEQ ID | RNA1{[LR](A)[sP].[dR](G)[sP].[LR](A)[sP].[d | TTGTGTC | SEQ ID NO: |
| AGACATA | NO: 496 | R](C)[sP].[LR](A)[sP].[dR](T)[sP].[dR](A)[sP]. | TGGGTAT | 224 |
| CCCAGAC | | [dR](C)[sP].[LR]([5meC])[sP].[dR](C)[sP].[dR] | GTCT | |
| ACAA-3′ | | (A)[sP].[dR](G)[sP].[LR](A)[sP].[dR](C)[sP].[L | | |
| | | R](A)[sP].[dR](C)[sP].[LR](A)[sP].[LR](A)}$$$ | | |
| | | $V2.0 | | |
| 5′- | SEQ ID | RNA1{[LR](G)[sP].[dR](A)[sP].[LR]([5meC])[s | TTTGTGT | SEQ ID NO: |
| GACATAC | NO: 497 | P].[dR](A)[sP].[LR](T)[sP].[dR](A)[sP].[dR](C) | CTGGGTA | 225 |
| CCAGACA | | [sP].[LR]([5meC])[sP].[dR](C)[sP].[LR](A)[sP] | TGTC | |
| CAAA-3′ | | .[dR](G)[sP].[dR](A)[sP].[dR](C)[sP].[LR](A)[s | | |
| | | P].[dR](C)[sP].[dR](A)[sP].[LR](A)[sP].[LR](A) | | |
| | | }$$$$V2.0 | | |
| 5′- | SEQ ID | RNA1{[LR](A)[sP].[dR](C)[sP].[dR](A)[sP].[d | GTTTGTG | SEQ ID NO: |
| ACATACC | NO: 498 | R](T)[sP].[LR](A)[sP].[dR](C)[sP].[dR](C)[sP]. | TCTGGGT | 226 |
| CAGACAC | | [LR]([5meC])[sP].[dR](A)[sP].[LR](G)[sP].[dR] | ATGT | |
| AAAC-3′ | | (A)[sP].[LR]([5meC])[sP].[dR](A)[sP].[LR]([5m | | |
| | | eC])[sP].[dR](A)[sP].[dR](A)[sP].[LR](A)[sP].[ | | |
| | | LR]([5meC])}$$$$V2.0 | | |
| 5′- | SEQ ID | RNA1{[LR]([5meC])[sP].[dR](A)[sP].[dR](T)[s | CGTTTGT | SEQ ID NO: |
| CATACCC | NO: 499 | P].[LR](A)[sP].[dR](C)[sP].[dR](C)[sP].[dR](C) | GTCTGGG | 227 |
| AGACACA | | [sP].[LR](A)[sP].[dR](G)[sP].[dR](A)[sP].[LR]([ | TATG | |
| AACG-3′ | | 5meC])[sP].[dR](A)[sP].[dR](C)[sP].[LR](A)[s | | |
| | | P].[dR](A)[sP].[dR](A)[sP].[LR]([5meC])[sP].[ | | |
| | | LR](G)}$$$$V2.0 | | |
| 5′- | SEQ ID | RNA1{[LR](A)[sP].[dR](T)[sP].[LR](A)[sP].[dR | CCGTTTG | SEQ ID NO: |
| ATACCCA | NO: 500 | ](C)[sP].[dR](C)[sP].[dR](C)[sP].[LR](A)[sP].[ | TGTCTGG | 228 |
| GACACAA | | dR](G)[sP].[dR](A)[sP].[dR](C)[sP].[LR](A)[sP | GTAT | |
| ACGG-3′ | | ].[dR](C)[sP].[LR](A)[sP].[dR](A)[sP].[dR](A)[s | | |
| | | P].[dR](C)[sP].[LR](G)[sP].[LR](G)}$$$$V2.0 | | |
| 5′- | SEQ ID | RNA1{[LR](T)[sP].[dR](A)[sP].[dR](C)[sP].[dR | GCCGTTT | SEQ ID NO: |
| TACCCAG | NO: 501 | ](C)[sP].[LR]([5meC])[sP].[dR](A)[sP].[dR](G) | GTGTCTG | 229 |
| ACACAAA | | [sP].[dR](A)[sP].[LR]([5meC])[sP].[dR](A)[sP]. | GGTA | |
| CGGC-3′ | | [dR](C)[sP].[dR](A)[sP].[LR](A)[sP].[dR](A)[s | | |
| | | P].[dR]([5meC])[sP].[dR](G)[sP].[LR](G)[sP].[ | | |
| | | LR]([5meC])]$$$$V2.0 | | |
| 5′- | SEQ ID | RNA1{[LR](A)[sP].[dR](C)[sP].[dR](C)[sP].[d | GGCCGTT | SEQ ID NO: |
| ACCCAGA | NO: 502 | R](C)[sP].[LR](A)[sP].[dR](G)[sP].[dR](A)[sP]. | TGTGTCT | 230 |
| CACAAAC | | [dR](C)[sP].[LR](A)[sP].[dR](C)[sP].[dR](A)[s | GGGT | |
| GGCC-3′ | | P].[dR](A)[sP].[LR](A)[sP].[dR]([5meC])[sP].[ | | |
| | | dR](G)[sP].[dR](G)[sP].[LR]([5meC])[sP].[LR] | | |
| | | ([5meC])}$$$$V2.0 | | |
| 5′- | SEQ ID | RNA1{[LR]([5meC])[sP].[dR](C)[sP].[dR](C)[s | GGGCCGT | SEQ ID NO: |
| CCCAGAC | NO: 503 | P].[LR](A)[sP].[dR](G)[sP].[dR](A)[sP].[dR](C) | TTGTGTC | 231 |
| ACAAACG | | [sP].[LR](A)[sP].[dR](C)[sP].[LR](A)[sP].[dR]( | TGGG | |
| GCCC-3′ | | A)[sP].[dR](A)[sP].[LR]([5meC])[sP].[dR](G)[s | | |
| | | P].[dR](G)[sP].[dR](C)[sP].[LR]([5meC])[sP].[ | | |
| | | LR]([5meC])}$$$$V2.0 | | |
| 5′- | SEQ ID | RNA1{[LR]([5meC])[sP].[dR](C)[sP].[dR](A)[s | TGGGCCG | SEQ ID NO: |
| CCAGACA | NO: 504 | P].[dR](G)[sP].[LR](A)[sP].[dR](C)[sP].[dR](A) | TTTGTGT | 232 |
| CAAACGG | | [sP].[dR](C)[sP].[LR](A)[sP].[dR](A)[sP].[dR]( | CTGG | |
| CCCA-3′ | | A)[sP].[dR](C)[sP].[LR](G)[sP].[dR](G)[sP].[d | | |
| | | R](C)[sP].[dR](C)[sP].[LR]([5meC])[sP].[LR]( | | |
| | | A)}$$$$V2.0 | | |
| 5′- | SEQ ID | RNA1{[LR]([5meC])[sP].[dR](A)[sP].[dR](G)[s | TTGGGCC | SEQ ID NO: |
| CAGACAC | NO: 505 | P].[dR](A)[sP].[LR]([5meC])[sP].[dR](A)[sP].[ | GTTTGTG | 233 |
| AAACGGC | | dR](C)[sP].[dR](A)[sP].[LR](A)[sP].[dR](A)[sP | TCTG | |
| CCAA-3′ | | ].[dR]([5meC])[sP].[dR](G)[P].[LR](G)[sP].[d | | |
| | | R](C)[sP].[dR](C)[sP].[dR](C)[sP].[LR](A)[sP]. | | |
| | | [LR](A)}$$$$V2.0 | | |
| 5′- | SEQ ID | RNA1{[LR](A)[P].[dR](G)[sP].[dR](A)[sP].[d | ATTGGGC | SEQ ID NO: |
| AGACACA | NO: 506 | R](C)[sP].[LR](A)[sP].[dR](C)[sP].[dR](A)[sP]. | CGTTTGT | 234 |
| AACGGCC | | [dR](A)[sP].[LR](A)[sP].[dR]([5meC])[sP].[dR] | GTCT | |
| CAAT-3′ | | (G)[sP].[dR](G)[sP].[LR]([5meC])[sP].[dR](C)[ | | |
| | | sP].[dR](C)[sP].[dR](A)[sP].[LR](A)[sP].[LR](T | | |
| | | )}$$$$V2.0 | | |
| 5′- | SEQ ID | RNA1{[LR](A)[P].[dR](C)[sP].[dR](A)[sP].[d | GGATTGG | SEQ ID NO: |
| ACACAAA | NO: 507 | R](C)[sP].[LR](A)[sP].[dR](A)[sP].[dR](A)[sP]. | GCCGTTT | 235 |
| CGGCCCA | | [dR](C)[sP].[LR](G)[sP].[dR](G)[sP].[dR](C)[s | GTGT | |
| ATCC-3′ | | P].[dR](C)[sP].[LR]([5meC])[sP].[dR](A)[sP].[ | | |
| | | dR](A)[sP].[dR](T)[sP].[LR]([5meC])[sP].[LR]( | | |
| | | [5meC])}$$$$V2.0 | | |
| 5′- | SEQ ID | RNA1{[LR]([5meC])[sP].[dR](A)[sP].[dR](C)[s | AGGATTG | SEQ ID NO: |
| CACAAAC | NO: 508 | P].[dR](A)[sP].[LR](A)[sP].[dR](A)[sP].[dR]([5 | GGCCGTT | 236 |
| GGCCCAA | | meC])[sP].[dR](G)[sP].[LR](G)[sP].[dR](C)[sP | TGTG | |
| TCCT-3′ | | ].[dR](C)[sP].[dR](C)[sP].[LR](A)[sP].[dR](A)[ | | |
| | | sP].[dR](T)[sP].[dR](C)[sP].[LR]([5meC])[sP].[ | | |
| | | LR](T)}$$$$V2.0 | | |
| 5′- | SEQ ID | RNA1{[LR](A)[sP].[dR](C)[sP].[dR](A)[sP].[d | CAGGATT | SEQ ID NO: |
| ACAAACG | NO: 509 | R](A)[P].[LR](A)[sP].[dR]([5meC])[sP].[dR]( | GGGCCGT | 237 |
| GCCCAAT | | G)[sP].[dR](G)[sP].[LR]([5meC])[sP].[dR](C)[ | TTGT | |
| CCTG-3′ | | sP].[dR](C)[sP].[dR](A)[sP].[LR](A)[sP].[dR](T | | |
| | | )[sP].[dR](C)[sP].[dR](C)[sP].[LR](T)[sP].[LR]( | | |
| | | G)}$$$$V2.0 | | |
| 5′- | SEQ ID | RNA1{[LR]([5meC])[sP].[dR](A)[sP].[LR](A)[s | TCAGGAT | SEQ ID NO: |
| CAAACGG | NO: 510 | P].[dR](A)[sP].[dR]([5meC])[sP].[dR](G)[sP].[ | TGGGCCG | 238 |
| CCCAATC | | LR](G)[sP].[dR](C)[sP].[dR](C)[sP].[dR](C)[s | TTTG | |
| CTGA-3′ | | P].[LR](A)[sP].[dR](A)[sP].[LR](T)[sP].[dR](C) | | |
| | | [sP].[dR](C)[sP].[dR](T)[sP].[LR](G)[sP].[LR]( | | |
| | | A)}$$$$V2.0 | | |
| 5′- | SEQ ID | RNA1{[LR](A)[sP].[dR](A)[sP].[dR](A)[sP].[dR | CTCAGGA | SEQ ID NO: |
| AAACGGC | NO: 511 | ](C)[sP].[LR](G)[sP].[dR](G)[sP].[dR](C)[sP].[ | TTGGGCC | 239 |
| CCAATCC | | dR](C)[P].[LR]([5meC])[sP].[dR](A)[sP].[dR]( | GTTT | |
| TGAG-3′ | | A)[sP].[dR](T)[sP].[LR]([5meC])[sP].[dR](C)[s | | |
| | | P].[dR](T)[sP].[dR](G)[sP].[LR](A)[sP].[LR](G) | | |
| | | }$$$$V2.0 | | |
| 5′- | SEQ ID | RNA1{[LR](A)[sP].[dR](A)[sP].[dR]([5meC])[s | ACTCAGG | SEQ ID NO: |
| AACGGCC | NO: 512 | P].[dR](G)[sP].[LR](G)[sP].[dR](C)[sP].[dR](C | ATTGGGC | 240 |
| CAATCCT | | )[sP].[dR](C)[sP].[LR](A)[sP].[dR](A)[sP].[LR]( | CGTT | |
| GAGT-3′ | | T)[sP].[dR](C)[sP].[dR](C)[sP].[dR](T)[sP].[LR | | |
| | | ](G)[sP].[dR](A)[sP].[LR](G)[sP].[LR](T)}$$$$ | | |
| | | V2.0 | | |
| 5′- | SEQ ID | RNA1{[LR](A)[sP].[dR]([5meC])[sP].[dR](G)[s | CACTCAG | SEQ ID NO: |
| ACGGCCC | NO: 513 | P].[LR](G)[sP].[dR](C)[sP].[dR](C)[sP].[dR](C | GATTGGG | 241 |
| AATCCTG | | )[sP].[LR](A)[sP].[dR](A)[sP].[LR](T)[sP].[dR]( | CCGT | |
| AGTG-3′ | | C)[sP].[dR](C)[sP].[dR](T)[sP].[LR](G)[sP].[d | | |
| | | R](A)[sP].[dR](G)[sP].[LR](T)[sP].[LR](G)}$$$ | | |
| | | $V2.0 | | |
| 5′- | SEQ ID | RNA1{[LR]([5meC])[sP].[dR](G)[sP].[dR](G)[s | CCACTCA | SEQ ID NO: |
| CGGCCCA | NO: 514 | P].[dR](C)[sP].[LR]([5meC])[sP].[dR](C)[sP].[ | GGATTGG | 242 |
| ATCCTGA | | dR](A)[sP].[dR](A)[sP].[LR](T)[sP].[dR](C)[sP | GCCG | |
| GTGG-3′ | | ].[dR](C)[sP].[dR](T)[sP].[LR](G)[sP].[dR](A)[ | | |
| | | sP].[dR](G)[sP].[dR](T)[sP].[LR](G)[sP].[LR]( | | |
| | | G)}$$$$V2.0 | | |
| 5′- | SEQ ID | RNA1{[LR](G)[sP].[dR](G)[sP].[dR](C)[sP].[d | ACCACTC | SEQ ID NO: |
| GGCCCAA | NO: 515 | R](C)[sP].[LR]([5meC])[sP].[dR](A)[sP].[dR]( | AGGATTG | 243 |
| TCCTGAG | | A)[sP].[LR](T)[sP].[dR](C)[sP].[dR](C)[sP].[d | GGCC | |
| TGGT-3′ | | R](T)[sP].[LR](G)[sP].[dR](A)[sP].[LR](G)[sP]. | | |
| | | [dR](T)[sP].[dR](G)[sP].[LR](G)[sP].[LR](T)}$ | | |
| | | $$$V2.0 | | |
| 5′- | SEQ ID | RNA1{[LR](G)[sP].[dR](C)[sP].[dR](C)[sP].[d | AACCACT | SEQ ID NO: |
| GCCCAAT | NO: 516 | R](C)[sP].[LR](A)[sP].[dR](A)[sP].[LR](T)[sP]. | CAGGATT | 244 |
| CCTGAGT | | [dR](C)[sP].[dR](C)[sP].[dR](T)[sP].[LR](G)[s | GGGC | |
| GGTT-3′ | | P].[dR](A)[sP].[dR](G)[sP].[dR](T)[sP].[LR](G) | | |
| | | [sP].[dR](G)[sP].[LR](T)[sP].[LR](T)}$$$$V2. | | |
| | | 0 | | |
| 5′- | SEQ ID | RNA1{[LR]([5meC])[sP].[dR](C)[sP].[dR](C)[s | TAACCAC | SEQ ID NO: |
| CCCAATC | NO: 517 | P].[LR](A)[sP].[dR](A)[sP].[LR](T)[sP].[dR](C) | TCAGGAT | 245 |
| CTGAGTG | | [sP].[dR](C)[sP].[dR](T)[sP].[LR](G)[sP].[dR]( | TGGG | |
| GTTA-3′ | | A)[sP].[dR](G)[sP].[dR](T)[sP].[LR](G)[sP].[d | | |
| | | R](G)[sP].[dR](T)[sP].[LR](T)[sP].[LR](A)}$$$ | | |
| | | $V2.0 | | |
| 5′- | SEQ ID | RNA1{[LR]([5meC])[sP].[dR](C)[sP].[dR](A)[s | CTAACCA | SEQ ID NO: |
| CCAATCC | NO: 518 | P].[dR](A)[sP].[LR](T)[sP].[dR](C)[sP].[dR](C) | CTCAGGA | 246 |
| TGAGTGG | | [sP].[dR](T)[sP].[LR](G)[sP].[dR](A)[sP].[dR]( | TTGG | |
| TTAG-3′ | | G)[sP].[dR](T)[sP].[LR](G)[sP].[dR](G)[sP].[d | | |
| | | R](T)[sP].[dR](T)[sP].[LR](A)[sP].[LR](G)}$$$ | | |
| | | $V2.0 | | |
| 5′- | SEQ ID | RNA1{[LR]([5meC])[P].[dR](A)[sP].[dR](A)[s | CCTAACC | SEQ ID NO: |
| CAATCCT | NO: 519 | P].[LR](T)[sP].[dR](C)[sP].[dR](C)[sP].[dR](T) | ACTCAGG | 247 |
| GAGTGGT | | [sP].[LR](G)[sP].[dR](A)[sP].[dR](G)[sP].[dR]( | ATTG | |
| TAGG-3′ | | T)[sP].[LR](G)[sP].[dR](G)[P].[LR](T)[sP].[d | | |
| | | R](T)[sP].[dR](A)[sP].[LR](G)[sP].[LR](G)}$$$ | | |
| | | $V2.0 | | |
| 5′- | SEQ ID | RNA1{[LR](A)[sP].[dR](A)[sP].[LR](T)[sP].[dR | CCCTAAC | SEQ ID NO: |
| AATCCTG | NO: 520 | ](C)[sP].[dR](C)[sP].[dR](T)[sP].[LR](G)[sP].[ | CACTCAG | 248 |
| AGTGGTT | | dR](A)[sP].[dR](G)[sP].[dR](T)[sP].[LR](G)[sP | GATT | |
| AGGG-3′ | | ].[dR](G)[sP].[dR](T)[sP].[dR](T)[sP].[LR](A)[s | | |
| | | P].[dR](G)[sP].[LR](G)[sP].[LR](G)$$$$V2.0 | | |
| 5′- | SEQ ID | RNA1{[LR](A)[sP].[dR](T)[sP].[LR]([5meC])[s | GCCCTAA | SEQ ID NO: |
| ATCCTGA | NO: 521 | P].[dR](C)[sP].[dR](T)[sP].[LR](G)[sP].[dR](A) | CCACTCA | 249 |
| GTGGTTA | | [sP].[dR](G)[sP].[dR](T)[sP].[LR](G)[sP].[dR]( | GGAT | |
| GGGC-3′ | | G)[sP].[dR](T)[sP].[dR](T)[sP].[LR](A)[sP].[dR | | |
| | | ](G)[sP].[dR](G)[sP].[LR](G)[sP].[LR]([5meC]) | | |
| | | }$$$$V2.0 | | |
| 5′- | SEQ ID | RNA1{[LR](T)[sP].[dR](C)[sP].[dR](C)[sP].[d | AGCCCTA | SEQ ID NO: |
| TCCTGAG | NO: 522 | R](T)[sP].[LR](G)[sP].[dR](A)[sP].[dR](G)[sP]. | ACCACTC | 250 |
| TGGTTAG | | [dR](T)[sP].[LR](G)[sP].[dR](G)[sP].[dR](T)[s | AGGA | |
| GGCT-3′ | | P].[dR](T)[sP].[LR](A)[sP].[dR](G)[sP].[dR](G) | | |
| | | [sP].[dR](G)[sP].[LR]([5meC])[sP].[LR](T)}$$ | | |
| | | $$V2.0 | | |
| 5′- | SEQ ID | RNA1{[LR]([5meC])[sP].[dR](C)[sP].[dR](T)[s | CAGCCCT | SEQ ID NO: |
| CCTGAGT | NO: 523 | P].[LR](G)[sP].[dR](A)[sP].[dR](G)[sP].[dR](T) | AACCACT | 251 |
| GGTTAGG | | [sP].[LR](G)[sP].[dR](G)[sP].[dR](T)[sP].[dR]( | CAGG | |
| GCTG-3′ | | T)[sP].[LR](A)[sP].[dR](G)[sP].[dR](G)[sP].[L | | |
| | | R](G)[sP].[dR](C)[sP].[LR](T)[sP].[LR](G)}$$$ | | |
| | | $V2.0 | | |
| 5′- | SEQ ID | RNA1{[LR]([5meC])[sP].[dR](T)[sP].[LR](G)[s | CCAGCCC | SEQ ID NO: |
| CTGAGTG | NO: 524 | P].[dR](A)[sP].[dR](G)[sP].[dR](T)[sP].[LR](G) | TAACCAC | 252 |
| GTTAGGG | | [sP].[dR](G)[sP].[dR](T)[sP].[dR](T)[sP].[LR]( | TCAG | |
| CTGG-3′ | | A)[sP].[dR](G)[sP].[dR](G)[sP].[LR](G)[sP].[d | | |
| | | R](C)[sP].[dR](T)[sP].[LR](G)[sP].[LR](G)}$$$ | | |
| | | $V2.0 | | |
| 5′- | SEQ ID | RNA1{[LR](T)[sP].[dR](G)[sP].[dR](A)[sP].[d | TCCAGCC | SEQ ID NO: |
| TGAGTGG | NO: 525 | R](G)[sP].[LR](T)[sP].[dR](G)[sP].[dR](G)[sP] | CTAACCA | 253 |
| TTAGGGC | | .[dR](T)[sP].[LR](T)[sP].[dR](A)[sP].[dR](G)[s | CTCA | |
| TGGA-3′ | | P].[dR](G)[sP].[LR](G)[sP].[dR](C)[sP].[dR](T | | |
| | | )[sP].[dR](G)[sP].[LR](G)[sP].[LR](A)}$$$$V2 | | |
| | | 0 | | |
| 5′- | SEQ ID | RNA1{[LR](G)[sP].[dR](A)[sP].[dR](G)[sP].[d | TTCCAGC | SEQ ID NO: |
| GAGTGGT | NO: 526 | R](T)[sP].[LR](G)[sP].[dR](G)[sP].[dR](T)[sP]. | CCTAACC | 254 |
| TAGGGCT | | [dR](T)[sP].[LR](A)[sP].[dR](G)[sP].[dR](G)[s | ACTC | |
| GGAA-3′ | | P].[dR](G)[sP].[LR]([5meC])[sP].[dR](T)[sP].[ | | |
| | | dR](G)[sP].[dR](G)[sP].[LR](A)[sP].[LR](A)}$$ | | |
| | | $$V2.0 | | |
| 5′- | SEQ ID | RNA1{[LR](A)[sP].[dR](G)[sP].[dR](T)[sP].[L | ATTCCAG | SEQ ID NO: |
| AGTGGTT | NO: 527 | R](G)[sP].[dR](G)[sP].[dR](T)[sP].[dR](T)[sP]. | CCCTAAC | 255 |
| AGGGCTG | | [LR](A)[sP].[dR](G)[sP].[dR](G)[sP].[LR](G)[s | CACT | |
| GAAT-3′ | | P].[dR](C)[sP].[dR](T)[sP].[dR](G)[sP].[LR](G | | |
| | | )[sP].[dR](A)[sP].[LR](A)[sP].[LR](T)}$$$$V2. | | |
| | | 0 | | |
| 5′- | SEQ ID | RNA1{[LR](G)[sP].[dR](T)[sP].[dR](G)[sP].[d | TATTCCA | SEQ ID NO: |
| GTGGTTA | NO: 528 | R](G)[sP].[LR](T)[sP].[dR](T)[sP].[dR](A)[sP]. | GCCCTAA | 256 |
| GGGCTG | | [dR](G)[sP].[LR](G)[sP].[dR](G)[sP].[dR](C)[s | CCAC | |
| GAATA-3′ | | P].[dR](T)[sP].[LR](G)[sP].[dR](G)[sP].[dR](A) | | |
| | | [sP].[dR](A)[sP].[LR](T)[sP].[LR](A)}$$$$V2.0 | | |
| 5′- | SEQ ID | RNA1{[LR](T)[sP].[dR](G)[sP].[dR](G)[sP].[d | CTATTCC | SEQ ID NO: |
| TGGTTAG | NO: 529 | R](T)[sP].[LR](T)[sP].[dR](A)[sP].[dR](G)[sP]. | AGCCCTA | 257 |
| GGCTGGA | | [dR](G)[sP].[LR](G)[sP].[dR](C)[sP].[dR](T)[ | ACCA | |
| ATAG-3′ | | P].[dR](G)[sP].[LR](G)[sP].[dR](A)[sP].[dR](A | | |
| | | )[sP].[dR](T)[sP].[LR](A)[sP].[LR](G)}$$$$V2. | | |
| | | 0 | | |
| 5′- | SEQ ID | RNA1{[LR](G)[sP].[dR](G)[sP].[dR](T)[sP].[d | TCTATTC | SEQ ID NO: |
| GGTTAGG | NO: 530 | R](T)[sP].[LR](A)[sP].[dR](G)[sP].[dR](G)[sP]. | CAGCCCT | 258 |
| GCTGGAA | | [dR](G)[sP].[LR]([5meC])[sP].[dR](T)[sP].[dR] | AACC | |
| TAGA-3′ | | (G)[sP].[dR](G)[sP].[LR](A)[sP].[dR](A)[sP].[d | | |
| | | R](T)[sP].[dR](A)[sP].[LR](G)[sP].[LR](A)}$$$ | | |
| | | $V2.0 | | |
| 5′- | SEQ ID | RNA1{[LR](G)[sP].[dR](T)[sP].[dR](T)[sP].[LR | TTCTATTC | SEQ ID NO: |
| GTTAGGG | NO: 531 | ](A)[sP].[dR](G)[sP].[dR](G)[sP].[LR](G)[sP].[ | CAGCCCT | 259 |
| CTGGAAT | | dR](C)[sP].[LR](T)[sP].[dR](G)[sP].[dR](G)[sP | AAC | |
| AGAA-3′ | | ].[dR](A)[sP].[LR](A)[sP].[dR](T)[sP].[dR](A)[s | | |
| | | P].[dR](G)[sP].[LR](A)[sP].[LR](A)}$$$$V2.0 | | |
| 5′- | SEQ ID | RNA1{[LR](T)[sP].[dR](T)[sP].[LR](A)[sP].[dR | CTTCTATT | SEQ ID NO: |
| TTAGGGC | NO: 532 | ](G)[sP].[dR](G)[sP].[LR](G)[sP].[dR](C)[sP].[ | CCAGCCC | 260 |
| TGGAATA | | dR](T)[sP].[dR](G)[sP].[LR](G)[sP].[dR](A)[sP | TAA | |
| GAAG-3′ | | ].[LR](A)[sP].[dR](T)[sP].[LR](A)[sP].[dR](G)[s | | |
| | | P].[dR](A)[sP].[LR](A)[sP].[LR](G)}$$$$V2.0 | | |
| 5′- | SEQ ID | RNA1{[LR](T)[sP].[dR](A)[sP].[dR](G)[sP].[d | CCTTCTA | SEQ ID NO: |
| TAGGGCT | NO: 533 | R](G)[sP].[LR](G)[sP].[dR](C)[sP].[dR](T)[sP]. | TTCCAGC | 26 |
| GGAATAG | | [dR](G)[sP].[LR](G)[sP].[dR](A)[sP].[dR](A)[s | CCTA | |
| AAGG-3′ | | P].[dR](T)[sP].[LR](A)[sP].[dR](G)[sP].[dR](A) | | |
| | | [sP].[dR](A)[sP].[LR](G)[sP].[LR](G)}$$$$V2. | | |
| | | 0 | | |
| 5′- | SEQ ID | RNA1{[LR](A)[sP].[dR](G)[sP].[dR](G)[sP].[d | TCCTTCT | SEQ ID NO: |
| AGGGCTG | NO: 534 | R](G)[sP].[LR]([5meC])[sP].[dR](T)[sP].[dR]( | ATTCCAG | 262 |
| GAATAGA | | G)[sP].[dR](G)[sP].[LR](A)[sP].[dR](A)[sP].[d | CCCT | |
| AGGA-3′ | | R](T)[sP].[dR](A)[sP].[LR](G)[sP].[dR](A)[sP]. | | |
| | | [dR](A)[sP].[dR](G)[sP].[LR](G)[sP].[LR](A)}$ | | |
| | | $$$V2.0 | | |
| 5′- | SEQ ID | RNA1{[LR](G)[sP].[dR](G)[sP].[dR](G)[sP].[d | TTCCTTC | SEQ ID NO: |
| GGGCTG | NO: 535 | R](C)[sP].[LR](T)[sP].[dR](G)[sP].[dR](G)[sP]. | TATTCCA | 263 |
| GAATAGA | | [dR](A)[sP].[LR](A)[sP].[dR](T)[sP].[LR](A)[sP | GCCC | |
| AGGAA-3′ | | ].[dR](G)[sP].[LR](A)[sP].[dR](A)[sP].[dR](G)[ | | |
| | | sP].[dR](G)[sP].[LR](A)[sP].[LR](A)}$$$$V2.0 | | |
| 5′- | SEQ ID | RNA1{[LR](G)[sP].[dR](G)[sP].[dR](C)[sP].[d | CTTCCTT | SEQ ID NO: |
| GGCTGGA | NO: 536 | R](T)[sP].[LR](G)[sP].[dR](G)[sP].[dR](A)[sP]. | CTATTCC | 264 |
| ATAGAAG | | [LR](A)[sP].[dR](T)[sP].[dR](A)[sP].[dR](G)[s | AGCC | |
| GAAG-3′ | | P].[LR](A)[sP].[dR](A)[sP].[dR](G)[sP].[LR](G | | |
| | | )[sP].[dR](A)[sP].[LR](A)[sP].[LR](G)}$$$$V2. | | |
| | | 0 | | |
| 5′- | SEQ ID | RNA1{[LR](G)[sP].[dR](C)[sP].[dR](T)[sP].[d | TCTTCCT | SEQ ID NO: |
| GCTGGAA | NO: 537 | R](G)[sP].[LR](G)[sP].[dR](A)[sP].[LR](A)[sP]. | TCTATTC | 265 |
| TAGAAGG | | [dR](T)[sP].[dR](A)[sP].[dR](G)[sP].[LR](A)[s | CAGC | |
| AAGA-3′ | | P].[LR](A)[sP].[dR](G)[sP].[dR](G)[sP].[LR](A | | |
| | | )[sP].[dR](A)[sP].[LR](G)[sP].[LR](A)}$$$$V2. | | |
| | | 0 | | |
| 5′- | SEQ ID | RNA1{[LR]([5meC])[sP].[LR](T)[sP].[dR](G)[s | TTCTTCC | SEQ ID NO: |
| CTGGAAT | NO: 538 | P].[dR](G)[sP].[dR](A)[sP].[LR](A)[sP].[dR](T) | TTCTATTC | 266 |
| AGAAGGA | | [sP].[dR](A)[sP].[LR](G)[sP].[LR](A)[sP].[dR]( | CAG | |
| AGAA-3′ | | A)[sP].[LR](G)[sP].[dR](G)[sP].[dR](A)[sP].[L | | |
| | | R](A)[sP].[dR](G)[sP].[LR](A)[sP].[LR](A)}$$$ | | |
| | | $V2.0 | | |
| 5′- | SEQ ID | RNA1{[LR](T)[sP].[dR](G)[sP].[LR](G)[sP].[L | GTTCTTC | SEQ ID NO: |
| TGGAATA | NO: 539 | R](A)[sP].[dR](A)[sP].[dR](T)[sP].[LR](A)[sP].[ | CTTCTATT | 267 |
| GAAGGAA | | LR](G)[sP].[dR](A)[sP].[dR](A)[sP].[dR](G)[sP | CCA | |
| GAAC-3′ | | ].[LR](G)[sP].[dR](A)[sP].[LR](A)[sP].[LR](G)[ | | |
| | | sP].[dR](A)[sP].[LR](A)[sP].[LR]([5meC])}$$$ | | |
| | | $V2.0 | | |
| 5′- | SEQ ID | RNA1{[LR](G)[sP].[dR](G)[sP].[LR](A)[sP].[d | GGTTCTT | SEQ ID NO: |
| GGAATAG | NO: 540 | R](A)[P].[LR](T)[sP].[dR](A)[sP].[dR](G)[sP]. | CCTTCTA | 268 |
| AAGGAAG | | [dR](A)[sP].[LR](A)[sP].[dR](G)[sP].[dR](G)[s | TTCC | |
| AACC-3′ | | P].[LR](A)[sP].[dR](A)[sP].[dR](G)[sP].[dR](A) | | |
| | | [sP].[LR](A)[sP].[LR]([5meC])[sP].[LR]([5meC | | |
| | | ])}$$$$V2.0 | | |
| 5′- | SEQ ID | RNA1{[LR](G)[sP].[dR](A)[sP].[dR](A)[sP].[L | AGGTTCT | SEQ ID NO: |
| GAATAGA | NO: 541 | R](T)[sP].[dR](A)[sP].[dR](G)[sP].[LR](A)[sP]. | TCCTTCT | 269 |
| AGGAAGA | | [LR](A)[sP].[dR](G)[sP].[LR](G)[sP].[dR](A)[s | ATTC | |
| ACCT-3′ | | P].[LR](A)[sP].[LR](G)[sP].[dR](A)[sP].[dR](A) | | |
| | | [sP].[dR](C)[sP].[LR]([5meC])[sP].[LR](T)}$$$ | | |
| | | $V2.0 | | |
| 5′- | SEQ ID | RNA1{[LR](A)[sP].[LR](A)[sP].[dR](T)[sP].[dR | CAGGTTC | SEQ ID NO: |
| AATAGAA | NO: 542 | ](A)[sP].[dR](G)[sP].[LR](A)[sP].[dR](A)[sP].[L | TTCCTTC | 270 |
| GGAAGAA | | R](G)[sP].[LR](G)[sP].[dR](A)[sP].[dR](A)[sP]. | TATT | |
| CCTG-3′ | | [dR](G)[sP].[LR](A)[sP].[dR](A)[sP].[dR](C)[s | | |
| | | P].[LR]([5meC])[sP].[LR](T)[sP].[LR](G)}$$$$ | | |
| | | V2.0 | | |
| 5′- | SEQ ID | RNA1{[LR](A)[sP].[dR](T)[sP].[LR](A)[sP].[dR | TCAGGTT | SEQ ID NO: |
| ATAGAAG | NO: 543 | ](G)[sP].[LR](A)[sP].[dR](A)[sP].[dR](G)[sP].[ | CTTCCTT | 271 |
| GAAGAAC | | LR](G)[sP].[dR](A)[sP].[LR](A)[sP].[LR](G)[sP | CTAT | |
| CTGA-3′ | | ].[dR](A)[sP].[dR](A)[sP].[LR]([5meC])[sP].[d | | |
| | | R](C)[sP].[dR](T)[sP].[LR](G)[sP].[LR](A)}$$$ | | |
| | | $V2.0 | | |
| 5′- | SEQ ID | RNA1{[LR]([5meC])[sP].[dR](T)[sP].[LR](A)[s | GAGTACA | SEQ ID NO: |
| CTATCCA | NO: 544 | P].[LR](T)[sP].[dR](C)[sP].[dR](C)[sP].[LR](A) | TGGATGG | 272 |
| TCCATGT | | [sP].[dR](T)[sP].[dR](C)[sP].[dR](C)[sP].[LR]( | ATAG | |
| ACTC-3′ | | A)[sP].[dR](T)[sP].[dR](G)[sP].[dR](T)[sP].[LR | | |
| | | ](A)[sP].[dR](C)[sP].[LR](T)[sP].[LR]([5meC])} | | |
| | | $$$$V2.0 | | |
| 5′- | SEQ ID | RNA1{[LR](T)[sP].[dR](A)[sP].[LR](T)[sP].[dR | TGAGTAC | SEQ ID NO: |
| TATCCAT | NO: 545 | ](C)[sP].[dR](C)[sP].[LR](A)[P].[LR](T)[sP].[d | ATGGATG | 273 |
| CCATGTA | | R](C)[sP].[dR](C)[sP].[LR](A)[sP].[dR](T)[P]. | GATA | |
| CTCA-3′ | | [dR](G)[sP].[dR](T)[sP].[LR](A)[sP].[dR](C)[s | | |
| | | P].[dR](T)[sP].[LR]([5meC])[sP].[LR](A)}$$$$ | | |
| | | V2.0 | | |
| 5′- | SEQ ID | RNA1{[LR](A)[sP].[dR](T)[sP].[LR]([5meC])[s | GTGAGTA | SEQ ID NO: |
| ATCCATC | NO: 546 | P].[dR](C)[sP].[LR](A)[P].[dR](T)[sP].[dR](C) | CATGGAT | 274 |
| CATGTAC | | [sP].[dR](C)[sP].[LR](A)[sP].[dR](T)[sP].[dR]( | GGAT | |
| TCAC-3′ | | G)[sP].[dR](T)[P].[LR](A)[sP].[dR](C)[sP].[d | | |
| | | R](T)[sP].[dR](C)[sP].[LR](A)[sP].[LR]([5meC] | | |
| | | )}$$$$V2.0 | | |
| 5′- | SEQ ID | RNA1{[LR](T)[sP].[LR]([5meC])[sP].[dR](C)[s | GGTGAGT | SEQ ID NO: |
| TCCATCC | NO: 547 | P].[LR](A)[sP].[dR](T)[sP].[dR](C)[sP].[dR](C) | ACATGGA | 275 |
| ATGTACT | | [sP].[LR](A)[sP].[dR](T)[sP].[dR](G)[sP].[dR]( | TGGA | |
| CACC-3′ | | T)[sP].[LR](A)[sP].[dR](C)[sP].[dR](T)[sP].[dR | | |
| | | ](C)[sP].[LR](A)[sP].[LR]([5meC])[sP].[LR]([5 | | |
| | | meC])}$$$$V2.0 | | |
| 5′- | SEQ ID | RNA1{[LR]([5meC])[sP].[dR](C)[sP].[LR](A)[s | GGGTGAG | SEQ ID NO: |
| CCATCCA | NO: 548 | P].[LR](T)[sP].[dR](C)[sP].[dR](C)[sP].[LR](A) | TACATGG | 276 |
| TGTACTC | | [sP].[dR](T)[sP].[dR](G)[sP].[dR](T)[sP].[LR]( | ATGG | |
| ACCC-3′ | | A)[sP].[dR](C)[sP].[dR](T)[sP].[dR](C)[sP].[L | | |
| | | R](A)[sP].[dR](C)[sP].[LR]([5meC])[sP].[LR]([ | | |
| | | 5meC])}$$$$V2.0 | | |
| 5′- | SEQ ID | RNA1{[LR]([5meC])[sP].[dR](A)[sP].[LR](T)[s | TGGGTGA | SEQ ID NO: |
| CATCCAT | NO: 549 | P].[LR]([5meC])[sP].[dR](C)[sP].[LR](A)[sP].[ | GTACATG | 277 |
| GTACTCA | | dR](T)[sP].[dR](G)[sP].[dR](T)[sP].[LR](A)[sP | GATG | |
| CCCA-3′ | | ].[dR](C)[sP].[dR](T)[sP].[dR](C)[sP].[LR](A)[s | | |
| | | P].[dR](C)[sP].[dR](C)[sP].[LR]([5meC])[sP].[ | | |
| | | LR](A)}$$$$V2.0 | | |
| 5′- | SEQ ID | RNA1{[LR](A)[sP].[LR](T)[sP].[dR](C)[sP].[dR | ATGGGTG | SEQ ID NO: |
| ATCCATG | NO: 550 | ](C)[P].[LR](A)[sP].[dR](T)[sP].[dR](G)[sP].[d | AGTACAT | 278 |
| TACTCAC | | R](T)[sP].[LR](A)[sP].[dR](C)[sP].[dR](T)[sP].[ | GGAT | |
| CCAT-3′ | | dR](C)[sP].[LR](A)[sP].[dR](C)[sP].[dR](C)[sP | | |
| | | ].[dR](C)[sP].[LR](A)[sP].[LR](T)}$$$$V2.0 | | |
| 5′- | SEQ ID | RNA1{[LR](T)[sP].[LR]([5meC])[sP].[dR](C)[s | GATGGGT | SEQ ID NO: |
| TCCATGT | NO: 551 | P].[LR](A)[sP].[dR](T)[sP].[dR](G)[sP].[dR](T) | GAGTACA | 279 |
| ACTCACC | | [sP].[LR](A)[sP].[dR](C)[sP].[dR](T)[sP].[dR]( | TGGA | |
| CATC-3′ | | C)[sP].[LR](A)[sP].[dR](C)[sP].[dR](C)[sP].[L | | |
| | | R]([5meC])[sP].[dR](A)[sP].[LR](T)[sP].[LR]([ | | |
| | | 5meC])}$$$$V2.0 | | |
| 5′- | SEQ ID | RNA1[LR](T)[sP].[LR]([5meC])[sP].[dR](C)[s | GGTTTGT | SEQ ID NO: |
| TCCACAC | NO: 553 | P].[dR](A)[sP].[dR](C)[sP].[dR](A)[sP].[dR](C) | TCAGTGT | 552 |
| TGAACAA | | [sP].[dR](T)[sP].[dR](G)[sP].[dR](A)[sP].[dR]( | GGA | |
| ACC-3′ | | A)[sP].[dR](C)[sP][dR](A)[sP][LR](A)[sP].[L | | |
| | | R](A)[sP].[LR]([5meC])[sP].[LR]([5meC])}$$$ | | |
| | | $V2.0 | | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[MOE](G) is a 2′-O-methoxyethyl-RNA guanine nucleoside,
[MOE](T) 2′-O-methoxyethyl-RNA thymine nucleoside,
[MOE](A) 2′-O-methoxyethyl-RNA adenine nucleoside,
[MOE]([5meC] 2′-O-methoxyethyl-RNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[mR](G) is a 2′-O-methyl RNA guanine nucleoside,
[mR](U) is a 2′-O-methyl RNA DNA uracil nucleoside,
[mR](A) is a 2′-O-methyl RNA DNA adenine nucleoside,
[mR]([C] is a 2′-O-methyl RNA DNA cytosine nucleoside,
Further details regarding how to read a HELM sequence are provided at www.pistoiaalliance.org/helm-tools/.

1. An antisense oligonucleotide Unc-13 homolog A (UNC13A) splice modulator, wherein said antisense oligonucleotide splice modulator is 8 to 40 nucleotides in length and comprises a contiguous nucleotide sequence of at least 8 nucleotides in length which is complementary to the UNC13A precursor-mRNA.

2. The antisense oligonucleotide splice modulator of claim 1, wherein the UNC13A precursor-mRNA has the sequence of SEQ ID NO: 1.

3. The antisense oligonucleotide splice modulator of claim 1 or claim 2, wherein the antisense oligonucleotide splice modulator is capable of increasing the expression of Unc-13 homolog A (UNC13A) in a TDP-43 depleted cell.

4. The antisense oligonucleotide splice modulator of claim 3, wherein UNC13A is encoded by the nucleotide sequence of SEQ ID NO: 2, or a fragment or variant thereof.

5. The antisense oligonucleotide splice modulator of claim 3 or claim 4, wherein the UNC13A protein has the sequence of SEQ ID NO: 3, or a fragment or variant thereof.

6. The antisense oligonucleotide splice modulator of any one of claims 1-5, wherein the antisense oligonucleotide splice modulator is capable of decreasing expression of an UNC13A mutant polypeptide in a TDP-43 depleted cell.

7. The antisense oligonucleotide splice modulator of claim 6, wherein the UNC13A mutant polypeptide is a splicing variant of UNC13A.

8. The antisense oligonucleotide splice modulator of claim 7, wherein the UNC13A mutant polypeptide comprises a polypeptide sequence encoded by an additional exon, when compared to the wild-type UNC13A polypeptide sequence.

9. The antisense oligonucleotide splice modulator of claim 8, wherein the UNC13A mutant polypeptide comprises an insertion, when compared to the wild-type UNC13A polypeptide sequence.

10. The antisense oligonucleotide splice modulator of any one of claims 6-9, wherein the UNC13A mutant polypeptide is encoded by the nucleotide sequence of SEQ ID NO: 4, or a fragment or variant thereof.

11. The antisense oligonucleotide splice modulator of any one of claims 6-10, wherein the UNC13A mutant polypeptide has the sequence of SEQ ID NO: 5, or a fragment or variant thereof.

12. The antisense oligonucleotide splice modulator of any one of claims 6-9, wherein the UNC13A mutant polypeptide is encoded by the nucleotide sequence of SEQ ID NO: 6, or a fragment or variant thereof.

13. The antisense oligonucleotide splice modulator of any one of claims 6-10, wherein the UNC13A mutant polypeptide has the sequence of SEQ ID NO: 7, or a fragment or variant thereof.

14. The antisense oligonucleotide splice modulator of claim 6, wherein expression of the UNC13A mutant polypeptide is decreased due to nonsense mediated degradation of the mature mRNA.

15. The antisense oligonucleotide splice modulator of any one of claims 6-14, wherein the contiguous nucleotide sequence is complementary to a splice enhancer site in the UNC13A precursor-mRNA.

16. The antisense oligonucleotide splice modulator of any one of claims 1 to 15, wherein the contiguous nucleotide sequence is complementary to SEQ ID NO554.

17. The antisense oligonucleotide splice modulator of any one of claims 1 to 16, wherein the contiguous nucleotide sequence is complementary to SEQ ID NO 555.

18. The antisense oligonucleotide splice modulator of any one of claims 1 to 17, wherein the contiguous nucleotide sequence is complementary to SEQ ID NO 556.

19. The antisense oligonucleotide splice modulator of any one of claims 1 to 18, wherein the contiguous nucleotide sequence is complementary to SEQ ID NO 557.

20. The antisense oligonucleotide splice modulator of any one of claims 1 to 18, wherein the contiguous nucleotide sequence is complementary to SEQ ID NO 558.

21. The antisense oligonucleotide splice modulator of one of claims 1 to 20, wherein the contiguous nucleotide sequence is complementary to a sequence selected from SEQ ID NOs 8-279.

22. The antisense oligonucleotide splice modulator of claim 21, wherein the contiguous nucleotide sequence is complementary to a sequence selected SEQ ID NO: 62; SEQ ID NO: 66, SEQ ID NO: 68, SEQ ID NO: 70, SEQ ID NO: 72, SEQ ID NO: 73, SEQ ID NO: 74, SEQ ID NO: 76, SEQ ID NO: 112, SEQ ID NO: 114, SEQ ID NO: 115, SEQ ID NO: 117, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 162, SEQ ID NO: 163, SEQ ID NO: 178, SEQ ID NO: 179, SEQ ID NO: 183, SEQ ID NO: 187, SEQ ID NO: 189, SEQ ID NO: 192, SEQ ID NO: 193, SEQ ID NO: 214, SEQ ID NO: 247, SEQ ID NO: 252, SEQ ID NO: 254, SEQ ID NO: 258, SEQ ID NO: 273, SEQ ID NO: 276, SEQ ID NO: 277, SEQ ID NO: 278, and SEQ ID NO: 279.

23. The antisense oligonucleotide splice modulator of claim 22, wherein the contiguous nucleotide sequence is complementary to a sequence selected from SEQ ID NO: 66, SEQ ID NO: 70, SEQ ID NO: 72, SEQ ID NO: 73, SEQ ID NO: 74, SEQ ID NO: 76, SEQ ID NO: 122, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 146, SEQ ID NO: 151, SEQ ID NO: 152, SEQ ID NO: 154, SEQ ID NO: 155, SEQ ID NO: 156, SEQ ID NO: 162, SEQ ID NO: 163, SEQ ID NO: 273, SEQ ID NO: 276, SEQ ID NO: 277, and SEQ ID NO: 279.

24. The antisense oligonucleotide splice modulator of any one of claims 1 to 23, wherein the contiguous nucleotide sequence is a sequence selected from SEQ ID Nos 280-551, or at least 10 contiguous nucleotides thereof.

25. The antisense oligonucleotide splice modulator of claim 24, wherein the contiguous nucleotide sequence is a sequence selected from SEQ ID NO: 334; SEQ ID NO: 338; SEQ ID NO: 340; SEQ ID NO: 342; SEQ ID NO: 344; SEQ ID NO: 345; SEQ ID NO: 346; SEQ ID NO: 348; SEQ ID NO: 384; SEQ ID NO: 386; SEQ ID NO: 387; SEQ ID NO: 389; SEQ ID NO: 394; SEQ ID NO: 397; SEQ ID NO: 398; SEQ ID NO: 399; SEQ ID NO: 400; SEQ ID NO: 401; SEQ ID NO: 402; SEQ ID NO: 403; SEQ ID NO: 405; SEQ ID NO: 406; SEQ ID NO: 407; SEQ ID NO: 408; SEQ ID NO: 409; SEQ ID NO: 410; SEQ ID NO: 411; SEQ ID NO: 412; SEQ ID NO: 413; SEQ ID NO: 414; SEQ ID NO: 415; SEQ ID NO: 418; SEQ ID NO: 423; SEQ ID NO: 424; SEQ ID NO: 425; SEQ ID NO: 426, SEQ ID NO: 427, SEQ ID NO: 428, SEQ ID NO: 434, SEQ ID NO: 435, SEQ ID NO: 450, SEQ ID NO: 451, SEQ ID NO: 455, SEQ ID NO: 459, SEQ ID NO: 461, SEQ ID NO: 464, SEQ ID NO: 465, SEQ ID NO: 486, SEQ ID NO: 519, SEQ ID NO: 524, SEQ ID NO: 526, SEQ ID NO: 530, SEQ ID NO: 545, SEQ ID NO: 548, SEQ ID NO: 549, SEQ ID NO: 550, and SEQ ID NO: 551, or at least 10 contiguous nucleotides thereof.

26. The antisense oligonucleotide splice modulator of claim 25, wherein the contiguous nucleotide sequence is a sequence selected from SEQ ID NO: 338, SEQ ID NO: 342, SEQ ID NO: 344, SEQ ID NO: 345, SEQ ID NO: 346, SEQ ID NO: 348, SEQ ID NO: 394, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 418, SEQ ID NO: 423, SEQ ID NO: 424, SEQ ID NO: 426, SEQ ID NO: 427, SEQ ID NO: 428, SEQ ID NO: 434, SEQ ID NO: 435, SEQ ID NO: 545, SEQ ID NO: 548, SEQ ID NO: 549, and SEQ ID NO: 551, or at least 10 contiguous nucleotides thereof.

27. The antisense oligonucleotide splice modulator of any one of claims 1 to 26, wherein the contiguous nucleotide sequence is at least 12 nucleotides in length.

28. The antisense oligonucleotide splice modulator of any one of claims 1 to 27, wherein the contiguous nucleotide sequence is at least 13, 14, 15, 16, 17, 18, 19, 20, 21, 22, 23, 24, 25, 26, 27, 28, 29, 30, 31 or 32 nucleotides in length.

29. The antisense oligonucleotide splice modulator of any one of claims 1 to 28, wherein the contiguous nucleotide sequence is the same length as the antisense oligonucleotide splice modulator.

30. The antisense oligonucleotide splice modulator of any one of claims 1 to 29, wherein the antisense oligonucleotide splice modulator is isolated, purified or manufactured.

31. The antisense oligonucleotide splice modulator of any one of claims 1 to 30, wherein the contiguous nucleotide sequence comprises one or more modified nucleosides.

32. The antisense oligonucleotide splice modulator of any one of claims 1 to 31, wherein the antisense oligonucleotide splice modulator is a morpholino antisense oligonucleotide.

33. The antisense splice modulator oligonucleotide of claim 31 or claim 32, wherein the one or more modified nucleosides, is a 2′ sugar modified nucleoside, such as a 2′ sugar modified nucleoside independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), or any combination thereof.

34. The antisense oligonucleotide splice modulator of claim 33, wherein the 2′ sugar modified nucleoside is an affinity enhancing 2′ sugar modified nucleoside.

35. The antisense oligonucleotide splice modulator of any one of claims 1 to 34, wherein the contiguous nucleotide sequence of the antisense oligonucleotide splice modulator comprises 2′-O-methoxyethyl-RNA (2′-MOE) nucleosides.

36. The antisense oligonucleotide splice modulator of claim 35, wherein all the nucleosides of the contiguous nucleotide sequence are 2′-O-methoxyethyl-RNA (2′-MOE) nucleosides, optionally linked by phosphorothioate internucleoside linkages.

37. The antisense oligonucleotide splice modulator of any one of claims 31 to 36, wherein one or more of the modified nucleosides is a locked nucleic acid nucleoside (LNA), such as an LNA nucleoside selected from the group consisting of constrained ethyl nucleoside (cEt), and ß-D-oxy-LNA.

38. The antisense oligonucleotide splice modulator of claim 37, wherein the contiguous nucleotide sequence of the antisense oligonucleotide splice modulator comprises or consists of LNA nucleosides and DNA nucleosides.

39. The antisense oligonucleotide splice modulator of any one of claims 1 to 38, wherein the contiguous nucleotide sequence is at least 75% complementary to the UNC13A precursor-mRNA sequence.

40. The antisense oligonucleotide splice modulator of claim 39, wherein the contiguous nucleotide sequence is at least 80%, at least 85%, at least 90% or at least 95% complementary to the UNC13A precursor-mRNA sequence.

41. The antisense oligonucleotide splice modulator of any one of claims 1 to 38 wherein the contiguous nucleotide sequence is fully complementary to the UNC13A precursor-mRNA.

42. The antisense oligonucleotide splice modulator of any one of claims 1 to 40, wherein the contiguous nucleotide sequence comprises 1, 2, 3 or more mismatches to the UNC13A precursor-mRNA sequence.

43. The antisense oligonucleotide splice modulator of any one of claims 1 to 42, wherein the Gibbs free energy of the antisense oligonucleotide splice modulator to a complementary target RNA is lower than about −10ΔG, such as lower than about −15 ΔG, such as lower than about −17 ΔG.

44. The antisense oligonucleotide splice modulator of any one of claims 1 to 43, wherein the antisense oligonucleotide splice modulator does not comprise a region of more than 3, or more than 4, contiguous DNA nucleosides.

45. The antisense oligonucleotide splice modulator of any one of claims 1 to 44, wherein the antisense oligonucleotide splice modulator is not capable of mediating RNAseH cleavage.

46. The antisense oligonucleotide splice modulator of any one of claims 1 to 45, wherein the antisense oligonucleotide splice modulator or contiguous nucleotide sequence thereof is a mixmer or a totalmer.

47. The antisense oligonucleotide splice modulator of any one of claims 1 to 46, wherein the cytosine bases present in the antisense oligonucleotide splice modulator or contiguous nucleotide sequence thereof are independently selected from the group consisting of cytosine and 5-methyl cytosine.

48. The antisense oligonucleotide splice modulator of any one of claims 1 to 47, wherein the cytosine bases present in the antisense oligonucleotide splice modulator or contiguous nucleotide sequence thereof are 5-methyl cytosine.

49. The antisense oligonucleotide splice modulator of any one of claims 1 to 48, wherein one or more of the internucleoside linkages positioned between the nucleosides on the contiguous nucleotide sequence are modified.

50. The antisense oligonucleotide splice modulator of any one of claims 1 to 49, wherein at least about 75%, at least about 80%, at least about 85%, at least about 90%, at least about 95%, or about 100% of the internucleoside linkages positioned between the nucleosides on the contiguous nucleotide sequence are modified.

51. The antisense oligonucleotide splice modulator of any one of claims 1 to 50, wherein one or more, or all of the modified internucleoside linkages comprise a phosphorothioate linkage.

52. The antisense oligonucleotide splice modulator of any one of claims 1 to 51, wherein all the internucleoside linkages present in the antisense oligonucleotide splice modulator are phosphorothioate internucleoside linkages.

53. The antisense oligonucleotide splice modulator of any one of claims 1 to 52, wherein the antisense oligonucleotide splice modulator is covalently attached to at least one conjugate moiety.

54. The antisense oligonucleotide splice modulator of any one of claims 1 to 53, wherein the antisense oligonucleotide splice modulator is in the form of a pharmaceutically acceptable salt.

55. The antisense oligonucleotide splice modulator of claim 54, wherein the pharmaceutically acceptable salt is a sodium salt or a potassium salt.

56. A pharmaceutical composition comprising the antisense oligonucleotide splice modulator of any one of claims 1 to 55, and a pharmaceutically acceptable diluent, solvent, carrier, salt and/or adjuvant.

57. A method, such as an in vivo or in vitro method, for increasing UNC13A expression in a cell, said method comprising administering an antisense oligonucleotide splice modulator of any one of claims 1 to 55, or the pharmaceutical composition of claim 56, in an effective amount to said cell.

58. The method of claim 57, wherein said cell expresses aberrant or exhibits depleted levels of TDP-43.

59. A method for treating or preventing a disease in a subject comprising administering a therapeutically or prophylactically effective amount of an antisense oligonucleotide splice modulator of any one of claims 1 to 55, or the pharmaceutical composition of claim 56 to a subject suffering from or susceptible to the disease.

60. The antisense oligonucleotide splice modulator of any one of claims 1 to 55, or the pharmaceutical composition of claim 56, for use as a medicament.

61. The antisense oligonucleotide splice modulator of any one of claims 1 to 55, or the pharmaceutical composition of claim 56, for use in the treatment or prevention of disease in a subject.

62. The antisense oligonucleotide splice modulator of any one of claims 1 to 55 or the pharmaceutical composition of claim 56, for the preparation of a medicament for treatment or prevention of a disease in a subject.

63. The method of any one of claims 57 to 59 or the antisense oligonucleotide splice modulator for use of any one of claims 60 to 62, wherein the disease is a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

64. The method or antisense oligonucleotide splice modulator for use of claim 63, wherein the disease is a neurological disorder selected from the group consisting of amyotrophic lateral sclerosis (ALS) and frontotemporal lobar degeneration (FTLD).


SEQUENCE LISTING (564 sequences)

SEQ ID NO: 1 - DNA, 87019 bp, synthetic construct
  misc_feature 1..87019: note = UNC13A precursor-mRNA
  source 1..87019: mol_type = other DNA
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggaggtaggaggcgaggctgggggtcgggggacccgtgtggacgcgggctggggtcggggtccgcgtggatgcggctcccagggggttcctggggggtgacccgagtccaggtggctggacttttgagaggggtagcccttgtcgacgcagctgggccccggagggggtgatccgtgtagacgcggcagggctctcgggggtcctggtggccgctgctggaggggtcgtgtggacgcggctgaacgcggaggcccgtgtggacgtggacgggtatccggggttctacccggaaacgatctagactggggcgtctgtgtagaccaggtttttagagggtcagtgaggaagagtctgggctcggtagtttgattgaagcctgcaggggtgcttggaaggggtctggtgttgtgggctgggttacgggtcctcatggacgcggacccggctggaaattcgaaggagagtgcggtgggagggttctccaagccgtggggatgtaagatataggtagaggggcttggccatcagggcatccacgagttctaggactctcccagccctaggtgtaaagaggactggagtggggggagtgtggagacaggagacccctcccagcgtccagagagggctgtgactggaggtctgggggcgcttttccagttaactctttggcctcccgagtgagggggttggaggaatgtccagaaggtcttcccgctccctgccccctccggttctcccaaacacgtttcagggccttctcggcagggagcctacttcttggaagagcaatgtctcctggtccccctcctggcccatcctcacctcacccactttgcactggagagagggagggagggagacgtgaggtggggggggagctattttggggccagaacgccatctgcacagaggcccacgcccaggcccacgctctgcgttggggttggaggagggagggagacgtgggaggggcatgaccccccaccccacacctttatggctctggggtcgtggggaagccagcccaaccgaacccaggcgttctgggcactcccgtttgagaaatcaaactctgggagaccttgggcggcaggggtggatgtggaccgccgttccacgcctacagagctctgaacccctggttcttaaggactggggtccccccgtcactcccacccatttcaagaaggctggggtgggggaaggggtgtgtctgccaagcctgagactgcaggcttcgggagacctcgggcgacaggaagcgtgcgtgtgctgaactcctgctgcctggacacagggtgggcaggccggggagccgggaaccgcccccgccgcccctcctcctccctcccctccccctcggccgattcattcacaaaccctgccccgcccgcccgctccccgccgcggcagcttccgagtgcaaatccggccgggctgggccggcgtggacgcgggtgttcttaaagggacaggggcctactttctctaagctcgtttctgctggacggggctgggggtggtggaagaagggtccctagagtcaaaggtcaactcctccatcccctagcctccaggcctatggagttaggggagtagggaagtgaaggagacccctaggcgacccccaccgcctaggcctgaggacggaatgggaggagaacttgaatgtccagaccccctactgacctctccccctgctctaagtagccgggctcttaaaggtggcactggaggaggttccgacgtgggtgttcttggctgactcctctatcctctctcttctccccccagcatcccgccccaccccgcctcacatcctccaagttgggcttctctggaaggtttgcctagtggagtgggggggggggggggggggcggggatctcacccttaactctgacgcaaacaggaggctcggctgcgggggtggcattaagggggggcagcaggaggcaagagggatgaggcttccagaaacagagacgttgggggaggactgtgctggattctgtggactttacagggaggggcccggctgagtggagaggaatcgctgggaggggcaaggctggggaagaggttgacaggggtgggaggtaaggccctcaggtgtaaagggaggtgtgaggttggagtgaggaggagtaggaggcagggaggggtggggagagggagaagtgctgggtgaggccagtctcccagggagaggtgggatggtgcccagagaggggaggggctgggatggctgagcacgagagatgttggggtgggcttggcaggagtgaagctaggctgagatgggttgccgtgttgcctgagctcagctgaggaaatacctgtcccccctcccctccaccggggacccacaggcgtttgccaatccctacacacattcatacccagacttgttcctgtgtggacacaaatgcttaggatgtatgaaggctctacgcagaccatgtgcctgggaaggcacattccgtaggcacacgtgtgtactcaaatgcctgtgtgaacacaagcatacacgtgtatataggacacagtctgccgctacagatatagtcccagcaacacacgtgtgagtgtgtgtgtctgccagcacgcatatagatgtatccacgtgtctgatgaatgctcacaatatacatgtcacataaagatccacccatgtccagccgagcgcggtggctcaggcctgtaatctcatcactttgactttgggaggccaaggtgggcagatcacttgaggtcaggagttcaagaccagcctggccaacatggtgaaactttgtcactgctaaaaaataaaaaaaattagccaggtgtggtggcgcgcacctgtagtctcagctactcgggaggctgaggaaggagaatcacttgaataggggaggtggaggttgcagtgagctgagatcctgccactgtactccagcctgggcaacacagtgagactctgtctcaaaaaaaaagattcactcatgtcctaacactcagacatgtacccatacatatctgttaaggtccccaaccccatgaagcacatatatattcatataagcacatgtcaaagccatgtgtgtatgcacatgacaacacttgcctggacattcaaagcagcccctttcaacaagtggatgatgatgcatacaagtaaatacacatttgagtatttcattgcacacaagtgcatacatgtatgcagataaacaaatacagctgccacagaactgcatccatgtacacatgcaaacatcaatatgtgtgttttccacctaacatatcacccacaagctcacacacaccagtgtgtctctgatgtagtgacaccccactcctagacacacatgtgcagactcccacctgcacttaaatggtgcacatactctgcacacatgcacactcatatccatgtagaggcaggcacctgcctgccctggtaagatgagccaactactgctgcaaatacccaagcaagtacaggcaaatgcaagctcttgtgtatacacccagcaccaacacatagcagaccaccctgtatggtgtatgggccttatctctgcccgcttcatttatttattcatgtggtgccccgctgcttcccaacatcgtaatccactgactaggtcttggcaggatcctgaggctgtgaatggtaaatgccacctggttccttcatgaagtcacttattttatttatgcaacaagcattaattgagcacctactgtataccaggcactgttcctggctctgggacccagccctattcctagccatcactcctgcacgtcccatggggttagaatgaaagagatgagggagggctaagggagctgaggagtggggtatccagggctctcttcctctgggaagcctaccctgattccaccaccaccatttacttgagtctcagaagatgtgatgagggggagatgagcaggagggagatggtgctctgggtggagggaacagcatgagcaaaagcatggtggtgtgaagggttacttccagatcttcctatgtgcctcacagcattttgcacaggccacttgatgttttgttttgttttgagacagagtctcactcaaagcctgttactcaggctgcagtgcagtggcatgatctcagcttgctgcagccttgacctccagggctcaagtgatcctcctgcctcagcctcccaagtagctgggagtataggcatgcaccaccacacccggctgacttttttctttttaattttttgcagaagcagggttttgccctgctgcccaggctggtcttgaactcctgggctcaagtgattcaccctcctcagcctcccaaagtgctaggattacaggtgtgagccaccacgcccaactgatgttttaaaggctctataaacatgttatgaataaatgacatgaacaaatcagtgaatttaggtgagggtgggtttgcttcacaacttacaataatatcaacaacaaaccccatttatccagcttttttgtttgtttgtttgttttttgagacacggtctcactctgtcacccggactgcagtgtagtggtgtgatctcggttcactgaaacctccaccttccaagttcaagcgattttcctgcctcagcctcccgagtagctgggattacaggcacgtgctaccacacctagctagtttttgtatttttagtagagatggggtttctcatctctactaaaaatacaaatacatctctagtggccaggctggtcttgaactcctgacttcaagtgatcctcttgcctcggcctcccaatgtactgagattacaggctcgagccaccgtgcccagcccagcatttcataggcatgatgttaaggatttttatgcccatgccctctttaactcttacagtagccccattgtacagatgaggaaaactgaggctcagggagctttaacaacttgcctggtggaacagagccagaggtggagctgactgactctagagcctactggggttggcagaagtttgagccttctctcagaccccaggacctcggagacagccaagtcccttctgtggagtgatgttctgccctgggattgccttcttctttctatttatttatttattggagacagagtctggctctgttgcccaagctggagtgcagtggcacaatttcagttcactgtaacctcctcctcccgggttcaagcaattctcctgcctcagcctcctgagtagctgagattacagatgcacaccatcatgcccagctaattttttgtatttttagtggagatggggtttcaccatgctagccaggctggcctcgaactcctgaccaccaagtgatctacctgctttggcctcccaaagtgctgggattacaggcatgagccactgtgcccagccaggattgctttcttctctcatgcatctagtaaacagacatgtcacaatctctccattcatcggggctattcccctgctccttgcctaagaagccttcacacatccctccgttcctggctactcagtccctgataccctccagctaggaggagctgcatctaggtcttagctcctaccgttctcatgggatgccctccagagttgcatcttcagggctgcagcctaaccctttccctaacctggcctcacttttcttccacatacaatggacctcttctgcaaggtgccaataagggcatcctgggtccagcaggcctgagtgtgaattgctaagtccaccacttcctggcaagtctcacatcctctgcattccttggtgtgctcatctgtggaatgggcacaggagtagcatccaccatctacgtttgccagaactctctaatgaggcttgcagagcccctggcatgactcctggcctacgggaaggcccaataatcgtgagcaaaagacatgaaaaatcaatgatgggccggccccggtggctcacgcctgtaatcccagcactttgggaggcctaggcgggcagatctcctgagctcaggagttcaagaccaccctgggcaacatggtgaaaccctgtctttactaaaatacaaaaaaattagctgggtatggtggcgcgcgcctgtagtcccagctactcagggggctgaggcacgagaatcgcttaagccccgaaggcggaggttgcagtgagccgaggtcgcgccactgcactccagcttgggctacagagtgagactccgtcaaaaaaaaaaaaaaaaaaaaatcaatgatgatgtttttattaatggagactaaaagctttctggaaggcggttctgagtgcaggacatttctgagagtccactgcattctggggcccgaggagcgctccaagaggggtcccccagggccactccaggagggagcctagttcgttgagggtacacgggtgggaccgagcattcagcaccgtggacagcgcctgcccgcgcctctgggggacagctcttagctcagcctcccaggattaatggaggaggtgacgtcattccttccgtgaattactatttaatgagcacctactataggctaggcctacttctaagtggttcatggtaaacaacacacacacaaatcccttattcccgaagctctctggaactccaggttcttggtgttttttacactgaaggctcagtctggctctggtctatttgtgtcaacggaggataagggtcctcatttagtgtctcccagatgcgaaaccagcacagctgaggtcttttatcctaaagcccatccatctcccgctgaaatgccctccaccaacatcacacacaaaacgatcaaatctcctataatctatttctggattctctatgcttcctcccttcccccaagctaatcctggggcgggtgggctttcagaagccctagacggctggatctccacctcctgtctccccaccccatcccacgtggcaggtcagggagccagtgccccaagactgcagtcccccctaattagaatccagggtcagtcagccatgtggttcacgcctgtaattccaacactttgggaggccgaggggatggcttgagccttgaagccagccatagcaacatagcaagaccccatctctacaaaaaaaaaaaaaaaaaaaaaggaatagccaagcatggtgatgcgcacctgcagtctcagctactcgagaggctgagtggggaggatcgcttgaacctgggagatcgaggctgcagtgagccatgatcacaccactgccctccagcctggagcctgggcaacagagagagaccctgtcaaaaaaaaaaaaaaaaaaaaaaagaaaagaaaagaaaaaaaaagaagaagaagaagagaaaagaaagaaaaagaaaagaatccagggtattcaaatcctagaatccaggccagggacacctttagaacacaaccaaataataaaaacagtaaagatatcagtaataataataatagtataaatgacactataatcattctgcagaaggggtgagggaggaagaggagggaagaagaggaggagaaaggagagggaagggagatcagccactccgtctccccattttccagatgcagaaacggaggcgcgaagccaggctgcaggggtgtcccgcagcctgaactcctgcctcagcctccccgggtacggccactacattcatcagtcagcgcttctgggggccccaaagacctttgaggccgcatgacgcggtgtcttcacccgctcgtcgctggggacagcagtgggcacccccctcatccccgcccccctgtccgccggcgcgcctcccacgccctccgcgcgcccacgctataaatagcctcatgcgtgtgacccgaagcccgctggggaagggtggcgcttccgccccgctgaccctgatacagcaacccggcgcaggcggggaggtggcgggatgtgacgacgaagacggtgactcagggaccatttactgaccggggaagacagggagagagactgggttccttctctgagcccccttcctcctccaagcctctcccccgataccaccctcccctctgcgcccatcccccttcccgaatcccctccctcaccgcgaacacccttccctcctgagaaccccctcgccactctcggccccctttcctgtccccttttccctctgagacccctctcccactgggagccccagtgatgaaaaatcatggatgtcctcaacctggacctcggcagggagctaagtgcaggagcctggcttcagctatcagagggtacagccctccagccggtcccccactgcctgaggaccccaacggagaccattcccctctagccaagagaggctgcaggaaggagctgaggcttagggaagaggagccggactgaccaagggaggggcacaagaagggcagagacagtgagagagagaaggagagaacagagaccaaaagaagacgaccagagtggagagaaatagaacggggaggagggagatgagagacagggattgggaaagagagagagagaacagatcaaggagctactgggctccccgctgagcctaaatccatggtttataaaacagggcacagttggaggagcttcaagtgagcccagcccagtctagaccccagtgagccccttgtatcaccccccagagcctgtcactttcccacccccatatgcatccctacttttctcccccaactgcctctcctctccccagtgtctcctccacctgcctgccctccctagtggaaatctttccccctctctcttttattattattattattattatatattttttgagacagagtcttgctctgtcgcccaggctggagtgcaatggcgtgatcttggctcactgcaacctccgcctcctgggttcaagcgattctcctgcctcagcctcccgagtagctgggactacaggcgtgcaccatcatgcccggctaatttttgtatttttaatagagatggggttttaccatgttgcccaggctggtctcgaactcctgacctcatgtgatccacctgcctcggcctcaaagtgctgggattacagatgtgggctaccacgcccagcctctctcccttttaaattattatttttagacatgggatcttactacattgcccaggctggtcttgaactcctgggctcaagggatcctcctgccttagccttcctagtagctgggattacaggcatgtgcctctatgcctggtactctcgctgtctccttgtgtctctctatttctctccatctgtccagactctcctctcttgctttcatgcccatcatttcttcctctctcctctcccttcctctccctgtatgagtccctccagctgtgggtgcctttctcagcctctgtcagcatcccctccacccccgatcccgtcacattctacctcttgccccttcccctgcccacccccttcacagagagtccccacgcctgcaggctggacctcctccaggagccaggcccaaagctcctgctgggcacattcctgagtgttcctgagcctctttcagtcccaggggtgaggtgctgggtgggtgcgctccctgggggctgggtggggctgaggatgtgagcgaacaaaccagacctagaaaaaaaaaagacagagatggaagcagagatgaaggcccagagagaggggttcacttgcccagggtcacacagctcaggaatctgaacctggctcaaaccagcattcacttctctcaaggcccacccaagggctgcagtgcagtggcacaatctcagctcactgcaacctctgccttccaggttaaagggattctcctgcctcagcctcccgagtagctgggattagaggtgcgtgccaccacacccggctaatttttgttattttaggagagacgaggtttccccatgttgttcaggctggtctcgaactcctgacgtcaagtgatccacctgcctcggcctcccaaagtgctgggattacaggcatgagccaccgcgcctggccaggaacagtctttactgacatgtgtttagggccatcgcacttcccccgctcttcacgggaattctctcttcactggctccactcaggtgccttttgtgagacagaaattcaagcgatgaggacagagatacaaacccgggatgggaggctgtgacatctgcccatagaaacacaattctgtttctaggaatttatccttcaactcgctagtacacgctcttcaagacatactgaaaaaacatgaaggattagaacagtgggtgtcgaatgctaccatttgtcttggaaatcaagcaaaacgaacgtgggtatggttgccgacgcatggaccatctccagtggaacaaggaactgccaacgttgattgcatttgggagggagatgaagggctgggatggaaagagctttattctcagtgcctcatcttttggttgttttggattttacaacatgtgcatgcattgcctacacaaacaaagacactaatttgaaaaggaaaaggggagaagaaaagagggaggaaagaaaaggggccggtgtggcagctcacacctgtcatcccagcaccttgggaggccaaggcggatggatcacctgagatcaggagtttgagaccagcctggccgacatatactgaaaccctgtctctactaaaaaatacaaaaattagctgggcatggtggcgcacgcctgtagtcccagctacttgggaagctgaggcaggagaattgcttgaatctgggaggtagaagttgcggtgagctgagatggcaccactgcactccagcctgggcaatagagcaagactccgtccctaaaaaaaaaaagaaaaaaaaaaagaaaagaaagaaaggaaagggagggaagagctcagtgtgggagaagattgggggtgggagtagagaattaatggaggaaaaagatgtgggggacaaaagcaaggtgtgagaggtgttggcaatggaaactgaatacctgttgagaagagccaggagggggcgacatggctttgaatctcttggggctgtgagattatgtgaaatgtgagaagactggacctcagtggggtgcctgacatatattcaggttacacctcccacaagcaggtggggctggacaatctctgtcaaccctccagccaggacccaagtgtctccctgtacattacttttggttgttgttgagacaagaatctcactctgctgcccaggctggagtgcagtggcatgatcttggctcactgcaatctctgcctcctgggttcaagtgattctcctacctcagcctcccaagtagctgagactataggcgcctgccaccacacctggcaaatttttgtatttttagtagagacaaggattcaccatgttggccaggctggtcttgaactcctgacctcaggtgatccacctgcttcagccacctaaagtgctggggttataggtgtgagccaccgtgcctcgccctccacatgcattttgagagccagctctctgggttgctttgtcccaactcacccagccttctttgaggcttccccaaaccctgggagcatttgacattgcagactgagctggacaatcaagggagaggtcttgctgacctgtcggggctggtggagtcatcagagattgtctttatcctcgccttcttcttctccacctcctcaacttcccctcttcttctggggcaatcctggagggcttcttgtaggaggcctctcccaccatctgcccagctctttctttctaatcttgttcaattgtgggtaaaggcacagctagttgggagcttggggagtttgatggggacaggggatgcagggagagggaggggcagggaatgctctcagggtccaaatttccttccattctagggttgcctgtgctcatctgtctgatggcttctctctctccctctcatccctgcctcctctttcctacctctcctcctccccttgtctctgtctctgacattgcttctatctttcttttttaacatctgtctcttttgctttttatctttgcctgtctctcctctgtgtctctctccttatgtctccatcttggtgtgtctctgaatctctccgtatctctgcctcctcctccccacatctctccccacctcccttccctatctctgtctctctccacagtcaaaaaagccaagtttgatggtgcccaaggtaagtggcttctcttgctgtcctgtcccgtgttgtctgtctgtctgtctgtctcttggagagactgggagggggtcccagcccagacttagagttgggctttatgtcttctgagcatcaggagaggggtcagaagattaaatagggggaggggcagccattggcctggggtccctgcagtttgtatgggggtccccgggtctccccagccatccttggagagtctgtaaggggtggggcttgccctgagccagtggctcttcccagggtcctaagaggtagaatggggactcttagatggggctacaggtgggaaaaatgaggctcagagaggcgaaaggagtttctttcttttcttcttcttcttttttttttttttttgagacagagtctccttctgttgcccaggctggagtgcagtggcgtgatctcggctcactgcaacctctgcctctgggttcaagcaattctcctgcctcagcctcccaagtagctgggattacaggcgccctctaccacgcccggctaatttttttatttttagtagagatggggtttcgccatgttggccaggctggtctcaaacttctgaccttaaatgatccgcccgcctcggcctcccaaagtgctaggattgcaggcgtgagctaccgtgccctgcctttttattattattatttttatttaaaatttttattttttgaggctggagtgccgtggtgcaatcatgttttactacagcctcaacctcccaggctcaagcaatcctcccacctcagccttccaagtagctgggaccacaggcgtgcaccactgcacccagctaattatttttttgtattttttagtagagatagggttttgccaagttgcccaggctggtcttgaactgctgggctcaagcaatcctccccccttgacctcccaaagtgctggcattataggcgcgagccactgcacccagcctgcaaagggggtttcttacagtcaattttgttccccttttctgggacctcagtctttttagctgaaaaccaatggcatggtcatttcgaagcagctggatgaagtatcccaaaattccacgtctccccacactgaggcccctcccttgtgccctgagggtggggttgtgggttagggggtgagtggcccagaaattcctgaggggatcacagcagatggtctagagcttgggaaggggcattggagagtccctgagccccagcgctgacccctactctcactgccacagagaaattcaacacgtacgtgaccctgaaagtgcagaatgtcaagagcacgaccatcgcggtgcggggcagccagcccagctgggagcaggatttcatgttgtgagtctgcagcgtctggcatgacctcactccctggcatagcccagcacctcgctggcccctcagggcagagctggacactggggaatcccgggtgactcccctctgtcctccctcttcccctgtccaccttatcaccttcacctccaaaacacatccccaatccaatcacttcccttggtctccgcagccaccagccaagtccctgctgcccgccagcctccgccctggcctcctgtgccatccacctcctacaatctgttctcctccaggcagccagggggatatttttaaaactgcaaatcacatcctgtcgctcccatgttccaaacttgtccgtggctcatcgacctcacaataaaacccacagccttccccgtgattcccaccaggttctcgctctatctccctcacctgccccagccacacccacctctgcccctttctccaacacttcccagcttgttcttgccccagggcccttgcacttgctctcctgtccccagctggctcttctccttcaggtctcagcttgtgtcacctcctcagtgaagccctccctgaccactacctccaagctacaatttcagggcatcatgccttcccaggccttgccaccaattttgtttgtttgtttgtttgttttgaggcagagcctcactctgttgcccaggctggagtacagtggcataatctcggctcacttcaacctccacctcccaggttcaagtgatcctcctgcctcagcctcccaactagctgggattacaagcgtgtgccaccacacccggctaatttttgtgttttaagtagagatggtgtttcactatggtggccaggctagtctcaaactcctggactcaggtgatccgcccacctcagcctcccaaggtgctgggattatagatgtgagccaccatgcctggccaatggaatttttttccttttttttttgttttgttttgagacagagcctcactctgtggcccaggctggagtgtagtggtgcaatctcggctcactgcaacctctgcctcctgggttcaagctattctcctgccttagcctcccgagtagctgggactacatgtgcgtgccaccatgcccggctaatttttgtgtatttagtaaagaccgggtttcaccatgttggccaggctggtcttgaacttttaacctcaggtgatccacccgcctcggcctcccaaagtgctgggattataggcatgagccaggagccaccaatcccagccaacagatatatatatcagatatatatatacttttttttttttttttgagactgagttttgcttttgttgcccaggctggagtgcaatggcgtgatctcggctcaccgcaacctccgcctcctgggttcaagcaattctcctgcctcagcctcccgagtagctggaattacagacatgcaccaccacgcccggctaattttgtatttttagtggagatggggtttctccatgttggtcaggctggtcttaaattcccaacctcaggtgatccgcccatctcggcctcccaaagtgctgggattacaggcgtgagccaccgtggttggccatattttaaaaatatatttcttaagataaatctttggtaaactgtaaattcagtgaatctcacatttcaccaagtttcctgctcccccgaattcacatatgtctagggcgaggcctgcagggaaagtgctttgaggtgggatcgtgtcctcacatcattagatggggatgacactgggaaggacagaggtgacccgaatgtccttatcctgtaggtaaagggagctatggagggtatttgagcctcttcctcacttacccagcatggtcagtgttggagggcccaaggaagggctcagaaagatgaaggtgcaggcgatgtgtaacatctgtccccacgccatccttcccggctttgaatcctggttcttttgtgataccccgagctcttaacttcccccactggcctcagtttcccctctataaaatgagaaccaccatgttaccctaccacctaggacactcccaggcctaggtttgaattgcagtatgctgtgtgttctcaggcgaatcaactaaactccctgggcccccgtttcctccctcctgctccctcgacgccccctctcctttttctgcagcgagattaaccgtctggatttgggactgacggtggaggtgtggaataagggtctcatctgggacacaatggtgggcactgtgtggatcccactgaggaccatccgccagtccaatgaggtgagtggcgggtggcctgagggtgaagaaggaggagtgccttgcagaagcccagagaccagtggactaaacaagatgggcatttatctcctatcccctgacaacatcaatgtagacacttgggattgccacgaaccttccaaagtcactagagcctatacaccttccatcttgctctgcctctctctccatgtggcttccaccttgtggccccaaatggctgctttaggctcaccattgcatcttcattccaaccaccgaaaggtgctggtggtgggggaggggggtggttcatgcacatctcattggccagaacttagacatgtggccaacaccttgctgcaagggattctgggaaatgtagtcctaggctggaaaatgtagtcctgggctgtgcttccctaggcacagctaaaaatgtattaccttggaaggagaaaagggataatataggcaattagcattttgtgtcccagatgggcagcagcaggaagcttacaccaactcataagaaggtgctaggtaacaaccctggcccctcagcctgtgcatcattgtcagcaggtagtagtgatgtctattttttatttaattatttatttttgagacagggtctcactctgttgcccaggctggagtgcattggcatgatcatggcccactgcagcctcgacttcccaggctcaagtgatcctcccacctcagcctcctgagtagctgggactgcaggtgcatgccaccaggccccccttattttttttgtatttttaatagagacagggttttgccatgttgcccaggctggtttcgaactcctgggctcaagcaatccacccacctcagcctcccaaagtgctggtattacaggtgcgagccaccatgccaggccgtggtcatgtctattcatctgtccccacagcccaatccctggcctcctaacctcaagtctcaacctctccatgctgaccccaggaaggccttattagctggagctctgatctgtcctcccctgtccatctgccttgggaggcccttcagatggcacctgcccactagcctcatctccccccacactctaacctctacccctcccagattccaccctctgaacctttgcatagtgtgttccctgagcttcaagtgtcctcctttccatcaccttctaaattcccatgtgcccacctccaggaagccctctctgacttctctctagatcctttagccctccttcctccctctgggctatgacctcatggggctgggggtgcctgtgtccagccctgtttccaagggctatgggctctcaggagccagggtgatgatggctgggcctcttggggaccctggcatcacccaatatgaaccaggcatagaggagggactggggactgttttttgtttgttcgtttttgtcattttattttattttattttattttattttattttattttattttattttattttactgtgagatggagtctcactctgtcacccaggctggagtgcaatggcacaatcttggctcactgcaacctctgcctccctggcttaagtgattctcctgcctcaacctcctgagtagctgggattacaggggcctgccaccacacccaactaatttttgcatttttagtagagatggggtttcgccatgttggccaggctggtcttgaactcctgacctcaagtgatccgcccaccttggcctcgcaaagtgctgggattacaggtgtgagtcaccgcacctggccagtgagtgtttgtttttgaatgaatgaatgcagagggacgaaatggttgcaacaatctaggtaagcgagtctaaaaggaagccttggctgggcatggtggctcacacctgtaatcccagcactttgggaggctgaggcaggtggattgcctgagctcagcagaccagcctgggcaacatggtgaaaccccgtctctactcaaaaatacaaaaaattagccaggagtgctggcacgtacctgtaatcccagcagctcaggaggctgagttaggagaatcacttgagcctgggaagcggaggttgcagtgagctgagatcacgccactgcactccagcctgggtgatagagagactctgtctcaaaaaaattaaattaggccgggcatggtggctcatgcctgtaatcccagcactttgggaggccgagatgggtggatcacgaggtcaggagattgagaccatcctggctaacacggtgaaaccccgtctctactaaatatacaaaaaatcagccaggcgtggtggcgggtgcctgtagtcccagctactcgggaagctgaggcaggagaatggcgtgaacccaggaggcagagcttgcagtgagccgagatcgcgccactgcactccagcctgggcgacagagcgagactctgtctcaaaaaaaaaaaaaaaagaaaagaaaagaaaaaagagagagaaaggaaggaaggaaaagaaaggaataaaggaaggaagggaggaagggaaagaaagaaaggaaggaagcagggagggagggagggaaggaaggaagggaaggaaggaaggacggaaggaaagatagagaggggacatgactttggttttggggtaggggtgagggggatgtctcatgcgagagtaggggccctggggactagtgaccagcagattcctgacccacacacctcctcccccaacccccaggagggccctggagagtggctgacgctggactcccaggtcatcatggcagacagtgagatctgtggcaccaaggaccccaccttccaccgcatcctcctggacacgcgctttgagctacccttaggtgagtacaggggcccgggaccttcacccagccccagccccagttgtggactcactcagctattccaggagagtgaacaggcctgggtaggtctagctgacccttcaggctgagaatgagggtccttcgccccggaggagagagagtttcttgggttgcagagaacagggatgtgaacctgctgggtgcgggccatgacctgggccccgtagcttcccagtgagctgcaagtgcttcacgaccgagggcgttcagggttgagctatcctggctgttccttggtgatgcctgagactgcaacagtgaactggggataggaggagagacagacagagacagagagacaaagagagacagagagagacatgtagacagaggctgagaacagatagtcccctcttcctttcctggaagcaccttgagtctctccacctcattcttctaagaacccatggctggccaggcatagcttctcatgtctgtaatcccagcactttgggaggccaaggggggaggatcatttgaggccaggagtttgagaccagcctgggccatctagtgagatcccatctctatgaaaaagttaaaaattcaccaggcatggtgctgggtttctgtagtcccaactatttgggaggctgaggcaagaggatggctcaagcccaggagtttgaggcttcagtgagttgtgattgtgccactgcactccagcctgggtgacagagcgagaccctgtctctaaaaaaataaaaataaaaacaatgggctgggcgtggtggctcatgcctataatcccagcactttgagaggccgaggcagatggatcacctgatgtcaggagtttgagagcagcctggccaacatggagaaaccctgtctctactcaaaatacaaaaaaattaactgggcatggtggtgcacacctgtaatcccagctactcgggaggctgaggcaggagaatcgcttgaacccaggaggtggaggttgcagtgaactgagattgtaccacttcactccagcctgggcaacagagtgagactccgtctcaaaaaacaatttaaaaattaaaaaattaaataaaaacaatggcttccccaccctcgtaaaggctaggaaccatctcctgtcatcctccaactattctccattcagctctgggaagaggggcgtcagaggcaccacggtacctccccttcatctccatccaccctcctgagaccgcagaccaacccacccctacttctgaggctttgccaaagaccccagaagccagggcccgggtggagctcagtaggcctggaggatggaggctgagcggcagggagggtcttccaggctcacagacagggctctgcttgcagacattcctgaagaggaggctcgctactgggccaagaagctggagcagctcaatgctatgcgggaccaggatgaagtgagtgttggggtgggaggggactgtttcttccttgtttctgtctctgtctccccatgattttcacttacagatgctgtttctctctaagcttgtctttccctgcatctctgtttctaactctttgtaacctgactgcatccatgacccatgtggtccagtctctagacaatgatggggtcaagaaacacacagaggccaggcgcagtggctcacgcctgtaatcccagcactttgagaggcgaaggtgggcagatcacctgaggtcaggagttcaagaccagcctggtcaacacagtgaaaccccgtctctactaaaaaaaaaaaaaaaaaaaaaaaattagccaggcttagtggctcacacctgtaatcccagcaatttgggaggctgtggcgggcagattatttgaggccaggagttcgagaccagcctggccaacacggtgaaaccccgtctctactaaaagtacaaaaaattagctaggcatggtggcgggcgcctgtcattccagctccttgggaggctgaggtgggagaatcacttgaatctgggaggcagaagttgcagtgagccgagatcacgctgctgcactccagcctgggcaacagagcaaggctctgtctcaaaataataatattgataataataataaaataaaaaattttacttttcatttggcggatacatgtgcaggtttgttacacgaatatattgcatgatgctaaggtctggaattccatggcattactcaaattgtaaacacagtacctaacgggtagttttttcactcttgtcctgctccctccttcctcccttctggagtccccaatgtctactgttcccatctttttttttttttcttttttgagacggagtctcgctgtgtcaccaggctggagtgcagtggcacgatcttggctcactgcaacctccacctcccgggttcaagtgattctcctgcctcggcctcccgggtaactgggactacaggcatgcgccaccatacctggctaatttttgtatttttagtagagacggggtttcaccgtttagctaggatggtctcgatctcttgacctcgtgatctgcccgcttcggcctcccaaagtgctgggattacaggtgtgagtcactgcacctggcccctagtcttttttctatctctgtttcttactttttctgtctctgtctctgtctctgtctttctctctctctctctctctctctctctctcgtgtgtgtgtgtgtctttctctccttttctctcttactccctgtatccttctcatagagagggagagaatttgggaggtagagggaagtcgggatgggacagaacagaggactatcctggtcccctggcctttggcccctctgagagaagccctttctccttcctccaccccttcagtattcgttccaagatgagcaagacaagcctctgcctgtccccagcaaccagtgctgtaagtacctcttgattcttccttgggtatcagctgaaatcctcatccctcccctaccccatgactgtcctgtctaaaggctcctccagtccggagatacaagtcctgagagtgcagtgtccacttacagtgtattagaaatcagcgttctgaggctgggcatggtggctcacccctgtaatcccagcactttgggaagccgaggtcaggagttcgagaccagcctggccaacagggcaaaaccccatctctactataaatacaaaaaaaatgaacaggcatggtggtgggtgcctgtaatcccagctactcagaaggctgaggcaggagaattgcttgaacccaggaggcagaatctgcagtgggccaagatcatgccactgtactccagcctgggtgacagagcaagactctgactcaaaaaaagaaggaaggaaagaaagagaaagaaagagagaaagagagagagagaaagaaagaaaggaaggaaggagaggagaggaaagaaagaaagaaaaagaaagaaaagaaaagagaaaagaaaagaaaggaagggaaggaaagaaaggaaaggaaggaaaggaaagagaagagttctgagacttgggcttgatgaaagacaagaaggaagaaaggaaggaaggaaggagagttctgagacttgggcttgattcccaccttgctatgtggtcatgggcaggccagtgtccctcttcgatatttaggggccctctctttgaaatagcgcatctgacagccccctcactctgagggctgttggaaggatctgggacatctagagacatgaaaaacacttcgtagatgcttcacttccattcatcagatcctggatgagggcgtataactcattccacaaactattcatggggccagcacaaaccccgagcagcccacgtcagatggtgcagacagacacaggcagaagatcctgccagcacatgtacaatgggcgcacgtacaacagacgaaggctgtttcactaacccattttcttagatggagaaactgaggcccaagaggagatgtgatttacccaaggccacacacccggcaactggcagagccaggatttgaacccaggtctgagggactccaactttgcttccctttccactgcaccttgtctgttaatgatgatgatgatcacgttcctcttcctatttccatcaaggctgcagttgtcaagattttttatcttcctgggccttgtggacactcagatgcatccagaccaggtcttttcatggatggaggagctgtctccctccttaaaatgacttcccattgctctgtggtggaggggaagtagcaaatcatatagcctcagggcagagctcccacctgctgacaccaaaaccccactgcactctttagcctccttctcctttcccatttactttcttttcttcttctctttttttttttcttggagatagagtctccctctgtcacccagactggactgcaatagtactatcttggctcactgcagcctctgcctcccggatttgagtgattcttgtgcctcagcctcttgagcagtggggattacaggcacatgccaccacgcccagctaatttttgtatttttagtagagatggggttttgccatgttggccaggctggtctcaaattttttacctcatgtgatctgcccacctcagcttcccaaagtgctgggattacaggtgtgagccaccatgcctggcaccttttccatttgcttttccatcttccttttctcccctctcacagctctccctgtatctgtgtctccctcaccatgtgtccaggagggtggtaatggaaccggggttgaggagggcttgggatactccagctggggtaccctgtttccacactaacttggggagtgatattgagaaagttccttggcttccctggccagggaggggtcatcactgcctggaaggaattacaaacccccgcttggtgaatggggtcattgagaggttgcaatgggggcggggccaggcagagtggaggcaggaaggcaatggcagagagcccctgggcagatggactagagctaggggcaggttcattttgggaagtggagaccaccactcagtatttacccacaagtcctcccttggagtattttgtcaccctatttctcagagaaagcagccaatggtcagaggagccaggattctggtccaggacatttttctgaagtgccaggtactgcacgtgataggcaaaggtggttctcgccctggccctgatgcgcagaggctctgtgactttgggctgctcactccatccctctgagccccatagtttcacccataaaatgagtagaatgataaaatccacccaacaggggtgtttcaaaccttagggtttccaatttagtgtgtctttccttatccaagagctgggagaggaaaagggtggagactgggagactgctaaccctagacatcatagtcagaaaaccctgggcccaaatacccactttgttacttcctgactgagcctttcctctcattgaaaactgagccaggcacagtggctcatgcctgtaaatcccagctactcggcctcacttgagaccacgtgtttgagaccagcctgaccaacatagcaagatccatctccacacaaaaaaacaaaaagaaaaccaccaagaaccaaaacattagctgaacatggtggtgtgtgcctgtaatctcagttactcaggaggctgaggtgggaggatcacttgagcttggaaattggaggctgcagtaagccgtgattgcaccactctactccaggctggatgacagagtgagaccgtgtttcaaaaaaaaaattaaagaaaagaaaaaaataaacaaacagggatatcccaccttcctcccaaggtggagagcagagacacagcaagtgtcagcaatgaaggtgatactcagggaaggtactcaggacaagagggacttttggggtggggggagcagttggggagagtgaggacagcaggggagcccccttcctacacccacccctctgtctgctcccttttattatttctgccccttctctttgtaggcaactggaattattttggctggggtgagcagcacagtaagtactcagcctgattgatgttctgggagttacatgggaaggtgggcaccttggtgatcccaaggcccctccccactaccacacagggaagcaaggagcctgtgacgagcacgtgggggccagagggctcagcaagacccctttcaagctttcctgtaccctgttcattagtgcctgtcttgggaggcctgccatctgctggggagtgggcaccgtcattggggtggggtggggggggttagcctggaggagtggacatttagaccaacaacctgcttgtctccagctgggcactgtgacccccgcaccccactccgccgtataacctcacctcctctcctgtagcccctgccttttggtcttcctatcttccttctccacttggaaatgtgtccagtgtcactcagagggtagtgggtgggcggggtctctaacagaacggcaggccagggacctcagggagatgcacacagggacctcagcaccccccaggtccacccacactcacacctcaccttccaccttcatttgctctcccagggaataaggcaagccaccccctccctaaattaaatgtccattttccttttcctttttttttttttttttgagacggagtctcactctatcacccaggctggagggcagtggcacgatctcagctcaccgcaagctctgcctccagggttcacgccattctcctgcctcagcctcccgagtagctgggactaaaggcgtccgccaacacgcccggctaattttttgtatttttagtagagacggggtttcaccgtgttagccaggagggtctcgatctcctgacctcgtgatccacctgccttggcctcccaaagtgctgggattacaggcatgagccatggcacctggccctaattaaatgtccgtttttctacctaacagcggttcccaacctttttgacacaagggaccagtttcatggaagacaatttttttatggactggggttgggggatggtttctggatgattcaagttcactgcatttgttgtgcactttatttttattattattattattacattatagtatgtaatgaaataatgatacaactcatcatcatgtagaagcagtgggagccctgaacttgttttcctgcaattagacagtcccatcttagggtgatgggggacagtgacagatcatcaggcattagattctcataaggagcacacaacctagatcccgcacatgcacagttcacagtagggttcacgctcccgtgagaatctttttttttttttgaaacagagtcttgctctgttgcccaggctggatggagtacagtggcgcaatctcgcctcactgcaacctctgcctccgagttcaagtgattctcgtacctgagcctcccaagtagctgggattacaggcgcgtgccaccatgcccagctaattttttgtatttttagtagagatggggtttcaccatgttggccaggctggttgtgaactcctgacttcaggtgatccacctgccttggcctcccaaagggctgggattacaagtttgagccactgcgcccggccttctatgagaatctaatgctgccgctgatctgacaggaggtggaactcaggcggtaatgtgagtgatggggagcaactgtaaatacagatgaaacttcactcgctcacccaaccgctgcccacctcctgctgtgcgccccagctcctaacaggccacagaccagatccatgccctaaagagttgctccctttctcccataccccgcctcttctagagaaatccagtcctctattctttctttccttctttcttttttcttttttttgagatggagtcttgctctgtcacccagactggagtatagtggtgcgatcactgctcactgcaacctccacctcccgggttcaagcagttctcttgcctcagcctcccgagtagttgggattacaggtgcacgccactatgcctggttaatttttgtatttttagtagagatggggtttcaccatgttggccaggctggtcttgaactcctgacctcaagcaatcctcccgccttggcctcccaaagtgctgggattataggtgtgagctaccatgcctggccagtcctctattttgaggataaatctgcctccttctctctccttcaactggggagtccttcactccttcctcatatcctggtggacaaacctaactccaggaccagtgaaactgttgacaaacactttggtccaccacaatttatcaaaaacaagttggttaaaaatcaacttaggccagccacactggctcacacctgtaattccaacactttgggaggctgaggcgggagggacgcttgagctccaaagtctgaaatcagcctgggcaacagagcaagactccatctctacaaaaaaaaaaaaaaagggccgggtgtggtggcacgcccctgtagtcccagctatttgggaggctgaggtgggagggttgtttgagcccaggaggttgggactgcagtgagccatgattgtgctactagactttagcctaggcaacagtgtgagaccctgtctctaaaaaataaatagattaaaataaataaataaaatcaattttgccaaaatgaatgcttggcagacagggcaagctcgataacaaccaacttgccacaaacgaacagtacggtcaaaagcaaagatagaccaggcgtggcagctcacaccataatcccagcactttgggaggccgaggtaggcagatcacttgaggtcaggagttcgagaccagcctggccaacgtgacgaaaccccatctctactaaaaatacaaaaattagccaggcatggtggtgcatgcctttaatcccagctactcgggaggctgaggcaggagaatcatttgaacctgggaggtggaggctgcagtgagccgagatcgtaccattgcactccagcctgggtgacagagcgagactctgtctcaaaaaaaaaaaaaacccaaaaaccaacaaacaaacaaaacaaaacaccaatgcagctaaagctgatagataattgggtcaaaaaggaaagacaattgaaccgaaaacagaccgttgggttaaaacaaaagtggttgaaaatagccagaagcaggcaatttagtcaagagctacttagtagaaagcagagcctgggcaaaagcaaaaagaatccaactcaaagtaatttacccttcataagcagataatttgtcaggaaaaaaaaaaactgggtaaacatgtgcacagcctcatgcaatttggctggaaacatctcagcccggtcaaggacaattggatagaaagaatgctggccggccgaggtggctcacggctaatcccagcactgtgggaagctgaggcgggaggatcgcttgagcccaggagttcaagatcagcctgggcaatatagggagatcccatctctacaaaataataaataaataaaattagctgggtgtggtggtgtgtgcctgtggtcccagctactgtggaggctgagatgcgaggatctcttgagcccaggaggtcgaggtttcagtgagttttgatcgcgccactgcgctccagcccaagtgagagagaccctgtctaaatgaatggatgaatacagcaaaagtctgaaggtttcactcattgctctgagattttggggtctgggggttttttcatttttttacatcctcttccccttacccgtacttctcatgttttcttcatggactactttttctttttttctggatacagggtctcattctgacacccagactgcagtgcagaggcgtaatcatagctcactgctacctcgacctcctgggctccagcaatgctcctgccataccctcccgaatagctgggactacaggcgcccaccaccacgcccagctaatttttaaatttttttaaagagatggggccttactatgttgcccaggctgatcttgaactcctgggctcaagtgatcctcctgccttggcctcccaaagtgctgggattacaggcatgagccactttgcctggcccatggactaggttttactttacttttattttattttattttttggagacagggtcttgtgtcacccagcccctggagtgcagaggtgtgatcttggctcacagcaatctccacctcctgggttccagagattctcctgcctcagcctcccaaggagctgggattacaggcacatgccaccacacctggctaatttttgtatttttagtagagacagggtcttggtatgttgcccaggctggtcttgaactcctggcctcaagtaatctgcccgcctccgcctcccaaagtgctgggattacaggtgtgagccactgtgcccggcctttgctttacttttaaaataaagataactttatcattctttttagttaaaaaattaaaaagattgcattattgaaatcttttttttcagaaggtacagaaaagaataaagaagaaaatgtacaatccccaattatgccaccacccagacatgttgcttttagaattggaatttttttttttaggatttagaaaacaaactgttcttttttctaaggaaaccaaactaaaccaaaatatcttttagttcttattaaacatgaagtttggcaactctaccacattccattttagcaatagattccaagtgtgtggtggctttgccaagctaagaatctaatcctttttgaaaaagacagaaccatgtctcagcatcttcttggttcttgcatatttgttaagttcaggtctggggtcgtcaccctcagatatcaggaaatctctaagggttaaagagcgttccgtccactttgaactcctgatcttcttccaacttccccctgactgtctgtgtgactcctgttatgggaaactaactgctcagtggaaaaaaatcccatgtcattttcagagatcctgaccttcagcaaagttgttcttcacatttgagctgaaggatgccctctactctgtccctgttttcttcacattgtaggtcctaccctagcccctagactttaggtccaatttgacccacataaagcaaagcagggttttcacctcccttgttctgaagcctatatctctattaacatgacccacaattctttatccatactagtaaaaaattctcttcttcggtagcggcaccgtaagtctgggaaattgtattggctcccatcatgtgcactctctcttcctcaccctcttcccataccgcctctctcttccagacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcaggtaggtgctgcggagtgcagcatgcatgtgggtcctgctgtgtctcctggagtggagagaggggtacggggctggacagagctggaggtggagttccagaagggcagaattcacccatccaggagggcagaattcacattttcttgtgggaattaaccatttttttctttttttttttcttttcttttttttttagacagagtcttgctctgtcacccaggctggagtgcagtggcaccatttcggctcactgcaacctccaccacccagactcaagcgattctcctgcctcagcctccccagtagctgggactacaggcacaggccgccacaccaagctaatttatttttattttttgtggagatgggggtcttgctccattgcctgggctagtcttgaacccctttttattcctatagcatcctcgaggctggccagggaagagggtctcaggcgctaagattggaggaggtatctaatcgggaaatgataaggttcgttgaatgactaagtgaatggttagaagagggctgagtggtggcccctatgtccccctgcctgtatttagacagagacagatattgctgttgacgttgagcacgatcctgggttcacataagcagcacataacagagggcctgacctggccagacagtggggggcatttgcacaagtaagtgatgtttcagtgggtaccagagggtgggtaggagtttgctagataaagcagacaaggaagagaccacggcaagtgcacaggtttatggtgttggagagacaacgaggtggccagcgtggccggaatggaggaggcggggagaagtggagggaggtatgcagggggagtttggagcgtatgggccacacagggctagcacagattttattccaaggcactagggagccatggtaggatttagagcaggggagggaggtgattggatttacaattccctgtggcttccaagggaagcaggaactgtggagacagcatctggtagagacagcagaggctggcctgggttggggcccggggtacggggaagtggatgcatttcccatactttttccaggtggaggacaggagacctgctgcggggctgtggtgatggcgctgaccctgagttcccagcacagtcaattgcctgttccccacagaccgcaaatgccacaaaagtgggaatttttttttttttttttttttttttgagacggaatttcgctcttgttgcccaggctggagtgcaatggcatgatcccagctcactgcaacctccacctcccgggttcaagtgattcttctacctcagcctcccgagtagctgggattacaggcatacgccaccatgcgcagctaatttttgtatttttggtagagacagggtttcaccatgttggccaggctggtcttgaactcctgacctcaggtgatctgcccacctcagcctcccaaagtgctgggattacaggcgtgagccaccgcacctggccaaaagcggggatttttgtcttttctgatcatggctgtgttttccagcccctagagcaggacctggcacatcgtaggtgctcagtcggtgatgagtcaatgcctgtgtgactggggctcaggtgggcagtgactcggtaccccagtccctgaaccctgttccatccccacagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagaggtaacaggacggggccaaggggccagaggggctgccacaaggtcacagggtcagcagggctatgatggggtcagcgtggacctgggaagttgctggggctgaagttgtcacaggctcttgggtttcttaaagggatcagaggtgacagggcagagtagggaggtcaaagagggttcaaggggccgtgaaggagacttagagtttgtgatcttagtggttgctggggtcggagaggtcaagatcctgggcaaatggggggccagaggggctgggctggtgaaaaggaagccgaggaacagtgaagtgtcacagaagcattgggtgtgtgggaaggggtgaggggcactgaggtgggccaggggactcacgtgggtcctaatgtctctgaggggtcagagataccatggggagctatgcatcacttgcacagctggagggtcaagggaagttcaggggtcacggagagccagcccagagcctcatagcgttccctgcctcagtttcaggccgagagaggatgaggaaggccaggaggggcaggactccatgtccagggccaaggccaactggctgcgtgccttcaacaaggtgcggatgcagctgcaggaggtgagtgacacgcccagccatgcccctgccacacttagccaggccacgcccaatggcaggcccacacatgttttggccacacccacacctatattggccacgcccaacaccccacccgtacccaagcctagcctgggcatgcccagtgcagtgcccacactgtgcccatgccccagccactcgagcccttccctggccccaccctcattcagcctggcctcacccaatgtcaccgatgccccacccactctgacccttccttagccacatacacacacagcccagggtatccccatggcccgtccacaccagcctggctatgctaactcccaaaccatgcaccaagtctggccccacacatccccagcccagcctccttcccaatgtcagcctggggtttctagatctagagggaggagagaaaactagggctgaaactgggctgaagtcatggttagggatcaaggtcagggttagttcagccatcaaagatcagaggtcagggttagaattggtgttaggcaaaggggtcagtgcttagggccaaattggaggtcattgtcagggttagggaaaggtctggggccggctcagtaggggagtaggaatcaagggaagcaatcagcatcaggggcaagctgactgtttagatgggcagcagggttgaggccattcgtttattccttcattcagctcaccctgggctggggacagctgtgttgctcttcagagttaccctccaaactgcagtgggattggcaggaggagattctaaacagactcccaaaggtttagaccatcaaatatcagagccacaaaattaccaaacgaaatttcttaacagttttaaaatagaaatctattttttgactcagcagtacatacatatggttctaaatgagacaggtggaagaggacacagtgaaaaggctgttggataacttccactaggtcaagaaattaaattgaaaaaatagcagctactattggaactatgtgatatacaagaaaaacaaactatggaactagtaatatgacaagaaaaagacacatggaaggctctctgggtttcataggaaaagcataaaactaatccaaggcaatcaggaaaactccctggaggagatgacacttaaggagaagtgatggccgggcgtggttgctcaagcctgtaatcccagcactttgggaggccgaggtgggtggatcacgaggtcaggagatcgagaccatcctggctaacatggtgaaaccccgtctctagtgaaagtacaaaaaaattagccggctgtggttgcaggcgcctgtagtcccagctactcgggaggctgaggcaggagaatggcgtgaacccgggaagcggagctggcagtgagccgagatcgcgccactgcacaccagcctgggtgacagagcgagactccatttcaaaaaaaaaaaaaaaaaaaaaaagagaagtgatatccaaaatatttatttcaacaatatttcaggagctcatgcctgtaatcccagcaatttgggaggtcgaggcgggaggctcacttgcactcaggagtttgagaccagcctgggcaacgtagtgagacctcatctctaaaagaaaaaaataaaaaatatttcagagccggatgcagtggctcacgcctgtaatcccagcactttgggaggcagaggtgggtggatcacctgaggtcaggagttcgagaccagcctggccaacatagtgaaaccccacctctactaaaaatacaaaaaattaattgggtgtggtggtgggcacctgtaatcccagctactaggaaggctgaggcaggagaatcgcttgaacccaggaggtggaggttgcagtgagctgagattgcgccactgcactccagcctgggcaacaaaagggaaactccgtctcaaacaaacaaacaaacaaaatatatatatatatatttatttcaggagctgagtgtggtggcttgtgtgtgtggtcccagctactcaggaggctgaagcaggaagatcacatgaggccaggagttcaagaccagtctgggcaatatagcaagaccctgtctttaaaaaaaaaccaaacaccccctcaatatttcagaacagaagagtgctgcctgtgttcctcatagggccacccctccccccatgttaaccctttagttacaagatggagggaaggtaggggctgtggaccccacgtctatgctgagacccgaatgataaatagaggctggttggctgcagaaggtgggaagaggtacaagctgaagtcacaactcctggaaagagcccgaggcaagagagcagatgaacaagtgagactatagggggtttagggaataatcaatgaagagactgaggtccattgaggtggttgaaagagaagagcttctttctcccccagcccatcttacagatggacaaactaaggcccagagatagccacggagcttcctaaaatcactcagccaggactaggacccaggccatctgactcccagcccagaggggagtcagaagtgggtgtggagcctgctctggggaggggagggacagccacactgaccatatctgtctgtctgtcctgcaggcccggggagaaggagagatgtctaaatccctatggttcaaaggcgggtaagtaatgaaactgtgagcggtgggaggaagatttgaaccccaagtccaaggctggtttgcgcctgagcctcagcccagagaggaggaggaggggttccatttagacaggggacagattgagcttgggcaaaatacgtcactttcataaggcacacagccatacgcagacacaaaagacagtaatctttgaagacagggcggggtgcggtggctcatgcctgtaatcctagcactttgggaggccgaggcgggaggatcatttgaggtcaggagttcgaaaccagcctggccaacatggtgaaaccctgtctctactaaaaacacaaaaattagctgggcatgctggcaggcgcctgtaatcccagctacttgggatgctgaggcaagataattacttgaacccgagaggcagaggttgtagtgagctgagattgcaccactgcactccagcctgggagacagagcgggacactgtctcaaaaataaacaaacaaataaataaataaataaatgtaagataaagcagacactatgaggaactagacactattgggaaatgtacactatatatttgtaatcctggagcaaaatttttgcgttgattctatttttaaatctctttgattattgctacagttgtgggctctttaattaaagatagttgtttagtcttcagtggagaatgtggtcatgtctaacctcttatgttatggattttcaggttagaggatgcacaagatattttaaaaatatgctcaggttttaaaatagcagcacgatagtgaccttccttgtacgtccttggcaaacaggcagggagacagcagctgggactcctaaataaatgagcaggtaccctagctagagagccttggatgcgggtcctgaaatgcggattcctgtgcacgtgatttatgaaggatgtgctctcgggagaaacctgtcagacaaggggaggctgccgagaaaagatgtggtttaggagtctctaagcctcagttggctcctgcagaaagctttgtaggttgaatagccctgcagggcttgtctggagtcattggctgcaggcacctatctcactggccagtgaaaaccttttcggggccaggcttggtggctcacgcctgtaatcccaacactttgggaggctgaagcaggtggatcgcctgacgtcaggagttcaagaccagcctggccaacatggcaaaatcccgtctctactaaaatatacaaaaaattagccaggtgtggtggtgcatgcttgtaatcccagctatttgggaggctgaggcaggagaatcacttgaggctaggagttcaagaccagcctgggcaacatagcgagacccccatctctacaaaagaatttttttttaattagctatgtgtgggccgggtgcagtggctcatgcctgtaatcccagcactttgggaggcctaggcggttgatcacctgaggtcaggagtctgagaccagcctggtcaacatggtgaaaccccatatctacgaaaaatacagaaaattagccaggtgtcgtggtgcgcacctgtaatcccagctacttgggaggctgagataggagaattgcttgaacccaggaggcagaggttgcagtgagccaagatcataccattgcattccagcctgggcgacagagcgagactccatctcaaaaaaaaaaaaaaaaaaaaaatttgctgggtgtggcggtgtacacctatggtcccagctactcaggaggctaaggtgggaggattgctgtagcccaggaggtggaggctacagtgagccgtgattgtgccactgcactccagcctgggtgacagagtgagatcttgtttcgaaagatggctgggcatggtggctcacgcctgtaatctcagcactttgggaggccaaggtgggcggatcgcctgaggtcaggagttcgagaccagcctggccaacatggtgaaagcttgtttctactaaaaatacaaaaattagccaggcgttgtggtgggcacctgtaatcccagctacttggggagctgaggcaggagaattgcttaaacctgggaggcagaggttgcaatgagccaagaaaggtttcatctatattttattttatttttgatacagagtctcgctctgtcacccaggctggattgcagtggcgtgatctcgactcactgcaacctctgcctcctggttcaagcaattatcctgcttcaacctcctgagtagctgggactacaggcatgcactaccatgcctggctaatttttgtatttttagtagagacggggtttcaccatgttggtcaggctggtctcgaactcctaacctcaggtgatccgcccgcctcggcctcccaaagtgttgggattattacaggcgtaagccaccactcctggcctcatttatattttagtatattatttagatttgccttaattcacgttttcattcaaggtgggcaattacatcatctccccatttttcagatgaggagggtgaggccgggtgtctccatttcttaggactgctgtcacaaagtaccacagacgggtagcttaaaaccacagcaatgtattctctcacagttctggaggccagaagtctgaaatcaaggcattggtagagccatgctccctctgaaatctgtagaagagaattcctcccctgcctcttctagcttctagtttgcttgaattccttggcccccgagtattggtttgtagatacgtcgctttctgcctctgctgtcacagagccaccttctccccatgtatctcttcttctaaggacaccagtcatattagattaagtgtctaccctactccagtatgaccttatcttaactaattacatctgtgacaaccccgtttccaagtaaggtcaccttctgaggtagtgggggttagggcatcagcgtatcttttttttgagggacccaattcaaccccttaacacctgggggaggttgaatgttctccccagggctctatgtgaggaatagggacatctgggccctcagtgtccctctgtgtctttcagcccagggggcggtctcatcatcatcgacagcatgccagacatccgcaagaggaaacctatcccactcgtgagcgacttggtaagcaccctcgccctttactccctgagcagagcaggcctgctataagcagtgcacaacctgtggaactgcccatggtcaacctggggttgggggccagctaggcattctctgagactaatgtagtctgatttctacccccatgtgctgcgtggacacttcaggccatggtgagtgatggcgacatgggcctcttgggatcttctccccaaactcccccactgggatcttctaacaggcccccaattcatggattatgggggaaccttgccatgtcccactgtgttggatgctgtgagtcaggggtgcggggctgctcactttccagggtcccttctaactcaaagcctctgtcacctcctaattctttgttgggtctggcccggatgctgtcggtgacctgtttccaacttgcggttgtggctccaaaatgtggctgctggccccatccgtggctgtgaactctcctggtggtgatctcatggatgtcaacccccccgactccctcttctgtgacctccccagtccctggtccagtccaggaaagcgggcatcacctcggccttggcctccagcacgttgaacaacgaggagctggtgcgtggggcacctctttttccccgggtcagggatggactgggctaggagcttctggaattgtccaagcctctgtgggtccccgggaagaggaagggtgtgtgtgtgacacagactggcagaaggcagcgtggattccgacagcgtggaggcaggggagggcctgggactttaccgcgctcagtgatggggcggaatggggcaggacagggacagcgccaggttaggcgcccccaaaccgcaccccatccctccctgcagaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgtgagcgcagggcctgagcgcagggccacgggttggcgtggggagccccggggcttgcatggcggggaaaggctgaagggcaaggcgatgggtggggcaccatggggcggggcaaggggcggggcgtcgtgaggcaggggagggactcgctgaaagggaggggtggggttaaaggcgcaggactccctggtggaagggctagggggtcgaggggtggggaaagaagctgtgagggtgtcagagaggggtggggcagggctcagggactagctaggttgtgcaaaggacaggcttagtgagaagaggcagctcccctggataaaatgggttagctaggggtagctctgagaggaagaggggctcagagagggaaggggttcagactgcgagaggactgtggagggggggattggaagggggaggggactcagaggaggtgggaccaagagaaagggaggggtctgagagatggaaggggctcaggaatgtggaggggactcgcaggggtggagatcagagacgaggaggggattcagaggagtggggctcagagaggcagcgggactcagaggggtgggactcagagagggggcggagattcagaggggtgggacgcagagggggcggggactcagaggggtggggcttagggaagaggccttagaggggtggggctccaaaaaccagtggggactccaaggggtgcggggcagagaagggcaggacgcagggacatggcgactcagaggagcatggctccgagaggaggtggaagggtgaagggtctgggtggggggtacagaaagcaggtaggaaaccggcttcagggggtgacacctgagtccccccggttgatgagtaggggcggggctctcgccttggtgaggggccactatgcagggcgcgctggggtcagcgccggcctcgggcgtcctcaggggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccggtgagggggcgtgggccgccatagggcccctccttctccaccctctgagacctgtctcatgccctgtcccatggcctggtccctctcctttgcagcgcgcactcactcggtcgggtgcattccatctcatctccgtgtgtgcacgcatatggggggacatgcgtgcctccaaacgcctgttcatggcactgtatctgggtgcgtcttggatggtgccctccagaatgagactccaagaagaggaattgagggtgcatggtttatctggaaagtgaccccagaaagtcctggtgaggggcaaggacttgagacagggatggaaggagtcctgaaggagatgccgttgagcagacaatgctgtgggcaagggggtcagcccatcctggggccactggggacaatgtcgagcatgcagctcagtgtggccccacttaggggcgaggatactggcttttctccacacccagcctcatgtgggtttccccataaacagacctgaggatgaggatttcaggttatgtgggttatccaggaagtgaccccaggacggagctggggtggtaagtcagggagggaaggaaccctgcagggggcaccaacgagcaggagatctcggggggcaatgggttcttgtaagtgtctgacctcagggagttcacacctgagagaggtcctacccatgcgcaaggcagctggggcaccttcccatccttcatggtttggggactccttcccagcactttgggaggctgaggctgaggcaggcggatcacctgaggtcaggaattcgagaccgacctggccaacatggtgaaaccccatctctactaaaaatgcaaaacgattggccggatggggtggcacgtgcgggtaatcccagctacttgggaggctgaggcaggagaattgcttgaacccgggaggcggaggttgcactccagcctgggtgacgagcaaaactctgtctcaaaaacaaacaaacaaacaaacaaacacacaaaaaacctctctggcaagcccagcgtgccctgccattgctctccagccatgctgcaggtgtcccaattatcagcctgcagcgtgtggcagtgactgtccccagcatgcctcgcttcttgagtgcacacctgtgtatgccctctgtgtcttccagtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccacttgtaagtgcctggctgggtccctggccccatgtggggcatcctgctggggcagccaaaagagggctccaaggacaggactcacactggctgcctgacccctgcctctgcctcctcctcccgccacagtgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctgggtgagagaagctgggttctgaagcgggtggggacgggtcccagggatccagccagagcatggagcccacgaagagcagagagatgtgttctcaggaggagtttggtcgaggagcataatctgatgggggaggcagggtctaggagcccagtttgatgggagcaggaagcacccaggccaggagggtgagcagagcccaggaacctcttagttcagcttaggagggagggtggggacagaatctccatgttctagtctaatgggggaggcaagaagcaagaactgggaatcctagtctgaggggagagacagagacctaggaactaaatctaagaaagaagattgagctttaagaatccaggctggggccaggtatggtggctcacgcctgtaatcccagcactttgggaagctgaggcaggcagatcacatgaggtcaggagttcgagaccagcctggctaacatggtgaaaccccgtctccactaaaaatacaaaaattagccaggcgtggtggtgggtgcctgtagtcccagctacttgggaggctgaggcaggagaatcgcttgaacccaggaagcggaggttgcagtgagctgagactgcgccactgcactccagcctaggcaacgagagcaaaactcccactcaaaaaaaaaaaaaaaagaatccaggctgggggcgccgggcagtggctcacatctgtaattaatcctatcactatgggaggctgaggcaggccaatcacttgagtccaggagttcaagaccagcctgggcaacatagtgaaaccccatctctataaaagatacaaaaattagctaggcagggtgacactcacctgtggtcccacctactctggaggctaaggtgggaggatcccctgagcctgagaggtaaaggctgcagtgagctgtgatcacactgctgcactccagcccagggaacagagtgagaccctttctcaaaaaacaaatatggccgggtgcagtgtctcacgcctataatcccaacagtttgggacggctgaggcaggcagatcacttgaggtcaggagtttgagaccagattgtccaacatggcgagatcaagcccctgcactccagcctgggcaacaaaggaagactccgtctcaaaaaaaaaaaaaaaaaacaaaagaaaaagaaaaaaaaaatggaatccaggctgggcatggtggctcccacctgttatcccaacatattggttgggtggctgagctgggagggtcacttgagcccaggagttcgagaccagcctgggcaacataacaaggcctttgtctccattaaaaaaaaaaaaatagctgggtgtggtgatgcgtgcctgtagtcccagccacttgggaggctgaggtggaagcatcatttgtacctgggaggttgaagctgcagtgagctatgatcatgccactgcactccagcctgggcgatagagcaagactctgtgtccccccgaccccccaccacaagaaaagaatccagtctaaagagggaggtattgtttgggaatcctaatctgagatggctgctacagatcaaagaaccctgtgtaaggaagttgagtagaatctggggagtccaatctaatggaggaagcagggtgtatgagcctaatctgagggggtagaaaggacccagcctgagagaggtgagcagagcccggaaacctcttagattggaggacatctagatatcccattctgatgggggaggtgggagacaagaccagggggtcctagtctgaggagagaggcacagacccaggaacagtgtctaagatgtgtagtctccggggagcatgatctgatgggggaggcagggcctgggagctcagactgggaaagaagtggggcctagggctgctagtctaaggagggaggcacatagccaggaacttaatctgagtgaagaagcaaggtctgggatccttgtttgaaggaagagacagggcttaaggaattctagcctaaacagggagactcaagcctagggagccaatcagaagagagacaccaaagccttagtttccggaagtctgggaggactacagaatcgtgtatctattaaaaattgccttgggaggccgaggcgggcggatcacctgaggtcaggagttcgagaccagcctggccaacatggtgaaacctcgtctctactaaaaataaaaaattaggtgggtgtgatggcacatgcctgtaatcccagctactcgggaggctgaggcaagacaattgcttgaactcaggaggtggagattgcagtgagccaagattgcgccaccgcactctaacctggaccacagatctcaaaaaaaaataaaataaaaaaaaatttgcccatggctggccgggcgcggtgactcacgcctgtaattccagcactttgggaggctgaggtaggtggatcacctgaggtaaggagttcgagaccagcctggccaacatggtgaaaccccgtctctactaaaagtataaaaattagctgggcacggtggcaggcacctgtaattccagctagttgggaggctgagacaggagaatcgcttgaacccaggaggtggaggttgcagtgagctgagatcgtgccactgcactccagcctgggtgacaagagcaagactccctcttaaaaaaaaaaaaattgcccactgctatagtgggaagttctgacactgtcttctttttctgccctgcagacaagcgaactgacaaatctgccgtgtcgggtgccatccggctccacatcagtgtggagatcaaaggcgaggagaaggtggccccgtaccatgtccagtacacctgtctgcatgaggtgagggtcattgctcggcccctcccatgccacttccactcaccattcctgcctgcccagctcttcctctttctggccacaccatccacactctcctggccctctgagactgcccgccatgccattccctttacctggaaaactcctccctatccatcaaagtccagattcagggtcacctcctctgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatgaattaatgaataaacaggcagatggatgatgtaagctgccccagaccctgggacctctgacccccggcgaccccttgcactctccatgacactttctctcccatggtggcagaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacgtgagactgcaggctggggtgcaggaaacaagagggaggtcaccaagtgggcactggcaggtctggggtgggagggattctcagggggaagacccagagaggaagtgggcgtggtggtggtgggttccgtaattcccctggagatcaggagagcgctgtgattgacacctggagtgacatggaagagagggaagacttagaagcagagatagaggctgggcacagtggctcaagcctgtaatcccagcattttaggaggctgaggtgggcggatcactccaggtccaggagtacaagaccagcctggccaacatggtgaaaccccatctctactaaaaatacaaaagttatccaggtgtggtggcttgggcctgtaatcccagctactcgggaagttagggtgggagaattgcttgaacctggggggcaggggttgcagtgagccgagattgcgccactaaactccagcctgggtgacagagtgagactccatctcaaaaaaaaaaaaaaaagaaagaaagaaaaaaagaagcagaattagagcaactcaaatggatataaagatggaggggagaggagatgcggagacccaaaggacagaatcaggcaggggatggtttaggaggtgacagagacccactcacagatgctatcttggaggcattctaggggggatcttggtccatcctggctggacccctggcctagtgcctcctgctccctctctccagccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttggggtcagtcctggggactgggattggagaattagggagagcccaactttatgccggtcagatctgatgtgacttctgggtccccagtctagacctgattgctgtgtgactttccaccagtcacttcccgtctctgagcctccatttattcctctgtagaatgctatagaatggaggtcgggcgcagtggctcatgcctgtaatcccagcactttgggaggccgaggctggagaatcacttgaggccaggagttcgagaccagcctggccaacacggtgaaaacccgtctcttctaaaaatccaaaagaaaattagctgggtgtggtggcctgtgcctgtagtcccagccacttgggaggctgaggcaggagaattgcttgagcctaggagtcggaggttgcagtgagctgagactgcgccactacactccagcctgggcaacagagcaagactctgtctcaaaacaaaaaaaaaaatgccatagaatggaaatggacattatgagggtggtggatgaggtatacttactgtacagtaggcgctcagccactatggcggccatgtcctgcctccatccatcctccctcctccccttcctcctcagaaagagcgcttcgtgaaactcctggaccagctgcataactccctgcggattgacctctccatgtaccgggtaggaagtgtgtgcatgagaatttgctcaccccacgcacatgtgtgtgcctggaggggaaagctaccagcaggtgtgtgcatgggaggacccatcttcgcatgtacgatgtgttcctccagcatgtgtgcacctgcatgggcactacctgccgtaatgtgcttggtgcatccaagcagatccccagttgcttcgaggtgctctaatcaacccccagcctgaaaccggggagcctttgtatccccctgaaaagcagtctcccatcctccccacgccttcttccataaccccacatctcttcctgctcccaacagaataacttcccagccagcagcccggagagactccaggacctcaaatccactgtggaccttctcaccagcatcaccttctttcggatgaaggtaggaaaggatccagttgcctttgctctccaaggagggttctctaggatccccagctcccaggacaagtgacattcctcctggagagtggcagcctttctcagtaaccttttcctttcataccacttcatttccttaaacccaaaatgactcgcagtcacttcctggggacctcctgtcacagctgtggccgcctatgcctctctccctcccttcaggtacaagaactccagagcccgccccgagccagccaggtggtaaaggactgtgtgaaagcctgccttaattctacctacgagtacatcttcaataactgccatgaactgtacagccgggagtaccagacagacccggtgagactccagatgggtcagaagggaacagtgatgccaactagacacaggcttcagtcccagccctgccccttcccgagggatttaactcttctgaacctcagtatcttcttctataaaatggggtcattaacccacatctcaggattagggagaagagttcatgagctgtaagtttagataaaatatagtcggcactcagtacatttggttgcgtagttgagtttggcataaaaaaaaattttttttttttgagataaagtttcactcttgcttcccaggctggagtgcagtggtgtgatctcagctcactgcaacctctgcctcccaggtttaaatgattctcctgcctcagactcccgagtagctgggattacaggtgcccaccaccacacctagttaattttttgtatttttagtaaaaacggggtttcaccatgttggccaggctggtctcgaactccttgacctcaggtgatccacctgcctcggcctcccaaagtactgagattataggcgtgagccaccactcccagcctggcataaaaatttatgtctggcatacagtcactgttcagtaaatgttgagttgaattgaatttatttatttattttgagatggggtcttgctctgttgcccaggctggagtgcagtgatgcaatcatggctcactgcagcctcgacctcccaggctcaagtgatcctcccacctcagcctcccaagtagctgggactataggcacgcaccaccatgacaggctagtttttgtatttttgtggagatggcatctcactatgttgcccaggctgatctcctgggctcaagcgatcctcctgcctcagcctcccaaagtgctgggattacaggcatgagccaccacctgtaaggagcttatagcctattatgagggaaagatgtacaaactggaaatttacaaacagggatggttagtattgagatagtgggatctcaggaggctgtgggagctggaggcagcccctacccaggttctgataggatataacacataagctgtgttcagttcaatttgatttcattctgttcctttacattcagttgactagagttggaatcagtctcactctcatttgaattgagttgaggtgaactggattaaattaggctataaggagcttatagcctattatgagggaaagatgtacaaactggaaatttacaaacagggatggttagtattgagatagtgggatctcaggaggctgtgggagctggaggcagcccctacccaggttctgataggatataacacataagctgtgttcagttcaatttgatttcattctgttcctttacattcagttgactagagttggaatcagtctcactctcatttgaattgagttgaggtgaactggattaaattagattgtttagttcaaccataatcagtttagttatatttatttctatttggttgacttaagttaaaattgttcttgacggccaggcacagtggctcacgcctgtaatcccagcactttgagaggccgaggtgggtggatcatgaggtcaggagatcgagaccatcctggctaacacagtgaaaccccgtctctactaaaaatacaaaaaaattagccgggcgtggtggcgggcacctgtagtcccagatacttgggaggctgaggcaggagaatggtgtgaacctgggaggcagagcttgcagtgagctgagatcgcatcactgcactccggcctgggtgacagagcgagactccgtctcaaaaaaaaaaaaaaaattgttcttgacaaattagtgagccaggctgggctcagtggctcacacctgtaatgtcagcactttaggaggctgaggcaggaggatcccttgaggccaggagctcgagaccagtttgggcaacatagcgagaccctatctttgcaaaatatacaaaaattagccaggtgtggtggcgcacctgtagtcctggctactttggaggctgaggtggaaggatcacttgagcccaggagttcgagtctatggtgagccatgattgcaccactgcgctccagcctgggtgacagaatgagactctgtctcttgaaagaaaaaaaaaaaaaatagagagaagaacaagttggttgattgcattgagttttattgagtacaaattcattcatttccatttagttcatttacattcaagtcaagttgttctcaactgggttaaattgtcttgagttaggatgaactgggttgttggattagatgcaattgatttcagttgaattgggttgaattgggctaatttcagctgattatattcatttccattgattaacaaaggtgggttgaattgccctgcgtatgttcagttgggtttagataggtagaatcggattgggttgacttgggtagagttgagttcgagtcgatttatttccattattttgatttgggttgatgttgatcttggacaagttgattaggttcaatccaacggaattcatttgagtttaattcaatccagttaggttgaattgcactccattcagtgtgattggcccaagttcagttccattgatttaactcagaggaagaacttacttccgttaagctaagtgggcttaatttgcattcatttcatttccatttggtttagtggagctgggtttgttcaactaaatttggtttcatttatttccattcacttttagagtggagtctaaaaatcttaagctttatcaagctgcattgacatgagctctgtttgggccagtttatgagttcaattcctctctatatagatttcttacctgaaccatacacatgatgatgggatgcattcctctaaaacctcttgttcttcagtaataaactgaccgaggcaccaacctctggaccccctataaccgaggtctccactgtccatttcaggccaagaagggggaagttctcccagaggaacaggggcccagcatcaagaacctcgacttctggtccaagctgattaccctcatagtgtccatcattgaggaagacaagaattcctacactccctgcctcaaccagtgagttgtgtatctgtgtagttaattatctgggtgtcatcgtgtgtatgtatacaactttgtgtcttgtgtgtcaagatgattttgtctaattacacaaatgtgcacctgggggtaattgtgggtctgattatacctgtgcaaaattcaaatatgggtacaatttgcaaacataaacataaaattgtgtgtatgttcacaatcactggtataatcagacatgtattttgtatgtattcatttgcatgcataactgtgtgtacatatgtgtataattttttatgtgtatgtaggttaattaataatagtgttgctcaagtattactatgtgctaccctaatacatatcatacatttaaattgcattatatcttatatgtatggatgtaagtaattttgtatattgttgtggtcagtgggttttgggtttaatttgtatatgtgtaaaatcattgaatgcaattcaacgtacaatattgtacaatatacaatattgtgtattgtgtatgaatatgggcacagttttcatttgtggatttatatgtctgtagaattttcatctgtgttagctgaatgagctactgtgttctatttctctgccagatgagtgctagagaggactctagctttggggcgttaacatatcctggttagggaattaagatcagggatttggatggattgaagggtgggtggatgaatggatggattaataaacagatggggtcaggcacagtggctcacgcctgtaatcccagcactttgggaggccaaggtgggtggatcatttgaggtcaggagtttgagaccagcctggccaacatggtgaaaccctatctttactaaaaatacaaaaattggccacgcgcggtggctcacccctgtaatcccagcactttgggaggccgaggcaggcggatcatgaggtcagatggagaccatcctggctaacatggtgaaaccttgtctctactaaaaatacaaaaattagccgggagtggtggtgggcgcctgtagtcccagctactcgggaggctgaggcaggagaatggcgtgaacccaggaggtggagcttgcagtgagccgagattggccactgcactccagcctgggcaacagagtgagactccgtctcaaaaaaaacagaaaaaacaaaacaaaaattaaccgggcgtggtggtacacgtctgtaatcccagctagtgggagactgaggcaggaaaattgcttgaacccaggaggcagaggttgcagtgagtcgagattgcgccactgcactccagcctggggtgacagagcgagactccgtctctaaataaataagtaaataacagatggacagatagattggtagaaggatgactgtgtggataaaggaacggatgggccaggtgtggcagctcacgcttgtaatcccagcactttgggaggccgaggcgggaggatcacctgaggtcaggagtttgagaccagcctggccaacatggcaaaaccccatctctactaaaaatataaaaaattagccgggcatagtggctcgcgcctgtaatcccagctacttgggaggctgaggcacgagaatcatttgagcccgggtggtggaggttgcagtgagctgaaatcgcaccactgcactccagcctgggtgacagagtgagactccgtctctaaataaaaaaaaaggaatggatggatgggtggacagatggatgggtaaatggatgggtggatagctgcataagtgagtaaataaatggatagagggatggatggataggtggatggatgaatagatagatgggtcatttaatgggtgggtggatagttgtatgagtgggcgaatgaatggatggaaggacagataaataggtggatgatggaaggatggacggatggatggatgaacagatagacgggtaggtggctggatggacagttggatgaatgggtggagggatagatggatgaacaggtagatgagtggttgagtggatggatgaatagttggatgaatgagcaaatgaatggatggagggatggatggatagatgaatagatagatggataggtgaacggatgggtggatagttgcataattaggtgaatgaatggaaagagggatggatggatggatgggtggatagatgaatagatagatgggtaggtggatggatggatagttggatgaatgggtagagggatagatggatgaacagatagatgggtggttgagtggatggatgaatagttggatgaatgagtgaatgagtggatggagggatggatggataaatagatagatggataggtgaatgaatgggtggatagttgcataagtaggtgaatgaatggatagagggatggatggatgggtggatggatgaatagatagatgggtaggtggagggatagatagttgcatgagtgggtgaatgaattgatgaagggatggttggatgggtggataatgggtggatggatgaatagatagatgggtacatggatgggtggacagttgcatgagtgggtgaatgaatggatggaaggatggatgaatggatggaggaatagataaatgagttgttgaatgtatgggtggatagttgcatgagtggatgaatgaatgaatggagggacaaatggatgagtgggtggaggctcaaagggaaacaaagccttagtgcagagtcagtcagcagtggctatgagggaacctgaacccaaatccctctaccctaccctacactcctctgtggggcagggaaggacaaagcagcccatcccatctgccttctgcctgccaaagttttatacttccatgactttgccaggtttccccaggagctgaatgtgggtaaaatcagcgctgaagtgatgtggaatctgtttgcccaagacatgaagtacgccatggagggtgagtttcccaccccagtgttccccagcctggccacaccaggtggctgtacctctgtgtcccctccaaggctgtgagcaccctataataccctcatgcctgagttggaaatcagtgatggccagagtaggcagcctctctgccgagacagagggtggcagacccttccagctgtgccatcctctttgtccctggccagagcacgacaagcatcgtctatgcaagagtgccgactacatgaacctccacttcaaggtgaaatggctctacaatgagtatgtgacggaacttcccgccttcaaggaccgcgtgcctgagtaccctgcgtaagtcccctgccccaggcccaacccaagccagaactgtagcactgacagctagaaggaaagccagagaggaagactgacctacttagggttacctgggcatcaggggcatgggtggggcttgaatccagtgttctccactctcagccattgagcagttgactcctacagcccagaaaattcagtcaacagcaacaggaaccatgtgactttctcattttatccttttgacaattctatgagttaaggtgggatcattttaattcctattttgcggaggagaaattggagagttaaagatgccaaatggccaggcatggtggcacatgcctgtagccccggcaatctgggagactgaggcaggacttgcttgagcccaggagttcgagaccagcctggaaaagatagtcagatcccatctctaaaattaatgaatgggtcaggcacagtggcttgtgcctgtaatcccaggactttgggaggccgaagtgggcagatcacctgaggtcaggagttcgagaccagcatagccaatagggcgaaaccccagctctacaaaaatacaaaaattggccgggcgcagtggctcatgcctgtaatctcagcactttgggaggccaaggtgggtggatcaccagaggtcaggagtttgagaccggcctggccaacatggtgagaccctgtctctactaaaaatacaaaaattagccaggtgtggtggcagatgcctgtaatcccagctacttgggaggctgaggcatgagaattgcttgaacccaggaggcggaggttgcagtgagccgagattgcaccactggattccagctggggcaacaaagtgagactccgtctcaaaaacaaagaaaaaaattatccgggtgtgatggcacacacctgtagtcccagctactcgggaggccgaagcaggaggatcgcttgaactcaggaggcagaggttgcagtaagcggaaatcgtgtcactgcactccagcctgggcgacagagtgagactccatttcaaaaaataaataaatataaaaataaaatgaatgagtgaataaatgccacattccttgcccacggtgactcccagtacatgacagacccgagatttgaactcgggacatctgattcctgggctgtgtgttggaggatagtacggtgccgccggttcctgagaattgtggtgtcgttctctgctcccagcagcacaggaggatggctagtcttctgttgtacaagaaacgaaagctctggctggatgcggtggctcattcctgtaatcccaacattttgggaggccaaggtgggaggatcacttgaggccaggagttcaaggcctgcctgggcaacacagggaaatcctgtctctacaaagaagacaaaaaaaaaaattagccgggcatggtggcatgtgcctgtagtgccagctattcgggaagctgaggtgggaggattgcttgaacccagcaggtcaaggctgcagtgagctgtgatcgcaccactgcactttagcctgggcaacagaacaagacccaggaaggaaggaaggaaggaagaaggaaggaaggaaggaagggagggagggagggaaggaaggaaaggagggagggagggagggagggagaaaacaagagctcagagaataaacccacttgccatgggcagtttatctgaagaaggaaggaaggaaggaaggaaggaaggagggagggagggagggaaggatggacggaggactcagagaggactcagagaataaacccacttgccatgggcagcttatctgaactaggattcccacaggcctgggccctgttgtaaaaggggccactcaatctatttcatgagcaaagtaactgaggttcagtgcaggaggcgagcagaggagcagccactcaaggctgcagggagagctccaggcagagcaggagcagccatagctccactcgttagtgggtcagaaccaggcggcgcagaggttgaggaggacaagaggtggcagagccccaccccggctcttcattccagatggtttgaacccttcgtcatccagtggctggatgagaatgaggaggtgtcccgggatttcctgcacggtgccctggagcgagacaagaaggatggggtaagaaggcccacccacaaccaagttcctaatcttcccactggggtcaatttaggtggaaatttgcccttgagatgaggccagtctctggtaggtgtccgccatctttgtcttgctcatggtttttaaaatcttttgttcattcatcaactacaaaatgtgagttccatcaagccggacacctccaggcaccccatggcttctgcgtccacctctagagttgactctaagtctacctcgccctctgtccatctcaccccggggaaattggtctctggctctccccgtggagagtgggagagttgggctctagggattctctggtgctgatctcctccttcctcttggcctccttccccctgcagttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaaggtgtgggagagtgggtagggttggggctaggggacagggtcagggttagggaattgaagtctgagttggagatgggctaaggttgaggttgaggttggagttggtgatgtcattgaggttgggattgaagttggagtttgggttgaggttggagttgaagtttgggttgaggttgaggttgagatgaggttggagttgaggtttgaatggaggttggagttggggttagggtttaaattgagattggagttgagattggggttggagttgaggtgtagggtaaggttggagttgaggtgtgggttgagatcagggttggaattgaggtcattagtgcgagttggcaatgctgtttggttggagtttgaggctgtgctgggtcttgatttggaattgagattggagtgagtgctgagattgatgcacaatttgagtgagtcttggggtgagtttgagggttgggatcattggggttgaggtttgattcattggcattaggctgtgttgaccttgggaattacaagtaggaattggcttttgatttgagatcaggtcatcttgtggttgaggctgggttaatatgatgcttggttgctctgaaaaagagctgggtctacataaaatgatcatcttcatcgagcggtatctcctctaggtcttgatggtcatcttgagtggagaatttgcctagcccaaaggcccatcttaggtcctaatagggtttgggttcatcactgatctcactagtggccttgatgggacgactggcgcctgccttggtgccagcctctcctctcacccacactctgtgtcttccccagaccatcagtaatgtgctcctccagtatgcagacatcatctccaaggactttgcctcctactgctccaaggagaaggagaaagtggtgacctgagggggcccctgacctatctccctcctcagccccattcagcatccccaggactcccttctccccagggccctgacacctggggctatgtatgtccacctgtgtgtgatctgactggattgtctgtgtgtttagaggtgcttgtttgaatgtgtgtgatcagagtgtatctagttgtgtgtgtctgagtgtatgggaagtaagtgcatgtgtctgactgtgtattgtctgtgttgactggtatactctgcgtgtgtgtgtgtgtgtgagtctgtgcacatctgagtgtgcgtgtctgatcatgttttgtctggctttgttaacagggtatggcatgaatgtgtgtgtgagtccaactgtgcacgtctgagtgtgcgtgtgtgattgtgtttcgtctggttgtgttaactgggtataacatgagtgtgtgtatctgtgtgattgtgtttcgtctggctgtgttaactgggtataacatgaatgtgtgtatctgtgtgtgtctgtacatatctgagtgtgggtatctagccatgtttcatctgtgttaaccgggtatagtgtgagtgtatgtatgtgtgagtatgtgtgttcaactatacaacacatctaggtgtgtgtctcatattttatctagctgtgttaactggttatagtgtgagtgtgtgtatgcttgtgtgtgtgtccagctgtgcatgtctgggtgtgtgtgaccgtgttttgtctggctgagttaactgcatatagtgtgagtgtgtgtgtgtgtttgtgcacgtctgggtgggtgtgtctcaccaggtttggtatggcggtgttgaatgtgcgtggtgtgagtgtgtgtctatggctgtgtctggctgtgcatgtctgagtgtgtgtatctgtggggggggtctcgtgtgtatatctcggggtgagggcacacgccttcagtgtgtcactgtctggctgttgcaaatgaagccttcagggtgtgagctgcaaagctttgggctcttgcaggaaactctgctactcccagagttcccagcccctctcatggaccccccaccaggcctggggtcgtgggattacttgagtcctccttgcccccaccagtccccagagatgcccgccccaacaacacgcccaggaacctgagcacaaaagctcccttccatttgcaggtgaccccatgggaccccagcttcctccagacccagaccctgaaaccatcccaaggtccctggcggtgggtaggggaggctctggggagaggtcctgaggcttgactcccctctctgttcccacccacagccctgcattctcatgaataacactcaacagctacgagttcagctggagaagatgttcgaagccatgggaggaaaggaggtgaggcagggccgaggcagggctggagaagggatggggcacccccaccactgtgtccctgcaggcccctgagctcatatgccaccaggcctggggatgaattccagctcaaccatggccttaaacccaggcttgcagcataacggtctgtgcctcagtttcccctgcagccccttgggatccctttccctgggtcagatgagaaaatgcccagtacatgtggatacttactgaacctggcctggtgtcccttctccctccttagctggatgctgaagccagtgacatcctgaaggagcttcaggtgaaactcaataacgtcttggatgagctcagccgggtgtttgctaccaggtgggagcatctccagggctgggggcagtggggagggaacagccttcagccctctaagcctggggcatctggaggctcagcagatgtagaggtgaagattgcaggctcagagttagacaaggctgagttcaaatcttggctttactgcttactagctgtgtggccttgggtgagtcacttaacttctctgagccttatttttttttttattgctctgtaaatttggggtgatgacaataatagtacctgggtaggattttgagaatgaaatgaacaatgcgggcaaatgtcttatccctttgcctggttctgcagttactattttccactctttttttttttttgagacgaagtctcgttctgtcaccaggctggagtgcagtggcgcgatctcggctcactgcaccctccacttcctgggttcaagtagctgggactacaggtgcatgccaccacacccagctaatttttgtatttttagtagagacagggtttcaccatgttggccagggtggtctcgatctcttgacctcgtgatccgcccgccttggcctcccaaagtgcttggattaaaggcgtgagccaccgcatctggcctgttctccattcttgctcggttatgtgctgtgatgcaaatgatatgacctcaaggaccacagatcagcatctgtgtcctcatccagcccttcctgtggtctgagcccttcccttctgtgtccccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaagtgagctgggccaggtggactgggctggccggggaggggctggggcagggccatagagacccagggactggctggagagggtgtgcagggaatgtttgctgggtggtcactggatagttggatggctgagtagatggtggcatgaaggggatggaaagttgggtgggtaaataattggaaggattggtgagtaaatatttgtgtggatgaatgaatggtggatgaataggtgacatgagggatgggtaggtgggtggacgcatggattaaaagataaatgtttagaaggatagagggaggatagatgttttaaaaagatggatgggtggatagttgggtaaatatttggatggattaataaatgttttgatgcatagatggattgatgcttagatggatggatgtctggcaggctggcttgatggatggttggatggacgatggatggatggatggatggatggatggatggatggatggataaatggttggatatgtggataaatatttggatggatgaataaatgttttgatgcataaatggatggacagatgggtggatggatgaataaatggatgggtgggtagatggatggatagataggtggataaatatttggatgaatgaataaatgttttgatgtatagatggattgatgcatagatgtctgtctggctggctggatggatggatggatgaatggatggatggataggtcaatagatatttagatcattgaataaatgttttaatgcataaatggatggacagatgggtggatggatgaatggatggttggatgggtgggtgggtagatggatggatgctggatagatataggtggataaatatttggatggataaataaatgttttgatgcataaatggttggacagatgggggggtgggtggatgagtgggtcgatggatggatggattaataggtgagaaatatttggatggatgaataaatgttttgatgcatagatggatggacagatggatgaatggacaggtggatggatagataaatggataagtggatggatgggtagaaggatgcatggaaggatggatggatgaacagatggatggatggatggatagaaaaagaaatagagaattaaggaccactgggggagggatggattggtgggtgactggatcagttggtggatggatcttggtggactgcctgtctccttcaacccctatccatccaaccacaatctctttgctgttttccctttcaagtctgccctcctctgaccattcccctcctgttcctcttgggcatggccttctccctcatagtccctgatctccatccttcctgtttcggttcatcccccacactgttctttcaaacatgaaagtctggctgtgtctccctcttgaacactccatggctccccactacccccatcctgataaaacccaagccttcctcccagacattggggccccttcccatctggtccctgctgactagtccaaccaccactcactcttctcttcatgcatcagatatcatagccccatcaaaccacccaggggtccctgtacaggctgtgggccctctttcctatctgtggaatgccttgcccacctgttaagggaaggtgatctgtgggtgggggcgagctgggccctctctcagacctgcccctcgtccccagcctgaccctctttgccaaaatctgtgagaagactgtgctgaagcgagtgctgaaggagctgtggaagctggttatgaacaccatggagaaaaccatcgtcctgccgcccctcactgaccagacggtgagacctgcagggggcccgaggggacatttaggccacctccctggcgagagcccagaaaacttggtgcctagaggctggggggtaagaacaaaggcatccggtctcagagaggtcatccaggctcaagggccattcaagggtcatggaagccaccagaggtcagtggggggccattcagaggtcagagagttcacacaggggttaaagatcatcgaagagttaaagaggtcattcagagtccattgtattttctctggggtcaaagacatcaggtagagtcaagagaccactaaagtcatagaggtcacatgtaggtcaaaatagctttcaaaggtcagaggtcatctagaaaacaggtcaattttgggatcaaggttatccttgagccacggaaggcatagacattggccaggcaccgtggctcacgcctgcaatcccagcactttgggaggctcgaggcgggcagattgcttgaggtcaggagttcgagaccagcctgggcaacatggtgaaatctcgtctctactaaaaatacaaaaattagctgggtgtgatcctgtgatcctggcttcttgggaaactgaggcacgaaaactgtttgaacctaagaggtagaggctgcagtgagctgagatggcgccactgcacactccagcctgggcaacagaacgagaccctttctcaaaaaaacaaaaaaaaaaaagagaaggcatagacatcaatgggtttttctaagggctttggggctcattcaaaagccaaagaggggagacaccatggacttcagacatcagagatcaatagaccaaggggctcctggaagtcagccagaggtcatggagggcatccaaggtcagagaggtcaagcagagatcattggccatccagagaccaggaccgatagaccagaggccataatctctagggaccaaggggacccaaacatctgtagagggatgtagctgggcgtgggtccctccgggaggcatcaagaacctgaggacagcaatggcatgtgggctgtgggcacattagggagtggggaggtctttcccaggtggaccacggaggcttggggagggtgagcgggggccatagatgggcgacgatggtcggggaggctcggacttcccatccttccacctcttctgggctgggctggggcaggggtcttgcccctccgtgctgccatctctgccccctccctggccttcccgggagactcatggtaccttattcccccctcccccaccgccccgcccccatcacggatgacagatgatcgtaagtagagtcccgtctgtccgtctgtctgtccgtcctctctgtgtctggactcggcccaccgcgtcgctgcacccctctccccacccacccagcgccccctcctcacccaccatcctccctcccgcctagatgtgcctgtgtctgtctggggcggggcatggggagggggcggggctgggagggagggagaccacgcccaccctccctgtcccactgaagtccagaggagccccactacccactcccaaaccccaagcccagctcctccttcgcagcggacctgccgtctaagggtagaatccaaccttgctcccgtgcccagatgtcaccagacgccctgaagctagaaagaggatcaaccccaggattcctctgacctcctctttgggctctgaactgtcagcagcttcccagaatgcaggtcctcaccatgagggtctctacttctgattcctctgaaattccatgctccagaccattccttggtaccccaccaccctgtctacccaactcccgccatccacaccgacacacaccaatcccagacatacacccaggtgcccaagtgggagacttttgtcatggctttgatgtcaggtagacttggggtcaaatcctagcccagccactgtctctctctgtgaccttgggaagtcacttgacctcccagcctctgtttccttgtctggaaaatggggctcattgcatcacctccctgagggtagttctaagaaccaaatggcatgatgcattaaaatactcatctcagtattgcctgaacccaggaagcagaggttgcagtgagccgagatcgcgccactgcactctcagtgaccggcccagaggcagagctaatggtggtggtggtggtggtggtggtggtggtgatatgcagacatgtgtaagagcagagacaaaacaacttcagattttccctttgtgggcaagacaaggctgttacaagatttcactttgttaaaagtttacaagggctcttacttcctggtagctgcagcctgggtctttggctataaccacttggtggcagcatacccaaccacaggcaatgcatttgtaggcagggggaataaaggaattcgcatttaatgactccccatatgccaggccttctcatagctcctgtttcagctcaccttcatgataaccctttgagtgaggtggatattattatccccattttgcagatgaggaaagctgaggctcagagaggagtcacttgttcaaggtcatatagtaaatatatgccagaatcaggattcaaattcagatctgtctgaatatgcagcccatttgctttcctgttaacccaagaaccaagagtctcatgtgtacagtaaatgtaaatggattatattttggcagtggcttttttcccttctttctgactttctttagctctgtatctgtatctttctctctctttcctgtctctgcctctcacaccatttaagagtgagtcactaaagcgcatgcacgtgtgtgcacacacacagacacacacacaaacacactatccccatgcaacccagtcaccagtaccctgtgcagtgtggattcccccatccccatgaatattcctgtgagcatcccagcttcccaccctgcacgacttgccaggttgatcaccctgtgacattatctctgcaggggaacctcttgagaaaacatggcaagggattagaaaaggtaatgagggcctcgtctcactgaggccctgagctccagtgtctgggcatgtgtgtgcatgtgtgtgcgtctgtgcacctgggcaggaagggcagctgggggtcgtgggtaggagatagctctcctaaccaagtcctgcaaaaccagaagtcagagatgaagtgattctccagggacagaacgtgcagtgtgtgtgtccacatgtgaactaatgtgtgtgcacaaacagctcccatcctcccacagcaatacccacagggctcatctgagccctgggctcagctgaaattctgtgccttcttgaggagcctacacgctagatcaccaccctggactgagacgtgttgttttttacgtagatttctttttgttgctgtgctaatgggtgtttgttgttaaggagatgggagctacagcaacacgcaagcagctgtcttgctgtacacttgtgcagaagatatcgttgtacatttgtgtctacaaatggagtcttagctttgtgaatgtgggtcacaggggttccgtactctctctgccagccctttgcggggcacattcagtgtcccaggcaccctatatgtggatgtgaatccgtgtggggtcaagaagggtgtgtgggtttttgtgtgtgatataattgtgtacgtgcctgcaagtgtttaggagtgcacgtatgtagtttagtgtatgggttcttcagcactgagtgaacttacagtatatgttaatatgttttttgtagacttgggtgctgggatataggggcagccccttagggaatggaggaggagacagattcctagccctgtctcatctccccacctccccaggtgccccatcctggggcaggctgactgtgggatgaggctgcttccagcctcctgggtaccccgagagtgtgctggcagtcaaggagtctgacctctaaacccctgctaggggaccagaggaggtgctagctggggagggggcccaccattagacccaggacctggggaggagctcctgggctgggaagtgagactgtgctttgagggggcagggaagtcccactgagtatcctcccaacagtcaccagaactctgacctccacctgattctctccacagggcagggtgaaattgccaagccactcagacgtaagaccggcctctcactgtctgtctggctcccatcccactcccccccaccccacccctgtgcccccttccacctctgtggttcgtgccgtccgagggcgtactgtgaacctgtctgttggtctcttcttcctttgtctgtctggcttgctcgtggggggtggtgggtggggggttggtgggtagggcagagggtacccaggaaggcccctgagccctgccagactacaggtgggtgcagcaggggaaactgaggcccagtccgctggacccctgagtaactgtgccttgtaagaacctcaggatcacagacaagaaataagaatagtaacagccaagatgataatagtaatacctctaggaatgaccagatggccagctggtagactatgaacataattccaaatatttccacagtaaatgggccccctccccacctagcctcacccaggacccccagaccaccccaggacccagtaagacaagacttgttacctcaatactcagccccaccctgtcccccaagttaccatgggtatccctgttagccacagtgggaattgaggctcagagcattcagggacaagcctggggccatccagcagtgacagaggaggctagacacatgccttccaggctgggaccccgtctcccttagcattgcccgtgactttgagaccaagtgataattagcgaacactgagcaaagacttgttcagtccagactaagccacaaagtggatataattttcctcatctagatgggaaaactgaggctcagagaggttaagtgccttgcccagggccacacagcaggactgcaatttgaacccaggcccatctggcttgtcagagccatcaccacatcaccatcttgctagtgtctgtttgggggtggggtggaggacggatatggaggtgtttgtatagagggaggatttttcctttaaaaggtcagactggctggcaatgtggctcacgcctgtaatcccagcactttgggaggctgaggtaggaggatcgattgagcccaggaattcaagaccagcctggacaacacaacaagaccccatctctatgaaaaaaatttcaaaaactagctggatatggtggctcacacgtgtagtcccagctactggggaggcgggaggattgcttgagcccaggagtttgaggctgcaatgagctatgatcatgccactgcactccagcctgggcaacagagggagactctgtctcaaaaaaaaaaaaaaagaaaagaaaaatcagactgaaattcctcccagattacaggggaaatttccctgcagaggggatgctccttgaggaggaggaagtgtttgaccagagccattttctgcaacctcagcctagcctattgtgttcccaaagctccctttccctggaaaccctgcccacacccacaaacaccaccgtctgaggctcccccaacccactgcccctcacagtctggtccctgtctgtttctcctgttacacaagcctgggtggggctgagagggtgggggactatcctcacaggaccgggattttcagggacaaggtcaccacatcctcctcagttcttcctggggaattccagacctctgtccctgggacacaattattgccctgcccagagcctttgggcaagaaccaagatggtctctggggctggagtgtcagctgtagcacccctgccctcacccagctgtatgttgttacctgcagggaacccagatgatcttcaatgcagccaaggagctgggtcagctgtccaaactcaaggtgaggagtggggatagattatggcgtgagggactgccccccaaactcatgtggcttccaggttgccaccatcctgggggaggggtcccaggatgatgacatgcattacaggctcaaaggacagaaactctactcactgaagcacaagatatttgctccccagtgtgtcagaaagatgtgaatggttcagcttcaggtacagcttgatcaagagggcaagtcatttattcaaccaatattttttgagcacctgctgtgtaccagtcacatattgggtgctgggatgatgaaaacatagaccctcagcccagctcctccaagttgaacagggatgagacccatagacacagattccaaaggtggagcccactccacccacatggcccctctccccatccatccaggatcacatggtacgagaagaagccaagagcttgaccccaaagcagtgcgcggttgttgagttggccctggacaccatcaaggtgagaggccctacccctccccatcccaggtgggggatgtagagggtgtggctgctgtggtcatggacttaggctcgcctcggacccacattaatttgttcaatttatggacactccctcgttcccctgtctgtgccagagctgggctggctaatgctggagccttaaggagccccaggtcggggaagactgctggatacagacattctcagccttgtaggattaggcttggggtagagggaaggatgtgggctgggggagcccaaagagtgagaaggtgtagagggaaggatgtaaaggacctcgtgccaggcatttcttgaggtgtagaagggtagagggaaggatgggggctgggcgagcccaaggtgggagaagccaggccttcctggaagacggcatgtgagaggtgggttctgaggaatgcataggagttcactaggcaaagcaaaggaagaggtgagaaagtagtagcgggaagaattgggagtggggacagcagggctacccagccctatgagtgggtagaggttcccaccatcctcacctctccccaccctgtcctccccagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggtaagggtacccagaccgcatcaccctttcccgctccgcggagacagccccgccccaagcctgaacggctgccattggctcactcccctgtgacgtcatggatggggcggaattgacatctctgcaccaagtccagggaccaatgagcgatcaaaggttcatgggggcggggcctctgacgtcacaggcctggaggtgatcgtcaacccctgggaaaatctgtgatgtcctggggccagacaatgattaacagctctgggatggaatgtctttgacatcaaggaccagggtaggattgatgactcttaggtgtggaacctgtgacgtcgtagggccacaaggtgattgtcatctcctgggaaaatcaatggtgtcccagggccaatgaatgattgtcagctcctgagggacctggacagtcatgggtccagggaatcgttgacagctctgggagacttcaggagtgattgacagctcaggatgggcaggacctctgatgtcaaggggtcaaggaatgattggcaggtccatgggtgggaactttgacgtcatggtcccagagagtggttgaactcccctgaggagggacctctgtgatgtcatggggctaaggaatgattgacagctccgagggcgggagggacctcgacttggtgggggagggtggacggagagggagttcccatttctcaaactctctctttctctctgtattttgcttcggtctcctgtccctctctgagcgcacacacacggcagctggggctggatggagggcaggattccctggggacccccccttttcaaaattggtcccgacaaggggaagaagtttggcggcttgaactcaggagggggagggagaatcgccatcgacacccagcctggggcgaggggagcaaagccctgtggcctccaacggcccccttctcccccgatccagccagcccaagggccctccgtgggcgctgggtctctgagctcagcttcccttccagtcctggctgcttttagtgtccctggtgtccctggagcaggaggctgtctgtctctccgtctgtctgcagctgacgtttctctctctcttgtttttcctccctctcactcgccgtcccctctccgcgcctcccctgccccctccacacacactcctccatcctcacctctcactggctcgtctctccccgctccccatgcttcccacctctctcccccttccacccccacccccaattagtccatggtggaaaaggtactaggtttacccttagtgaagacatttatcctgagaagggtacgtgcctccgcctcccgcccgccgcccgcgcctggtgcccggtgcctgcgcccctcctggccccggctagtaaaaggatcgtccttctgcctcaagggcctgcagggccgccgcccaggcctgagcggcttggagggcccaggcgggtgagggggaaggacgatttgctggttctacatggggaagaagcgattccagtgggaatcccagaatccatttctcctgatatgacatattttttaagccagatcctgggaccccgggacaggcacctgtcaggttaagagctgggctctgattccagcccagccactccacgggtaggcaccttgggcaagaatctgtctccctgagcctcagttttctcatctgtaaaatggggacagtcaaggaccctacttcataggattattgttattgatattattatttagagacaaggtctccttctgtcacctaggctggaatgcagtggtgcaatcactgctcactgcagcctccctcctgtgctcaagtgatcctcctgccccagcctcccgaggagctgggactacaggcacacgtcacatggctagctaatttttaattttttttgtagagaggtggcctcactttgttgcccaggctgatctcagtgaatacttatcaggctctaaggctgagcaggaccttgtgccaggcatttcttgaagaatctgtgtttggttttgttgttgttgtttgagacagagtctcactctgtcacccaggctggaatgcagtggggaaatctcagctcactgcaacctccgtccgcctcctggtttcaagcgattcttgtgcctcagcctcctgagtagctgggattacaggcccgcaccaccacgcctggctaatttttgtatttttagtagagacggggttttgccatgttggccaggctggtcttgaactcttggcctcaagtggtccacccatcttggcctcggccttccaaagtgctaggatgacaggtgtaatcccagcgcctggccattttgtttttaatttgaggcagacgctgtcacccaggctggagtgcaatggcgaaatctcaggtcactgcagccgccacctcctgggctatgttgcccaggctggtctcgaactcctggcctcaaatgatcctcccaccttggcctcccaaagtgctgggattacaggcatgagcctccatgcctagccttcttggggaaactgaaacagatttctttaactgccccacgaggggtacatcattccacagatgagatactgagagccaaaggagattcaaccacttgtccgaggaaccttcccctgcaggggtcccgggacttggggagtggggagtggggttggaagaagcactgagaggcccaggaaagacccttccccgcgaagccagcaaagcacgggatgggggcacccagcactgcctccctccccccttcttactagctttggccttggtctgacccctcacctgacccaccactgtccaattccccctggcacccccggcaccccagtctggcccggattgagcttgctatgaagcatcggcctgggggagggcggggcggctgagcatgtgactggtggcggggggtgggggtggaatgggagcctgaggcaacaaggaagacggcctggggcgtgcagcaaagaccggggccacataccccagtactggggtgatgggggtctccaggcccctgtgctccccaaccctcagtcacttcctgtccagcgtgaggtaacccaatcagcgtttgactgacccaacctcccaccctcacctctgactttccccttcttctccccgtgcatggggtggtccaggagtggggagttgcctggtggggggtggggaggggttggtggaggtgctggcggtcacaggtccacaggccccacaggatgcccttttgagtcacagaaggcgggaccgggagagattcttttaagctgccccatcagtgatccccattggcaccatggaggacacggctggtggccccaaacagaagcccaccacaccatgattcaagcagacaagggaactttttgactccataatggaaaaattaggggttgaggcctggcgcggtggtgcatgcctgtaatcccagcactttgggaggccaagttgggcggatcacttgaagtcaggagtttgagaccagcctggccaacatggtgaaaccccatctctactaaaaataacaaaaattagccaggcgtgttggtggatgcctgtaatcccagctactcaggaggctgaggcaggagaatcgctttaacctgggaggcggaggttgcagtgagccgagatggtgccattacactccagcctggtgacacagcaagactctatctcaaaaacaaaaaagaaggccaggtgcggtggctcatgcctgtaatcccagcactttgggaggccgaggcgggcagatcaccaggtcaagagatcgagaccatcctggccaacatggtgaaaccccgtctcaaaactacaaaaattagctgggtgtggtggtgtgcacctgtagtcccagctactcaggaggctgaggcaggagaatcgcttgaacctgggaggccgaggttgcagtgagccgagatttcgccattgcactccagcctgggcgacaaagcaagactccatctcaaaaaaaagaaaaaaaaaaaaaaaaaaaaaagaaacttaagattctgcactagaagggaaataggggtaaggaaaataaaagatgccgctatgagctgtcacatcaggagggatgaggattctgggccaggtgatgcaatgaggtgacctgtggggctgggtcagagttaacaagggcaagaatggggtgagtggggagaggtaatagtaacctattggaaagggccactattggaaggggtgttgcaggtcacaaaaaccactttgattatctcagagtgaggtgggagccatggaagagtttggttttgctttgttttgttttttgagacagtcttgctctgtcacccagactgaagtgcagtggcgggatcatagctcactgcagcctcaaccagctgggattaagcgatcctcctgcctcagcctcctagatagctgggactacaggtgcatgcatcaccacacctggctaattttcttttctctctcttttttttttgagacagggtctctctctgtcacccaggctggagtgcagtggtgcaatctcagctcactgcaacctctgcctcccaggttcaagtgattctcctgtctcagcttcccaagtagctgggattacaggcgtgcactaccacacccggctaatttttgtatttttagtagagacagggtttcgccatgttggccaggatgggcttgaactcttgacctcaagttatctgcccacctcagcctcccaaaatgctaggattacaagcatgagcctcccaaagtactggggttacaggcatgagccaccactgcccgacccatggaagagttttgagcagaggagggacatgatctgacttttaaatgttatttatttattttttaattttctgagacagggtcttgctctgtcacccaggttgaagtgcagtggtgcgatctcgtctcactgcagcctggacccttctgagctcaagtgttcctcccacctcagccaccagagtagctggggctacaagcacgccccaccatggccactaatttttgtatttattgtagagatggggttttgccatgttgcccaggctagtctcaaactcctgagctcaagtgagtttgcctcaccctcccaaaatgctgggattacagctgtgagccaccgcgccctgccgacatgatctgacttaataggatttttctggctggtatactgagagtagatgaaagagtggaaggaagaccagcaaggaggctactgcaatagtccaggcatgggatgctgggggcaggtgtaggatacagggaaaagacatggctctgcttctggatgtattttgaaggtagagctggcaggattttccagtttatgggcacctagggaggaagtgaagacaaagaagagtgtcaaatattgaggtgcagggtcagtttgaggacccacggaggagaaaatgacaaaagggggttggagggagtgacaactatgtaggatgagaatcgagttggtgacattgttgcagccaagaggacaaacagccacaggaggccagggtgccagatgctgccaagatgtaatgtaacatgaagactgaattttgacaacatggcaatcaatggaataagaaaaaagaaaagaattgcattaggctcagagaggttaagcaatctacccaagatcacacagcaaggcagtaaaaggccagaacccaggattcttggcttcccaagagcacctcccatcctggccccccatcagtgcctccctatcagggctgatgacctggtgccgccagcacaggtgggtgggaggccgtcaaggtccctcctggtgggtgggaaagaacagagctcacgctgaccatcctcccctgccctgcccaggcttgggtgtagaagaccctgtgggtgaagtctctgtccatgttgagctgttcactcatccaggaactggggaacacaaggtcacagtgaaaggtgagtggtctgaggtgggagggacttgctaggttctaaaacagaaaccaaactcaaactggcttaagcaacaccctttttttgttgttgttgtttaatggtccatgaaacggaaacgtccaaaggtggccaccttcaggcatggctggatccagcagtgtaaacagttttgtaagactgaatcccactttctccatctctcagccctgctttccttttcttggcttttgttgactagaacacagattgggctgtttttgacatggcttaaataagacagaattaggccaggcacggtggctcatgcctgtaatcccagcactttgggaggccaaagctggcggatcaggccaggagttcagaccagcctggccaacatggtgaaaccctgtttctactaaaaattcaaaaagtagctgggtgtggtggcgtgtgcttgtaatcccagctactcaggaggctgaggtgggaggatcgcttgaacccgggaggtggagtttgcagtgagctgagattgcgccactgcactccagcctgggtgacagagtgagactccatgtaaacaaacaaacaaaaatgatagaattggtttttctctgggataacagtttaagtagcaaccagcctcgagctgatatggaggcttcgcagtgttggtgatctgtgctgctgccatcttttgttgcattttcatccctaatgtgttgttgttgttgtttgtttgttttagaggccagagtctcactctgttgcccaggttggagcgctgtggtgcaatcacagcacactgcagcctcaaactcctgggctctagcgatcctcctgcctcagcttccggagtcgctgggattgcaggcatgcagcatcacacctggctactttttaaattatatttttgtagagatggtgtctcactatggtatccaggctggtctcaaactcctggcctcaagcagtcctcccacctgggcctcccaaagtgctgggattccagacgtgagccaccatacctggacccaaatgtgtttttcctccactatagggtcacaaagagctcagcccccacaagcattgcagactgcggctagaggaaggagaaagtttggggttcactccctgccttttaagccacagtggagaagtagtacacgtgatttttttctctcacctcattggccaaaacatagtcatatgaccacacttagcttcatggcaatgctgggaaatgtagtctttatttcagacagggtttttggcggcgggcgaggggtggggtgtttttattttttattttttttagacaggatcttgctttgtcacacaggctggagtgcagtggcacaatctcagctcactgcaactgccgccacccaggctcaagcaatcctcctgctttagccccacaagtatgtgggactacaggcgcgtgccaccacgcccagctgatttttgtatttttcgtagagacagggtttcaccatgttgcccaggctggtctcaaactcctggactcaagtgatccacccgccttggcctcccgaagtggtgggattacaagcgtgagccactgcgcccagccttgggcaggttttgagagccacaaacatctactttttccaactgggagaacctgacctgtcttaccctctacttcctccaacagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttgtgagtttgggatgctttgaagagcatgggggcaagggagggggcatccgccaggtgaaccccaaagccagctaggcaggaggtatctggaatctgggcccgggaatgggaggggccacattctaggtgggggtgctggaaagggagaccccttagtagtgcaggaatgacatcagggaggggacacgaggatggaggtcacctttgggggctggcagatccaaggggaggcaatgagtagagtggggtaacttcatgatgaagacttgcagaggggagggaaagtctgggcaggaagtgcatggtgggcagggccattgctggggctggagactctgtctggtgaaggaatgagggtggcagtgatgacgcttatgagcggggggctgagggacctggggacaggactgaggttgcatgggggtggggactactggagtctacattgagccttgtcaggaccgttggggcagcccatctggaggagtcggctctggggacaggagtggagtgtagatggatcccaggcttgggggtgggtgtcacgtggggtggaggtggggtcacattggatggaggttcttggagttgggagtgggcctaggctgcgggtatcaggatagggtctagaggatgctgatgatgtggacagcataaaaaagtctgctctggccgagtgtggtggctcacacctgtcatcccagcactctgggacgccgaggcagacagatcacctgaggtcaggagttcaagaccagcctggccaacatggtgaaaccccgtctctactaaaaatacaaaaaaaaaaaaaaaaattagccgggtgtggtggcgcacgcctgtgatcccagctactcgggatgctgaggcagaagaatcgcttgagtccgggaggcggagggtgcagtgagccaagatcgagccattgcactccagcctgggcaacaaaagcgaaactccacctcaaaaaaaaaaaaaaagtctgctctggccgagcatggtggctcacgcctgtaagctcaacactttgggaggccaaggtgggcagataacttgaggccaggagttcgagaccagcctggccaacatggtgaaactccatctctattaaaaatataaaatatggctgggtgtggtggctcacacctgtaatcccagcactttgggaggccaaggcgggcagatcacgaggtcaggagatcgagaccatcctggctaacacggtgaaaccccgtctctactaaaaatacaaaacctagccaggcgtggtggcgggcgcctgtagtcccagctactcgggaggctgaggcaggagaatggcgtgaacccgggaggcagagcttgcagtgagccgagatcgcaccactgcactccagcctgggcaacagagcaagactccgtctaaaagaaatatatattatataattatatataaaatatgtatttatattatataattatatataaaatatgtatttatattatattatataatatataataaatatatatcatataaatatatttttatatattatatataaatatatatatatttatatacatatataatatatatttatattatatataaaattatatatagtatatattatatagtataaataaaattatatatagtatatattatattatatataaaagtatatatattatatatttcatatattatatattgtatataatatagtatatacaaaaaatattagctgtgtgttgtggtgggcgcctgtaatcccagctacgcgggaggctgagacaggagaatcgcttgaacccaggaggtggaggctgcaatgacccaagatggcgctactgcagtccagcctgggctacagagtgagactcaaaaaaataaaaaatacagaaattagccgggtgtggtggcaggcgcttgtaatcccagctactccagagggtgaggggtacgagaatcgcttgaacccaggaggcacaggttgcagtgagccatgattgtaccacggcactccagcctgggcgacagagcaagactctgcctcaaaaaaaaaaaaaaaaaaaaagtctgttcacgggggtagatggatggtggtggttagcaagggtagagttggggtcatccggggtagatccttgggtcgagtgtcagcctcacagagcactggggcagggcaatgttgttggatgttcttaggagacagaagtgcagaagaaagtagtcccagctacctgggaggctggggcaggaagatcgcttgagctcaggagtttaaggctgcaatgagctatgattgtgccactgcactccagcctgggtgaccgacatagcgagaccctgtctttaaaaaaacaaaaaactatgggccaggcgcggtggctgacgcttgtaattccaacactttgggaggccaaggcagtggatcacctgaggttaggagttcgagaccagcgtggccaacatggtgagacccccccccccgccacccccatctgtactaaaaacacaaaaaatcagcccggtgtggtggtgcatgcctgtaatcccagctactagggaggctgaggcagaagaatcgtttgaacccgggagacagaggttgcagtgagccgcgattgtgccactgcactccagcctgggtgacagagggagatgcccactcaaaaaataaaaataaataaaaataaataaaaatgcacaggggtaagggtaaatcagagctggttagctggtattatctagggggcctgggagctcttatgggacaagattgaagccagttccatgtcgtgtcagcacagggctcttggtcagaaggttttggggcgattggcccctttaggggatactcaggaggagaagcatggtctagtgggaggtcagatgtcagccagggtgggccggagtacttcagggatagaggtacaacctgggaggtggacaagagggagaagctgggcgtgaccctgtaggaggggctcttatccagctggcgtcagggcaggtcactgggactgcttaggggcaggagcacaggtgaggggcatgcgggaggcggtgtcacccatggtgaggcaccagggcaggctcatgtctggagggcatcttggaagtaagaggggtgggctgggggtgtggggggctggggaaagggccacgtcgtgggtctagaggagagcctggttaaagagcttctgggtgggacattaggggcagggccgtgcgggcagcagttcagccggcagagggcacaggcgcgggtatctcagggtgggagcatggccttgcagggcggtgtctgaggggcgggagctggatgtaaaactatgcggtcatttttcggtgggtggaaaaatgggaagaacaggcgtgggcgtggcccagctcggggtcagctgctgtgggcgcggccaagcacagggttgtaggtcaagtaacggggcaggggcggggctagcgtagcgtgggaggtgacaggtgtgggcaccccggtgtgggcgtggcccgcaaatgcggtggggacggggccgtgggcggggcatcacagtaggtgtggcctgtctcacgttccgcccctccccacgcagcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 2 - DNA, 9985 bp, synthetic construct
  misc_feature 1..9985: note = UNC13A wild-type mature mRNA sequence
  source 1..9985: mol_type = other DNA
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 3 - protein, 1703 aa, synthetic construct
  REGION 1..1703: note = UNC13A wild-type protein sequence
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYSREYQTDPAKKGEVLPEEQGPSIKNLDFWSKLITLIVSIIEEDKNSYTPCLNQFPQELNVGKISAEVMWNLFAQDMKYAMEEHDKHRLCKSADYMNLHFKVKWLYNEYVTELPAFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIISKDFASYCSKEKEKVPCILMNNTQQLRVQLEKMFEAMGGKELDAEASDILKELQVKLNNVLDELSRVFATSFQPHIEECVKQMGDILSQVKGTGNVPASACSSVAQDADNVLQPIMDLLDSNLTLFAKICEKTVLKRVLKELWKLVMNTMEKTIVLPPLTDQTMIGNLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQGLGVEDPVGEVSVHVELFTHPGTGEHKVTVKVVAANDLKWQTSGIFRPFIEVNIIGPQLSDKKRKFATKSKNNSWAPKYNESFQFTLSADAGPECYELQVCVKDYCFAREDRTVGLAVLQLRELAQRGSAACWLPLGRRIHMDDTGLTVLRILSQRSNDEVAKEFVKLKSDTRSAEEGGAAPAP

SEQ ID NO: 4 - DNA, 10113 bp, synthetic construct
  misc_feature 1..10113: note = UNC13A mutant mature mRNA short exon
  source 1..10113: mol_type = other DNA
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagctgcctgggtttcctggaaagaactcttatccccaggaactagtttgttgaataaatgctggtgaatgaatgaatgattgaacagatgaatgagtgatgagtagataaaaggatggatggagagatggaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 5 - protein, 840 aa, synthetic construct
  REGION 1..840: note = UNC13A (mutant short exon) theoretical protein sequence
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHELPGFPGKNSYPQELVC

SEQ ID NO: 6 - DNA, 10163 bp, synthetic construct
  misc_feature 1..10163: note = UNC13A mutant mature mRNA long exon
  source 1..10163: mol_type = other DNA
gcccccggtgctgaaccaagatggccggtggcggccgggccccggcgtgagccaagcgcgggctgcagccgggagatgccccagcccagcggccgctgagcccgacccgacagagccggcccggccgcctccggcccacctgcgagctcggagacatgtctctgctttgcgttggagtcaaaaaagccaagtttgatggtgcccaagagaaattcaacacgtacgtgaccctgaaagtgcagaatgtcaagagcacgaccatcgcggtgcggggcagccagcccagctgggagcaggatttcatgttcgagattaaccgtctggatttgggactgacggtggaggtgtggaataagggtctcatctgggacacaatggtgggcactgtgtggatcccactgaggaccatccgccagtccaatgaggagggccctggagagtggctgacgctggactcccaggtcatcatggcagacagtgagatctgtggcaccaaggaccccaccttccaccgcatcctcctggacacgcgctttgagctacccttagacattcctgaagaggaggctcgctactgggccaagaagctggagcagctcaatgctatgcgggaccaggatgaatattcgttccaagatgagcaagacaagcctctgcctgtccccagcaaccagtgctgcaactggaattattttggctggggtgagcagcacaacgatgaccccgacagtgcagtggatgatcgtgacagtgactaccgcagtgaaacgagcaacagcatcccgccgccctattatactacgtcacaacccaacgcctcagtccaccaatattctgttcgcccaccacccctgggctcccgggagtcctacagtgactccatgcacagttacgaggagttctctgagccacaagccctcagccccacgggtagcagccgctatgcctcttccggggagctgagccagggaagctctcagctgagcgaggacttcgaccctgacgagcacagcctgcagggctccgacatggaggatgagcgggaccgggactcctaccactcctgccacagctcggtcagctaccacaaagactcgcctcgctgggaccaggatgaggaagagctggaggaggacctggaggacttcctggaggaggaggagctgcctgaagatgaggaggagctggaggaggaggaggaggaggtgcctgacgatttgggcagctatgcccagcgtgaagacgtagctgtggctgagcccaaagacttcaaacgcatcagcctcccgccagctgccccagggaaggaggacaaggccccagtggcacccaccgaggcccccgacatggccaaggtggcccccaagccagccacgcccgacaaggtgcctgcagctgagcagatccctgaggctgagccacccaaggacgaggagagtttcaggccgagagaggatgaggaaggccaggaggggcaggactccatgtccagggccaaggccaactggctgcgtgccttcaacaaggtgcggatgcagctgcaggaggcccggggagaaggagagatgtctaaatccctatggttcaaaggcggcccagggggcggtctcatcatcatcgacagcatgccagacatccgcaagaggaaacctatcccactcgtgagcgacttggccatgtccctggtccagtccaggaaagcgggcatcacctcggccttggcctccagcacgttgaacaacgaggagctgaaaaaccacgtttacaagaagaccctgcaagccttaatctaccccatctcgtgcacgacgccacacaacttcgaagtgtggacggccaccacgcccacctactgctacgagtgcgaggggctgctgtggggcatcgcgaggcagggcatgcgctgcaccgagtgcggtgtcaagtgccacgagaagtgccaggacctgctcaacgccgactgcctgcagcgggctgcggagaagagctccaagcacggggcggaggaccggacacagaacatcatcatggtgctcaaggaccgcatgaagatccgggagcgcaacaagcccgagatcttcgagctcatccaggagatcttcgcggtgaccaagacggcgcacacgcagcagatgaaggcggtcaagcagagcgtgctggacggcacgtccaagtggtccgccaagatcagcatcaccgtggtctgcgcccagggcttgcaggcaaaggacaagacaggatccagtgacccctatgtcaccgtccaggtcgggaagaccaagaaacggacaaaaaccatctatgggaacctcaacccggtgtgggaggagaatttccactttgaatgtcacaattcctccgaccgcatcaaggtgcgcgtctgggacgaggatgacgacatcaaatcccgcgtgaaacagaggttcaagagggaatctgacgatttcctggggcagacgatcattgaggtgcggacgctcagcggcgagatggacgtgtggtacaacctggacaagcgaactgacaaatctgccgtgtcgggtgccatccggctccacatcagtgtggagatcaaaggcgaggagaaggtggccccgtaccatgtccagtacacctgtctgcatgagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatggaacctgttccacttcgtgaccgacgtgcagaacaatggggtcgtgaagatcccagatgccaagggtgacgatgcctggaaggtttactacgatgagacagcccaggagattgtggacgagtttgccatgcgctacggcgtcgagtccatctaccaagccatgacccactttgcctgcctctcctccaagtatatgtgcccaggggtgcctgccgtcatgagcaccctgctcgccaacatcaatgcctactacgcacacaccaccgcctccaccaacgtgtctgcctccgaccgcttcgccgcctccaactttgggaaagagcgcttcgtgaaactcctggaccagctgcataactccctgcggattgacctctccatgtaccggaataacttcccagccagcagcccggagagactccaggacctcaaatccactgtggaccttctcaccagcatcaccttctttcggatgaaggtacaagaactccagagcccgccccgagccagccaggtggtaaaggactgtgtgaaagcctgccttaattctacctacgagtacatcttcaataactgccatgaactgtacagccgggagtaccagacagacccggccaagaagggggaagttctcccagaggaacaggggcccagcatcaagaacctcgacttctggtccaagctgattaccctcatagtgtccatcattgaggaagacaagaattcctacactccctgcctcaaccagtttccccaggagctgaatgtgggtaaaatcagcgctgaagtgatgtggaatctgtttgcccaagacatgaagtacgccatggaggagcacgacaagcatcgtctatgcaagagtgccgactacatgaacctccacttcaaggtgaaatggctctacaatgagtatgtgacggaacttcccgccttcaaggaccgcgtgcctgagtaccctgcatggtttgaacccttcgtcatccagtggctggatgagaatgaggaggtgtcccgggatttcctgcacggtgccctggagcgagacaagaaggatgggttccagcagacctcagagcatgccctattctcctgctccgtggtggatgttttctcccaactcaaccagagctttgaaatcatcaagaaactcgagtgtcccgaccctcagatcgtggggcactacatgaggcgctttgccaagaccatcagtaatgtgctcctccagtatgcagacatcatctccaaggactttgcctcctactgctccaaggagaaggagaaagtgccctgcattctcatgaataacactcaacagctacgagttcagctggagaagatgttcgaagccatgggaggaaaggagctggatgctgaagccagtgacatcctgaaggagcttcaggtgaaactcaataacgtcttggatgagctcagccgggtgtttgctaccagcttccagccgcacattgaagagtgtgtcaaacagatgggtgacatccttagccaggttaagggcacaggcaatgtgccagccagtgcctgcagcagcgtggcccaggacgcggacaatgtgttgcagcccatcatggacctgctggacagcaacctgaccctctttgccaaaatctgtgagaagactgtgctgaagcgagtgctgaaggagctgtggaagctggttatgaacaccatggagaaaaccatcgtcctgccgcccctcactgaccagacgatgatcgggaacctcttgagaaaacatggcaagggattagaaaagggcagggtgaaattgccaagccactcagacggaacccagatgatcttcaatgcagccaaggagctgggtcagctgtccaaactcaaggatcacatggtacgagaagaagccaagagcttgaccccaaagcagtgcgcggttgttgagttggccctggacaccatcaagcaatatttccacgcgggtggcgtgggcctcaagaagaccttcctggagaagagcccggacctgcaatccttgcgctatgccctgtcgctctacacgcaggccaccgacctgctaatcaagacctttgtacagacgcaatcggcccagggcttgggtgtagaagaccctgtgggtgaagtctctgtccatgttgagctgttcactcatccaggaactggggaacacaaggtcacagtgaaagtggtggctgccaatgacctcaagtggcagacttctggcatcttccggccgttcatcgaggtcaacatcattgggccccagctcagcgacaagaaacgcaagtttgcgaccaaatccaagaacaatagctgggctcccaagtacaatgagagcttccagttcacgctgagcgccgacgcgggtcccgagtgctatgagctgcaggtgtgcgtcaaggactactgcttcgcgcgcgaggaccgcacggtggggctggccgtgctgcagctgcgtgagctggcccagcgcgggagcgccgcctgctggctgccgctcggccgccgcatccacatggacgacacgggcctcacggtgctgcgaatcctctcgcagcgcagcaacgacgaggtggccaaggagttcgtgaagctcaagtcggacacgcgctccgccgaggagggcggtgccgcgcctgcgccttagcgcgggcggtcggccgagcggcactgcgcctgcgcggagggcgctgggcggggagggacggggcttgcgccttggtgggacctccccaggggcggggctcggggggctccacgccaagggtgggctgcgcctacgcccttgactcagctttcccttttggggaattaggaatggaggatgccccgccctctcgggaggccacgcccaagggcgcgacgaaggaaggagccacatccccaacttgaggccacgcccccagcacctagggggcattttgagctgggatgggggaaacctcgtccctatggaggaggccacatcccggggctctggtaccgggaggcaccacctcatgtcccctggaaaagccataagatgggacccagacccctgggaccccagaccaattgccaagtatggaaatctcagctccctcgagggggggccctgggcaaggggtagggctctctggagcgcccctctaggtggcctggggactggagggaccaggatgctggttggagggccccggaataccggagtccctttagatatttgtgcaaaaaataaatggggggaggggggaggatgggatttcaaaagcacatgcgcccttgggcgcccaaaccctgggggccgaggggacggctctggttccccacgctgcccctacttccctttgggagtttgcctctccctctcccccaacaaacccagtcctcatatcatagagttcaacacacccatttgacagatggcaaaactgaggcttaaagagctgcttgagacttggccaaggttccaggtgccataccctctgtgcccctcccttaggcctgtgtgccccatggaagggtgggctgagatcgggatgacctgacacagctccctattgctgctaattccccctcggcctcctccaaggggtgggaattccaggccaagacccctacttcgcctttccttctccggctgccaagcaggacctttgccctcagccctttctcctgggatctccatgggggatgccatgagggcctcccaccacaaaagagaatttgggatcccctggtcccaggtttctccatcccttcttccttttccagaattttccaaataggaaagaacagaaggagaccagaaactctaggggggagaaagagaatgagagaaagagaatgagagagagagaaacacaaacacagtgacacagtgagagcttagtctccaagagcctattcattgattcaaacacccaagccacaggatacctcagatggccctcttgccagctggaagctctttctccaatgagcaaagttacagtgacctggctggagttacctggtgcacataggaccttaggggaaagttcagcgtggactacacttgctctgggatctgcttttccacatgtgtgtatggcacgcctttttctgctggattgggaaggacaagattttgctgtgctagggagaaatgaaaacggggtgagctgagtagctgggtttctggaggatagaacatcagatggggaggctttccgaggtgaagaatgagagggaaccacttactagagagaaaagagctccaggcctggggaacagcacgtgcgaaggccaggagagaagaactgttgaaacaacgagaagggtggcacggctggagctgagccagcaagggggatcgtgaggagccttggggttggggagatctgcagaagcatcagaccaggcagggcctcgtacgcagtcctgaggagttttacttttattctaagacagttggggagctccaggagctgttttaagttggggagagactggattccagcctgcaaaagctgttttgtgaagactaaaaccagtgaggagaggtggaggtgctttggggacactgaaatggattcttggaaagattctgaaggctgtgttgaaaagacacctatagctgtggggacatgactataatcccagcatttggggagaccgaggctggcagatcacttaaggtcaggagtttgagaccagcctggccaacatggcgaaaccccatctctgctaaaaatacaaaaattagctgggtgcagtggtgcatgcctgtagtcccagctactcaggagactgaggcgggagaattgtttgaaccctggaggcagaggttgtagtgagtcgtgatcacacaactgcactccagcctgggcaacagaacaatactccattccctcccctctaccccaccaaaaaaaaaaaaaaatcctgcccttagatgagctctagggctgctgagtacagttgtcccagttgcacagtgcccaagggtttggcattgctaagaaggccacgtgcaaatcctagatattgagtgttgtatgtttgtgacgttggtttcccgacatgtgaatggcccaagtgtctggaagaagtggcgccactttctaatttgcttggagatgttgcatgtcccttaaattcagacaggtgcaggtaactggaggttctgaaccaaaggttaaaatgcaaattctcatacagggttgggaagttgtagccagggataagcttatgtgactgttatatggactgaggagcagatgtgaatttcgaaccatgacatggctgagggtaggggtcgggtggatggatgattcagggttgtaacccatagagcccaaaggggaagtgatctgtgacctggggtgagggtgatctggaagatttttggatggctggaaagaaatggggaagtcgagctgcctgagagagccaagttatttcccaaaagattccttaggagtctttctgttcaagacctccgtgtgtgtgtgtgtgtgtttagggttccccagcaatggcccaggcatgtgaaggaaacaagcttcttcagggaatatttgttgaatgagttttcctgactcccaggctagaactgtttttgcaatttccaccctcttttctttcccccagagaactcctattcgtccttcaaaacccatcacggaaacccctcttggagaaaaccctccttccttcccctcaggactttcccagccaccgtctctcctccagtccagcctgatgccatgggactgggggtttctctgtccagctctgtttctcccagactggggtctgaggactctcaggacccccaactttacctagcacaggctgggcacaagtgggtgacagggagtctacgcctagtggaattatgtattggggcagggtcagtgtgagaatacacatccgcatgcatgtctgtccatgtctgtccgtaccaaccttccccttccacacggacctgggcacataggaggtgtctgagcctgacacatgggacagagagtggacatggctgagacacggacagagaaaagacaaggagtccagggggctgaaagccttttgaaatcaggaagttcctgtattggcagaacaaagcccagagaggagcagggctttcctcaacgccacccagcaagtggacacagagcccggccttggatgacacctccagggttctgaaccctggacctcgctttatgcaaggagctggccccacatttccatgaatcggggaaacagcacaagaaggttggcctgtggcagggcaagggttaaaggggtgacattgagggatgcctcagagtcaaagtcccctgaccaagaggaatagagtagaaaacacagagacagagggtgagatcacgccccgatgaggacggagagagacagagatggagagagacatagaggtggaaatatacagagaaagataaatgcagagaccaaggcagggagtgtcgggggaagtaaagagggtgtcctgaagaaagaaggatctgttcactcttaccagtctgtcctcgaatgatttgcataaaatgaggaggtgcctgtccacacccccaattcctctctcaggccccagagcctgagacctcaccatgcccccatcagagatgcaaaaaactaaacacccaactagaaatccttgggacctctctcggctgggatctcagagcctttctgtcccctacccctaccccatgtgctgtcgattttgcagatggggacaacctggggcctcccggaactctgccaccctggggaagttgggggagggccttagtcccggatcacaaccccgtctgctccccagaatcctttcctaagaatcgttgaggaccaaagttgtctttgctgacacgtgttgcttttctctttgccttttattgtttcagagaaaaatcaagttgactgtgtcaagtaacaccccaccccttacccccgtccagccatagtggctctctggagacacaggtcacaggcggagggtcccctgatcatccccaaccacacagccagggggacttgacccctgtccacccctgtctcgtgctccctcagacccccacaaaccggccaagcagtccggggaggcttcccctccacacaactcttagcatgtgattgcagatgtgaaatcaaaacgttgtttgttttttgttttgttttgattctaccccgtcggtccagtgtctgcacagacgccttcatttctctgtaaatatgtgacttggaacaaatgtttaacacaaacgagaagtggtcatgaatgcatggtgttgagatgttttgcactattctgactttttggtctctgtaaaaatattttattaacagcagacattaaaaaaagaaaaaccacacaca

SEQ ID NO: 7 - protein, 825 aa, synthetic construct
  REGION 1..825: note = UNC13A (mutant long exon) theoretical protein sequence
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHEP

SEQ ID NO: 8 - DNA, 18 bp, synthetic construct
  misc_feature 1..18: note = Synthetic oligonucleotide
  source 1..18: mol_type = other DNA
tggatggatggacagatg

SEQ ID NO: 9 - DNA, 18 bp, synthetic construct
  misc_feature 1..18: note = Synthetic oligonucleotide
  source 1..18: mol_type = other DNA
atggatggatggacagat

SEQ ID NO: 10 - DNA, 18 bp, synthetic construct
  misc_feature 1..18: note = Synthetic oligonucleotide
  source 1..18: mol_type = other DNA
gatggatggatggacaga

SEQ ID NO: 11 - DNA, 18 bp, synthetic construct
  misc_feature 1..18: note = Synthetic oligonucleotide
  source 1..18: mol_type = other DNA
tgatggatggatggacag

SEQ ID NO: 12 - DNA, 18 bp, synthetic construct
  misc_feature 1..18: note = Synthetic oligonucleotide
  source 1..18: mol_type = other DNA
atgatggatggatggaca

SEQ ID NO: 13 - DNA, 18 bp, synthetic construct
  misc_feature 1..18: note = Synthetic oligonucleotide
  source 1..18: mol_type = other DNA
ggatgatggatggatgga

SEQ ID NO: 14 - DNA, 18 bp, synthetic construct
  misc_feature 1..18: note = Synthetic oligonucleotide
  source 1..18: mol_type = other DNA
tggatgatggatggatgg

SEQ ID NO: 15 - DNA, 18 bp, synthetic construct
  misc_feature 1..18: note = Synthetic oligonucleotide
  source 1..18: mol_type = other DNA
atggatgatggatggatg

SEQ ID NO: 16 - DNA, 18 bp, synthetic construct
  misc_feature 1..18: note = Synthetic oligonucleotide
  source 1..18: mol_type = other DNA
agatggatgatggatgga

SEQ ID NO: 17 - DNA, 18 bp, synthetic construct
  misc_feature 1..18: note = Synthetic oligonucleotide
  source 1..18: mol_type = other DNA
tagatggatgatggatgg

SEQ ID NO: 18 - DNA, 18 bp, synthetic construct
  misc_feature 1..18: note = Synthetic oligonucleotide
  source 1..18: mol_type = other DNA
ctagatggatgatggatg

SEQ ID NO: 19 - DNA, 18 bp, synthetic construct
  misc_feature 1..18: note = Synthetic oligonucleotide
  source 1..18: mol_type = other DNA
gctagatggatgatggat

SEQ ID NO: 20 - DNA, 18 bp, synthetic construct
  misc_feature 1..18: note = Synthetic oligonucleotide
  source 1..18: mol_type = other DNA
ggctagatggatgatgga

SEQ ID NO: 21 - DNA, 18 bp, synthetic construct
  misc_feature 1..18: note = Synthetic oligonucleotide
  source 1..18: mol_type = other DNA
tggctagatggatgatgg

SEQ ID NO: 22 - DNA, 18 bp, synthetic construct
  misc_feature 1..18: note = Synthetic oligonucleotide
  source 1..18: mol_type = other DNA
gtggctagatggatgatg

SEQ ID NO: 23 - DNA, 18 bp, synthetic construct
  misc_feature 1..18: note = Synthetic oligonucleotide
  source 1..18: mol_type = other DNA
cgtggctagatggatgat

SEQ ID NO: 24 - DNA, 18 bp, synthetic construct
  misc_feature 1..18: note = Synthetic oligonucleotide
  source 1..18: mol_type = other DNA
tcgtggctagatggatga

SEQ ID NO: 25 - DNA, 18 bp, synthetic construct
  misc_feature 1..18: note = Synthetic oligonucleotide
  source 1..18: mol_type = other DNA
ttcgtggctagatggatg

SEQ ID NO: 26 - DNA, 18 bp, synthetic construct
  misc_feature 1..18: note = Synthetic oligonucleotide
  source 1..18: mol_type = other DNA
attcgtggctagatggat

SEQ ID NO: 27 - DNA, 18 bp, synthetic construct
  misc_feature 1..18: note = Synthetic oligonucleotide
  source 1..18: mol_type = other DNA
gattcgtggctagatgga

SEQ ID NO: 28 - DNA, 18 bp, synthetic construct
  misc_feature 1..18: note = Synthetic oligonucleotide
  source 1..18: mol_type = other DNA
agattcgtggctagatgg

SEQ ID NO: 29 - DNA, 18 bp, synthetic construct
  misc_feature 1..18: note = Synthetic oligonucleotide
  source 1..18: mol_type = other DNA
tagattcgtggctagatg

SEQ ID NO: 30 - DNA, 18 bp, synthetic construct
  misc_feature 1..18: note = Synthetic oligonucleotide
  source 1..18: mol_type = other DNA
gtagattcgtggctagat

SEQ ID NO: 31 - DNA, 18 bp, synthetic construct
  misc_feature 1..18: note = Synthetic oligonucleotide
  source 1..18: mol_type = other DNA
gggtagattcgtggctag

SEQ ID NO: 32 - DNA, 18 bp, synthetic construct
  misc_feature 1..18: note = Synthetic oligonucleotide
  source 1..18: mol_type = other DNA
tgggtagattcgtggcta

SEQ ID NO: 33 - DNA, 18 bp, synthetic construct
  misc_feature 1..18: note = Synthetic oligonucleotide
  source 1..18: mol_type = other DNA
gtgggtagattcgtggct

SEQ ID NO: 34 - DNA, 18 bp, synthetic construct
  misc_feature 1..18: note = Synthetic oligonucleotide
  source 1..18: mol_type = other DNA
ggtgggtagattcgtggc

SEQ ID NO: 35 - DNA, 18 bp, synthetic construct
  misc_feature 1..18: note = Synthetic oligonucleotide
  source 1..18: mol_type = other DNA
tggtgggtagattcgtgg

SEQ ID NO: 36 - DNA, 18 bp, synthetic construct
  misc_feature 1..18: note = Synthetic oligonucleotide
  source 1..18: mol_type = other DNA
ttggtgggtagattcgtg

SEQ ID NO: 37 - DNA, 18 bp, synthetic construct
  misc_feature 1..18: note = Synthetic oligonucleotide
  source 1..18: mol_type = other DNA
gttggtgggtagattcgt

SEQ ID NO: 38 - DNA, 18 bp, synthetic construct
  misc_feature 1..18: note = Synthetic oligonucleotide
  source 1..18: mol_type = other DNA
tgagttggtgggtagatt

SEQ ID NO: 39 - DNA, 18 bp, synthetic construct
  misc_feature 1..18: note = Synthetic oligonucleotide
  source 1..18: mol_type = other DNA
atgagttggtgggtagat

SEQ ID NO: 40 - DNA, 18 bp, synthetic construct
  misc_feature 1..18: note = Synthetic oligonucleotide
  source 1..18: mol_type = other DNA
gatgagttggtgggtaga

SEQ ID NO: 41 - DNA, 18 bp, synthetic construct
  misc_feature 1..18: note = Synthetic oligonucleotide
  source 1..18: mol_type = other DNA
ggatgagttggtgggtag

SEQ ID NO: 42 - DNA, 18 bp, synthetic construct
  misc_feature 1..18: note = Synthetic oligonucleotide
  source 1..18: mol_type = other DNA
tggatgagttggtgggta

SEQ ID NO: 43 - DNA, 18 bp, synthetic construct
  misc_feature 1..18: note = Synthetic oligonucleotide
  source 1..18: mol_type = other DNA
atggatgagttggtgggt

SEQ ID NO: 44 - DNA, 18 bp, synthetic construct
  misc_feature 1..18: note = Synthetic oligonucleotide
  source 1..18: mol_type = other DNA
gatggatgagttggtggg

SEQ ID NO: 45 - DNA, 18 bp, synthetic construct
  misc_feature 1..18: note = Synthetic oligonucleotide
  source 1..18: mol_type = other DNA
agatggatgagttggtgg

SEQ ID NO: 46 - DNA, 18 bp, synthetic construct
  misc_feature 1..18: note = Synthetic oligonucleotide
  source 1..18: mol_type = other DNA
tagatggatgagttggtg

SEQ ID NO: 47 - DNA, 18 bp, synthetic construct
  misc_feature 1..18: note = Synthetic oligonucleotide
  source 1..18: mol_type = other DNA
atagatggatgagttggt

SEQ ID NO: 48 - DNA, 18 bp, synthetic construct
  misc_feature 1..18: note = Synthetic oligonucleotide
  source 1..18: mol_type = other DNA
gatagatggatgagttgg

SEQ ID NO: 49 - DNA, 18 bp, synthetic construct
  misc_feature 1..18: note = Synthetic oligonucleotide
  source 1..18: mol_type = other DNA
ggatagatggatgagttg

SEQ ID NO: 50 - DNA, 18 bp, synthetic construct
  misc_feature 1..18: note = Synthetic oligonucleotide
  source 1..18: mol_type = other DNA
tggatagatggatgagtt

SEQ ID NO: 51 - DNA, 18 bp, synthetic construct
  misc_feature 1..18: note = Synthetic oligonucleotide
  source 1..18: mol_type = other DNA
atggatagatggatgagt

SEQ ID NO: 52 - DNA, 18 bp, synthetic construct
  misc_feature 1..18: note = Synthetic oligonucleotide
  source 1..18: mol_type = other DNA
gatggatagatggatgag

SEQ ID NO: 53 - DNA, 18 bp, synthetic construct
  misc_feature 1..18: note = Synthetic oligonucleotide
  source 1..18: mol_type = other DNA
ggatggatagatggatga

SEQ ID NO: 54 - DNA, 18 bp, synthetic construct
  misc_feature 1..18: note = Synthetic oligonucleotide
  source 1..18: mol_type = other DNA
tggatggatagatggatg

SEQ ID NO: 55 - DNA, 18 bp, synthetic construct
  misc_feature 1..18: note = Synthetic oligonucleotide
  source 1..18: mol_type = other DNA
atggatggatagatggat

SEQ ID NO: 56 - DNA, 18 bp, synthetic construct
  misc_feature 1..18: note = Synthetic oligonucleotide
  source 1..18: mol_type = other DNA
catggatggatagatgga

SEQ ID NO: 57 - DNA, 18 bp, synthetic construct
  misc_feature 1..18: note = Synthetic oligonucleotide
  source 1..18: mol_type = other DNA
acatggatggatagatgg

SEQ ID NO: 58 - DNA, 18 bp, synthetic construct
  misc_feature 1..18: note = Synthetic oligonucleotide
  source 1..18: mol_type = other DNA
tacatggatggatagatg

SEQ ID NO: 59 - DNA, 18 bp, synthetic construct
  misc_feature 1..18: note = Synthetic oligonucleotide
  source 1..18: mol_type = other DNA
gtacatggatggatagat

SEQ ID NO: 60 - DNA, 18 bp, synthetic construct
  misc_feature 1..18: note = Synthetic oligonucleotide
  source 1..18: mol_type = other DNA
agtacatggatggataga

SEQ ID NO: 61 - DNA, 18 bp, synthetic construct
  misc_feature 1..18: note = Synthetic oligonucleotide
  source 1..18: mol_type = other DNA
gagtacatggatggatag

SEQ ID NO: 62 - DNA, 18 bp, synthetic construct
  misc_feature 1..18: note = Synthetic oligonucleotide
  source 1..18: mol_type = other DNA
tgagtacatggatggata

SEQ ID NO: 63 - DNA, 18 bp, synthetic construct
  misc_feature 1..18: note = Synthetic oligonucleotide
  source 1..18: mol_type = other DNA
gtgagtacatggatggat

SEQ ID NO: 64 - DNA, 18 bp, synthetic construct
  misc_feature 1..18: note = Synthetic oligonucleotide
  source 1..18: mol_type = other DNA
ggtgagtacatggatgga

SEQ ID NO: 65 - DNA, 18 bp, synthetic construct
  misc_feature 1..18: note = Synthetic oligonucleotide
  source 1..18: mol_type = other DNA
gggtgagtacatggatgg

SEQ ID NO: 66 - DNA, 18 bp, synthetic construct
  misc_feature 1..18: note = Synthetic oligonucleotide
  source 1..18: mol_type = other DNA
tgggtgagtacatggatg

SEQ ID NO: 67 - DNA, 18 bp, synthetic construct
  misc_feature 1..18: note = Synthetic oligonucleotide
  source 1..18: mol_type = other DNA
atgggtgagtacatggat

SEQ ID NO: 68 - DNA, 18 bp, synthetic construct
  misc_feature 1..18: note = Synthetic oligonucleotide
  source 1..18: mol_type = other DNA
gatgggtgagtacatgga

SEQ ID NO: 69 - DNA, 18 bp, synthetic construct
  misc_feature 1..18: note = Synthetic oligonucleotide
  source 1..18: mol_type = other DNA
agatgggtgagtacatgg

SEQ ID NO: 70 - DNA, 18 bp, synthetic construct
  misc_feature 1..18: note = Synthetic oligonucleotide
  source 1..18: mol_type = other DNA
gagatgggtgagtacatg

SEQ ID NO: 71 - DNA, 18 bp, synthetic construct
  misc_feature 1..18: note = Synthetic oligonucleotide
  source 1..18: mol_type = other DNA
agagatgggtgagtacat

SEQ ID NO: 72 - DNA, 18 bp, synthetic construct
  misc_feature 1..18: note = Synthetic oligonucleotide
  source 1..18: mol_type = other DNA
gagagatgggtgagtaca

SEQ ID NO: 73 - DNA, 18 bp, synthetic construct
  misc_feature 1..18: note = Synthetic oligonucleotide
  source 1..18: mol_type = other DNA
ggagagatgggtgagtac

SEQ ID NO: 74 - DNA, 18 bp, synthetic construct
  misc_feature 1..18: note = Synthetic oligonucleotide
  source 1..18: mol_type = other DNA
tggagagatgggtgagta

SEQ ID NO: 75 - DNA, 18 bp, synthetic construct
  misc_feature 1..18: note = Synthetic oligonucleotide
  source 1..18: mol_type = other DNA
atggagagatgggtgagt

SEQ ID NO: 76 - DNA, 18 bp, synthetic construct
  misc_feature 1..18: note = Synthetic oligonucleotide
  source 1..18: mol_type = other DNA
gatggagagatgggtgag

SEQ ID NO: 77 - DNA, 18 bp, synthetic construct
  misc_feature 1..18: note = Synthetic oligonucleotide
  source 1..18: mol_type = other DNA
tggatggagagatgggtg

SEQ ID NO: 78 - DNA, 18 bp, synthetic construct
  misc_feature 1..18: note = Synthetic oligonucleotide
  source 1..18: mol_type = other DNA
atggatggagagatgggt

SEQ ID NO: 79 - DNA, 18 bp, synthetic construct
  misc_feature 1..18: note = Synthetic oligonucleotide
  source 1..18: mol_type = other DNA
catggatggagagatggg

SEQ ID NO: 80 - DNA, 18 bp, synthetic construct
  misc_feature 1..18: note = Synthetic oligonucleotide
  source 1..18: mol_type = other DNA
gcatggatggagagatgg

SEQ ID NO: 81 - DNA, 18 bp, synthetic construct
  misc_feature 1..18: note = Synthetic oligonucleotide
  source 1..18: mol_type = other DNA
agcatggatggagagatg

SEQ ID NO: 82 - DNA, 18 bp, synthetic construct
  misc_feature 1..18: note = Synthetic oligonucleotide
  source 1..18: mol_type = other DNA
aagcatggatggagagat

SEQ ID NO: 83 - DNA, 18 bp, synthetic construct
  misc_feature 1..18: note = Synthetic oligonucleotide
  source 1..18: mol_type = other DNA
aaagcatggatggagaga

SEQ ID NO: 84 - DNA, 18 bp, synthetic construct
  misc_feature 1..18: note = Synthetic oligonucleotide
  source 1..18: mol_type = other DNA
aaaagcatggatggagag

SEQ ID NO: 85 - DNA, 18 bp, synthetic construct
  misc_feature 1..18: note = Synthetic oligonucleotide
  source 1..18: mol_type = other DNA
taaaagcatggatggaga

SEQ ID NO: 86 - DNA, 18 bp, synthetic construct
  misc_feature 1..18: note = Synthetic oligonucleotide
  source 1..18: mol_type = other DNA
ataaaagcatggatggag

SEQ ID NO: 87 - DNA, 18 bp, synthetic construct
  misc_feature 1..18: note = Synthetic oligonucleotide
  source 1..18: mol_type = other DNA
gataaaagcatggatgga

SEQ ID NO: 88 - DNA, 18 bp, synthetic construct
  misc_feature 1..18: note = Synthetic oligonucleotide
  source 1..18: mol_type = other DNA
agataaaagcatggatgg

SEQ ID NO: 89 - DNA, 18 bp, synthetic construct
  misc_feature 1..18: note = Synthetic oligonucleotide
  source 1..18: mol_type = other DNA
tagataaaagcatggatg

SEQ ID NO: 90 - DNA, 18 bp, synthetic construct
  misc_feature 1..18: note = Synthetic oligonucleotide
  source 1..18: mol_type = other DNA
gtagataaaagcatggat

SEQ ID NO: 91 - DNA, 18 bp, synthetic construct
  misc_feature 1..18: note = Synthetic oligonucleotide
  source 1..18: mol_type = other DNA
agtagataaaagcatgga

SEQ ID NO: 92 - DNA, 18 bp, synthetic construct
  misc_feature 1..18: note = Synthetic oligonucleotide
  source 1..18: mol_type = other DNA
gagtagataaaagcatgg

SEQ ID NO: 93 - DNA, 18 bp, synthetic construct
  misc_feature 1..18: note = Synthetic oligonucleotide
  source 1..18: mol_type = other DNA
tgagtagataaaagcatg

SEQ ID NO: 94 - DNA, 18 bp, synthetic construct
  misc_feature 1..18: note = Synthetic oligonucleotide
  source 1..18: mol_type = other DNA
atgagtagataaaagcat

SEQ ID NO: 95 - DNA, 18 bp, synthetic construct
  misc_feature 1..18: note = Synthetic oligonucleotide
  source 1..18: mol_type = other DNA
gatgagtagataaaagca

SEQ ID NO: 96 - DNA, 18 bp, synthetic construct
  misc_feature 1..18: note = Synthetic oligonucleotide
  source 1..18: mol_type = other DNA
tgatgagtagataaaagc

SEQ ID NO: 97 - DNA, 18 bp, synthetic construct
  misc_feature 1..18: note = Synthetic oligonucleotide
  source 1..18: mol_type = other DNA
gatggatggagagatggg

SEQ ID NO: 98 - DNA, 18 bp, synthetic construct
  misc_feature 1..18: note = Synthetic oligonucleotide
  source 1..18: mol_type = other DNA
aggatggatggagagatg

SEQ ID NO: 99 - DNA, 18 bp, synthetic construct
  misc_feature 1..18: note = Synthetic oligonucleotide
  source 1..18: mol_type = other DNA
aaggatggatggagagat

SEQ ID NO: 100 - DNA, 18 bp, synthetic construct
  misc_feature 1..18: note = Synthetic oligonucleotide
  source 1..18: mol_type = other DNA
aaaggatggatggagaga

SEQ ID NO: 101 - DNA, 18 bp, synthetic construct
  misc_feature 1..18: note = Synthetic oligonucleotide
  source 1..18: mol_type = other DNA
aaaaggatggatggagag

SEQ ID NO: 102 - DNA, 18 bp, synthetic construct
  misc_feature 1..18: note = Synthetic oligonucleotide
  source 1..18: mol_type = other DNA
taaaaggatggatggaga

SEQ ID NO: 103 - DNA, 18 bp, synthetic construct
  misc_feature 1..18: note = Synthetic oligonucleotide
  source 1..18: mol_type = other DNA
ataaaaggatggatggag

SEQ ID NO: 104 - DNA, 18 bp, synthetic construct
  misc_feature 1..18: note = Synthetic oligonucleotide
  source 1..18: mol_type = other DNA
gataaaaggatggatgga

SEQ ID NO: 105 - DNA, 18 bp, synthetic construct
  misc_feature 1..18: note = Synthetic oligonucleotide
  source 1..18: mol_type = other DNA
agataaaaggatggatgg

SEQ ID NO: 106 - DNA, 18 bp, synthetic construct
  misc_feature 1..18: note = Synthetic oligonucleotide
  source 1..18: mol_type = other DNA
tagataaaaggatggatg

SEQ ID NO: 107 - DNA, 18 bp, synthetic construct
  misc_feature 1..18: note = Synthetic oligonucleotide
  source 1..18: mol_type = other DNA
gtagataaaaggatggat

SEQ ID NO: 108 - DNA, 18 bp, synthetic construct
  misc_feature 1..18: note = Synthetic oligonucleotide
  source 1..18: mol_type = other DNA
agtagataaaaggatgga

SEQ ID NO: 109 - DNA, 18 bp, synthetic construct
  misc_feature 1..18: note = Synthetic oligonucleotide
  source 1..18: mol_type = other DNA
gagtagataaaaggatgg

SEQ ID NO: 110 - DNA, 18 bp, synthetic construct
  misc_feature 1..18: note = Synthetic oligonucleotide
  source 1..18: mol_type = other DNA
tgagtagataaaaggatg

SEQ ID NO: 111 - DNA, 18 bp, synthetic construct
  misc_feature 1..18: note = Synthetic oligonucleotide
  source 1..18: mol_type = other DNA
atgagtagataaaaggat

SEQ ID NO: 112 - DNA, 18 bp, synthetic construct
  misc_feature 1..18: note = Synthetic oligonucleotide
  source 1..18: mol_type = other DNA
gatgagtagataaaagga

SEQ ID NO: 113 - DNA, 18 bp, synthetic construct
  misc_feature 1..18: note = Synthetic oligonucleotide
  source 1..18: mol_type = other DNA
tgatgagtagataaaagg

SEQ ID NO: 114 - DNA, 18 bp, synthetic construct
  misc_feature 1..18: note = Synthetic oligonucleotide
  source 1..18: mol_type = other DNA
gtgatgagtagataaaag

SEQ ID NO: 115 - DNA, 18 bp, synthetic construct
  misc_feature 1..18: note = Synthetic oligonucleotide
  source 1..18: mol_type = other DNA
agtgatgagtagataaaa

SEQ ID NO: 116 - DNA, 18 bp, synthetic construct
  misc_feature 1..18: note = Synthetic oligonucleotide
  source 1..18: mol_type = other DNA
gagtgatgagtagataaa

SEQ ID NO: 117 - DNA, 18 bp, synthetic construct
  misc_feature 1..18: note = Synthetic oligonucleotide
  source 1..18: mol_type = other DNA
tgagtgatgagtagataa

SEQ ID NO: 118 - DNA, 18 bp, synthetic construct
  misc_feature 1..18: note = Synthetic oligonucleotide
  source 1..18: mol_type = other DNA
atgagtgatgagtagata

SEQ ID NO: 119 - DNA, 18 bp, synthetic construct
  misc_feature 1..18: note = Synthetic oligonucleotide
  source 1..18: mol_type = other DNA
aatgagtgatgagtagat

SEQ ID NO: 120 - DNA, 18 bp, synthetic construct
  misc_feature 1..18: note = Synthetic oligonucleotide
  source 1..18: mol_type = other DNA
gaatgagtgatgagtaga

SEQ ID NO: 121 - DNA, 18 bp, synthetic construct
  misc_feature 1..18: note = Synthetic oligonucleotide
  source 1..18: mol_type = other DNA
tgaatgagtgatgagtag

SEQ ID NO: 122 - DNA, 18 bp, synthetic construct
  misc_feature 1..18: note = Synthetic oligonucleotide
  source 1..18: mol_type = other DNA
atgaatgagtgatgagta

SEQ ID NO: 123 - DNA, 18 bp, synthetic construct
  misc_feature 1..18: note = Synthetic oligonucleotide
  source 1..18: mol_type = other DNA
gatgaatgagtgatgagt

SEQ ID NO: 124 - DNA, 18 bp, synthetic construct
  misc_feature 1..18: note = Synthetic oligonucleotide
  source 1..18: mol_type = other DNA
acagatgaatgagtgatg

SEQ ID NO: 125 - DNA, 18 bp, synthetic construct
  misc_feature 1..18: note = Synthetic oligonucleotide
  source 1..18: mol_type = other DNA
aacagatgaatgagtgat

SEQ ID NO: 126 - DNA, 18 bp, synthetic construct
  misc_feature 1..18: note = Synthetic oligonucleotide
  source 1..18: mol_type = other DNA
gaacagatgaatgagtga

SEQ ID NO: 127 - DNA, 18 bp, synthetic construct
  misc_feature 1..18: note = Synthetic oligonucleotide
  source 1..18: mol_type = other DNA
ttgaacagatgaatgagt

SEQ ID NO: 128 - DNA, 18 bp, synthetic construct
  misc_feature 1..18: note = Synthetic oligonucleotide
  source 1..18: mol_type = other DNA
attgaacagatgaatgag

SEQ ID NO: 129 - DNA, 18 bp, synthetic construct
  misc_feature 1..18: note = Synthetic oligonucleotide
  source 1..18: mol_type = other DNA
gattgaacagatgaatga

SEQ ID NO: 130 - DNA, 18 bp, synthetic construct
  misc_feature 1..18: note = Synthetic oligonucleotide
  source 1..18: mol_type = other DNA
tgattgaacagatgaatg

SEQ ID NO: 131 - DNA, 18 bp, synthetic construct
  misc_feature 1..18: note = Synthetic oligonucleotide
  source 1..18: mol_type = other DNA
atgattgaacagatgaat

SEQ ID NO: 132 - DNA, 18 bp, synthetic construct
  misc_feature 1..18: note = Synthetic oligonucleotide
  source 1..18: mol_type = other DNA
aatgattgaacagatgaa

SEQ ID NO: 133 - DNA, 18 bp, synthetic construct
  misc_feature 1..18: note = Synthetic oligonucleotide
  source 1..18: mol_type = other DNA
gaatgattgaacagatga

SEQ ID NO: 134 - DNA, 18 bp, synthetic construct
  misc_feature 1..18: note = Synthetic oligonucleotide
  source 1..18: mol_type = other DNA
tgaatgattgaacagatg

SEQ ID NO: 135 - DNA, 18 bp, synthetic construct
  misc_feature 1..18: note = Synthetic oligonucleotide
  source 1..18: mol_type = other DNA
atgaatgattgaacagat

SEQ ID NO: 136 - DNA, 18 bp, synthetic construct
  misc_feature 1..18: note = Synthetic oligonucleotide
  source 1..18: mol_type = other DNA
aatgaatgattgaacaga

SEQ ID NO: 137 - DNA, 18 bp, synthetic construct
  misc_feature 1..18: note = Synthetic oligonucleotide
  source 1..18: mol_type = other DNA
gaatgaatgattgaacag

SEQ ID NO: 138 - DNA, 18 bp, synthetic construct
  misc_feature 1..18: note = Synthetic oligonucleotide
  source 1..18: mol_type = other DNA
tgaatgaatgattgaaca

SEQ ID NO: 139 - DNA, 18 bp, synthetic construct
  misc_feature 1..18: note = Synthetic oligonucleotide
  source 1..18: mol_type = other DNA
atgaatgaatgattgaac

SEQ ID NO: 140 - DNA, 18 bp, synthetic construct
  misc_feature 1..18: note = Synthetic oligonucleotide
  source 1..18: mol_type = other DNA
aatgaatgaatgattgaa

SEQ ID NO: 141 - DNA, 18 bp, synthetic construct
  misc_feature 1..18: note = Synthetic polynucleotide
  source 1..18: mol_type = other DNA
gaatgaatgaatgattga

SEQ ID NO: 142 - DNA, 18 bp, synthetic construct
  misc_feature 1..18: note = Synthetic polynucleotide
  source 1..18: mol_type = other DNA
tgaatgaatgaatgattg

SEQ ID NO: 143 - DNA, 18 bp, synthetic construct
  misc_feature 1..18: note = Synthetic polynucleotide
  source 1..18: mol_type = other DNA
gtgaatgaatgaatgatt

SEQ ID NO: 144 - DNA, 18 bp, synthetic construct
  misc_feature 1..18: note = Synthetic polynucleotide
  source 1..18: mol_type = other DNA
ggtgaatgaatgaatgat

SEQ ID NO: 145 - DNA, 18 bp, synthetic construct
  misc_feature 1..18: note = Synthetic polynucleotide
  source 1..18: mol_type = other DNA
tggtgaatgaatgaatga

SEQ ID NO: 146 - DNA, 18 bp, synthetic construct
  misc_feature 1..18: note = Synthetic polynucleotide
  source 1..18: mol_type = other DNA
ctggtgaatgaatgaatg

SEQ ID NO: 147 - DNA, 18 bp, synthetic construct
  misc_feature 1..18: note = Synthetic polynucleotide
  source 1..18: mol_type = other DNA
gctggtgaatgaatgaat

SEQ ID NO: 148 - DNA, 18 bp, synthetic construct
  misc_feature 1..18: note = Synthetic polynucleotide
  source 1..18: mol_type = other DNA
tgctggtgaatgaatgaa

SEQ ID NO: 149 - DNA, 18 bp, synthetic construct
  misc_feature 1..18: note = Synthetic polynucleotide
  source 1..18: mol_type = other DNA
atgctggtgaatgaatga

SEQ ID NO: 150 - DNA, 18 bp, synthetic construct
  misc_feature 1..18: note = Synthetic polynucleotide
  source 1..18: mol_type = other DNA
aatgctggtgaatgaatg

SEQ ID NO: 151 - DNA, 18 bp, synthetic construct
  misc_feature 1..18: note = Synthetic polynucleotide
  source 1..18: mol_type = other DNA
aaatgctggtgaatgaat

SEQ ID NO: 152 - DNA, 18 bp, synthetic construct
  misc_feature 1..18: note = Synthetic polynucleotide
  source 1..18: mol_type = other DNA
taaatgctggtgaatgaa

SEQ ID NO: 153 - DNA, 18 bp, synthetic construct
  misc_feature 1..18: note = Synthetic polynucleotide
  source 1..18: mol_type = other DNA
ataaatgctggtgaatga

SEQ ID NO: 154 - DNA, 18 bp, synthetic construct
  misc_feature 1..18: note = Synthetic polynucleotide
  source 1..18: mol_type = other DNA
aataaatgctggtgaatg

SEQ ID NO: 155 - DNA, 18 bp, synthetic construct
  misc_feature 1..18: note = Synthetic polynucleotide
  source 1..18: mol_type = other DNA
gaataaatgctggtgaat

SEQ ID NO: 156 - DNA, 18 bp, synthetic construct
  misc_feature 1..18: note = Synthetic polynucleotide
  source 1..18: mol_type = other DNA
tgaataaatgctggtgaa

SEQ ID NO: 157 - DNA, 18 bp, synthetic construct
  misc_feature 1..18: note = Synthetic polynucleotide
  source 1..18: mol_type = other DNA
ttgaataaatgctggtga

SEQ ID NO: 158 - DNA, 18 bp, synthetic construct
  misc_feature 1..18: note = Synthetic polynucleotide
  source 1..18: mol_type = other DNA
gttgaataaatgctggtg

SEQ ID NO: 159 - DNA, 18 bp, synthetic construct
  misc_feature 1..18: note = Synthetic polynucleotide
  source 1..18: mol_type = other DNA
ttgttgaataaatgctgg

SEQ ID NO: 160 - DNA, 18 bp, synthetic construct
  misc_feature 1..18: note = Synthetic polynucleotide
  source 1..18: mol_type = other DNA
tttgttgaataaatgctg

SEQ ID NO: 161 - DNA, 18 bp, synthetic construct
  misc_feature 1..18: note = Synthetic polynucleotide
  source 1..18: mol_type = other DNA
gtttgttgaataaatgct

SEQ ID NO: 162 - DNA, 18 bp, synthetic construct
  misc_feature 1..18: note = Synthetic polynucleotide
  source 1..18: mol_type = other DNA
agtttgttgaataaatgc

SEQ ID NO: 163 - DNA, 18 bp, synthetic construct
  misc_feature 1..18: note = Synthetic polynucleotide
  source 1..18: mol_type = other DNA
ctagtttgttgaataaat

SEQ ID NO: 164 - DNA, 18 bp, synthetic construct
  misc_feature 1..18: note = Synthetic polynucleotide
  source 1..18: mol_type = other DNA
actagtttgttgaataaa

SEQ ID NO: 165 - DNA, 18 bp, synthetic construct
  misc_feature 1..18: note = Synthetic polynucleotide
  source 1..18: mol_type = other DNA
aactagtttgttgaataa

SEQ ID NO: 166 - DNA, 18 bp, synthetic construct
  misc_feature 1..18: note = Synthetic polynucleotide
  source 1..18: mol_type = other DNA
gaactagtttgttgaata

SEQ ID NO: 167 - DNA, 18 bp, synthetic construct
  misc_feature 1..18: note = Synthetic polynucleotide
  source 1..18: mol_type = other DNA
ggaactagtttgttgaat

SEQ ID NO: 168 - DNA, 18 bp, synthetic construct
  misc_feature 1..18: note = Synthetic polynucleotide
  source 1..18: mol_type = other DNA
aggaactagtttgttgaa

SEQ ID NO: 169 - DNA, 18 bp, synthetic construct
  misc_feature 1..18: note = Synthetic polynucleotide
  source 1..18: mol_type = other DNA
caggaactagtttgttga

SEQ ID NO: 170 - DNA, 18 bp, synthetic construct
  misc_feature 1..18: note = Synthetic polynucleotide
  source 1..18: mol_type = other DNA
ccaggaactagtttgttg

SEQ ID NO: 171 - DNA, 18 bp, synthetic construct
  misc_feature 1..18: note = Synthetic polynucleotide
  source 1..18: mol_type = other DNA
cccaggaactagtttgtt

SEQ ID NO: 172 - DNA, 18 bp, synthetic construct
  misc_feature 1..18: note = Synthetic polynucleotide
  source 1..18: mol_type = other DNA
ccccaggaactagtttgt

SEQ ID NO: 173 - DNA, 18 bp, synthetic construct
  misc_feature 1..18: note = Synthetic polynucleotide
  source 1..18: mol_type = other DNA
tccccaggaactagtttg

SEQ ID NO: 174 - DNA, 18 bp, synthetic construct
  misc_feature 1..18: note = Synthetic polynucleotide
  source 1..18: mol_type = other DNA
atccccaggaactagttt

SEQ ID NO: 175 - DNA, 18 bp, synthetic construct
  misc_feature 1..18: note = Synthetic polynucleotide
  source 1..18: mol_type = other DNA
tatccccaggaactagtt

SEQ ID NO: 176 - DNA, 18 bp, synthetic construct
  misc_feature 1..18: note = Synthetic polynucleotide
  source 1..18: mol_type = other DNA
ttatccccaggaactagt

SEQ ID NO: 177 - DNA, 18 bp, synthetic construct
  misc_feature 1..18: note = Synthetic polynucleotide
  source 1..18: mol_type = other DNA
cttatccccaggaactag

SEQ ID NO: 178 - DNA, 18 bp, synthetic construct
  misc_feature 1..18: note = Synthetic polynucleotide
  source 1..18: mol_type = other DNA
tcttatccccaggaacta

SEQ ID NO: 179 - DNA, 18 bp, synthetic construct
  misc_feature 1..18: note = Synthetic polynucleotide
  source 1..18: mol_type = other DNA
ctcttatccccaggaact

SEQ ID NO: 180 - DNA, 18 bp, synthetic construct
  misc_feature 1..18: note = Synthetic polynucleotide
  source 1..18: mol_type = other DNA
actcttatccccaggaac

SEQ ID NO: 181 - DNA, 18 bp, synthetic construct
  misc_feature 1..18: note = Synthetic polynucleotide
  source 1..18: mol_type = other DNA
aactcttatccccaggaa

SEQ ID NO: 182 - DNA, 18 bp, synthetic construct
  misc_feature 1..18: note = Synthetic polynucleotide
  source 1..18: mol_type = other DNA
gaactcttatccccagga

SEQ ID NO: 183 - DNA, 18 bp, synthetic construct
  misc_feature 1..18: note = Synthetic polynucleotide
  source 1..18: mol_type = other DNA
agaactcttatccccagg

SEQ ID NO: 184 - DNA, 18 bp, synthetic construct
  misc_feature 1..18: note = Synthetic polynucleotide
  source 1..18: mol_type = other DNA
aagaactcttatccccag

SEQ ID NO: 185 - DNA, 18 bp, synthetic construct
  misc_feature 1..18: note = Synthetic polynucleotide
  source 1..18: mol_type = other DNA
aaagaactcttatcccca

SEQ ID NO: 186 - DNA, 18 bp, synthetic construct
  misc_feature 1..18: note = Synthetic polynucleotide
  source 1..18: mol_type = other DNA
gaaagaactcttatcccc

SEQ ID NO: 187 - DNA, 18 bp, synthetic construct
  misc_feature 1..18: note = Synthetic polynucleotide
  source 1..18: mol_type = other DNA
ggaaagaactcttatccc

SEQ ID NO: 188 - DNA, 18 bp, synthetic construct
  misc_feature 1..18: note = Synthetic polynucleotide
  source 1..18: mol_type = other DNA
tggaaagaactcttatcc

SEQ ID NO: 189 - DNA, 18 bp, synthetic construct
  misc_feature 1..18: note = Synthetic polynucleotide
  source 1..18: mol_type = other DNA
ctggaaagaactcttatc

SEQ ID NO: 190 - DNA, 18 bp, synthetic construct
  misc_feature 1..18: note = Synthetic polynucleotide
  source 1..18: mol_type = other DNA
cctggaaagaactcttat

SEQ ID NO: 191 - DNA, 18 bp, synthetic construct
  misc_feature 1..18: note = Synthetic polynucleotide
  source 1..18: mol_type = other DNA
tcctggaaagaactctta

SEQ ID NO: 192 - DNA, 18 bp, synthetic construct
  misc_feature 1..18: note = Synthetic polynucleotide
  source 1..18: mol_type = other DNA
ttcctggaaagaactctt

SEQ ID NO: 193 - DNA, 18 bp, synthetic construct
  misc_feature 1..18: note = Synthetic polynucleotide
  source 1..18: mol_type = other DNA
tttcctggaaagaactct

SEQ ID NO: 194 - DNA, 18 bp, synthetic construct
  misc_feature 1..18: note = Synthetic polynucleotide
  source 1..18: mol_type = other DNA
gtttcctggaaagaactc

SEQ ID NO: 195 - DNA, 18 bp, synthetic construct
  misc_feature 1..18: note = Synthetic polynucleotide
  source 1..18: mol_type = other DNA
ggtttcctggaaagaact

SEQ ID NO: 196 - DNA, 18 bp, synthetic construct
  misc_feature 1..18: note = Synthetic polynucleotide
  source 1..18: mol_type = other DNA
gggtttcctggaaagaac

SEQ ID NO: 197 - DNA, 18 bp, synthetic construct
  misc_feature 1..18: note = Synthetic polynucleotide
  source 1..18: mol_type = other DNA
tgggtttcctggaaagaa

SEQ ID NO: 198 - DNA, 18 bp, synthetic construct
  misc_feature 1..18: note = Synthetic polynucleotide
  source 1..18: mol_type = other DNA
ctgggtttcctggaaaga

SEQ ID NO: 199 - DNA, 18 bp, synthetic construct
  misc_feature 1..18: note = Synthetic polynucleotide
  source 1..18: mol_type = other DNA
cctgggtttcctggaaag

SEQ ID NO: 200 - DNA, 18 bp, synthetic construct
  misc_feature 1..18: note = Synthetic polynucleotide
  source 1..18: mol_type = other DNA
ctgcctgggtttcctgga

SEQ ID NO: 201 - DNA, 18 bp, synthetic construct
  misc_feature 1..18: note = Synthetic polynucleotide
  source 1..18: mol_type = other DNA
gctgcctgggtttcctgg

SEQ ID NO: 202 - DNA, 18 bp, synthetic construct
  misc_feature 1..18: note = Synthetic polynucleotide
  source 1..18: mol_type = other DNA
agctgcctgggtttcctg

SEQ ID NO: 203 - DNA, 18 bp, synthetic construct
  misc_feature 1..18: note = Synthetic polynucleotide
  source 1..18: mol_type = other DNA
cagctgcctgggtttcct

SEQ ID NO: 204 - DNA, 18 bp, synthetic construct
  misc_feature 1..18: note = Synthetic polynucleotide
  source 1..18: mol_type = other DNA
ccagctgcctgggtttcc

SEQ ID NO: 205 - DNA, 18 bp, synthetic construct
  misc_feature 1..18: note = Synthetic polynucleotide
  source 1..18: mol_type = other DNA
tccagctgcctgggtttc

SEQ ID NO: 206 - DNA, 18 bp, synthetic construct
  misc_feature 1..18: note = Synthetic polynucleotide
  source 1..18: mol_type = other DNA
ttccagctgcctgggttt

SEQ ID NO: 207 - DNA, 18 bp, synthetic construct
  misc_feature 1..18: note = Synthetic polynucleotide
  source 1..18: mol_type = other DNA
cttccagctgcctgggtt

SEQ ID NO: 208 - DNA, 18 bp, synthetic construct
  misc_feature 1..18: note = Synthetic polynucleotide
  source 1..18: mol_type = other DNA
tcttccagctgcctgggt

SEQ ID NO: 209 - DNA, 18 bp, synthetic construct
  misc_feature 1..18: note = Synthetic polynucleotide
  source 1..18: mol_type = other DNA
ctcttccagctgcctggg

SEQ ID NO: 210 - DNA, 18 bp, synthetic construct
  misc_feature 1..18: note = Synthetic polynucleotide
  source 1..18: mol_type = other DNA
tctcttccagctgcctgg

SEQ ID NO: 211 - DNA, 18 bp, synthetic construct
  misc_feature 1..18: note = Synthetic polynucleotide
  source 1..18: mol_type = other DNA
gtctcttccagctgcctg

SEQ ID NO: 212 - DNA, 18 bp, synthetic construct
  misc_feature 1..18: note = Synthetic polynucleotide
  source 1..18: mol_type = other DNA
tgtctcttccagctgcct

SEQ ID NO: 213 - DNA, 18 bp, synthetic construct
  misc_feature 1..18: note = Synthetic polynucleotide
  source 1..18: mol_type = other DNA
tatgtctcttccagctgc

SEQ ID NO: 214 - DNA, 18 bp, synthetic construct
  misc_feature 1..18: note = Synthetic polynucleotide
  source 1..18: mol_type = other DNA
gtatgtctcttccagctg

SEQ ID NO: 215 - DNA, 18 bp, synthetic construct
  misc_feature 1..18: note = Synthetic polynucleotide
  source 1..18: mol_type = other DNA
ggtatgtctcttccagct

SEQ ID NO: 216 - DNA, 18 bp, synthetic construct
  misc_feature 1..18: note = Synthetic polynucleotide
  source 1..18: mol_type = other DNA
gggtatgtctcttccagc

SEQ ID NO: 217 - DNA, 18 bp, synthetic construct
  misc_feature 1..18: note = Synthetic polynucleotide
  source 1..18: mol_type = other DNA
tgggtatgtctcttccag

SEQ ID NO: 218 - DNA, 18 bp, synthetic construct
  misc_feature 1..18: note = Synthetic polynucleotide
  source 1..18: mol_type = other DNA
ctgggtatgtctcttcca

SEQ ID NO: 219 - DNA, 18 bp, synthetic construct
  misc_feature 1..18: note = Synthetic polynucleotide
  source 1..18: mol_type = other DNA
tctgggtatgtctcttcc

SEQ ID NO: 220 - DNA, 18 bp, synthetic construct
  misc_feature 1..18: note = Synthetic polynucleotide
  source 1..18: mol_type = other DNA
gtctgggtatgtctcttc

SEQ ID NO: 221 - DNA, 18 bp, synthetic construct
  misc_feature 1..18: note = Synthetic polynucleotide
  source 1..18: mol_type = other DNA
tgtctgggtatgtctctt

SEQ ID NO: 222 - DNA, 18 bp, synthetic construct
  misc_feature 1..18: note = Synthetic polynucleotide
  source 1..18: mol_type = other DNA
gtgtctgggtatgtctct

SEQ ID NO: 223 - DNA, 18 bp, synthetic construct
  misc_feature 1..18: note = Synthetic polynucleotide
  source 1..18: mol_type = other DNA
tgtgtctgggtatgtctc

SEQ ID NO: 224 - DNA, 18 bp, synthetic construct
  misc_feature 1..18: note = Synthetic polynucleotide
  source 1..18: mol_type = other DNA
ttgtgtctgggtatgtct

SEQ ID NO: 225 - DNA, 18 bp, synthetic construct
  misc_feature 1..18: note = Synthetic polynucleotide
  source 1..18: mol_type = other DNA
tttgtgtctgggtatgtc

SEQ ID NO: 226 - DNA, 18 bp, synthetic construct
  misc_feature 1..18: note = Synthetic polynucleotide
  source 1..18: mol_type = other DNA
gtttgtgtctgggtatgt

SEQ ID NO: 227 - DNA, 18 bp, synthetic construct
  misc_feature 1..18: note = Synthetic polynucleotide
  source 1..18: mol_type = other DNA
cgtttgtgtctgggtatg

SEQ ID NO: 228 - DNA, 18 bp, synthetic construct
  misc_feature 1..18: note = Synthetic polynucleotide
  source 1..18: mol_type = other DNA
ccgtttgtgtctgggtat

SEQ ID NO: 229 - DNA, 18 bp, synthetic construct
  misc_feature 1..18: note = Synthetic polynucleotide
  source 1..18: mol_type = other DNA
gccgtttgtgtctgggta

SEQ ID NO: 230 - DNA, 18 bp, synthetic construct
  misc_feature 1..18: note = Synthetic polynucleotide
  source 1..18: mol_type = other DNA
ggccgtttgtgtctgggt

SEQ ID NO: 231 - DNA, 18 bp, synthetic construct
  misc_feature 1..18: note = Synthetic polynucleotide
  source 1..18: mol_type = other DNA
gggccgtttgtgtctggg

SEQ ID NO: 232 - DNA, 18 bp, synthetic construct
  misc_feature 1..18: note = Synthetic polynucleotide
  source 1..18: mol_type = other DNA
tgggccgtttgtgtctgg

SEQ ID NO: 233 - DNA, 18 bp, synthetic construct
  misc_feature 1..18: note = Synthetic polynucleotide
  source 1..18: mol_type = other DNA
ttgggccgtttgtgtctg

SEQ ID NO: 234 - DNA, 18 bp, synthetic construct
  misc_feature 1..18: note = Synthetic polynucleotide
  source 1..18: mol_type = other DNA
attgggccgtttgtgtct

SEQ ID NO: 235 - DNA, 18 bp, synthetic construct
  misc_feature 1..18: note = Synthetic polynucleotide
  source 1..18: mol_type = other DNA
ggattgggccgtttgtgt

SEQ ID NO: 236 - DNA, 18 bp, synthetic construct
  misc_feature 1..18: note = Synthetic polynucleotide
  source 1..18: mol_type = other DNA
aggattgggccgtttgtg

SEQ ID NO: 237 - DNA, 18 bp, synthetic construct
  misc_feature 1..18: note = Synthetic polynucleotide
  source 1..18: mol_type = other DNA
caggattgggccgtttgt

SEQ ID NO: 238 - DNA, 18 bp, synthetic construct
  misc_feature 1..18: note = Synthetic polynucleotide
  source 1..18: mol_type = other DNA
tcaggattgggccgtttg

SEQ ID NO: 239 - DNA, 18 bp, synthetic construct
  misc_feature 1..18: note = Synthetic polynucleotide
  source 1..18: mol_type = other DNA
ctcaggattgggccgttt

SEQ ID NO: 240 - DNA, 18 bp, synthetic construct
  misc_feature 1..18: note = Synthetic polynucleotide
  source 1..18: mol_type = other DNA
actcaggattgggccgtt

SEQ ID NO: 241 - DNA, 18 bp, synthetic construct
  misc_feature 1..18: note = Synthetic polynucleotide
  source 1..18: mol_type = other DNA
cactcaggattgggccgt

SEQ ID NO: 242 - DNA, 18 bp, synthetic construct
  misc_feature 1..18: note = Synthetic polynucleotide
  source 1..18: mol_type = other DNA
ccactcaggattgggccg

SEQ ID NO: 243 - DNA, 18 bp, synthetic construct
  misc_feature 1..18: note = Synthetic polynucleotide
  source 1..18: mol_type = other DNA
accactcaggattgggcc

SEQ ID NO: 244 - DNA, 18 bp, synthetic construct
  misc_feature 1..18: note = Synthetic polynucleotide
  source 1..18: mol_type = other DNA
aaccactcaggattgggc

SEQ ID NO: 245 - DNA, 18 bp, synthetic construct
  misc_feature 1..18: note = Synthetic polynucleotide
  source 1..18: mol_type = other DNA
taaccactcaggattggg

SEQ ID NO: 246 - DNA, 18 bp, synthetic construct
  misc_feature 1..18: note = Synthetic polynucleotide
  source 1..18: mol_type = other DNA
ctaaccactcaggattgg

SEQ ID NO: 247 - DNA, 18 bp, synthetic construct
  misc_feature 1..18: note = Synthetic polynucleotide
  source 1..18: mol_type = other DNA
cctaaccactcaggattg

SEQ ID NO: 248 - DNA, 18 bp, synthetic construct
  misc_feature 1..18: note = Synthetic polynucleotide
  source 1..18: mol_type = other DNA
ccctaaccactcaggatt

SEQ ID NO: 249 - DNA, 18 bp, synthetic construct
  misc_feature 1..18: note = Synthetic polynucleotide
  source 1..18: mol_type = other DNA
gccctaaccactcaggat

SEQ ID NO: 250 - DNA, 18 bp, synthetic construct
  misc_feature 1..18: note = Synthetic polynucleotide
  source 1..18: mol_type = other DNA
agccctaaccactcagga

SEQ ID NO: 251 - DNA, 18 bp, synthetic construct
  misc_feature 1..18: note = Synthetic polynucleotide
  source 1..18: mol_type = other DNA
cagccctaaccactcagg

SEQ ID NO: 252 - DNA, 18 bp, synthetic construct
  misc_feature 1..18: note = Synthetic polynucleotide
  source 1..18: mol_type = other DNA
ccagccctaaccactcag

SEQ ID NO: 253 - DNA, 18 bp, synthetic construct
  misc_feature 1..18: note = Synthetic polynucleotide
  source 1..18: mol_type = other DNA
tccagccctaaccactca

SEQ ID NO: 254 - DNA, 18 bp, synthetic construct
  misc_feature 1..18: note = Synthetic polynucleotide
  source 1..18: mol_type = other DNA
ttccagccctaaccactc

SEQ ID NO: 255 - DNA, 18 bp, synthetic construct
  misc_feature 1..18: note = Synthetic polynucleotide
  source 1..18: mol_type = other DNA
attccagccctaaccact

SEQ ID NO: 256 - DNA, 18 bp, synthetic construct
  misc_feature 1..18: note = Synthetic polynucleotide
  source 1..18: mol_type = other DNA
tattccagccctaaccac

SEQ ID NO: 257 - DNA, 18 bp, synthetic construct
  misc_feature 1..18: note = Synthetic polynucleotide
  source 1..18: mol_type = other DNA
ctattccagccctaacca

SEQ ID NO: 258 - DNA, 18 bp, synthetic construct
  misc_feature 1..18: note = Synthetic polynucleotide
  source 1..18: mol_type = other DNA
tctattccagccctaacc

SEQ ID NO: 259 - DNA, 18 bp, synthetic construct
  misc_feature 1..18: note = Synthetic polynucleotide
  source 1..18: mol_type = other DNA
ttctattccagccctaac

SEQ ID NO: 260 - DNA, 18 bp, synthetic construct
  misc_feature 1..18: note = Synthetic polynucleotide
  source 1..18: mol_type = other DNA
cttctattccagccctaa

SEQ ID NO: 261 - DNA, 18 bp, synthetic construct
  misc_feature 1..18: note = Synthetic polynucleotide
  source 1..18: mol_type = other DNA
ccttctattccagcccta

SEQ ID NO: 262 - DNA, 18 bp, synthetic construct
  misc_feature 1..18: note = Synthetic polynucleotide
  source 1..18: mol_type = other DNA
tccttctattccagccct

SEQ ID NO: 263 - DNA, 18 bp, synthetic construct
  misc_feature 1..18: note = Synthetic polynucleotide
  source 1..18: mol_type = other DNA
ttccttctattccagccc

SEQ ID NO: 264 - DNA, 18 bp, synthetic construct
  misc_feature 1..18: note = Synthetic polynucleotide
  source 1..18: mol_type = other DNA
cttccttctattccagcc

SEQ ID NO: 265 - DNA, 18 bp, synthetic construct
  misc_feature 1..18: note = Synthetic polynucleotide
  source 1..18: mol_type = other DNA
tcttccttctattccagc

SEQ ID NO: 266 - DNA, 18 bp, synthetic construct
  misc_feature 1..18: note = Synthetic polynucleotide
  source 1..18: mol_type = other DNA
ttcttccttctattccag

SEQ ID NO: 267 - DNA, 18 bp, synthetic construct
  misc_feature 1..18: note = Synthetic polynucleotide
  source 1..18: mol_type = other DNA
gttcttccttctattcca

SEQ ID NO: 268 - DNA, 18 bp, synthetic construct
  misc_feature 1..18: note = Synthetic polynucleotide
  source 1..18: mol_type = other DNA
ggttcttccttctattcc

SEQ ID NO: 269 - DNA, 18 bp, synthetic construct
  misc_feature 1..18: note = Synthetic polynucleotide
  source 1..18: mol_type = other DNA
aggttcttccttctattc

SEQ ID NO: 270 - DNA, 18 bp, synthetic construct
  misc_feature 1..18: note = Synthetic polynucleotide
  source 1..18: mol_type = other DNA
caggttcttccttctatt

SEQ ID NO: 271 - DNA, 18 bp, synthetic construct
  misc_feature 1..18: note = Synthetic polynucleotide
  source 1..18: mol_type = other DNA
tcaggttcttccttctat

SEQ ID NO: 272 - DNA, 18 bp, synthetic construct
  misc_feature 1..18: note = Synthetic polynucleotide
  source 1..18: mol_type = other DNA
gagtacatggatggatag

SEQ ID NO: 273 - DNA, 18 bp, synthetic construct
  misc_feature 1..18: note = Synthetic polynucleotide
  source 1..18: mol_type = other DNA
tgagtacatggatggata

SEQ ID NO: 274 - DNA, 18 bp, synthetic construct
  misc_feature 1..18: note = Synthetic polynucleotide
  source 1..18: mol_type = other DNA
gtgagtacatggatggat

SEQ ID NO: 275 - DNA, 18 bp, synthetic construct
  misc_feature 1..18: note = Synthetic polynucleotide
  source 1..18: mol_type = other DNA
ggtgagtacatggatgga

SEQ ID NO: 276 - DNA, 18 bp, synthetic construct
  misc_feature 1..18: note = Synthetic polynucleotide
  source 1..18: mol_type = other DNA
gggtgagtacatggatgg

SEQ ID NO: 277 - DNA, 18 bp, synthetic construct
  misc_feature 1..18: note = Synthetic polynucleotide
  source 1..18: mol_type = other DNA
tgggtgagtacatggatg

SEQ ID NO: 278 - DNA, 18 bp, synthetic construct
  misc_feature 1..18: note = Synthetic polynucleotide
  source 1..18: mol_type = other DNA
atgggtgagtacatggat

SEQ ID NO: 279 - DNA, 18 bp, synthetic construct
  misc_feature 1..18: note = Synthetic polynucleotide
  source 1..18: mol_type = other DNA
gatgggtgagtacatgga

SEQ ID NO: 280 - DNA, 18 bp, synthetic construct
  misc_feature 1..18: note = Synthetic polynucleotide
  source 1..18: mol_type = other DNA
catctgtccatccatcca

SEQ ID NO: 281 - DNA, 18 bp, synthetic construct
  misc_feature 1..18: note = Synthetic polynucleotide
  source 1..18: mol_type = other DNA
atctgtccatccatccat

SEQ ID NO: 282 - DNA, 18 bp, synthetic construct
  misc_feature 1..18: note = Synthetic polynucleotide
  source 1..18: mol_type = other DNA
tctgtccatccatccatc

SEQ ID NO: 283 - DNA, 18 bp, synthetic construct
  misc_feature 1..18: note = Synthetic polynucleotide
  source 1..18: mol_type = other DNA
ctgtccatccatccatca

SEQ ID NO: 284 - DNA, 18 bp, synthetic construct
  misc_feature 1..18: note = Synthetic polynucleotide
  source 1..18: mol_type = other DNA
tgtccatccatccatcat

SEQ ID NO: 285 - DNA, 18 bp, synthetic construct
  misc_feature 1..18: note = Synthetic polynucleotide
  source 1..18: mol_type = other DNA
tccatccatccatcatcc

SEQ ID NO: 286 - DNA, 18 bp, synthetic construct
  misc_feature 1..18: note = Synthetic polynucleotide
  source 1..18: mol_type = other DNA
ccatccatccatcatcca

SEQ ID NO: 287 - DNA, 18 bp, synthetic construct
  misc_feature 1..18: note = Synthetic polynucleotide
  source 1..18: mol_type = other DNA
catccatccatcatccat

SEQ ID NO: 288 - DNA, 18 bp, synthetic construct
  misc_feature 1..18: note = Synthetic polynucleotide
  source 1..18: mol_type = other DNA
tccatccatcatccatct

SEQ ID NO: 289 - DNA, 18 bp, synthetic construct
  misc_feature 1..18: note = Synthetic polynucleotide
  source 1..18: mol_type = other DNA
ccatccatcatccatcta

SEQ ID NO: 290 - DNA, 18 bp, synthetic construct
  misc_feature 1..18: note = Synthetic polynucleotide
  source 1..18: mol_type = other DNA
catccatcatccatctag

SEQ ID NO: 291 - DNA, 18 bp, synthetic construct
  misc_feature 1..18: note = Synthetic polynucleotide
  source 1..18: mol_type = other DNA
atccatcatccatctagc

SEQ ID NO: 292 - DNA, 18 bp, synthetic construct
  misc_feature 1..18: note = Synthetic polynucleotide
  source 1..18: mol_type = other DNA
tccatcatccatctagcc

SEQ ID NO: 293 - DNA, 18 bp, synthetic construct
  misc_feature 1..18: note = Synthetic polynucleotide
  source 1..18: mol_type = other DNA
ccatcatccatctagcca

SEQ ID NO: 294 - DNA, 18 bp, synthetic construct
  misc_feature 1..18: note = Synthetic polynucleotide
  source 1..18: mol_type = other DNA
catcatccatctagccac

SEQ ID NO: 295 - DNA, 18 bp, synthetic construct
  misc_feature 1..18: note = Synthetic polynucleotide
  source 1..18: mol_type = other DNA
atcatccatctagccacg

SEQ ID NO: 296 - DNA, 18 bp, synthetic construct
  misc_feature 1..18: note = Synthetic polynucleotide
  source 1..18: mol_type = other DNA
tcatccatctagccacga

SEQ ID NO: 297 - DNA, 18 bp, synthetic construct
  misc_feature 1..18: note = Synthetic polynucleotide
  source 1..18: mol_type = other DNA
catccatctagccacgaa

SEQ ID NO: 298 - DNA, 18 bp, synthetic construct
  misc_feature 1..18: note = Synthetic polynucleotide
  source 1..18: mol_type = other DNA
atccatctagccacgaat

SEQ ID NO: 299 - DNA, 18 bp, synthetic construct
  misc_feature 1..18: note = Synthetic polynucleotide
  source 1..18: mol_type = other DNA
tccatctagccacgaatc

SEQ ID NO: 300 - DNA, 18 bp, synthetic construct
  misc_feature 1..18: note = Synthetic polynucleotide
  source 1..18: mol_type = other DNA
ccatctagccacgaatct

SEQ ID NO: 301 - DNA, 18 bp, synthetic construct
  misc_feature 1..18: note = Synthetic polynucleotide
  source 1..18: mol_type = other DNA
catctagccacgaatcta

SEQ ID NO: 302 - DNA, 18 bp, synthetic construct
  misc_feature 1..18: note = Synthetic polynucleotide
  source 1..18: mol_type = other DNA
atctagccacgaatctac

SEQ ID NO: 303 - DNA, 18 bp, synthetic construct
  misc_feature 1..18: note = Synthetic polynucleotide
  source 1..18: mol_type = other DNA
ctagccacgaatctaccc

SEQ ID NO: 304 - DNA, 18 bp, synthetic construct
  misc_feature 1..18: note = Synthetic polynucleotide
  source 1..18: mol_type = other DNA
tagccacgaatctaccca

SEQ ID NO: 305 - DNA, 18 bp, synthetic construct
  misc_feature 1..18: note = Synthetic polynucleotide
  source 1..18: mol_type = other DNA
agccacgaatctacccac

SEQ ID NO: 306 - DNA, 18 bp, synthetic construct
  misc_feature 1..18: note = Synthetic polynucleotide
  source 1..18: mol_type = other DNA
gccacgaatctacccacc

SEQ ID NO: 307 - DNA, 18 bp, synthetic construct
  misc_feature 1..18: note = Synthetic polynucleotide
  source 1..18: mol_type = other DNA
ccacgaatctacccacca

SEQ ID NO: 308 - DNA, 18 bp, synthetic construct
  misc_feature 1..18: note = Synthetic polynucleotide
  source 1..18: mol_type = other DNA
cacgaatctacccaccaa

SEQ ID NO: 309 - DNA, 18 bp, synthetic construct
  misc_feature 1..18: note = Synthetic polynucleotide
  source 1..18: mol_type = other DNA
acgaatctacccaccaac

SEQ ID NO: 310 - DNA, 18 bp, synthetic construct
  misc_feature 1..18: note = Synthetic polynucleotide
  source 1..18: mol_type = other DNA
aatctacccaccaactca

SEQ ID NO: 311 - DNA, 18 bp, synthetic construct
  misc_feature 1..18: note = Synthetic polynucleotide
  source 1..18: mol_type = other DNA
atctacccaccaactcat

SEQ ID NO: 312 - DNA, 18 bp, synthetic construct
  misc_feature 1..18: note = Synthetic polynucleotide
  source 1..18: mol_type = other DNA
tctacccaccaactcatc

SEQ ID NO: 313 - DNA, 18 bp, synthetic construct
  misc_feature 1..18: note = Synthetic polynucleotide
  source 1..18: mol_type = other DNA
ctacccaccaactcatcc

SEQ ID NO: 314 - DNA, 18 bp, synthetic construct
  misc_feature 1..18: note = Synthetic polynucleotide
  source 1..18: mol_type = other DNA
tacccaccaactcatcca

SEQ ID NO: 315 - DNA, 18 bp, synthetic construct
  misc_feature 1..18: note = Synthetic polynucleotide
  source 1..18: mol_type = other DNA
acccaccaactcatccat

SEQ ID NO: 316 - DNA, 18 bp, synthetic construct
  misc_feature 1..18: note = Synthetic polynucleotide
  source 1..18: mol_type = other DNA
cccaccaactcatccatc

SEQ ID NO: 317 - DNA, 18 bp, synthetic construct
  misc_feature 1..18: note = Synthetic polynucleotide
  source 1..18: mol_type = other DNA
ccaccaactcatccatct

SEQ ID NO: 318 - DNA, 18 bp, synthetic construct
  misc_feature 1..18: note = Synthetic polynucleotide
  source 1..18: mol_type = other DNA
caccaactcatccatcta

SEQ ID NO: 319 - DNA, 18 bp, synthetic construct
  misc_feature 1..18: note = Synthetic polynucleotide
  source 1..18: mol_type = other DNA
accaactcatccatctat

SEQ ID NO: 320 - DNA, 18 bp, synthetic construct
  misc_feature 1..18: note = Synthetic polynucleotide
  source 1..18: mol_type = other DNA
ccaactcatccatctatc

SEQ ID NO: 321 - DNA, 18 bp, synthetic construct
  misc_feature 1..18: note = Synthetic polynucleotide
  source 1..18: mol_type = other DNA
caactcatccatctatcc

SEQ ID NO: 322 - DNA, 18 bp, synthetic construct
  misc_feature 1..18: note = Synthetic polynucleotide
  source 1..18: mol_type = other DNA
aactcatccatctatcca

SEQ ID NO: 323 - DNA, 18 bp, synthetic construct
  misc_feature 1..18: note = Synthetic polynucleotide
  source 1..18: mol_type = other DNA
actcatccatctatccat

SEQ ID NO: 324 - DNA, 18 bp, synthetic construct
  misc_feature 1..18: note = Synthetic polynucleotide
  source 1..18: mol_type = other DNA
ctcatccatctatccatc

SEQ ID NO: 325 - DNA, 18 bp, synthetic construct
  misc_feature 1..18: note = Synthetic polynucleotide
  source 1..18: mol_type = other DNA
tcatccatctatccatcc

SEQ ID NO: 326 - DNA, 18 bp, synthetic construct
  misc_feature 1..18: note = Synthetic polynucleotide
  source 1..18: mol_type = other DNA
catccatctatccatcca

SEQ ID NO: 327 - DNA, 18 bp, synthetic construct
  misc_feature 1..18: note = Synthetic polynucleotide
  source 1..18: mol_type = other DNA
atccatctatccatccat

SEQ ID NO: 328 - DNA, 18 bp, synthetic construct
  misc_feature 1..18: note = Synthetic polynucleotide
  source 1..18: mol_type = other DNA
tccatctatccatccatg

SEQ ID NO: 329 - DNA, 18 bp, synthetic construct
  misc_feature 1..18: note = Synthetic polynucleotide
  source 1..18: mol_type = other DNA
ccatctatccatccatgt

SEQ ID NO: 330 - DNA, 18 bp, synthetic construct
  misc_feature 1..18: note = Synthetic polynucleotide
  source 1..18: mol_type = other DNA
catctatccatccatgta

SEQ ID NO: 331 - DNA, 18 bp, synthetic construct
  misc_feature 1..18: note = Synthetic polynucleotide
  source 1..18: mol_type = other DNA
atctatccatccatgtac

SEQ ID NO: 332 - DNA, 18 bp, synthetic construct
  misc_feature 1..18: note = Synthetic polynucleotide
  source 1..18: mol_type = other DNA
tctatccatccatgtact

SEQ ID NO: 333 - DNA, 18 bp, synthetic construct
  misc_feature 1..18: note = Synthetic polynucleotide
  source 1..18: mol_type = other DNA
ctatccatccatgtactc

SEQ ID NO: 334 - DNA, 18 bp, synthetic construct
  misc_feature 1..18: note = Synthetic polynucleotide
  source 1..18: mol_type = other DNA
tatccatccatgtactca

SEQ ID NO: 335 - DNA, 18 bp, synthetic construct
  misc_feature 1..18: note = Synthetic polynucleotide
  source 1..18: mol_type = other DNA
atccatccatgtactcac

SEQ ID NO: 336 - DNA, 18 bp, synthetic construct
  misc_feature 1..18: note = Synthetic polynucleotide
  source 1..18: mol_type = other DNA
tccatccatgtactcacc

SEQ ID NO: 337 - DNA, 18 bp, synthetic construct
  misc_feature 1..18: note = Synthetic polynucleotide
  source 1..18: mol_type = other DNA
ccatccatgtactcaccc

SEQ ID NO: 338 - DNA, 18 bp, synthetic construct
  misc_feature 1..18: note = Synthetic polynucleotide
  source 1..18: mol_type = other DNA
catccatgtactcaccca

SEQ ID NO: 339 - DNA, 18 bp, synthetic construct
  misc_feature 1..18: note = Synthetic polynucleotide
  source 1..18: mol_type = other DNA
atccatgtactcacccat

SEQ ID NO: 340 - DNA, 18 bp, synthetic construct
  misc_feature 1..18: note = Synthetic polynucleotide
  source 1..18: mol_type = other DNA
tccatgtactcacccatc

SEQ ID NO: 341 - DNA, 18 bp, synthetic construct
  misc_feature 1..18: note = Synthetic polynucleotide
  source 1..18: mol_type = other DNA
ccatgtactcacccatct

SEQ ID NO: 342 - DNA, 18 bp, synthetic construct
  misc_feature 1..18: note = Synthetic polynucleotide
  source 1..18: mol_type = other DNA
catgtactcacccatctc

SEQ ID NO: 343 - DNA, 18 bp, synthetic construct
  misc_feature 1..18: note = Synthetic polynucleotide
  source 1..18: mol_type = other DNA
atgtactcacccatctct

SEQ ID NO: 344 - DNA, 18 bp, synthetic construct
  misc_feature 1..18: note = Synthetic polynucleotide
  source 1..18: mol_type = other DNA
tgtactcacccatctctc

SEQ ID NO: 345 - DNA, 18 bp, synthetic construct
  misc_feature 1..18: note = Synthetic polynucleotide
  source 1..18: mol_type = other DNA
gtactcacccatctctcc

SEQ ID NO: 346 - DNA, 18 bp, synthetic construct
  misc_feature 1..18: note = Synthetic polynucleotide
  source 1..18: mol_type = other DNA
tactcacccatctctcca

SEQ ID NO: 347 - DNA, 18 bp, synthetic construct
  misc_feature 1..18: note = Synthetic polynucleotide
  source 1..18: mol_type = other DNA
actcacccatctctccat

SEQ ID NO: 348 - DNA, 18 bp, synthetic construct
  misc_feature 1..18: note = Synthetic polynucleotide
  source 1..18: mol_type = other DNA
ctcacccatctctccatc

SEQ ID NO: 349 - DNA, 18 bp, synthetic construct
  misc_feature 1..18: note = Synthetic polynucleotide
  source 1..18: mol_type = other DNA
cacccatctctccatcca

SEQ ID NO: 350 - DNA, 18 bp, synthetic construct
  misc_feature 1..18: note = Synthetic polynucleotide
  source 1..18: mol_type = other DNA
acccatctctccatccat

SEQ ID NO: 351 - DNA, 18 bp, synthetic construct
  misc_feature 1..18: note = Synthetic polynucleotide
  source 1..18: mol_type = other DNA
cccatctctccatccatg

SEQ ID NO: 352 - DNA, 18 bp, synthetic construct
  misc_feature 1..18: note = Synthetic polynucleotide
  source 1..18: mol_type = other DNA
ccatctctccatccatgc

SEQ ID NO: 353 - DNA, 18 bp, synthetic construct
  misc_feature 1..18: note = Synthetic polynucleotide
  source 1..18: mol_type = other DNA
catctctccatccatgct

SEQ ID NO: 354 - DNA, 18 bp, synthetic construct
  misc_feature 1..18: note = Synthetic polynucleotide
  source 1..18: mol_type = other DNA
atctctccatccatgctt

SEQ ID NO: 355 - DNA, 18 bp, synthetic construct
  misc_feature 1..18: note = Synthetic polynucleotide
  source 1..18: mol_type = other DNA
tctctccatccatgcttt

SEQ ID NO: 356 - DNA, 18 bp, synthetic construct
  misc_feature 1..18: note = Synthetic polynucleotide
  source 1..18: mol_type = other DNA
ctctccatccatgctttt

SEQ ID NO: 357 - DNA, 18 bp, synthetic construct
  misc_feature 1..18: note = Synthetic polynucleotide
  source 1..18: mol_type = other DNA
tctccatccatgctttta

SEQ ID NO: 358 - DNA, 18 bp, synthetic construct
  misc_feature 1..18: note = Synthetic polynucleotide
  source 1..18: mol_type = other DNA
ctccatccatgcttttat

SEQ ID NO: 359 - DNA, 18 bp, synthetic construct
  misc_feature 1..18: note = Synthetic polynucleotide
  source 1..18: mol_type = other DNA
tccatccatgcttttatc

SEQ ID NO: 360 - DNA, 18 bp, synthetic construct
  misc_feature 1..18: note = Synthetic polynucleotide
  source 1..18: mol_type = other DNA
ccatccatgcttttatct

SEQ ID NO: 361 - DNA, 18 bp, synthetic construct
  misc_feature 1..18: note = Synthetic polynucleotide
  source 1..18: mol_type = other DNA
catccatgcttttatcta

SEQ ID NO: 362 - DNA, 18 bp, synthetic construct
  misc_feature 1..18: note = Synthetic polynucleotide
  source 1..18: mol_type = other DNA
atccatgcttttatctac

SEQ ID NO: 363 - DNA, 18 bp, synthetic construct
  misc_feature 1..18: note = Synthetic polynucleotide
  source 1..18: mol_type = other DNA
tccatgcttttatctact

SEQ ID NO: 364 - DNA, 18 bp, synthetic construct
  misc_feature 1..18: note = Synthetic polynucleotide
  source 1..18: mol_type = other DNA
ccatgcttttatctactc

SEQ ID NO: 365 - DNA, 18 bp, synthetic construct
  misc_feature 1..18: note = Synthetic polynucleotide
  source 1..18: mol_type = other DNA
catgcttttatctactca

SEQ ID NO: 366 - DNA, 18 bp, synthetic construct
  misc_feature 1..18: note = Synthetic polynucleotide
  source 1..18: mol_type = other DNA
atgcttttatctactcat

SEQ ID NO: 367 - DNA, 18 bp, synthetic construct
  misc_feature 1..18: note = Synthetic polynucleotide
  source 1..18: mol_type = other DNA
tgcttttatctactcatc

SEQ ID NO: 368 - DNA, 18 bp, synthetic construct
  misc_feature 1..18: note = Synthetic polynucleotide
  source 1..18: mol_type = other DNA
gcttttatctactcatca

SEQ ID NO: 369 - DNA, 18 bp, synthetic construct
  misc_feature 1..18: note = Synthetic polynucleotide
  source 1..18: mol_type = other DNA
cccatctctccatccatc

SEQ ID NO: 370 - DNA, 18 bp, synthetic construct
  misc_feature 1..18: note = Synthetic polynucleotide
  source 1..18: mol_type = other DNA
catctctccatccatcct

SEQ ID NO: 371 - DNA, 18 bp, synthetic construct
  misc_feature 1..18: note = Synthetic polynucleotide
  source 1..18: mol_type = other DNA
atctctccatccatcctt

SEQ ID NO: 372 - DNA, 18 bp, synthetic construct
  misc_feature 1..18: note = Synthetic polynucleotide
  source 1..18: mol_type = other DNA
tctctccatccatccttt

SEQ ID NO: 373 - DNA, 18 bp, synthetic construct
  misc_feature 1..18: note = Synthetic polynucleotide
  source 1..18: mol_type = other DNA
ctctccatccatcctttt

SEQ ID NO: 374 - DNA, 18 bp, synthetic construct
  misc_feature 1..18: note = Synthetic polynucleotide
  source 1..18: mol_type = other DNA
tctccatccatcctttta

SEQ ID NO: 375 - DNA, 18 bp, synthetic construct
  misc_feature 1..18: note = Synthetic polynucleotide
  source 1..18: mol_type = other DNA
ctccatccatccttttat

SEQ ID NO: 376 - DNA, 18 bp, synthetic construct
  misc_feature 1..18: note = Synthetic polynucleotide
  source 1..18: mol_type = other DNA
tccatccatccttttatc

SEQ ID NO: 377 - DNA, 18 bp, synthetic construct
  misc_feature 1..18: note = Synthetic polynucleotide
  source 1..18: mol_type = other DNA
ccatccatccttttatct

SEQ ID NO: 378 - DNA, 18 bp, synthetic construct
  misc_feature 1..18: note = Synthetic polynucleotide
  source 1..18: mol_type = other DNA
catccatccttttatcta

SEQ ID NO: 379 - DNA, 18 bp, synthetic construct
  misc_feature 1..18: note = Synthetic polynucleotide
  source 1..18: mol_type = other DNA
atccatccttttatctac

SEQ ID NO: 380 - DNA, 18 bp, synthetic construct
  misc_feature 1..18: note = Synthetic polynucleotide
  source 1..18: mol_type = other DNA
tccatccttttatctact

SEQ ID NO: 381 - DNA, 18 bp, synthetic construct
  misc_feature 1..18: note = Synthetic polynucleotide
  source 1..18: mol_type = other DNA
ccatccttttatctactc

SEQ ID NO: 382 - DNA, 18 bp, synthetic construct
  misc_feature 1..18: note = Synthetic polynucleotide
  source 1..18: mol_type = other DNA
catccttttatctactca

SEQ ID NO: 383 - DNA, 18 bp, synthetic construct
  misc_feature 1..18: note = Synthetic polynucleotide
  source 1..18: mol_type = other DNA
atccttttatctactcat

SEQ ID NO: 384 - DNA, 18 bp, synthetic construct
  misc_feature 1..18: note = Synthetic polynucleotide
  source 1..18: mol_type = other DNA
tccttttatctactcatc

SEQ ID NO: 385 - DNA, 18 bp, synthetic construct
  misc_feature 1..18: note = Synthetic polynucleotide
  source 1..18: mol_type = other DNA
ccttttatctactcatca

SEQ ID NO: 386 - DNA, 18 bp, synthetic construct
  misc_feature 1..18: note = Synthetic polynucleotide
  source 1..18: mol_type = other DNA
cttttatctactcatcac

SEQ ID NO: 387 - DNA, 18 bp, synthetic construct
  misc_feature 1..18: note = Synthetic polynucleotide
  source 1..18: mol_type = other DNA
ttttatctactcatcact

SEQ ID NO: 388 - DNA, 18 bp, synthetic construct
  misc_feature 1..18: note = Synthetic polynucleotide
  source 1..18: mol_type = other DNA
tttatctactcatcactc

SEQ ID NO: 389 - DNA, 18 bp, synthetic construct
  misc_feature 1..18: note = Synthetic polynucleotide
  source 1..18: mol_type = other DNA
ttatctactcatcactca

SEQ ID NO: 390 - DNA, 18 bp, synthetic construct
  misc_feature 1..18: note = Synthetic polynucleotide
  source 1..18: mol_type = other DNA
tatctactcatcactcat

SEQ ID NO: 391 - DNA, 18 bp, synthetic construct
  misc_feature 1..18: note = Synthetic polynucleotide
  source 1..18: mol_type = other DNA
atctactcatcactcatt

SEQ ID NO: 392 - DNA, 18 bp, synthetic construct
  misc_feature 1..18: note = Synthetic polynucleotide
  source 1..18: mol_type = other DNA
tctactcatcactcattc

SEQ ID NO: 393 - DNA, 18 bp, synthetic construct
  misc_feature 1..18: note = Synthetic polynucleotide
  source 1..18: mol_type = other DNA
ctactcatcactcattca

SEQ ID NO: 394 - DNA, 18 bp, synthetic construct
  misc_feature 1..18: note = Synthetic polynucleotide
  source 1..18: mol_type = other DNA
tactcatcactcattcat

SEQ ID NO: 395 - DNA, 18 bp, synthetic construct
  misc_feature 1..18: note = Synthetic polynucleotide
  source 1..18: mol_type = other DNA
actcatcactcattcatc

SEQ ID NO: 396 - DNA, 18 bp, synthetic construct
  misc_feature 1..18: note = Synthetic polynucleotide
  source 1..18: mol_type = other DNA
catcactcattcatctgt

SEQ ID NO: 397 - DNA, 18 bp, synthetic construct
  misc_feature 1..18: note = Synthetic polynucleotide
  source 1..18: mol_type = other DNA
atcactcattcatctgtt

SEQ ID NO: 398 - DNA, 18 bp, synthetic construct
  misc_feature 1..18: note = Synthetic polynucleotide
  source 1..18: mol_type = other DNA
tcactcattcatctgttc

SEQ ID NO: 399 - DNA, 18 bp, synthetic construct
  misc_feature 1..18: note = Synthetic polynucleotide
  source 1..18: mol_type = other DNA
actcattcatctgttcaa

SEQ ID NO: 400 - DNA, 18 bp, synthetic construct
  misc_feature 1..18: note = Synthetic polynucleotide
  source 1..18: mol_type = other DNA
ctcattcatctgttcaat

SEQ ID NO: 401 - DNA, 18 bp, synthetic construct
  misc_feature 1..18: note = Synthetic polynucleotide
  source 1..18: mol_type = other DNA
tcattcatctgttcaatc

SEQ ID NO: 402 - DNA, 18 bp, synthetic construct
  misc_feature 1..18: note = Synthetic polynucleotide
  source 1..18: mol_type = other DNA
cattcatctgttcaatca

SEQ ID NO: 403 - DNA, 18 bp, synthetic construct
  misc_feature 1..18: note = Synthetic polynucleotide
  source 1..18: mol_type = other DNA
attcatctgttcaatcat

SEQ ID NO: 404 - DNA, 18 bp, synthetic construct
  misc_feature 1..18: note = Synthetic polynucleotide
  source 1..18: mol_type = other DNA
ttcatctgttcaatcatt

SEQ ID NO: 405 - DNA, 18 bp, synthetic construct
  misc_feature 1..18: note = Synthetic polynucleotide
  source 1..18: mol_type = other DNA
tcatctgttcaatcattc

SEQ ID NO: 406 - DNA, 18 bp, synthetic construct
  misc_feature 1..18: note = Synthetic polynucleotide
  source 1..18: mol_type = other DNA
catctgttcaatcattca

SEQ ID NO: 407 - DNA, 18 bp, synthetic construct
  misc_feature 1..18: note = Synthetic polynucleotide
  source 1..18: mol_type = other DNA
atctgttcaatcattcat

SEQ ID NO: 408 - DNA, 18 bp, synthetic construct
  misc_feature 1..18: note = Synthetic polynucleotide
  source 1..18: mol_type = other DNA
tctgttcaatcattcatt

SEQ ID NO: 409 - DNA, 18 bp, synthetic construct
  misc_feature 1..18: note = Synthetic polynucleotide
  source 1..18: mol_type = other DNA
ctgttcaatcattcattc

SEQ ID NO: 410 - DNA, 18 bp, synthetic construct
  misc_feature 1..18: note = Synthetic polynucleotide
  source 1..18: mol_type = other DNA
tgttcaatcattcattca

SEQ ID NO: 411 - DNA, 18 bp, synthetic construct
  misc_feature 1..18: note = Synthetic polynucleotide
  source 1..18: mol_type = other DNA
gttcaatcattcattcat

SEQ ID NO: 412 - DNA, 18 bp, synthetic construct
  misc_feature 1..18: note = Synthetic polynucleotide
  source 1..18: mol_type = other DNA
ttcaatcattcattcatt

SEQ ID NO: 413 - DNA, 18 bp, synthetic construct
  misc_feature 1..18: note = Synthetic polynucleotide
  source 1..18: mol_type = other DNA
tcaatcattcattcattc

SEQ ID NO: 414 - DNA, 18 bp, synthetic construct
  misc_feature 1..18: note = Synthetic polynucleotide
  source 1..18: mol_type = other DNA
caatcattcattcattca

SEQ ID NO: 415 - DNA, 18 bp, synthetic construct
  misc_feature 1..18: note = Synthetic polynucleotide
  source 1..18: mol_type = other DNA
aatcattcattcattcac

SEQ ID NO: 416 - DNA, 18 bp, synthetic construct
  misc_feature 1..18: note = Synthetic polynucleotide
  source 1..18: mol_type = other DNA
atcattcattcattcacc

SEQ ID NO: 417 - DNA, 18 bp, synthetic construct
  misc_feature 1..18: note = Synthetic polynucleotide
  source 1..18: mol_type = other DNA
tcattcattcattcacca

SEQ ID NO: 418 - DNA, 18 bp, synthetic construct
  misc_feature 1..18: note = Synthetic polynucleotide
  source 1..18: mol_type = other DNA
cattcattcattcaccag

SEQ ID NO: 419 - DNA, 18 bp, synthetic construct
  misc_feature 1..18: note = Synthetic polynucleotide
  source 1..18: mol_type = other DNA
attcattcattcaccagc

SEQ ID NO: 420 - DNA, 18 bp, synthetic construct
  misc_feature 1..18: note = Synthetic polynucleotide
  source 1..18: mol_type = other DNA
ttcattcattcaccagca

SEQ ID NO: 421 - DNA, 18 bp, synthetic construct
  misc_feature 1..18: note = Synthetic polynucleotide
  source 1..18: mol_type = other DNA
tcattcattcaccagcat

SEQ ID NO: 422 - DNA, 18 bp, synthetic construct
  misc_feature 1..18: note = Synthetic polynucleotide
  source 1..18: mol_type = other DNA
cattcattcaccagcatt

SEQ ID NO: 423 - DNA, 18 bp, synthetic construct
  misc_feature 1..18: note = Synthetic polynucleotide
  source 1..18: mol_type = other DNA
attcattcaccagcattt

SEQ ID NO: 424 - DNA, 18 bp, synthetic construct
  misc_feature 1..18: note = Synthetic polynucleotide
  source 1..18: mol_type = other DNA
ttcattcaccagcattta

SEQ ID NO: 425 - DNA, 18 bp, synthetic construct
  misc_feature 1..18: note = Synthetic polynucleotide
  source 1..18: mol_type = other DNA
tcattcaccagcatttat

SEQ ID NO: 426 - DNA, 18 bp, synthetic construct
  misc_feature 1..18: note = Synthetic polynucleotide
  source 1..18: mol_type = other DNA
cattcaccagcatttatt

SEQ ID NO: 427 - DNA, 18 bp, synthetic construct
  misc_feature 1..18: note = Synthetic polynucleotide
  source 1..18: mol_type = other DNA
attcaccagcatttattc

SEQ ID NO: 428 - DNA, 18 bp, synthetic construct
  misc_feature 1..18: note = Synthetic polynucleotide
  source 1..18: mol_type = other DNA
ttcaccagcatttattca

SEQ ID NO: 429 - DNA, 18 bp, synthetic construct
  misc_feature 1..18: note = Synthetic polynucleotide
  source 1..18: mol_type = other DNA
tcaccagcatttattcaa

SEQ ID NO: 430 - DNA, 18 bp, synthetic construct
  misc_feature 1..18: note = Synthetic polynucleotide
  source 1..18: mol_type = other DNA
caccagcatttattcaac

SEQ ID NO: 431 - DNA, 18 bp, synthetic construct
  misc_feature 1..18: note = Synthetic polynucleotide
  source 1..18: mol_type = other DNA
ccagcatttattcaacaa

SEQ ID NO: 432 - DNA, 18 bp, synthetic construct
  misc_feature 1..18: note = Synthetic polynucleotide
  source 1..18: mol_type = other DNA
cagcatttattcaacaaa

SEQ ID NO: 433 - DNA, 18 bp, synthetic construct
  misc_feature 1..18: note = Synthetic polynucleotide
  source 1..18: mol_type = other DNA
agcatttattcaacaaac

SEQ ID NO: 434 - DNA, 18 bp, synthetic construct
  misc_feature 1..18: note = Synthetic polynucleotide
  source 1..18: mol_type = other DNA
gcatttattcaacaaact

SEQ ID NO: 435 - DNA, 18 bp, synthetic construct
  misc_feature 1..18: note = Synthetic polynucleotide
  source 1..18: mol_type = other DNA
atttattcaacaaactag

SEQ ID NO: 436 - DNA, 18 bp, synthetic construct
  misc_feature 1..18: note = Synthetic polynucleotide
  source 1..18: mol_type = other DNA
tttattcaacaaactagt

SEQ ID NO: 437 - DNA, 18 bp, synthetic construct
  misc_feature 1..18: note = Synthetic polynucleotide
  source 1..18: mol_type = other DNA
ttattcaacaaactagtt

SEQ ID NO: 438 - DNA, 18 bp, synthetic construct
  misc_feature 1..18: note = Synthetic polynucleotide
  source 1..18: mol_type = other DNA
tattcaacaaactagttc

SEQ ID NO: 439 - DNA, 18 bp, synthetic construct
  misc_feature 1..18: note = Synthetic polynucleotide
  source 1..18: mol_type = other DNA
attcaacaaactagttcc

SEQ ID NO: 440 - DNA, 18 bp, synthetic construct
  misc_feature 1..18: note = Synthetic polynucleotide
  source 1..18: mol_type = other DNA
ttcaacaaactagttcct

SEQ ID NO: 441 - DNA, 18 bp, synthetic construct
  misc_feature 1..18: note = Synthetic polynucleotide
  source 1..18: mol_type = other DNA
tcaacaaactagttcctg

SEQ ID NO: 442 - DNA, 18 bp, synthetic construct
  misc_feature 1..18: note = Synthetic polynucleotide
  source 1..18: mol_type = other DNA
caacaaactagttcctgg

SEQ ID NO: 443 - DNA, 18 bp, synthetic construct
  misc_feature 1..18: note = Synthetic polynucleotide
  source 1..18: mol_type = other DNA
aacaaactagttcctggg

SEQ ID NO: 444 - DNA, 18 bp, synthetic construct
  misc_feature 1..18: note = Synthetic polynucleotide
  source 1..18: mol_type = other DNA
acaaactagttcctgggg

SEQ ID NO: 445 - DNA, 18 bp, synthetic construct
  misc_feature 1..18: note = Synthetic polynucleotide
  source 1..18: mol_type = other DNA
caaactagttcctgggga

SEQ ID NO: 446 - DNA, 18 bp, synthetic construct
  misc_feature 1..18: note = Synthetic polynucleotide
  source 1..18: mol_type = other DNA
aaactagttcctggggat

SEQ ID NO: 447 - DNA, 18 bp, synthetic construct
  misc_feature 1..18: note = Synthetic polynucleotide
  source 1..18: mol_type = other DNA
aactagttcctggggata

SEQ ID NO: 448 - DNA, 18 bp, synthetic construct
  misc_feature 1..18: note = Synthetic polynucleotide
  source 1..18: mol_type = other DNA
actagttcctggggataa

SEQ ID NO: 449 - DNA, 18 bp, synthetic construct
  misc_feature 1..18: note = Synthetic polynucleotide
  source 1..18: mol_type = other DNA
ctagttcctggggataag

SEQ ID NO: 450 - DNA, 18 bp, synthetic construct
  misc_feature 1..18: note = Synthetic polynucleotide
  source 1..18: mol_type = other DNA
tagttcctggggataaga

SEQ ID NO: 451 - DNA, 18 bp, synthetic construct
  misc_feature 1..18: note = Synthetic polynucleotide
  source 1..18: mol_type = other DNA
agttcctggggataagag

SEQ ID NO: 452 - DNA, 18 bp, synthetic construct
  misc_feature 1..18: note = Synthetic polynucleotide
  source 1..18: mol_type = other DNA
gttcctggggataagagt

SEQ ID NO: 453 - DNA, 18 bp, synthetic construct
  misc_feature 1..18: note = Synthetic polynucleotide
  source 1..18: mol_type = other DNA
ttcctggggataagagtt

SEQ ID NO: 454 - DNA, 18 bp, synthetic construct
  misc_feature 1..18: note = Synthetic polynucleotide
  source 1..18: mol_type = other DNA
tcctggggataagagttc

SEQ ID NO: 455 - DNA, 18 bp, synthetic construct
  misc_feature 1..18: note = Synthetic polynucleotide
  source 1..18: mol_type = other DNA
cctggggataagagttct

SEQ ID NO: 456 - DNA, 18 bp, synthetic construct
  misc_feature 1..18: note = Synthetic polynucleotide
  source 1..18: mol_type = other DNA
ctggggataagagttctt

SEQ ID NO: 457 - DNA, 18 bp, synthetic construct
  misc_feature 1..18: note = Synthetic polynucleotide
  source 1..18: mol_type = other DNA
tggggataagagttcttt

SEQ ID NO: 458 - DNA, 18 bp, synthetic construct
  misc_feature 1..18: note = Synthetic polynucleotide
  source 1..18: mol_type = other DNA
ggggataagagttctttc

SEQ ID NO: 459 - DNA, 18 bp, synthetic construct
  misc_feature 1..18: note = Synthetic polynucleotide
  source 1..18: mol_type = other DNA
gggataagagttctttcc

SEQ ID NO: 460 - DNA, 18 bp, synthetic construct
  misc_feature 1..18: note = Synthetic polynucleotide
  source 1..18: mol_type = other DNA
ggataagagttctttcca

SEQ ID NO: 461 - DNA, 18 bp, synthetic construct
  misc_feature 1..18: note = Synthetic polynucleotide
  source 1..18: mol_type = other DNA
gataagagttctttccag

SEQ ID NO: 462 - DNA, 18 bp, synthetic construct
  misc_feature 1..18: note = Synthetic polynucleotide
  source 1..18: mol_type = other DNA
ataagagttctttccagg

SEQ ID NO: 463 - DNA, 18 bp, synthetic construct
  misc_feature 1..18: note = Synthetic polynucleotide
  source 1..18: mol_type = other DNA
taagagttctttccagga

SEQ ID NO: 464 - DNA, 18 bp, synthetic construct
  misc_feature 1..18: note = Synthetic polynucleotide
  source 1..18: mol_type = other DNA
aagagttctttccaggaa

SEQ ID NO: 465 - DNA, 18 bp, synthetic construct
  misc_feature 1..18: note = Synthetic polynucleotide
  source 1..18: mol_type = other DNA
agagttctttccaggaaa

SEQ ID NO: 466 - DNA, 18 bp, synthetic construct
  misc_feature 1..18: note = Synthetic polynucleotide
  source 1..18: mol_type = other DNA
gagttctttccaggaaac

SEQ ID NO: 467 - DNA, 18 bp, synthetic construct
  misc_feature 1..18: note = Synthetic polynucleotide
  source 1..18: mol_type = other DNA
agttctttccaggaaacc

SEQ ID NO: 468 - DNA, 18 bp, synthetic construct
  misc_feature 1..18: note = Synthetic polynucleotide
  source 1..18: mol_type = other DNA
gttctttccaggaaaccc

SEQ ID NO: 469 - DNA, 18 bp, synthetic construct
  misc_feature 1..18: note = Synthetic polynucleotide
  source 1..18: mol_type = other DNA
ttctttccaggaaaccca

SEQ ID NO: 470 - DNA, 18 bp, synthetic construct
  misc_feature 1..18: note = Synthetic polynucleotide
  source 1..18: mol_type = other DNA
tctttccaggaaacccag

SEQ ID NO: 471 - DNA, 18 bp, synthetic construct
  misc_feature 1..18: note = Synthetic polynucleotide
  source 1..18: mol_type = other DNA
ctttccaggaaacccagg

SEQ ID NO: 472 - DNA, 18 bp, synthetic construct
  misc_feature 1..18: note = Synthetic polynucleotide
  source 1..18: mol_type = other DNA
tccaggaaacccaggcag

SEQ ID NO: 473 - DNA, 18 bp, synthetic construct
  misc_feature 1..18: note = Synthetic polynucleotide
  source 1..18: mol_type = other DNA
ccaggaaacccaggcagc

SEQ ID NO: 474 - DNA, 18 bp, synthetic construct
  misc_feature 1..18: note = Synthetic polynucleotide
  source 1..18: mol_type = other DNA
caggaaacccaggcagct

SEQ ID NO: 475 - DNA, 18 bp, synthetic construct
  misc_feature 1..18: note = Synthetic polynucleotide
  source 1..18: mol_type = other DNA
aggaaacccaggcagctg

SEQ ID NO: 476 - DNA, 18 bp, synthetic construct
  misc_feature 1..18: note = Synthetic polynucleotide
  source 1..18: mol_type = other DNA
ggaaacccaggcagctgg

SEQ ID NO: 477 - DNA, 18 bp, synthetic construct
  misc_feature 1..18: note = Synthetic polynucleotide
  source 1..18: mol_type = other DNA
gaaacccaggcagctgga

SEQ ID NO: 478 - DNA, 18 bp, synthetic construct
  misc_feature 1..18: note = Synthetic polynucleotide
  source 1..18: mol_type = other DNA
aaacccaggcagctggaa

SEQ ID NO: 479 - DNA, 18 bp, synthetic construct
  misc_feature 1..18: note = Synthetic polynucleotide
  source 1..18: mol_type = other DNA
aacccaggcagctggaag

SEQ ID NO: 480 - DNA, 18 bp, synthetic construct
  misc_feature 1..18: note = Synthetic polynucleotide
  source 1..18: mol_type = other DNA
acccaggcagctggaaga

SEQ ID NO: 481 - DNA, 18 bp, synthetic construct
  misc_feature 1..18: note = Synthetic polynucleotide
  source 1..18: mol_type = other DNA
cccaggcagctggaagag

SEQ ID NO: 482 - DNA, 18 bp, synthetic construct
  misc_feature 1..18: note = Synthetic polynucleotide
  source 1..18: mol_type = other DNA
ccaggcagctggaagaga

SEQ ID NO: 483 - DNA, 18 bp, synthetic construct
  misc_feature 1..18: note = Synthetic polynucleotide
  source 1..18: mol_type = other DNA
caggcagctggaagagac

SEQ ID NO: 484 - DNA, 18 bp, synthetic construct
  misc_feature 1..18: note = Synthetic polynucleotide
  source 1..18: mol_type = other DNA
aggcagctggaagagaca

SEQ ID NO: 485 - DNA, 18 bp, synthetic construct
  misc_feature 1..18: note = Synthetic polynucleotide
  source 1..18: mol_type = other DNA
gcagctggaagagacata

SEQ ID NO: 486 - DNA, 18 bp, synthetic construct
  misc_feature 1..18: note = Synthetic polynucleotide
  source 1..18: mol_type = other DNA
cagctggaagagacatac

SEQ ID NO: 487 - DNA, 18 bp, synthetic construct
  misc_feature 1..18: note = Synthetic polynucleotide
  source 1..18: mol_type = other DNA
agctggaagagacatacc

SEQ ID NO: 488 - DNA, 18 bp, synthetic construct
  misc_feature 1..18: note = Synthetic polynucleotide
  source 1..18: mol_type = other DNA
gctggaagagacataccc

SEQ ID NO: 489 - DNA, 18 bp, synthetic construct
  misc_feature 1..18: note = Synthetic polynucleotide
  source 1..18: mol_type = other DNA
ctggaagagacataccca

SEQ ID NO: 490 - DNA, 18 bp, synthetic construct
  misc_feature 1..18: note = Synthetic polynucleotide
  source 1..18: mol_type = other DNA
tggaagagacatacccag

SEQ ID NO: 491 - DNA, 18 bp, synthetic construct
  misc_feature 1..18: note = Synthetic polynucleotide
  source 1..18: mol_type = other DNA
ggaagagacatacccaga

SEQ ID NO: 492 - DNA, 18 bp, synthetic construct
  misc_feature 1..18: note = Synthetic polynucleotide
  source 1..18: mol_type = other DNA
gaagagacatacccagac

SEQ ID NO: 493 - DNA, 18 bp, synthetic construct
  misc_feature 1..18: note = Synthetic polynucleotide
  source 1..18: mol_type = other DNA
aagagacatacccagaca

SEQ ID NO: 494 - DNA, 18 bp, synthetic construct
  misc_feature 1..18: note = Synthetic polynucleotide
  source 1..18: mol_type = other DNA
agagacatacccagacac

SEQ ID NO: 495 - DNA, 18 bp, synthetic construct
  misc_feature 1..18: note = Synthetic polynucleotide
  source 1..18: mol_type = other DNA
gagacatacccagacaca

SEQ ID NO: 496 - DNA, 18 bp, synthetic construct
  misc_feature 1..18: note = Synthetic polynucleotide
  source 1..18: mol_type = other DNA
agacatacccagacacaa

SEQ ID NO: 497 - DNA, 18 bp, synthetic construct
  misc_feature 1..18: note = Synthetic polynucleotide
  source 1..18: mol_type = other DNA
gacatacccagacacaaa

SEQ ID NO: 498 - DNA, 18 bp, synthetic construct
  misc_feature 1..18: note = Synthetic polynucleotide
  source 1..18: mol_type = other DNA
acatacccagacacaaac

SEQ ID NO: 499 - DNA, 18 bp, synthetic construct
  misc_feature 1..18: note = Synthetic polynucleotide
  source 1..18: mol_type = other DNA
catacccagacacaaacg

SEQ ID NO: 500 - DNA, 18 bp, synthetic construct
  misc_feature 1..18: note = Synthetic polynucleotide
  source 1..18: mol_type = other DNA
atacccagacacaaacgg

SEQ ID NO: 501 - DNA, 18 bp, synthetic construct
  misc_feature 1..18: note = Synthetic polynucleotide
  source 1..18: mol_type = other DNA
tacccagacacaaacggc

SEQ ID NO: 502 - DNA, 18 bp, synthetic construct
  misc_feature 1..18: note = Synthetic polynucleotide
  source 1..18: mol_type = other DNA
acccagacacaaacggcc

SEQ ID NO: 503 - DNA, 18 bp, synthetic construct
  misc_feature 1..18: note = Synthetic polynucleotide
  source 1..18: mol_type = other DNA
cccagacacaaacggccc

SEQ ID NO: 504 - DNA, 18 bp, synthetic construct
  misc_feature 1..18: note = Synthetic polynucleotide
  source 1..18: mol_type = other DNA
ccagacacaaacggccca

SEQ ID NO: 505 - DNA, 18 bp, synthetic construct
  misc_feature 1..18: note = Synthetic polynucleotide
  source 1..18: mol_type = other DNA
cagacacaaacggcccaa

SEQ ID NO: 506 - DNA, 18 bp, synthetic construct
  misc_feature 1..18: note = Synthetic polynucleotide
  source 1..18: mol_type = other DNA
agacacaaacggcccaat

SEQ ID NO: 507 - DNA, 18 bp, synthetic construct
  misc_feature 1..18: note = Synthetic polynucleotide
  source 1..18: mol_type = other DNA
acacaaacggcccaatcc

SEQ ID NO: 508 - DNA, 18 bp, synthetic construct
  misc_feature 1..18: note = Synthetic polynucleotide
  source 1..18: mol_type = other DNA
cacaaacggcccaatcct

SEQ ID NO: 509 - DNA, 18 bp, synthetic construct
  misc_feature 1..18: note = Synthetic polynucleotide
  source 1..18: mol_type = other DNA
acaaacggcccaatcctg

SEQ ID NO: 510 - DNA, 18 bp, synthetic construct
  misc_feature 1..18: note = Synthetic polynucleotide
  source 1..18: mol_type = other DNA
caaacggcccaatcctga

SEQ ID NO: 511 - DNA, 18 bp, synthetic construct
  misc_feature 1..18: note = Synthetic polynucleotide
  source 1..18: mol_type = other DNA
aaacggcccaatcctgag

SEQ ID NO: 512 - DNA, 18 bp, synthetic construct
  misc_feature 1..18: note = Synthetic polynucleotide
  source 1..18: mol_type = other DNA
aacggcccaatcctgagt

SEQ ID NO: 513 - DNA, 18 bp, synthetic construct
  misc_feature 1..18: note = Synthetic polynucleotide
  source 1..18: mol_type = other DNA
acggcccaatcctgagtg

SEQ ID NO: 514 - DNA, 18 bp, synthetic construct
  misc_feature 1..18: note = Synthetic polynucleotide
  source 1..18: mol_type = other DNA
cggcccaatcctgagtgg

SEQ ID NO: 515 - DNA, 18 bp, synthetic construct
  misc_feature 1..18: note = Synthetic polynucleotide
  source 1..18: mol_type = other DNA
ggcccaatcctgagtggt

SEQ ID NO: 516 - DNA, 18 bp, synthetic construct
  misc_feature 1..18: note = Synthetic polynucleotide
  source 1..18: mol_type = other DNA
gcccaatcctgagtggtt

SEQ ID NO: 517 - DNA, 18 bp, synthetic construct
  misc_feature 1..18: note = Synthetic polynucleotide
  source 1..18: mol_type = other DNA
cccaatcctgagtggtta

SEQ ID NO: 518 - DNA, 18 bp, synthetic construct
  misc_feature 1..18: note = Synthetic polynucleotide
  source 1..18: mol_type = other DNA
ccaatcctgagtggttag

SEQ ID NO: 519 - DNA, 18 bp, synthetic construct
  misc_feature 1..18: note = Synthetic polynucleotide
  source 1..18: mol_type = other DNA
caatcctgagtggttagg

SEQ ID NO: 520 - DNA, 18 bp, synthetic construct
  misc_feature 1..18: note = Synthetic polynucleotide
  source 1..18: mol_type = other DNA
aatcctgagtggttaggg

SEQ ID NO: 521 - DNA, 18 bp, synthetic construct
  misc_feature 1..18: note = Synthetic polynucleotide
  source 1..18: mol_type = other DNA
atcctgagtggttagggc

SEQ ID NO: 522 - DNA, 18 bp, synthetic construct
  misc_feature 1..18: note = Synthetic polynucleotide
  source 1..18: mol_type = other DNA
tcctgagtggttagggct

SEQ ID NO: 523 - DNA, 18 bp, synthetic construct
  misc_feature 1..18: note = Synthetic polynucleotide
  source 1..18: mol_type = other DNA
cctgagtggttagggctg

SEQ ID NO: 524 - DNA, 18 bp, synthetic construct
  misc_feature 1..18: note = Synthetic polynucleotide
  source 1..18: mol_type = other DNA
ctgagtggttagggctgg

SEQ ID NO: 525 - DNA, 18 bp, synthetic construct
  misc_feature 1..18: note = Synthetic polynucleotide
  source 1..18: mol_type = other DNA
tgagtggttagggctgga

SEQ ID NO: 526 - DNA, 18 bp, synthetic construct
  misc_feature 1..18: note = Synthetic polynucleotide
  source 1..18: mol_type = other DNA
gagtggttagggctggaa

SEQ ID NO: 527 - DNA, 18 bp, synthetic construct
  misc_feature 1..18: note = Synthetic polynucleotide
  source 1..18: mol_type = other DNA
agtggttagggctggaat

SEQ ID NO: 528 - DNA, 18 bp, synthetic construct
  misc_feature 1..18: note = Synthetic polynucleotide
  source 1..18: mol_type = other DNA
gtggttagggctggaata

SEQ ID NO: 529 - DNA, 18 bp, synthetic construct
  misc_feature 1..18: note = Synthetic polynucleotide
  source 1..18: mol_type = other DNA
tggttagggctggaatag

SEQ ID NO: 530 - DNA, 18 bp, synthetic construct
  misc_feature 1..18: note = Synthetic polynucleotide
  source 1..18: mol_type = other DNA
ggttagggctggaataga

SEQ ID NO: 531 - DNA, 18 bp, synthetic construct
  misc_feature 1..18: note = Synthetic polynucleotide
  source 1..18: mol_type = other DNA
gttagggctggaatagaa

SEQ ID NO: 532 - DNA, 18 bp, synthetic construct
  misc_feature 1..18: note = Synthetic polynucleotide
  source 1..18: mol_type = other DNA
ttagggctggaatagaag

SEQ ID NO: 533 - DNA, 18 bp, synthetic construct
  misc_feature 1..18: note = Synthetic polynucleotide
  source 1..18: mol_type = other DNA
tagggctggaatagaagg

SEQ ID NO: 534 - DNA, 18 bp, synthetic construct
  misc_feature 1..18: note = Synthetic polynucleotide
  source 1..18: mol_type = other DNA
agggctggaatagaagga

SEQ ID NO: 535 - DNA, 18 bp, synthetic construct
  misc_feature 1..18: note = Synthetic polynucleotide
  source 1..18: mol_type = other DNA
gggctggaatagaaggaa

SEQ ID NO: 536 - DNA, 18 bp, synthetic construct
  misc_feature 1..18: note = Synthetic polynucleotide
  source 1..18: mol_type = other DNA
ggctggaatagaaggaag

SEQ ID NO: 537 - DNA, 18 bp, synthetic construct
  misc_feature 1..18: note = Synthetic polynucleotide
  source 1..18: mol_type = other DNA
gctggaatagaaggaaga

SEQ ID NO: 538 - DNA, 18 bp, synthetic construct
  misc_feature 1..18: note = Synthetic polynucleotide
  source 1..18: mol_type = other DNA
ctggaatagaaggaagaa

SEQ ID NO: 539 - DNA, 18 bp, synthetic construct
  misc_feature 1..18: note = Synthetic polynucleotide
  source 1..18: mol_type = other DNA
tggaatagaaggaagaac

SEQ ID NO: 540 - DNA, 18 bp, synthetic construct
  misc_feature 1..18: note = Synthetic polynucleotide
  source 1..18: mol_type = other DNA
ggaatagaaggaagaacc

SEQ ID NO: 541 - DNA, 18 bp, synthetic construct
  misc_feature 1..18: note = Synthetic polynucleotide
  source 1..18: mol_type = other DNA
gaatagaaggaagaacct

SEQ ID NO: 542 - DNA, 18 bp, synthetic construct
  misc_feature 1..18: note = Synthetic polynucleotide
  source 1..18: mol_type = other DNA
aatagaaggaagaacctg

SEQ ID NO: 543 - DNA, 18 bp, synthetic construct
  misc_feature 1..18: note = Synthetic polynucleotide
  source 1..18: mol_type = other DNA
atagaaggaagaacctga

SEQ ID NO: 544 - DNA, 18 bp, synthetic construct
  misc_feature 1..18: note = Synthetic polynucleotide
  source 1..18: mol_type = other DNA
ctatccatccatgtactc

SEQ ID NO: 545 - DNA, 18 bp, synthetic construct
  misc_feature 1..18: note = Synthetic polynucleotide
  source 1..18: mol_type = other DNA
tatccatccatgtactca

SEQ ID NO: 546 - DNA, 18 bp, synthetic construct
  misc_feature 1..18: note = Synthetic polynucleotide
  source 1..18: mol_type = other DNA
atccatccatgtactcac

SEQ ID NO: 547 - DNA, 18 bp, synthetic construct
  misc_feature 1..18: note = Synthetic polynucleotide
  source 1..18: mol_type = other DNA
tccatccatgtactcacc

SEQ ID NO: 548 - DNA, 18 bp, synthetic construct
  misc_feature 1..18: note = Synthetic polynucleotide
  source 1..18: mol_type = other DNA
ccatccatgtactcaccc

SEQ ID NO: 549 - DNA, 18 bp, synthetic construct
  misc_feature 1..18: note = Synthetic polynucleotide
  source 1..18: mol_type = other DNA
catccatgtactcaccca

SEQ ID NO: 550 - DNA, 18 bp, synthetic construct
  misc_feature 1..18: note = Synthetic polynucleotide
  source 1..18: mol_type = other DNA
atccatgtactcacccat

SEQ ID NO: 551 - DNA, 18 bp, synthetic construct
  misc_feature 1..18: note = Synthetic polynucleotide
  source 1..18: mol_type = other DNA
tccatgtactcacccatc

SEQ ID NO: 552 - DNA, 17 bp, synthetic construct
  misc_feature 1..17: note = Synthetic polynucleotide
  source 1..17: mol_type = other DNA
ggtttgttcagtgtgga

SEQ ID NO: 553 - DNA, 17 bp, synthetic construct
  misc_feature 1..17: note = Synthetic polynucleotide
  source 1..17: mol_type = other DNA
tccacactgaacaaacc

SEQ ID NO: 554 - DNA, 216 bp, synthetic construct
  misc_feature 1..216: note = Target region 1
  source 1..216: mol_type = other DNA
gttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggata

SEQ ID NO: 555 - DNA, 203 bp, synthetic construct
  misc_feature 1..203: note = Target region 2
  source 1..203: mol_type = other DNA
ttccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggata

SEQ ID NO: 556 - DNA, 108 bp, synthetic construct
  misc_feature 1..108: note = Target region 3
  source 1..108: mol_type = other DNA
ctagtttgttgaataaatgctggtgaatgaatgaatgattgaacagatgaatgagtgatgagtagataaaaggatggatggagagatgggtgagtacatggatggata

SEQ ID NO: 557 - DNA, 32 bp, synthetic construct
  misc_feature 1..32: note = Target region 4
  source 1..32: mol_type = other DNA
gatggagagatgggtgagtacatggatggata

SEQ ID NO: 558 - DNA, 33 bp, synthetic construct
  misc_feature 1..33: note = Target region 5
  source 1..33: mol_type = other DNA
tgaatgaatgaatgattgaacagatgaatgagt

SEQ ID NO: 559 - DNA, 21 bp, synthetic construct
  misc_feature 1..21: note = TARDBP Primer 1
  source 1..21: mol_type = other DNA
cagctcatcctcagtcatgtc

SEQ ID NO: 560 - DNA, 21 bp, synthetic construct
  misc_feature 1..21: note = TARDBP Primer 2
  source 1..21: mol_type = other DNA
gatggtgtgactgcaaacttc

SEQ ID NO: 561 - DNA, 23 bp, synthetic construct
  misc_feature 1..23: note = TARDBP Probe
  source 1..23: mol_type = other DNA
cagcgccccacaaacacttttct

SEQ ID NO: 562 - DNA, 20 bp, synthetic construct
  misc_feature 1..20: note = UNC13A Primer 1
  source 1..20: mol_type = other DNA
gatcaaaggcgaggagaagg

SEQ ID NO: 563 - DNA, 19 bp, synthetic construct
  misc_feature 1..19: note = UNC13A Primer 2
  source 1..19: mol_type = other DNA
tggcatctgggatcttcac

SEQ ID NO: 564 - DNA, 30 bp, synthetic construct
  misc_feature 1..30: note = UNC13A Probe
  misc_feature 9..10: note = ZEN Quencher between nucleotides 9 and 10
  source 1..30: mol_type = other DNA
acctgtctgcatgagaacctgttccacttc